(19) United States
(12) Patent Application Publication
(10) Pub. No.: US 2021/0095251 A1
(43) Pub. Date: Apr. 1, 2021

(54) HUMAN ADIPOSE TISSUE PROGENITORS FOR AUTOLOGOUS CELL THERAPY FOR LIPODYSTROPHY

(71) Applicant: University of Massachusetts, Boston, MA (US)

(72) Inventor: Silvia Corvera, Westborough, MA (US)

(21) Appl. No.: 16/634,377

(22) PCT Filed: Aug. 10, 2018
(86) PCT No.: PCT/US2018/046266

§ 371 Date: Jan. 27, 2020

Related U.S. Application Data
(60) Provisional application No. 62/543,597, filed on Aug. 10, 2017.

(51) Int. Cl. C12N 5/077; A61K 35/35

(52) U.S. Cl. CPC C12N 5/0653; A61K 35/35

(57) ABSTRACT

Methods for preparation of human adipose stem cells, enriched populations thereof, and white adipose cells derived therefrom, for treatment of lipodystrophy.

### CLAIM OF PRIORITY

This application claims the benefit of U.S. Application No. 62/543,597, filed on Aug. 10, 2017. The entire contents of the foregoing are hereby incorporated by reference.

### FEDERALLY SPONSORED RESEARCH OR DEVELOPMENT

This invention was made with Government support under Grant No. DK089101 awarded by the National Institutes of Health. The Government has certain rights in the invention.

### TECHNICAL FIELD

This invention relates to methods for treatment of lipodystrophy using preparations of human progenitor cells capable of giving rise to adipose cells, and enriched populations thereof.

### BACKGROUND

Autologous cell therapies consist in the extraction of an individual's own cells, their modification outside the body, be it genetic or chemical, their expansion into large numbers of cells, and their re-introduction into the individual for therapeutic purposes. The best-known form of autologous cell therapy is CAR-T therapy, in which an individual's immune cells are extracted, genetically modified to enable their attack on cancer cells, and re-introduced into the patient. This therapy has recently been recommended for FDA approval. Autologous cell therapy, however, is not limited to immune cells, but can also be considered for regenerating and repairing tissue, using tissue progenitor cells. A unique advantage of autologous cell therapy is that is not plagued with the problems of transplant rejection and graft-versus-host disease, and therefore toxic immunosuppressive therapy is avoided.

### SUMMARY

Lipodystrophy is a disease in which individuals entirely lack or have insufficient adipose tissue under the skin (subcutaneous adipose tissue) (Robbins and Savage 2015, Hussain and Garg 2016). It can range in severity and age of onset, from children lacking visible fat (Congenital Generalized Lipodystrophy) to young adults that lose fat in their arms and legs over a period of years (Familial Partial Lipodystrophy). Individuals with lipodystrophy develop very severe metabolic disease, characterized by hyperlipidemia, type-2 diabetes, hyperinsulinemia, hetpatosteatosis and atherosclerosis. Patients with lipodystrophy succumb to metabolic disease in childhood or early adulthood. The use of human progenitor cells as autologous therapeutics for human lipodystrophy, without or with the use of genetic manipulation to induce the formation of adipose cells in-vitro, is described herein.

Thus, provided herein are methods for providing a population of adipose progenitor cells for treating a subject with lipodystrophy associated with a genetic mutation. The methods include:

obtaining primary adipose tissue from a subject who has a lipodystrophy associated with a genetic mutation comprising a first population of adipose cells, and
(i) culturing the primary adipose tissue in a protein-gel matrix in the presence of pro-angiogenic factors to induce the growth of a second population of cells;
(ii) isolating cells from the second population to form a third population of cells comprising an enriched population of adipose progenitor cells; and

maintaining the population of adipose progenitor cells under conditions and for a time sufficient for proliferation of the cells,

thereby providing a population of adipose progenitor cells for treating a subject with lipodystrophy associated with a genetic mutation.

In some embodiments, the second population of cells is subjected to enzyme digestion, e.g., with dispase, to produce the third population of cells.

In some embodiments, the methods further include mixing the genetically modified adipose progenitor cells with an injectable hydrogel.

In some embodiments, isolating cells from the second population comprises isolating CD45−CD29+CD34+CD24+CD144− cells from the second population.

Also provided herein are isolated, enriched populations of adipose progenitor cells made by a method described herein.

In some embodiments, the isolated, enriched population of adipose progenitor cells is in an injectable hydrogel.

In some embodiments, the methods include genetically modifying the second population of cells or the third population of cells to correct the genetic mutation, to provide a population of genetically modified adipose progenitor cells.

Also provided herein are isolated, enriched populations of genetically modified adipose progenitor cells produced by a method described herein, e.g., in an injectable hydrogel.

In some embodiments, the methods include maintaining the adipose progenitor cells in culture for a time and under conditions sufficient for the cells to differentiate into white adipose cells, e.g., cells that express ADIPOQ, PLIN1, and/or LEP.

In some embodiments, the adipose progenitor cells are maintained in an injectable hydrogel.

Also provided herein are isolated, enriched population of white adipose cells made by a method described herein.

Further, provided herein are methods for treating subjects who have lipodystrophy, e.g., CGL1 or CGL2. The methods include administering to the subject an isolated, enriched population of adipose progenitor cells; an isolated, enriched population of genetically modified adipose progenitor cells; and/or an isolated, enriched population of white adipose cells as described herein, e.g., made by a method described herein. In some embodiments, the cells are made from a first population of cells (e.g., adipose tissue) obtained from the subject to be treated.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### Sequence Listing

The instant application contains a Sequence Listing which has been submitted electronically in ASCII format and is hereby incorporated by reference in its entirety. Said ASCII copy, created on Aug. 9, 2017, is named 07917_0403WO1_SL.txt and is 453,025 bytes in size.

### DESCRIPTION OF DRAWINGS

FIG. 1: Image of a human explant cultured in MatriGel in the proprietary medium EGM2-MV (Lonza). Progenitor cells proliferate along with nascent capillary sprouts.

FIGS. 2A-C: A. Images of progenitor cells differentiating into adipocytes (HACAPS) after exposure to an adipogenic cocktail composed of methylisobutil-xanthine, dexamethasone and insulin (MDI) for 7-14 weeks. B. RT-PCR analysis of cDNA obtained from HACAPS before (MDI −) and after adipogenic differentiation (MDI+) for 2 weeks. Note log scale on x-axis for induction of the adipocyte-specific genes perilipin-1 (PLIN1), adiponectin (ADIPOQ), insulin responsive glucose transporter (Glut4) and Fatty Acid Binding Protein-4 (FABP4). C. HACAPS are metabolically active after 14 weeks in culture, as assessed by their staining with the micochondrial marker MitoTracker Red.

FIGS. 3A-F: Formation of functional adipose tissue from HACAPS. A. Phase image of cells for implantation. B. Suspension of cells in Matrigel. C. Dorsal area of mouse injected with Matrigel alone. D. Remnants of the hydrogel after 6 weeks. E. Dorsal area of mouse injected with HACAPS. F. Excised adipose tissue formed from HACAPS displaying vascularization (arrows).

FIGS. 4A-B: Adipose tissue formed from HACAPS is functional and metabolically active. A. Levels of human adiponectin in the mouse circulation was proportional to the size of the adipose tissue formed from HACAPS, demonstrating functional integration. B. Whole body glucose turnover was increased as a function of human adiponectin in the mouse circulation, which reflects that adipose tissue formed from HACAPS is metabolically active.

FIG. 5: The functional properties of HACAPS assessed by secretion of ADIPOQ into the media. Variation in the cell concentration, as well as in stoichiometric ratio of azide- and DBCO-terminated building blocks results in ideal hydrogel formulation for adipose tissue formation. In a first experiment, the number of cells was varied from 10,000 to 1,000,000 to determine the linear range of ADIPOQ production on a network of [amide-DBCO]:[CH2-N3]=1:1. Then, a fixed number (250,000) of cells were assayed in gel stiffness ranges of 2.5% and 5%, with three ratios of [amide-DBCO]: [CH2-N3] (0.6:1.0, 1:1, and 1:0.6). There is a clear superiority in the 0.6:1 ratio, with less variance between 2.5 and 5% gels.

FIG. 6: An exemplary method for production of human adipocytes from tissue-derived progenitors.

### DETAILED DESCRIPTION

During embryonic development, the formation of new adipocytes and the formation of the adipose tissue microvasculature are highly inter-dependent. Indeed, during embryonic development, the formation of the adipose tissue vascular network precedes the emergence of adipocytes (Han et al. Development. 2011; 138(22):5027-37). Also, in adult mice, expansion of adipose tissue is accompanied by formation of angiogenic foci (Nishimura et al. Diabetes. 2007; 56(6):1517-26). Another finding that supports a close relationship between vascular and pre-adipocyte development is the discovery that, in mice, adipose tissue capillary walls are a niche for adipocyte progenitors (Tang et al. Science. 2008; 322(5901):583-6; Tang et al., Cell Metabolism. 2011; 14(1):116-22). New adipocytes form from cells tightly embedded in the walls of newly formed human adipose tissue capillaries (Tran et al., Cell Metabolism. 2012; 15(2):222-9). These newly emerging adipocytes contain key adipocyte-specific markers, yet are connected to endothelial cells through tight junctions, supporting the concept that they are tightly associated with the capillary wall (Tran et al., 2012). Lineage-tracing studies using reporters driven by the VE-cadherin promoter clearly show that at least some adipocytes are derived from progenitors that at some point during development expressed VE-Cadherin (CD144) (Tran et al., 2012). Thus, in adult organisms the development of the adipose tissue microvasculature and the formation of new adipocytes may be interdependent.

Numerous studies report the presence of pluripotent stem cell populations in human adipose tissue, which hold great potential for clinical applications (see review by Baer and Geiger, Stem Cells Int. 2012; 2012:812693). For example, one of the therapeutic uses of adipose tissue is in reconstructive surgery, where adipose tissue is grafted for the purpose of contour filling or replacement of tissue following excision of tumors. However, this grafted tissue consists mostly of mature adipocytes, which are eventually reabsorbed. In an attempt to improve adipose tissue grafting, Yoshimura et al (Yoshimura et al., Breast J. 2010; 16(2):169-75; Yoshimura et al., Dermatol Surg. 2008; 34(9):1178-85; Yoshimura et al., Regen Med. 2009; 4(2):265-73) supplemented adipose tissue with cells derived from the SVF. The positive results obtained from this procedure, including longer graft duration, are attributed to the presence of precursor stem cells within the SVF. Production of true stem cells capable of regenerating adipocytes and their vasculature would greatly enhance the effectiveness of adipose tissue use for reconstructive purposes.

In addition, adipose tissue precursors may also be critical determinants of adipose tissue expandability, and their numbers may influence metabolic disease risk. In most published studies related to adipose tissue stem cells, cells were selected on the basis of their plating properties and growth in-vitro, and comprise a mixed population with no specific prospective molecular identity.

Adipose tissue is comprised of the parenchymal cells (adipocytes) and their stromal vascular support (extracellular matrix, vasculature). Adipocytes can be classified into “white” “brown” or “brite/beige” depending on their functional role. White adipocytes primarily store excess energy in the form of triglycerides, stored in a single large droplet within their cytoplasm. Mature adipocytes are made from stem-like cells called fat cell progenitors [1]. These progenitors are much smaller than mature fat cells, and can divide, forming more fat cell progenitors. As the needs of the body to store fat increase, some of the progenitors convert to mature fat cells; they start by increasing their uptake of lipids, making small lipid droplets, growing and forming one large lipid droplet, to become a mature fat cell. Mature fat cells can last many years once they are formed; it has been calculated that mature fat cells can live up to 10 years in human adults [2]. In addition to storing fat, mature fat cells also produce hormones, called “adipokines” that control many critical aspects of metabolism.

Lipodystrophy

Lipodystrophy is a disease in which individuals entirely lack or have insufficient adipose tissue under the skin (subcutaneous adipose tissue) (Robbins and Savage 2015, Hussain and Garg 2016). It can range in severity and age of onset, from children lacking visible fat (Congenital Generalized Lipodystrophy, CGL) to young adults that lose fat in their arms and legs over a period of years (Familial Partial Lipodystrophy, FPLD). Due to their inability to store fat, individuals with lipodystrophy develop very severe metabolic disease, characterized by hyperlipidemia, type-2 diabetes, hyperinsulinemia, hepatosteatosis and atherosclerosis. Patients with lipodystrophy also suffer from lack of the essential adipokines made by fat cells, which causes extreme hunger, compounding the deleterious effects of impaired fat storage. Patients with lipodistrophy are sometimes treated with the administration of leptin, an adipokine that decreases appetite in these patients and lessens the amount of food ingested [3]. This treatment lessens the symptoms but does not provide a cure or a mitigation of the metabolic disease and its consequences. There is currently no cure for lipodystrophy, and affected patients typically succumb to metabolic disease in childhood or early adulthood.

The failure to form mature fat cells in patients with lipodystrophy results from mutations in genes that are necessary for lipid uptake, or for triglyceride synthesis, or for lipid droplet formation (Robbins and Savage 2015, Hussain and Garg 2016). For example, in the most common case of Congenital Generalized Lipodystrophy, patients have mutations in the gene AGPAT2, which is necessary for the synthesis of triglycerides. In these patients, the progenitors are made, can divide, but cannot make a large lipid droplet because they can't synthesize triglycerides. In another case, patients have mutations in the gene BSCL2, which is required to form lipid droplets. Known gene mutations that cause severe lipodistrophy are present in about 1:500,000 individuals, but in many other cases the genes responsible for the disease have not been identified. It is also thought that Familial Partial Lipodystrophy is not diagnosed properly [4]. Therefore the frequency of this disease is likely to be much higher than reported.

Mouse models of lipodystrophy have been generated in which the genes responsible for human lipodystrophy have been deleted (Savage 2009, Gao, Wang et al. 2015). These mice display severe metabolic abnormalities that phenocopy human lipodystrophy. Importantly, implantation of a small amount of fat greatly reduces all metabolic consequences of the gene mutation [5]. This is probably due to lessened ectopic fat accumulation, as well as increased production of adipokines and their effects on whole body metabolism.

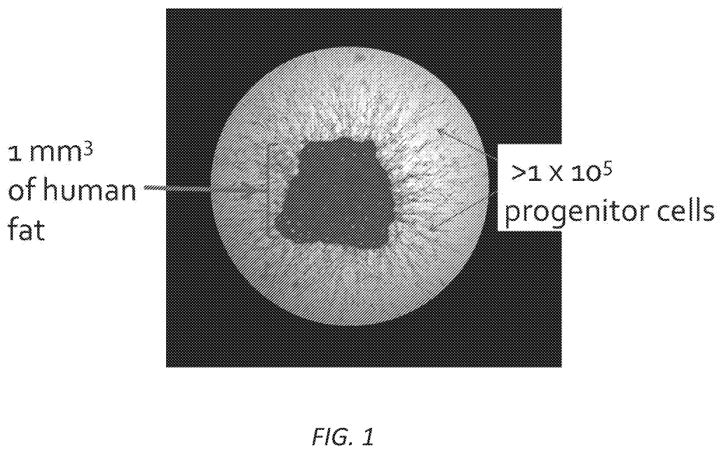

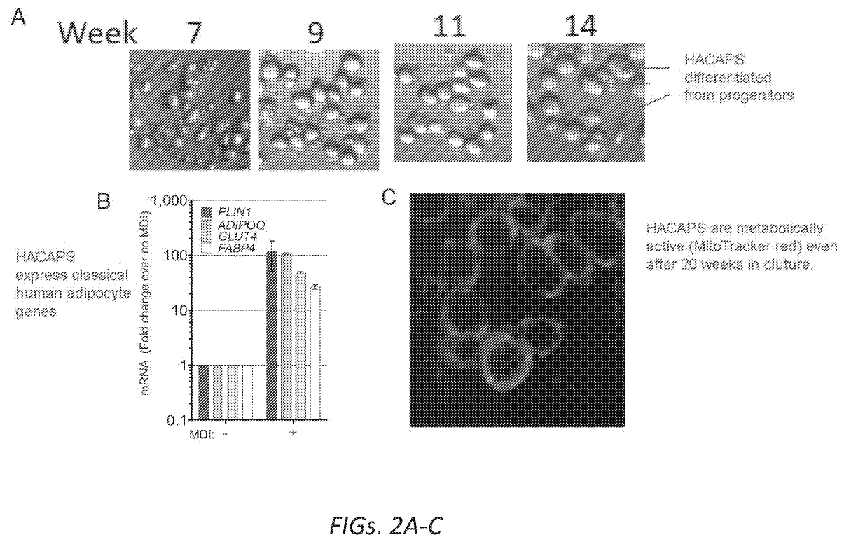

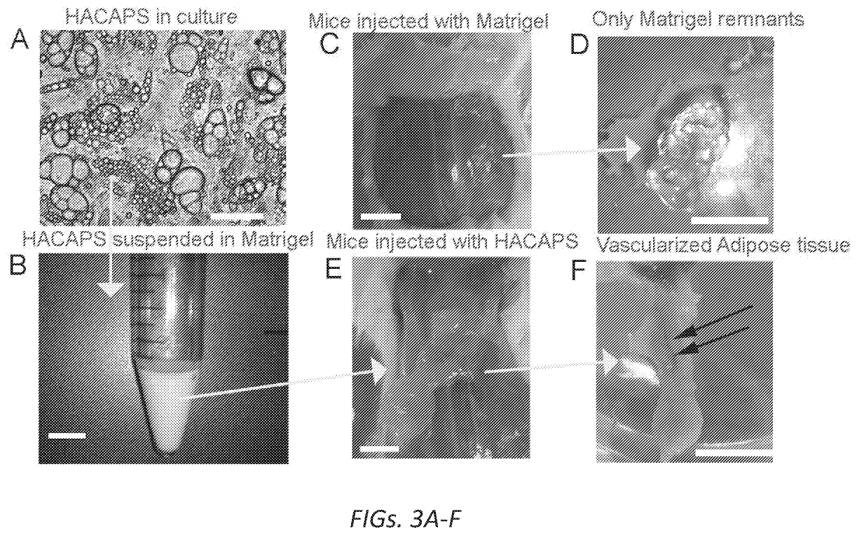

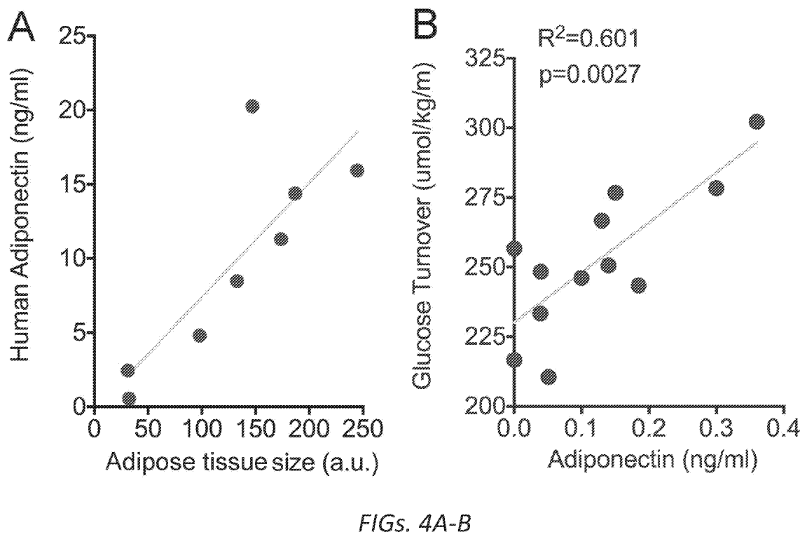

Described herein are methods that can be used to obtain human adipocyte progenitors in large numbers from subjects with lipodystrophy, genetically modify them to correct the genetic alteration, and to induce their development into adipocytes in vivo or in vitro (see Example 1, FIGS. 1, 2A-C). When these adipocytes are implanted into a subject, they continue along the path of differentiation to form mature fat cells, and in the process become vascularized by the host's blood vessels to form a new fat pad (see Example 1, FIGS. 3A-F). These fat pads, formed from human progenitors, are metabolically active, produce human adipokines (see Example 1, FIG. 4A), and improve glucose metabolism (see Example 1, FIG. 4B). These methods can be used to treat patients with lipodystrophy, by obtaining their progenitor cells, optionally correcting the underlying genetic defect in vitro, growing the repaired progenitors cells in large numbers, initiating their differentiation into adipocytes, and implanting them back into the patient, where they will continue to form healthy adipose tissue and mitigate metabolic abnormalities.

Genetic Mutations Associated with Lipodystrophy

Although not all of the described forms of lipodystrophy have identified genetic mutations, many do. In the vast majority of cases Congenital Generalized Lipodystrophy (CGL) is caused by biallelic mutations in either the gene encoding 1-acylglycerol-3-phosphate O-acyltransferase 2 (AGPAT2) [6] or the gene encoding seipin (BSCL2) (Magre et al., 2001), an endoplasmic reticulum protein involved in lipid droplet formation. In Familial Partial Lipodystrophy (FPLD), which is more common than CGL, adipose tissue pathology is caused by mutations in one of several genes (See Table 1).

TABLE 1
Genes Associated with Lipodystrophy

| Lipodystrophy | | RefSeq Gene | | RefSeq | |
|---|---|---|---|---|---|
| type | Gene | ID | # | ID | Disease-causing Mutations |
| CGL1 | AGPAT2 | NG_008090.1, | 1 | NM_006412.3 | NP_006403.2:p.Leu107AlafsTer279 |
| | | Range 5001-19317 | | | NM_006412.3(AGPAT2):c.755_763delTGAGGACCA |
| | | | | | (p.Met252_Thr254del) |
| | | | | | NM_006412.3(AGPAT2):c.713C > G (p.Ala238Gly) |
| | | | | | NM_006412.3(AGPAT2):c.676C > T (p.Gln226Ter) |
| | | | | | NM_006412.3(AGPAT2):c.662 − 2A > C |
| | | | | | NM_006412.3(AGPAT2):c.661 + 2T > G |
| | | | | | NM_006412.3(AGPAT2):c.643A > T (p.Lys215Ter) |
| | | | | | NM_006412.3(AGPAT2):c.589 − 2A > G |
| | | | | | NM_001012727.1(AGPAT2):c.366_492 + 910del1037 |
| | | | | | NM_006412.3(AGPAT2):c.570C > A (p.Tyr190Ter) |
| | | | | | NM_006412.3(AGPAT2):c.538delG |
| | | | | | (p.Asp180ThrfsTer73) |
| | | | | | NM_006412.3(AGPAT2):c.514G > A (p.Glu172Lys) |
| | | | | | NM_006412.3(AGPAT2):c.503G > A (p.Trp168Ter) |
| | | | | | NM_006412.3(AGPAT2):c.493 − 1G > C |
| | | | | | NM_006412.3(AGPAT2):c.492 + 1G > A |
| | | | | | NM_006412.3(AGPAT2):c.418_420delTTC |
| | | | | | (p.Phe140del) |
| | | | | | NM_006412.3(AGPAT2):c.406G > A (p.Gly136Arg) |
| | | | | | NM_006412.3(AGPAT2):c.377dupT (p.Pro128Alafs) |
| | | | | | NM_006412.3(AGPAT2):c.299G > A (p.Ser100Asn) |
| | | | | | NM_006412.3(AGPAT2):c.282delC (p.Ile94Metfs) |
| | | | | | NM_006412.3(AGPAT2):c.202C > T (p.Arg68Ter) |
| | | | | | NM_006412.3(AGPAT2):c.194G > A (p.Trp65Ter) |
| | | | | | NM_006412.3(AGPAT2):c.183 − 2A > G |
| | | | | | NM_006412.3(AGPAT2):c.182 + 1G > A |
| CGL2 | BSCL2 | NG_008461.1, | 2 | NM_001122955.3 | BSCL2, IVS4, G − A, +1 |
| | | Range 6868-24313 | | | BSCL2, 258-BP DEL/12-BP INS |
| | | | | | NM_032667.6(BSCL2):c.1125C > A (p.Va1375=) |
| | | | | | NM_032667.6(BSCL2):c.823C > T (p.Arg275Ter) |
| | | | | | NM_032667.6(BSCL2):c.814 − 2A > G |
| | | | | | NM_032667.6(BSCL2):c.793C > T (p.Arg265Ter) |
| | | | | | NM_032667.6(BSCL2):c.782dupG (p.IIe262Hisfs) |
| | | | | | NM_032667.6(BSCL2):c.672 − 2A > G |
| | | | | | NM_032667.6(BSCL2):c.672 − 2A > C |
| | | | | | NM_032667.6(BSCL2):c.672 − 3C > G |
| | | | | | NM_032667.6(BSCL2):c.671 + 5G > A |
| | | | | | NM_032667.6(BSCL2):c.652_662delGCGCACTTCAC |
| | | | | | (p.Ala218Trpfs) |
| | | | | | NM_032667.6(BSCL2):c.636delC (p.Tyr213Thrfs) |
| | | | | | NM_032667.6(BSCL2):c.634G > C (p.Ala212Pro) |
| | | | | | NM_032667.6(BSCL2):c.574 − 2A > G |
| | | | | | NM_032667.6(BSCL2):c.565G > T (p.Glu189Ter) |
| | | | | | NM_032667.6(BSCL2):c.412C > T (p.Arg138Ter) |
| | | | | | NM_001122955.3(BSCL2):c.538G > T (p.Glu180Ter) |
| | | | | | NM_032667.6(BSCL2):c.325dupA (p.Thr109Asnfs) |
| | | | | | NM_032667.6(BSCL2):c.317_321delATCGT |
| | | | | | (p.Tyr106Cysfs) |
| | | | | | NM_032667.6(BSCL2):c.315_316delGT |
| | | | | | (p.Tyr106Serfs) |
| | | | | | NM_032667.6(BSCL2):c.301_302insAA |
| | | | | | (p.Met101Lysfs) |
| | | | | | NM_032667.6(BSCL2):c.269C > T (p.Ser90Leu) |
| | | | | | NM_032667.6(BSCL2):c.263A > G (p.Asn88Ser) |
| | | | | | NM_032667.6(BSCL2):c.193delCinsGGA |
| | | | | | (p.Pro65Glyfs) |
| | | | | | NM_032667.6(BSCL2):c.192_193delCCinsGGA |
| | | | | | (p.Ser64Argfs) |
| | | | | | NM_032667.6(BSCL2):c.154_155dupTT |
| | | | | | (p.Tyr53Serfs) |
| | | | | | NM_032667.6(BSCL2):c.142C > T (p.Leu48Phe) |
| | | | | | GRCh37/hg19 11q12.3(chr11:61840997-62987330) × 1 |
| | | | | | GRCh38/hg38 11q12.3(chr11:62249520-62946093) × 3 |
| | | | | | GRCh38/hg38 11q12.3(chr11:62452571-62862781) × 3 |
| | | | | | GRCh38/hg38 11q12.3(chr11:62433886-63096003) × 3 |
| CGL3 | CAV1 | NG_012051.1, | 9 | NM_001172895.1 | c.112G > T (p.Glu38Ter) |
| | | Range 5001-41401 | | | |
| CGL4 | PTRF/CAVIN1 | NG_015845.1, | 8 | NM_012232.5 | IVS1DS, G − T, +1 |
| | | Range 5001-25872 | | | PTRF, 4-BP DEL, 518AAGA |
| | | | | | PTRF, 4-BP INS, 481GTGA |
| | | | | | PTRF, 1-BP DEL, 135G |
| | | | | | PTRF, 1-BP DUP, 362T |
| | | | | | PTRF, 1-BP DEL, 160G |
| | | | | | PTRF, 1-BP DEL, 525G |
| FPLD1 or | Unknown | ? | ? | | ? |
| Kobberling-type | | | | | |
| lipodystrophy | | | | | |
| FPLD2 or | LMNA | NG_008692.2, | 3 | NM_170707.3 | 3-BP DEL, 94AAG |
| Dunnigan type | | Range 4974-62517 | | | |
| lipodystrophy | | | | | |
| FPLD3 | PPARG | NG_011749.1, | 4 | NM_138711.3 | 3-BP DEL/1-BP INS, NT553 |
| | | Range 5001-151507 | | | |
| FPLD4 | PLIN1 | NG_029172.1, | 5 | NM_002666.4 | 2-BP DEL, 1191AG |
| | | Range 5001-20051 | | | |
| FPLD5 | CIDEC | NG_042291.1, | 6 | NM_022094.3 | c.556G > T (p.Glu186Ter) |
| | | Range 5001-18545 | | | |
| FPLD6 | LIPe | NG_034246.1, | 7 | NM_005357.3 | p.Ala507fsTer563 |
| | | Range 5001-30920 | | | |

### #, SEQ ID NO:

Subjects who have or are suspected of having lipodystrophy can be identified using methods known in the art. For example, subjects with CLG typically present with a near total absence of adipose tissue from birth or early infancy; severe insulin resistance; hypertriglyceridemia; hepatic steatosis; and early onset diabetes. Subjects with FPLD have abnormal subcutaneous adipose tissue distribution, typically beginning in late childhood or early adulthood with a slow loss of adipose from the upper and lower extremities, as well as the gluteal and trunk regions. See, e.g., Garg, “Acquired and inherited lipodystrophies.” New Eng. J. Med. 350: 1220-1234, 2004; Nolis et al., J Hum Genet. 2014 January; 59(1):16-23. The presence of a genetic mutation associated with lipodystrophy can be identified using methods known in the art, e.g., that can include amplifying and/or sequencing all or part of one or more of the genes in Table 1 that are known to be associated with lipodystrophy and identifying sequence that differs from wild type (e.g., a difference in the genomic sequence that alters expression or function of the encoded protein), or identifying the presence of known disease-causing mutations. A number of methods including commercially available assays can be used to detect mutations in these genes.

FPLD is associated with reduction of lower body subcutaneous adipose tissue, while truck and neck adipose tissue is preserved or even increased. Thus, the use of autologous adipose progenitors to treat lipodystrophy can include the repair of the genetic mutation that results in impaired differentiation into mature adipocytes in CGL, but may only require the expansion of adipocyte progenitors and their engraftment into lower body depots in the case of FPLD without genetically engineering the progenitors before implantation.

Studies in mouse models of CGL demonstrate that the mutation does not impair progenitor cell development, but impairs progression into a mature adipocyte. In the case of AGPAT2 and Seipin, the approach to repair the genetic defect can include the use of vectors that can introduce a functional copy of the defective gene into adipocyte progenitors, allowing their differentiation into mature adipocytes after proliferation in-vitro, as has been achieved in induced pluripotent stem cells derived from patients with lipodystrophy (Mori, Fujikura et al. 2016). Selective targeting of the normal gene into progenitors can be achieved, e.g., using viral vectors, preferably Adeno Associated Virus capsids, which have highly selective targeting features. In a complementary approach, lentiviral vectors can be used to transduce the cells, e.g., all progenitors obtained from patients with CGL.

Adipose Progenitor Cells and Adipocytes for Transplantation

As shown in US 2015/0259647, which is incorporated herein in its entirety, subcutaneous adipose tissue fragments embedded in Matrigel and incubated in the presence of angiogenic growth factors produce capillary networks that contain adipocyte progenitors. These adipocyte progenitors can be genetically modified, expanded and differentiated for use in treating subjects with lipodystrophy.

The methods include obtaining primary adipose tissue from a subject, preferably a human subject with lipodystrophy to be treated using the methods described herein, and generating an enriched population of adipocyte progenitor cells. The primary adipose tissue can be obtained using methods known in the art, e.g., by needle biopsy, surgical harvesting or lipoaspiration.

The tissues can be dissociated into single cells, using known methods, such as mechanical disruption, trituration, or enzymatic digestion or explanting. If enzymatic digestion is used, enzymes such as collagenase, hyaluronidase, dispase, pronase, trypsin, elastase and chymotrypsin can be used. Any method can be used so long as the cells obtained are viable. A heterogeneous mixture of primary cells is typically obtained from the tissue, likely including mature adipose cells, stem cells, and other cell types present in the adipose tissues, e.g., endothelial or epithelial cells.

The cells or tissue are then cultured in EGM2-MV (Lonza) or other media, e.g., a formulation consisting of Media 199 supplemented with glucose (e.g., 10 mM), ascorbic acid (e.g., 500 mM), hydrocortisone (e.g., 1 uM) and human recombinant FGF-2 (e.g., 0.1 nM) or other angiogenic growth factors. The cells that attach and proliferate are mixed, but are highly enriched for adipocyte progenitors.

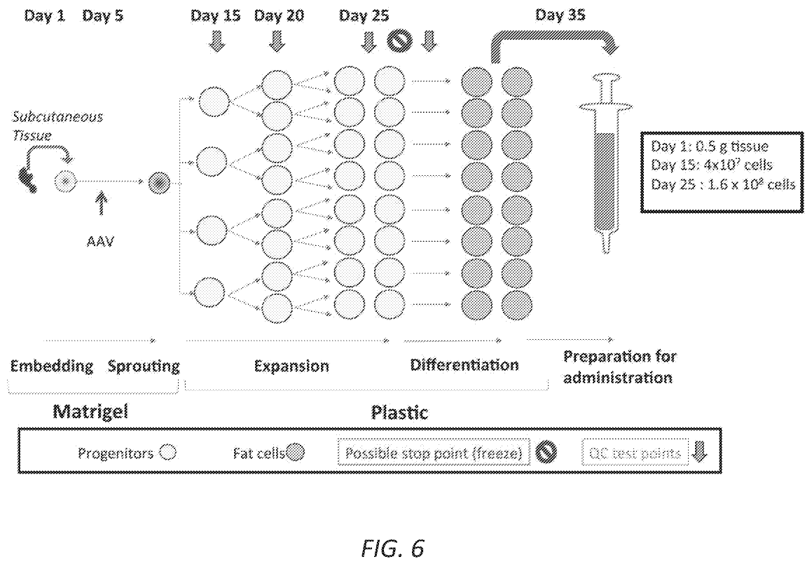

Alternatively, as shown in FIG. 6, and preferably when obtained as solid pieces of tissue, the tissue (2-10 g, e.g., 3-7 g, preferably about 5 g) is then cut, e.g., into 1 mm pieces, which are embedded in a protein-gel matrix; one preferred example of a matrix is MatriGel (a gelatinous protein mixture secreted by Engelbreth-Holm-Swarm (EHS) mouse sarcoma cells, marketed by Corning Life Sciences and by Trevigen, Inc., under the name Cultrex BME, that includes a complex mixture of basement membrane proteins such as laminin, type IV collagen, entactin/nitrogen and proteoheparan sulfate, and also contains growth factors; see Hughes et al., Proteomics. 2010 May; 10(9):1886-90; Benton et al., J Cell Physiol. 2009 October; 221(1):18-25; Kleinman and Martin, Semin Cancer Biol. 2005 October; 15(5):378-86; and Baatout and Cheta, Rom J Intern Med. 1996 July-December; 34(3-4):263-9), though other extracellular matrix substitutes and bioscaffolds can also be used, e.g., the PathClear® Grade Basement Membrane Extract (AMSBIO); StemXVivo™ Culture Matrix (R&D Systems); or StemAdhere™—Defined Matrix (Stemcell Technologies). The tissue is then cultured in medium formulated for endothelial cell growth, e.g., supplemented with glucose (10 mM), hydrocortisone (3 uM), ascorbic acid (1 mM), and human FGF-2 (0.1 nM). In some embodiments, the medium is supplemented with pro-angiogenic factors. In some embodiments, the pro-angiogenic factors comprise FGF-2, and one or more of VEGF, IGF1 and EGF; for example, FGF-2 and VEGF; FGF-2 and IGF1, or FGF-2 and hEGF; or FGF-2, VEGF, and IGF; FGF-2, VEGF, and EGF; FGF-2, IGF1, and EGF; or all of FGF-2, VEGF, IGF1, and EGF are used. In some embodiments, e.g., wherein the cells used are human cells, human pro-angiogenic factors are also used. In some embodiments, the medium is EGM™2-MV, a medium formulated for endothelial cell growth (Lonza Biologics) or a formulation consisting of Media 199 supplemented with glucose (e.g., 10 mM), ascorbic acid (e.g., 500 mM), hydrocortisone (e.g., 1 uM) and human recombinant FGF-2 (e.g., 0.1 nM) or other pro-angiogenic growth factors.

The cultures are maintained for a time sufficient for capillary growth to occur; capillary growth is recognized by the formation of branched structures comprised of at least three connected cells. After the culture dishes exhibit a desired amount of capillary growth, e.g., the point at which capillary tip cells reach the edge of the culture dish, the obtained adipose progenitor cells can be harvested and genetically modified.

The present methods then include harvesting the cells after recovery from the protein-gel matrix, e.g., matrigel, in a single cell suspension for clonal expansion. The protein-gel matrix is degraded by proteolysis, e.g., using dispase for Matrigel. Other proteases that degrade fibronectin, such as Granzyme-B or MMP-9 could also potentially be used. Because some cells attach to the bottom of the plate, mechanical or (preferably) enzymatic treatment (e.g., with a protease such as trypsin, thermolysin, pepsin, or collagenase; in some embodiments, trypsin/EDTA (e.g., Trypsin-Versene™ (Lonza)), TrypLE™ (Life Technologies) or Detachin Cell Detachment Solution (Genlantis)) can be used for detaching cells that remain adherent to the well, e.g., after the matrix is degraded treatment. For example, the cultures can be incubated in dispase at 37° C. for 1.5-2 hours, and then a Trypsin-Versene/EDTA mixture is used to stop the matrix (dispase) digestion and dislodge adherent cells. The cell suspension obtained is then placed in medium, e.g., EGM™-2 MV supplemented EBM-2 medium, and the cells are concentrated, e.g., by centrifugation, e.g., at 2000 rpm for 10 minutes at room temperature. At this point the cell suspension is heterogeneous, but adipocyte progenitors represent at least 50% of the clonally expandable population, and express the cell surface marker CD73, which can be used to further purify the cells.

This mixture of cells can optionally be sorted or immunoadsorbed based on the expression of one or more cell surface markers, e.g., CD29 and/or CD73, optionally plus CD44 and CD90, to produce enriched populations of cells, e.g., an enriched population of cells that are CD45−CD29+CD34+CD24+CD144− (white fat progenitor cells). Methods for sorting cells are known in the art, and include flow cytometry, e.g., fluorescence activated cell sorting (FACS), using fluorescently labeled antibodies that recognize the cell surface markers. When fluorescence detection is used, the primary antibodies can be labeled, or can be detected using labeled secondary antibodies. Suitable antibodies are known in the art and commercially available, e.g., from BD Biosciences. Other flow cytometric cell sorting methods can also be used, e.g., photoacoustic (PA), photothermal (PT), fluorescent, and Raman methods (see, e.g., Glanzha and Zharov, Methods. 2012 July; 57(3):280-96); photon flow cytometry strategies and applications; see, e.g., Tkaczyk and Tkaczyk, Cytometry A. 2011 October; 79(10):775-88; and microfluidic impedance-based flow cytometry (see Cheung et al., Cytometry A. 2010 July; 77(7):648-66). As an alternative, other methods such as magnetic cell sorting (MACS) and microfluidic cell sorting methods can also be used; see, e.g., Autebert et al., Methods. 2012 July; 57(3):297-307; Zhao et al., Molecules. 2012 May 25; 17(6):6196-236; Smith et al., Semin Reprod Med. 2011 January; 29(1):5-14; Bhagat et al., Med Biol Eng Comput. 2010 October; 48(10):999-1014; and Bernstein and Hyun, Stem Cell Res Ther. 2012 May 10; 3(3):17.

In some embodiments, optionally after the cells are enriched, e.g., by cell sorting, the cells are plated and then maintained in culture to proliferate. In some embodiments, the progenitor cells are resuspended in media (the cells can optionally be genetically modified at this time) and further cultured and passaged, and can be subjected to differentiation and/or clonal expansion. In some embodiments, the cells are grown in vitro, differentiated into white adipocytes that express ADIPOQ, PLIN1, and/or LEP by incubation in an adipogenic cocktail composed of methylisobutil-xanthine, dexamethasone and insulin (MDI) (e.g., synthetic glucocorticoid dexamethasone, the cAMP elevating agent 1-methyl-3-isobutyl xanthine (MIX), and pharmacological doses of insulin; see Hwang et al., Annu Rev Cell Dev Biol 13: 231-259), and then used for implantation in the present methods.

In some embodiments, the expanded progenitor cells are used to seed biocompatible hydrogel scaffolds, which may be implanted directly, or after the seeded cells within the scaffold have been induced to differentiate in vitro into adipocytes by incubation with MDI. In preferred embodiment, the hydrogel is based on catalyst-free, strain-promoted azide-alkyne cycloaddition crosslinking of cytocompatible building blocks with biorthogonal reactive end-groups, a gelling kinetics suited for formulating injectables and unaffected by cellular cargos, and precisely tunable degradation rates for modulating metabolic activities of encapsulated cells, e.g., as described in Xu, Feng et al. 2014, and U.S. Pat. No. 9,388,276, which is incorporated herein by reference in its entirety.

About 0.5 g of adipose tissue can give rise to almost 200 million cells. Because 5 million cells can improve metabolism in a 35 g mouse, we would expect therapeutic effects from 5,000 million cells for a 35 Kg human. We can derive this number of cells from 10 g of human tissue, which is approximately 2 tablespoons in volume and can be obtained through biopsy.

Correction of Genetic Mutations Associated with Lipodystrophy

In some embodiments, the progenitor cells can be genetically engineered to correct a genetic mutation associated with lipodystrophy, using methods known in the art.

In some embodiments, the cells can be genetically engineered to stably or transiently express one or more exogenous genes, and/or to lack or underexpress one or more endogenous genes. For example, the cells can be transfected with an exogenous nucleic acid sequence which includes a nucleic acid sequence encoding a selected protein or peptide, and produce the encoded product stably and reproducibly in vitro and in vivo, over extended periods of time. A heterologous amino acid can also be a regulatory sequence, e.g., a promoter, which causes expression, e.g., inducible expression or upregulation, of an endogenous sequence. An exogenous nucleic acid sequence can be introduced into a primary or secondary cell by homologous recombination as described, for example, in U.S. Pat. No. 5,641,670, the contents of which are incorporated herein by reference. The transfected primary or secondary cells may also include DNA encoding a selectable marker which confers a selectable phenotype upon them, facilitating their identification and isolation.

For example, the cells can be transfected with an exogenous nucleic acid sequence that includes a nucleic acid sequence encoding a selected protein or peptide, e.g., a gene or cDNA encoding a functional sequence as shown in Table 1, such that the cells produce the encoded product stably and reproducibly in vitro and in vivo, over extended periods of time. The corrective nucleic acid should encode a functional sequence corresponding to the mutant in the subject, such that a subject who has CGL as a result of a mutation in AGPAT2 (i.e., has CGL1) receives cells that are engineered to express a functional AGPAT2 sequence, a subject who has CGL as a result of a mutation in BSCL2 (i.e., has CGL2) receives cells that are engineered to express a functional BSCL2 sequence, and so on.

The exogenous nucleic acid encoding a functional sequence can be in addition to the mutant version in the genome of the cells, e.g., can be present separate from the mutant alleles such that the cell may include sequences encoding both (and possibly both mutant and functional protein); the mutant alleles can optionally be disrupted to prevent transcription and/or translation of the mutant nucleic acid or protein, e.g., by replacement of the mutant allele with a functional allele (e.g., replacement of the mutated region with a functional sequence or replacement of the entire mutant gene with a functional gene) such that no mutant protein or DNA sequence remains in the cell, or by disruption of the coding sequence or promoter sequence of the mutant allele to prevent transcription and/or translation. homologous recombination as described, for example, in U.S. Pat. No. 5,641,670, the contents of which are incorporated herein by reference, to effect replacement of the mutant allele with the functional sequence.

An exogenous nucleic acid sequence that corrects the disease-causing mutation can be introduced into a primary or secondary cell by any method known in the art that preserves cell viability. Typically, the cells are combined with the exogenous nucleic acid sequence to, e.g., stably integrate into their genomes, and treated in order to accomplish transfection. As used herein, the term “transfection” includes a variety of techniques for introducing an exogenous nucleic acid into a cell including calcium phosphate or calcium chloride precipitation, microinjection, DEAE-dextrin-mediated transfection, lipofection, electroporation or genome-editing using zinc-finger nucleases, transcription activator-like effector nuclease or the CRIPSR-Cas system, all of which are routine in the art (Kim et al (2010) Anal Bioanal Chem 397(8): 3173-3178; Hockemeyer et al. (2011) Nat. Biotechnol. 29:731-734; Feng, Z et al. (2013) Cell Res 23(10): 1229-1232; Jinek, M. et al. (2013) eLife 2:e00471; Wang et al (2013) Cell. 153(4): 910-918).

The genetic modification can be performed at any time in the process, e.g., before enrichment for progenitor cells, after enrichment but before expansion, after expansion but before differentiation, or after differentiation.

Before or after transfection, the cells can be allowed to undergo sufficient numbers of doublings to produce either a clonal cell strain or a heterogeneous cell strain of sufficient size to provide the therapeutic protein to an individual in effective amounts. The number of required cells in a transfected clonal cell strain is variable and depends on a variety of factors, including but not limited to the use of the transfected cells, the functional level of the exogenous DNA in the transfected cells, the site of implantation of the transfected cells (for example, the number of cells that can be used is limited by the anatomical site of implantation), and the age, surface area, and clinical condition of the patient.

The cells can also be modified to decrease expression of the endogenous gene comprising the genetic alteration that is associated with the lipodystrophy, e.g., using CRISPR/Cas9 or other gene knockout methods known in the art.

Thus, the methods can include determining what genetic alteration is associated with the lipodystrophy in the subject, obtaining cells from the subject, enriching and amplifying adipose progenitor cells from the subject, correcting the genetic alteration in the cells, and implanting the genetically modified cells into the subject.

Methods of Treating Lipodystrophy

As noted above, the present methods can be used to treat patients with lipodystrophy, by generating an enriched population of human adipose progenitor cells (preferably obtained from a patient to be treated), optionally correcting an underlying genetic defect in vitro, expanding the adipose progenitor cells to obtain sufficiently large numbers of cells, optionally initiating their differentiation into adipocytes, and implanting them back into the patient where they will integrate and form healthy adipose tissue. Preferably white adipose progenitor cells are used to treat subjects in the present methods. For clinical use, primary cells are preferably obtained from the same individual to whom the populations of cells are to be administered.

Methods known in the art can be used to administer the cells, e.g., as described in Tran and Kahn, Nature Reviews Endocrinology 6, 195-213 (April 2010); Attached Yoshimura et al (Yoshimura et al., Breast J. 2010; 16(2):169-75; Yoshimura et al., Dermatol Surg. 2008; 34(9):1178-85; Yoshimura et al., Regen Med. 2009; 4(2):265-73).

Hydrogel vehicles can be used for therapeutic purposes to promote the survival and functional maintenance of the cells following transplantation. Hydrogels are three-dimensional (3D) cross-linked networks formed by hydrophilic homopolymers, copolymers, or macromers that swell in aqueous solution and provide an appropriate microenvironment similar to the ECM, thus facilitating the migration, adhesion, proliferation, and differentiation of cells, and efficiently delivering nutrients and growth factors.

The cells produced as described herein can be introduced into an individual using various routes of administration and various sites (e.g., renal sub capsular, subcutaneous, central nervous system (including intrathecal), intravascular, intrahepatic, intrasplanchnic, intraperitoneal (including intraomental), intramuscularly implantation); when possible, implantation at a site that improves appearance of the subject is desirable. Once implanted in an individual, the transfected cells produce the product encoded by the exogenous functional nucleic acid, improving metabolic function in the subject.

### EXAMPLES

The invention is further described in the following examples, which do not limit the scope of the invention described in the claims.

### Example 1. Generation and Transplantation of Adipocyte Progenitor Cells

Previous findings have suggested that the niche (i.e. the specialized cellular environment required for survival and proliferation) for mouse adipocyte progenitor cells might be the microvasculature of adipose tissue. [7] A corollary to this concept is that in order for adipocyte progenitors to proliferate, the entire capillary network must proliferate. Using the methods described in US 2015/0259647, we tested this possibility by placing small fragments of human adipose tissue under pro-angiogenic conditions ex-vivo. This lead to growth of capillary branches ex vivo, as well as cells associated with the capillaries. The growth of capillaries was absolutely dependent on the presence of angiogenic growth factors, and was enhanced under conditions perfected for angiogenic growth (EGM-2-mv media, Lonza) (FIG. 1). We then discovered that cells contained within this capillary outgrowth could be differentiated into adipocytes upon exposure to an adipogenic cocktail consisting of DMEM-10% FBS, 0.5 mM 3-isobutyl-1-methylxanthine, 1 μM dexamethasone, and 1 μg/ml insulin (MDI). 72 hr later, the differentiation medium was replaced by DMEM-10% FBS, which was replaced every 48 hours until analysis. (FIG. 2A).

In addition, individual cells could be isolated from in-vitro grown capillary branches. Exposure of the explant to a cocktail of dispase 1 U/ml in DMEM for 1 h at 37° C. resulted in the recovery of a single cell suspension that could subsequently be plated in plastic culture dishes and subjected to differentiation. Upon differentiation, the canonical markers of adipocyte identity (adiponectin, perilipin, GLUT4 and leptin) were induced, as ascertained by RT-PCR (FIG. 2B). Thus, the methods can be used to obtain human adipocyte progenitor cells through expanding the vasculature of adipose tissue in-vitro and then preparing single cell suspensions (Human Adipose Capillary Progenitor Cells—HACAPS) from the expanded vasculature. These cells can be used for further expansion and therapeutic application for reconstructive surgery.

To determine whether HACAPS are useful for transplantation, we injected ex-vivo differentiated HACAPS into immunocompromised mice using Matrigel, a hydrogel formed from gelatinous protein mixture secreted by Engelbreth-Holm-Swarm (EHS) mouse sarcoma, as a vehicle. Four weeks later, the cells had successfully formed functional adipose tissue, which was vascularized, was metabolically active, produced human adipokines (FIGS. 3A-F), and improved glucose metabolism (FIG. 4). These results indicated that functional mature human fat cells could be generated from progenitors, and used to generate normal adipose tissue in vivo.

### Example 2. Hydrogel Vehicles for Human Use

Ideally, adipose progenitors for use in human therapy would be administered in a hydrogel vehicle that is more or less functionally equivalent to Matrigel, but with a fully defined composition. The properties of vehicles should include: 1) that they support viability of cells by allowing nutrient and oxygen exchange following transplantation, 2) that they allow new blood vessels to grow into the transplant, 3) That they allow the transplanted cells to expand in size and form their own extracellular matrix. What vehicles will fulfill these properties is not obvious; investigators have for decades developed numerous hydrogels primarily for use in cartilage and bone tissue engineering, but hardly any have been utilized in clinical regenerative medicine. Hydrogels specifically designed for the support of adipocyte progenitors and the formation of functional fat are not known. In the present studies, commercially available hydrogels (CellStart-ThermoFisher, Synthemax-Corning, VitroGel3D-TheWellBiosciences) failed to support adipocyte viability. An injectable hydrogel has been developed in which mechanical, degradative, and biochemical properties are precisely tunable (ClickGels). This hydrogel is based on catalyst-free, strain-promoted azide-alkyne cycloaddition crosslinking of cytocompatible building blocks with biorthogonal reactive end-groups, a gelling kinetics suited for formulating injectables and unaffected by cellular cargos, and precisely tunable degradation rates for modulating metabolic activities of encapsulated cells [8]; U.S. Pat. No. 9,388,276).

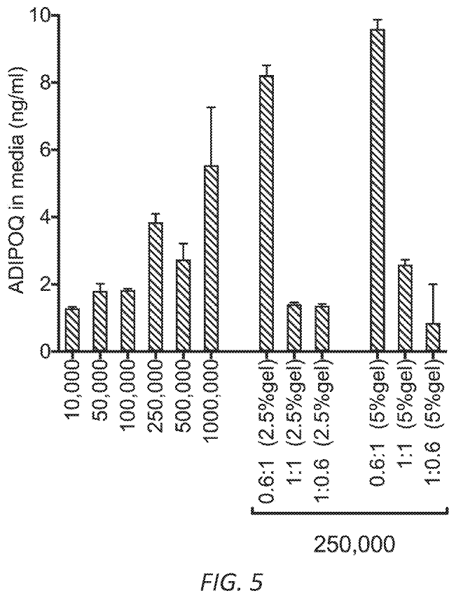

By adjusting the stoichiometric ratio of azide- and DBCO-terminated building blocks (degrees of SPAAC vs. physical crosslinks), and the concentration of cells in the gel, we developed a hydrogel (AdipoClickGel) that maintains viability and allows functional differentiation and stability of HACAPS (FIG. 5). Thus, the methods can include expanding adipocyte progenitors and using of AdipoClickGel for human autologous adipose tissue therapeutic engraftment.

### REFERENCES

1. Park, K. W., D. S. Halperin, and P. Tontonoz, Before they were fat: adipocyte progenitors. Cell Metab, 2008. 8(6): p. 454-7.
2. Amer, E., et al., Adipocyte turnover: relevance to human adipose tissue morphology.

Diabetes, 2010. 59(1): p. 105-9.
3. Brown, R. J., et al., Effects of Metreleptin in Pediatric Patients With Lipodystrophy. J Clin Endocrinol Metab, 2017. 102(5): p. 1511-1519.
4. Lewandowski, K. C., et al., Familial partial lipodystrophy as differential diagnosis of polycystic ovary syndrome. Endokrynol Pol, 2015. 66(6): p. 550-4.
5. Savage, D. B., Mouse models of inherited lipodystrophy. Dis Model Mech, 2009.

2(11-12): p. 554-62.
6. Agarwal, A. K., et al., AGPAT2 is mutated in congenital generalized lipodystrophy linked to chromosome 9q34. Nat Genet, 2002. 31(1): p. 21-3.
7. Tang, W., et al., White fat progenitor cells reside in the adipose vasculature. Science, 2008. 322(5901): p. 583-6.
8. Xu, J., E. Feng, and J. Song, Bioorthogonally cross-linked hydrogel network with precisely controlled disintegration time over a broad range. J Am Chem Soc, 2014. 136(11): p. 4105-8.

### Sequences

### Homo sapiens 1-acylglycerol-3-phosphate O-acyltransferase 2 (AGPAT2), RefSegGene on Chromosome 9

### NCBI Reference Sequence: NG_008090.1

>NG_008090.1:5001-19317 Homo sapiens 1-acylglycerol-3-phosphate O-acyltransferase 2 (AGPAT2), RefSegGene on chromosome 9 (SEQ ID NO:1)

### Homo sapiens BSCL2, Seipin Lipid Droplet Biogenesis Associated (BSCL2), RefSegGene (LRG 235) on Chromosome 11

### NCBI Reference Sequence: NG_008461.1

>NG_008461.1:6868-24313 Homo sapiens BSCL2, seipin lipid droplet biogenesis associated (BSCL2), RefSegGene (LRG 235) on chromosome 11 (SEQ ID NO:2)

### Homo sapiens Lamin A/C (LMNA), RefSegGene (LRG 254) on Chromosome 1

### NCBI Reference Sequence: NG_008692.2

>NG_008692.2:4974-62517 Homo sapiens lamin A/C (LMNA), RefSegGene (LRG 254) on chromosome 1 (SEQ ID NO:3)

### Homo sapiens Peroxisome Proliferator Activated Receptor Gamma (PPARG), RefSegGene on Chromosome 3

### NCBI Reference Sequence: NG_011749.1

>NG_011749.1:5001-151507 Homo sapiens peroxisome proliferator activated receptor gamma (PPARG), RefSegGene on chromosome 3 (SEQ ID NO:4)

### Homo sapiens Perilipin 1 (PLIN1), RefSegGene on Chromosome 15

### NCBI Reference Sequence: NG_029172.1

>NG_029172.1:5001-20051 Homo sapiens perilipin 1 (PLIN1), RefSegGene on chromosome 15 (SEQ ID NO:6)

### Homo sapiens Lipase E, Hormone Sensitive Type (LIPE), RefSegGene on Chromosome 19

### NCBI Reference Sequence: NG_034246.1

>NG_034246.1:5001-30920 Homo sapiens lipase E, hormone sensitive type (LIPE), RefSegGene on chromosome 19 (SEQ ID NO:7)

### Homo sapiens Caveolae Associated Protein 1 (CAVIN1), RefSegGene on Chromosome 17

### NCBI Reference Sequence: NG_015845.1

>NG_015845.1:5001-25872 Homo sapiens caveolae associated protein 1 (CAVIN1), RefSegGene on chromosome 17, (SEQ ID NO:8)

### Homo sapiens Caveolin 1 (CAV1), RefSeqGene on Chromosome 7

### NCBI Reference Sequence: NG_012051.1

>NG_012051.1:5001-41401 Homo sapiens caveolin 1 (CAV1), RefSegGene on chromosome 7 (SEQ ID NO:9)

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of providing a population of adipose progenitor cells for treating a subject with lipodystrophy associated with a genetic mutation, the method comprising:
obtaining primary adipose tissue from a subject who has a lipodystrophy associated with a genetic mutation comprising a first population of adipose cells, and
(i) culturing the primary adipose tissue in a protein-gel matrix in the presence of pro-angiogenic factors to induce the growth of a second population of cells;
(ii) isolating cells from the second population to form a third population of cells comprising an enriched population of adipose progenitor cells; and
maintaining the population of adipose progenitor cells under conditions and for a time sufficient for proliferation of the cells,
thereby providing a population of adipose progenitor cells for treating a subject with lipodystrophy associated with a genetic mutation.

2. The method of claim 1, wherein the second population of cells is subjected to enzyme digestion to produce the third population of cells.

3. The method of claim 2, wherein the second population of cells is subjected to dispase digestion.

4. The method of claim 1, further comprising mixing the genetically modified adipose progenitor cells with an injectable hydrogel.

5. The method of claim 1, wherein isolating cells from the second population comprises isolating CD45−CD29+CD34+CD24+CD144− cells from the second population.

6. An isolated, enriched population of adipose progenitor cells made by the method of claim 1.

7. The isolated, enriched population of adipose progenitor cells of claim 6, which is in an injectable hydrogel.

8. The method of claim 1, further comprising genetically modifying the second population of cells or the third population of cells to correct the genetic mutation, to provide a population of genetically modified adipose progenitor cells.

9. An isolated, enriched population of genetically modified adipose progenitor cells produced by the method of claim 8, which is in an injectable hydrogel.

10. The method of claim 1, comprising maintaining the adipose progenitor cells in culture for a time and under conditions sufficient for the cells to differentiate into white adipose cells.

11. The method of claim 10, wherein the white adipose cells express ADIPOQ, PLIN1, and/or LEP.

12. The method of claim 10, comprising maintaining the adipose progenitor cells in an injectable hydrogel.

13. An isolated, enriched population of white adipose cells made by the method of claim 11.

14. A method of treating a subject who has lipodystrophy, the method comprising administering to the subject the isolated, enriched population of adipose progenitor cells of claim 6.

15. The method of claim 14, wherein the subject has CGL1 or CGL2.

16. The method of claim 14, wherein the first population of cells is obtained from the subject to be treated.

17. (canceled)

18. (canceled)

19. (canceled)


SEQUENCE LISTING (9 sequences)

SEQ ID NO: 1 - DNA, 14317 bp, Homo sapiens
ccagccccgccgccctcgcaataaggggcctgagcgcgcgggggagaagcgggagcgggagcgggagcgagctggcggcgccgtcgggcgccgggccgggccatggagctgtggccgtgtctggccgcggcgctgctgttgctgctgctgctggtgcagctgagccgcgcggccgagttctacgccaaggtcgccctgtactgcgcgctgtgcttcacggtgtccgccgtggcctcgctcgtctgcctgctgcgccacggcggccggacggtggagaacatgaggcaaggccgggggccgccgggaggggccggggaaccgccgcgccgcttccgcttccctaactttcttctgggcttccctccttcctgccccgcccgtcccgccccgctccggggctccggggagagcgcgcctgggccggcggcaggcacaggagggggtcccggagtcagggggtcccggagtcacggggtcaaggagccggcgtcacagtgcccagcaccccaccccccgccctggccccgggcgtctacaccggtttcggcctccgccgcgtccctgcagcccggcgtggggttccgggggtggagcagctttcggagagaaagggggccggtccgcgctgggagaggctcagccctgcttcctgagcggggaaaccgaggccggggcggcggggacttgccccagggctattgatcagggcgcacgacggcgtgggggcacccctcggcctccctccgaccccctcctgactgtccgcccggtgatgggcgcacctgtggggcgggttcccctttctctccctttcctcttcgcagggaggtgggggacccggaggggacctgcttccgtgctggggagcgcgcccctggcctacagctccgggggcccagcttccaagcgaagggaaggaagagttaggactagggttcggcggggctgaaattttcccctggacgccttccagaaggtgggagcttggttttgccaggctggggttgggggccagaggtcagactgggctgccaaagacggcgggggctgcctgctcctgggcggggtggctgctggagaaggctcttgcctgccccctctccctgttagccgtccccaggctgttgctacatttctccctgccctgtttggactgcccagccagtggcttttgcaatggcctggggtgggggcattgtggccccagaggcccccttccccgtggttccaggggcagtgggtgagggtttgctagctccctgcactcgctgtccttgccagccctgggagggcactgaggctgctggaacgggcctacagtcatggcttccaacggagccccagaagctccaggaatatgaaggaggaccctgggcctgatggtgagcatggagagtccgagaagggcctgctggctgctcagccagacgtgtgtgaccagtggtgatgtgggccaggtggcccaggccggggtcccggggagcgcacactccagggctggcccaggtggtgactggcgctgacgtccccagtggagcacccttcagcgaggaaggcagtttgcagaaacagggagttcctgggggctgggctctgggcacctgtgctggaatctgtgaaggcctcacctgtggttctggccctgggatcccctgtaggcaggggtctctgtgaggggcccccctcagcccctggggtgcttcagagtaggggcgagggtgcggctttcccagctgaggaggggccagtctgtgggttccttggggtgccccccaccccgaggaaatggcttgtgtggttctgatgcctgcagggcatcctgtgctgggcgtgtcctggggtgtggacaggggacggggtcctgttcttttggcagaaatgatggggcacctgacttaggagaccctggagcagaccccactactgagccaggccacaggccacgggcctcttcatcggcggcctcctcgtgtgagaaagcctggcttgttctgcaggggcctctgcctggacttcctagctgttttccagggaccaaatcacagttcacctgctgccctttctatggacctggcaggctgggttggctggcactggccctgctgccccactgggccctgtccccatctcagcttggagattctggctaacctgggtgtcctcgggacctggggcctctgatggtgcttggatgggaccctcctgccgccctcacctctccagggcagcacccaggctcaggcctgtggagtagtggcttccgggacactggctactaacagcaggtgctttgttcctgctttggtccctgaagagaagggagggccagagggcagggcagtctggaggagaggcctggggctccatggctaccttcccccacacctctgcacgtgctccccaggccccagggtccctgatgacttccaagactcccagcaatggctaagagtggggctgttgcgaacgtgcgttccctccgtgggggccgtcgggaggggtgagaaaggcagatgaagagcaggcgctttgtcactccagggaagggacccggggggctggaagggaaggaggggatactcggcctctgttcctgctgtacgccacgggtgcctccagcctcaggaacagggattcgcgtgtgctctgtgttacaaataaggacactgaggctgagataagcaacttgaaactggggcagggtccccaagccaggatggggtggaactggggttcgaacccactgacagtgactccagagtacctgcttttccctttccccttgttcccagcccagcgcttggggctggtgaggcagccctgcggcaggcctgagctctgggaggggcgctccatcttgcccctgagcaatggctgccctggctgaggcccagcccctccccacggggttccaggcggcacccaagagacccccaccgtctacaccgtagttctgagcgtgtgagggacagtgatgggactagaacctctgaggcagccttcgttcccaccgggcttggggctgagaccacggaccccttccaggcagggggtgtcccctgagcccttccctgtgtgcgcagttacggtggagctgcaggggacgtttcctcaccaggggccgatgccctggtacatccattcgacagaggaaagcaaagcccagagcggctccgtgtcctacagctgggttcagctacaggatcagaaaaccctcaagacagcagcttaatccacagagaagtttactttttcatacagcaaaggagaccagatgggcagcctagggaaacagcagttagtttccggctcctttgacctctattcccaccattttcagggtcccagacagaaaaggggtgctcttcctcttaaggatgctccccgaattgaaccccacgctctgctggtatctggaggccgggacctagtggttgtggcagctcctggctgcagggcaggctcctgtccccatggagcacgtgcccacaccacacgcccagtgtggggagaacgcacaggggacttgggatgtcgggcaaggtttgacccagatttgccggccagtgcatggtcccgggtcctctgacagatgtccggtgatgacagatgccatcgggggccctgtgtcaccacagcactggggtcttagtcccaagggagaccgtgaccttggtcctccagagcgctccaggtgtctctgtgcaggcccagaaattggagctgtcctgactgagggatgggagggcttctctggccccacctccttcccgcccgccacacctgcactcctacacatccgctttgaggagtcacggggaccccccacatccgggtgtttgtgtgggctggaccccgcacccctatggacccatcccgccttgtgctgcagcttggatgacacttgtcggaaaacaccagattcctcactggggtccctgtccccggtggccccagcgagccccagcagagggggagggggctgccctgaatgagctcacgcagctgaagatgggcaagtcctggctcagagagctgctgggtctcctgggagtgtcccagcccaggaccaaggcaagggtcagcgagtggccttccacgaggacagcctggccagtggcctgggccctgacctagaaccgcctcccagctgcctgagccaggtgctggtgggattgggggcttaggcacattgaggggccttttgcttcgatttactttggacttttttttttattttttgagacaaggtctcgctctgttgcccaggttggtcttaaacccctgagctcaagtgatccgtctgcctcagcctcccaaagtgctggggtcacaggcatgagccaccgtgcccggccccacccattttggattcttccccccctcccccccaccccctagtcatccccatcacagccactgttggtggaggtcctgccctggcccgatggtgccccgggtgtctccgcctcatagaatgtaagcctgcacacccttccacaagagcctgggaggtcaaatgctttgcccgaggccatagctgggaagaagggaaatgggatccaaatccccgtcaggcccagagcctgagtgtgggagcagcccaggtgcctgggggctgagctcaggcggtctgaggaggctgggttcctgcagaccccagaggggtctcttacggggcttcgctcgaggccagtggactccagtctgagggctcctgaaaaagactgaggcagcacctgggctttccctcagcaggagcccactgctccgatgccaccagaagggcccagtctgggctcagcctcacttagttttgggtgctgggctgctccctgggcctcctgccttgctgggcaaagctggcacaagtcacccgcctggcagtggccagcagggctccctgagctccaggcttccggcaaccttggctttgcccctgctccccggggaggcaaggagtccaggacctggatttggggtccgtgtgactgtgtcctggtctggcacagcgacacctgccccaggctttgaggaaaggacagggcagcccctcgtccgcctgtggttggtgggaggggagtttgagtgaggcagggtgtttggcacaggacagggactgggctgggcaagctggtgggggaagcctggggacagtggctgctctgagcttcctgctgggggaggccggagcaagctcccagccctgctgaggggttgaggggactgcggagcccgggggagggagggagcctcaggatgagctcctagagtcctactgggggcccgcgccgccccagcacgactctgccagctccgaagcagggttccgaggggggctcaggcctgttctggcttccctgctgtgtggccttggggaagtcacttaactttgctgaaccttgaacctcgcattcctcatctgaagaggggggttatgatcgtacctaccctggggggttactgtcagaacaaaaggaggtgactcatgtagagcccttgtctcgaggctttgcacacaggaggtgcagggggcctctgtgcttagaaggcgatttttctctgcttgggttcccgggggtgtcctcggccgcaggagcctctgcagggcttagttcagtcccttcctctctgaggaaacactttttttgtgtgtgtgagatgaagtcttgctctgtcacccaggctggagtacaatggcaccatctcggctcaacgcaacctcctcctccctggttcgggtgattctcccgcctcagcctcccgagtagctgggattacaggcagctgccaccacgtctggctgatttttgtattgttaatagagacgggatttcaccatggtggccaggctggtcttgaactcctgacctcaggtgatctgcccgccttggcctcccaaagtcctgggattacaggcgtgagccacccgcccatccaggaaccgcttgtaaagtgagtctagactttcccaccagctttcagaaagctctgaactatgtacgggacaccgcgggggaagtgcggtggcctcacgtggctggtgcagatggccctttgtccccttgaggacatcaacttcaactttcttccacccccagggtatacctttctcccccagccacactgtgggctgcagagttctagagagccacagcctcccgctggcctggggcagcctccgtccgtccatctgtctggcctgaggaccaggggccggtggtgggcggcgggacacagcgcctgctcagcaccgcctgctgggcgggggtttgtaccccgtgtctttcacagctgagagaagtatcagtgcacgatccaacccctaagcagcttagattaaaaaaaaaaaaaaaaggaaaagttttggagctagagggtggtgctggccacacggcagtgaatgcgccgagtgccacggaaccgtgtgctgagaaatgggtacaattatattgtttaggtggtgcgtgttataccacaatcaaaacagaggagggctcggcgcgctggctccttcctgtcatcccagcactttgggaggccgaggcgggcggatcatgaggtcaggagtttgagaccagcctggccgacatggtgaaaccccgtctctactaaaaatacaaaaattagccgggtgtggtggcgcacaccagtagtcccaactacttgctaggctgaggcaggagaatcgcttgccgggaggtggaaatggcagtgagctgagatctcgccactgcactccagcctaggtgacagcaagactccgtctcaaaaaaaataaaaaataaatgaagatcttattttttaataatttcagatttacagagaagttgcaaagatattacgggggaccccatgtggccctcaccagtttcctcccttgttaacatcttacatcaccaagatgcagttgttaacactaagaaactagctggccgggcgcggtggctcacacctgtaatcccagcactttgggaggccgaggtgggaggatcacctgaggtcaggagttcgagaccagcctggccaacatggtgaaaccccgtctctactaaaaatacaaaaactagccaggcctggtggcaggcacctgtaatcccagctactcaggaggctgaggcaggagaattgcttgaacccgggaggcagaggttgcagtgagctgagatcacaccactgcactccagtctgggtgacagagtgagaccctgtctcaaaaataaaaaagtaaataaaagatggaactggccaatgtcactaccagagggaaccaatgtccctttcagatgtggcccgagcctcctgcaggcagctagctttggaattgcaggtgtgtcttggctgctgctagaaatcatacacatgtccttcacaggagatgagtgtggacctgtcctgttctttccccgcgccccagctgtctgtgaaatggaccctaatttactttccctctttctactggtggctatttaagcattttcaggttttcctaattcactgcacacttcagcaccacaggcttctcactccagccgtcgggcgaattcctggaagattctgagaggggaggggccaggtttcctcagggctctggccgggcctgccaacctgccctccacagaagtatcttgtctgtgcgactcctcactcctgccagccgctgggcctgttcccaacaccctggctggcacctcctgttatcaggcgttttaatctgtgccagggggaaggatgaggacagtctcttgtttccatttgcgcttctttgattggcgacacggccgtcttgctagcaccagacagttctgtgtttcctgttggtgaactgccggcttttgcctcctcgtcttcttggagcgtcacaggggtccgggaagctggattgcgcaaatgagtgtggaggcaaaacagccctgcagttcccctgagtgtgcgggtgctaccagcccgggtgtgccttctgggtgggaggggacggtggccggcgcggcctggagaggagatatctagagttggctgaggaaggcagatctgaggaggccccaggcttccaagtccagggacggacggctggactcatcaccagaattaaccattaggccccccttgactcagtttccccatctgcaacagggggtcatgagcattgccccatcagcctgaggtggtgctgccgcaggcctgggctcacacatggaagggcagagtgccagggctggggaggggacaggggtttgtgtcgcatgggaacagacgtcagccagagaaggtggagagttctggagatagagggaggagatggctgctagacaccgaatgtgcccagcgcccatgaactgtgcacccggaaaaggccaaggagatacattttatgttacgtgtattttaccacaatttttttttaaaggccgggcgagttggctcaggcctgtaagcccagcactttgggaggccgaggtcggcaggttgtttgagcctaggagttcgagaccagcctggccagagatggtgaaagcccatctgtaccaaaaatacaaaaactagcgaggtgtggtggctcacgtctgtggtcccagctagtcaggagcctgaggtgggaggagcccaggaggtggaggctggagtgagcccagattgtgacactgcacaatgtgacacattgtgtcacagagcaagacagagcaaggggagcctgggtgacagagcaagaccctgttaaaacaacaaaaatccccaccagtatggctttgagagaaggccccggctttgaatctctgctctgtggcctggggcagttactcagactcatctgtagaaagtgtggggcccttaggactgctgggggatcaaatggtggggggcactagtctgcatggagaggggagcagcactggcagccccccagtcagcgctggcacagccttgccccggccatccacccctgctggagcttcggagtggagctccgtcctcctggctccatccccgccggctgccctgtgatggggtgccctatctggggtgtgcccagagcttcctggcagcacagcagggcagaggttgggacgctcggagctggtcagagtcttcgggtaccccttggacatccctggtcccaaccatgacgggctgcttggacaaggccacctcacatttgcaggtgctccaagggcagcccccagtgggctgggcccctgccagagtgggggctgctgtggccagggctcaggtggcccagaccctggctctgctgctgctcctggtgtgacctcagacaagccccgagcccctcgccgcctcttcacggggaaagggaaccggcctgattctgtagggtaggggtgagagagggtctcggtctcggctcttttctggaagaacctttcggaggtcgtttcagaggtctccactggcgccgacactttcttcctgatgtggccctgccttgaggggccagtgaccttccccagggtgtgtggggccccatttcctctgctggggtgcagaggctgctctccccgacgtgggtccttcatagcggacgtgagagccagtgccagttcctttccggctgggtcccctgtggggtggatgagggctgggtgggccctggatgtggatttggaagtgggcgccttcccatgctcctagcatccgagttccagaggctctgggttttcctctgttgctggggagacggaggcagggcagccgtccaggtgggtgagccgggcccgggactctgtccgcttcaggggctccctcccctgtgtctcccggtctcctgccccgtgccaggagggcccctccccagcctcctccacaccccacccccaggccttcgcgccccagcctcggctgcgggatctgtgggacccgtgttcatggtggcctcccctgcagcatcatcggctggttcgtgcgaagcttcaagtacttttacgggctccgcttcgaggtgcgggacccgcgcaggctgcaggaggcccgtccctgtgtcatcgtctccaaccaccagagcatcctggacatgatgggtaggccgggcctcgggggtggcttctggggtttgagtggggccggctgagctggggctgtgtggggctgggtcccggggacgaggacacagggctgcctgtgcctgggcgagctcggcctcagtacctccctcagggccagacacagaggctcggaggccacacgacccgtccaggtagccagggagaaggcagggtgccaggcaggcctgtgggtgctcagcagctgtcttccagcgcacgctgtctccccctctctctctgtctctgtctctctgtctcccaggcctcatggaggtccttccggagcgctgcgtgcagatcgccaagcgggagctgctcttcctggggcccgtgggcctcatcatgtacctcgggggcgtcttcttcatcaaccggcagcgctctagcactgccatgacagtgatggccgacctgggcgagcgcatggtcagggagaacgtgagtgtgcagggccgggctcggtggggttggggtggggcctgggcggggccaggcaggggccagcttgtgacttggttttggcagaaaaaacaagacccccacatcatccatgctccgcaggtggggtcccacgccagacccctacatcatccatgctccgcagatggggtcccatgccagctgctttgcgaagtggggcttcttaggaggcgaggcggcgtggcctttctggggtggcctgggcgtgaggtcagtccaggctctcctctccctgcagctcaaagtgtggatctatcccgagggtactcgcaacgacaatggggacctgctgccttttaagaagggcgccttctacctggcagtccaggcacaggtaggctgagcccagccctccctgggtgggtgcaggctggggaggcggggtcaggctggcttaaggcagcaggtgaccaccagccgagctgaggacccttgacacacaagggactcctcccactgagttggggacagggcctccttgccccttcctgcacttgccccctgaccgaccagggagggcagccccttgggccttttgcccacacagagcccagagtgaccagctgcccttggtgggcacccagtgcttggcctgcgaggctgggcaggtagaggggtggggagccgggccgctcgcctctgctgcagagctctcaggaggctcatttctgggcctgtgcctggctggggtgggcttcagggcatgggcagtgggtctggacaagccaggcttttggtgtagtatcaggccagaccagagctcagggtgggtggagctcattgtaagcagcgcctccagactctggacttcctggtccccagcctccaggggtgcagtgcatcctagggtgtctgggagaggccagggtatagccattcccccagcacctgtcctgcctctgccgctgtgctgggaagagttgcgtcaggagaaggagcttttacccatctgggtggggtctcgctcagggcaggggtctcagctccttccctcacactggccactagtggcccccagtgcctggccctgtgcaaggtggtggggagggggccctccttccagaggctcccagctaaggccgtgaggcctggggagccccggaggcgctgggctgggtgagcatccttgggctctgatcccagccctgcccaggtgcggggcacacctgcccagcctggggctgatgtcacttccctttgggggcagagtgagggctggatcagctctgggagagcagaggggagacgggcggctgtgatggggtggctgggggtggccgcagtgcagcttcagccaggagacccacagcttttccgtccctgctcccggtccccagcctacctgctggccccctccctccctgctgccagtccccagcctacctgctggccccctccctccctgctcccagtccccagcctacctgctggcctcctccctccctgctgccagtccccagcctacctgctggcctcctcccttcctgctgccagtccccagcctacctgctggccccctccctccctgctcccagtccccagcctacctgctggccccctcccttcctgctgccagtccccagcctacctgctggccccctggaggttccctggctgcctcgggttaatgatcacccccaagtgcccttggatttgaccccggtgacttccaggaacaccagcaggctccacccctcccccgggctagggctgcctccctcactggcttcctgagatggagaggactgggagagaggcgtgggcagggaagagtgccataatctcctcctaaaccggacagggccctgcccccctggctagctgcagacccaatatggggacaggtgtgaaccaggcaaacacgtgctagctcaggagggcttcctagaggagatgagtaaaaatttgcaggatgagcttatggaactctgggccaaccagagcaggacacatatcccaggtcccaggggcaggacagccacaaggacccatggcagcagcgaaggcaggggtgggtgggccgcaggacagggctccccaaccacatgcagcctggggtgtgcctggcctgtccccagggctgcctcagctgtgcgtctccctgcctgcaggtgcccatcgtccccgtggtgtactcttccttctcctccttctacaacaccaagaagaagttcttcacttcaggtacccccacatgtgtgcacccggggtgtaggccccgcctgaccctacagtcacggagccccgggcccctcatcgttcccatttccgggtggcacccgtggcgtggccacacggtgaccatgtggcgaatgagtgactcaggctggagtcccagctgtgggcttcatggcctcatggccctgtgagatgggtggtgctgggcccatttttcagatgagaaaactgaggctccaagagacttcacacggatggaccccagctgtgcacacagcttgggggcctggagggcggcctctgagcccatgtgtccaggctgatgcccgcatagggtagatggaacccaggcagcccaggggcacaggctgcggggctcatgacctcttgccccttccagccagttcccagaacgtgggcacctggtgcccaggcaggacagtggggtcaaagttggagagcagtgggggaacccagctccatctctcccacagcccctcgccccgtatggagggcagaggccaggcagtgaggtaggggctgatcaggaagtggggctttctgggggcagtaacaggggagcgcagacttgtcgaaaatgcggcgtgaaggccaggcagccatggggtttactgcagcaacgttactgcaattaaaataacccaccacggctagggagtccaggggaagagcccggcctcgggcttcccagggaggggctgtggggggctggggagaggcgaggccagggcagcaggctgaggtgggccccagctccccacaggccactgaggtctgttgcttcccccacaggaacagtcacagtgcaggtgctggaagccatccccaccagcggcctcactgcggcggacgtccctgcgctcgtggacacctgccaccgggccatgaggaccaccttcctccacatctccaagaccccccaggagaacggggccactgcggggtctggcgtgcagccggcccagtagcccagaccacggcagggcatgacctggggagggcaggtggaagccgatggctggaggatgggcagaggggactcctcccggcttccaaataccactctgtccggctcccccagctctcactcagcccgggaagcaggaagccccttctgtcactggcctcagacacaggcccctggtgtcccctgcagggggctcagctggaccctccccgggctcgagggcagggactcgcgcccacggcacctctgggagctgggatgataaagatgaggcttgcggctgtggcccgctggtgggctgagccacaaggcccccgatggcccaggagcagatgggaggaccccgaggccagacgcacactgtccgagccctctgctcagccgcctgggacccaccagggtgcagctgggctccagggtccagcccacaagctgcatcaggctctctgggagaggaggggcctggagggccaggagtcccagactcacgcaccctgggccacagggagccgggaatcggggcctgctgctcctgctggcctggaagactctgtggggtcagcactgtactccgttgctgtttttttataaacacactcttggaagtggc

SEQ ID NO: 2 - DNA, 17446 bp, Homo sapiens
gctggtacctgaaggatgaaggatgctagaaggagctgctcagatcctggagcataccaactgcctggccagtgggggagcctagctgcctgcatccctgcgcacacccccactgtcgctagttcactgcctgcccctcccccattgcaggcatcctgactctgggctagagacctccccaacagagctgaggccaaggccgactccccctctcaaatggcgtggtctgggcctatgacggcccctgcagtggagtctgtactggctgcgggggaccctgctcatttgaaaatctgacatcagctgggcagtcgcccccctcctcctttcctccctctactctgacacagcacttagcacctgaatcttcgtttctctcccagggaccctccattttccatatccaggaaaatgtgatgcgccacaggtatcagcgtctggatcgccacttcacgttttagccacaagtgactcagtggaagatccagagtcaacagaggctcgtcaggaagatgtctacagaaaaggtagaccaaaaggaggaagctggggaaaaagaggtgtgcggagaccagatcaaaggaccggacaaagaggaggtaggggcccttgtggggacagtcagcagaaatatactggaaataggtgggctggcgtgaagggggcggggggatggattggttttaagaatttcattcattttgaatagattacttcccacgcccgcagcagtgtgcaaagatgtacacaacactggccttgcctctgaaggactttatctggggtgattagacgtctacaaaatagaagactgccaaaagctagtctagataatgtgctgtggggactctgagggagagtgtatccagttagggtcagaggaggtccacaatgctaccacgctgggaacagggcactaatctgcctgcgcaacaacgcaagaggcccaggttgccgcaaactgcaacatgctctaggcgaatgcccccgccttagatgcctacaggtcctaggagctgtgggcgataaaaatcagcgccagctggatgaccgcctcactcggccgcttcgcgggtgaaatggcctgtggtgaaggtgtgcacggagggctgtgattgcagtctcaagggagagccgagcggaggctgcagtcagcgcatcccctggggcacatcttggggcctgcagggaggcaggcgtctttcccctttggtcacggtaaccggagggaaacctgccgcagagagagccagtggcgagcgcaaggagacagcgacctcgcagctcgggaaggctccgccccgtcccgccccgccccgccgtgctggaccccgccctgggctgaagccccgcctcccacggctacaaaagcggccggcggagaggggcgggacttccccgggagccggaagtcccgtctcacggttgccctggcagcgcgcgaggctggtgagtcggcagccctgtggcagccggcgggctggtttccatggttgcacgattaggtaggggctccgggcgctttgcccacccccgagctggaggggactggcgggaagcggcggttggctgcagggcgctccgccgggcgtggggtgtagccgtgtcggtgagcgagctctgctcgaggcggagaggagaaactcgggaagcaattgtggctactctaaggctactggtttcgagtcaggccgtgcgaccttaggcaagtcgcacattctctccgtgcagctgcctagcagtgtggattcagtatcaccgtgtgtgtaaagctctttctaagaatcgtaaggtgcttattctttgttatatagctgtgaatgagaatcaaggtggtttgtcccagcatcaaatgactggagaaggaattccactgaggagatttagcactgggcgggagggaacacagtggaggaaagggaaggggtaattggcagagtggcacaatcatttggagaaaggccatatatatgaaacaatgactttgctatcctgctatctctcgttcctcaaagccagtccagtaagctgttttgtccccacctccctggggaaaggagtcactcttttccctcaccctctctttccaggaaccaccagctgctgcatcccatggccaggggtggcgtccaggtggcagagcagctaggaacgcaaggcctgaacctggggccagacaccctgctctcccggccatggtcaacgaccctccagtacctgccttactgtgggcccaggaggtgggccaagtcttggcaggccgtgcccgcaggctgctgctgcagtttggggtgctcttctgcaccatcctccttttgctctgggtgtctgtcttcctctatggctccttctactattcctatatgccgacagtcagccacctcagccctgtgcatttctactacaggtgagaggggccttctatcaaaatagaggactcctatgggaattgtaaggcccttggagttcattcagttcaatcccctcatttacagataaggaaaactggagccaggtgtgcttccagggcctctcaactttcacagcccaaggctagatttgggttgccagggctagggttctagttctacctctgattactgaattctggttaaaaaaaaaaaaaaaaaaaggaaaaatatccacttctatcattatgaggatcaattctattttggttggatcacccctctctcagggccaatgtagaattttgagccaactcaaccctttagtgcagaaccaacctggctttcagtagaggagctattaggaaagcagcagtttgctttttacattactgcctctcagtccctgaaagtcggctacacaatagagattatggaaacataagatgagtaggcgcataaacttgcttcccagagagtcttgcgtcttgactggccccttaggttccttctcattttacaaaccctgccaagtgccaaattctctcttttttgaggtggagtctctctctgttgcccaggctggagtgcagtggtgcagtgtctgctcactgcaacctcccaggttcaagcaattctcgtgcctcagcctcccaagtagctgggattacaggtgtgtgccaccacatctggctaatttttgtatttttagtagagatgggtttttttttgttttgttattgtttttgtttttgagatggagtcttgctctgtcgcccaggctggagtgcagtggtgccatgtcggctcactgcaagctccgcctcctgggctcacgccattctcctgcctcagcctcctgagtagctggtactacaggcacctgccaccatacccagctaatttttttttttttttttttttttttttagtagagatggggtttcaccttgttagccaggatggtctcaatctcctgaccttgtgatccacctgccttggcctcccaaagtgctggggagatggggtttcaccatgttggctaggctggtcttgaactcctgaatgcaggtgatccatccgccttggcctcccaaagtgctgggattacagacgtgagccactgtgccccaccaaggtctcttcttcttcttcttttttttttttttctttttgagacagagtctcgtgctgttgcctgggctggagtgcagtggcgcaatctcagctcactgcaacctctgcctccagattcaagccattctcctgccttgcctcagcctcccaagtaactgggattacaggtgcctgccaccacacctggctaatttttttttgtatttttagtagagatggggtttcactatgttggccaggctgatctcgaactcctgacctcgtgatccacccccttggcctcccaaagtgctgggattacaagcatgagccactgcgcccggcctagttctcttttcttaacagtggttgtaggacctatcaacagatgcagagaagggcgttataatcaatggtttaaagtcattttttcccttaagtccctggtccagagctatagtttctaaagcagagcttcttaaacttttaggaaatggatcttatggcccttcctgccataaaaacatatatacggccgggcgcggtggctcacacctgtaatcccagcactttgggaggccgaggcaggcggatcacaaggtcaggagattgagactatcctggctaacaccgtgaaaccctgtctctactaaaaataaaaaaattagccgggcgtggtggcaggcgcctgttgtcccagctacttgagaggctgaggcaggagaaatggcgtgaacctgggaagcggagcttgcagtgagccgagatcgtgccactgcactccagcctgggcgacagagcaagactccgtctcaaaaaaaaaaaaaaaaaaaaaacgtatatgcatgcccaggcatatgctgcacccacacccccacatgcaaatttgcctacaattggaggggttcatgagcttcttaataaccatctgtagatccttttggaggcgttttctcaaactaggacccagaccagagctaccaaatcagaatctcttggggtgggaccctggaatttccttttttttttttttgagatggagtttcacccttgttgcccaggctggagtgcagtggcgtgatcttggctcaccacaacctctgcctcccaggttcaggcgattcttctgcctcagcgtcccgagtaggtgggattacaggcatgcgccaccacgccccgctaattttgtatttttagtagagatggggtgtctccatgttggtcaggctggtcttaaactcctgacctcaggtgatccgccctcctcggcctccaaaagtgctgggattacaggcgtgagccgctgctgcttggccctggaatttacatttttatcaagctccttaagtctttagaaacactgaagtatgcaactatttaagtatctctagattcccatctaagaacatctgttgtaatgagaggacatacattgaagtcataggaagagaatgaaataatcctggtatcttgaatctcctggccacatttgttctccatctttgtaacagagagaagggtgcctgttctgagagaagggaggagggcaggagggagccctagggggcaaagaaggtgtatggatggactagtaagactaacttagcagagtatcctcctccaatctcatttaggaccgactgtgattcctccaccacctcactctgctccttccctgttgccaatgtctcgctgactaagggtggacgtgatcgggtgagtatgggaactagagagaggtttcattagagctttgatgactcaaaataggaagacttgagaaaggcctagagagaaggaattgagtaataagagactggccgggcacggtggctcacgcctgtaatcccagtactttgggaggctgaggtgggcagatcacctgaggttgggagttcgagaccagcctgaccaacgcggagaaaccccgtctctactaaaaatacaaaattagctgggcgtggtggtgcatgcctgtaatcccagctacttgggaggctgaggcaggagaatcacttgaatccgggaggaggaggtttcggtgagccgagatcacgccattgcactccagcctgggcaacaagaatgaagctctgtcttaaaaaaaaaaaggagactaagaggaaggctttgaaggaaaagaaattaaaattaattgtgtgtattgatggatctttctgcagcaggtgttagaaatgttctttctcaacatagtccctgacttacggtaattcaacttaagaatttttcaacttactgtggtgcaaaagcgacatacattccttagacactgtacttcaggtacctatacaaccattctgtttttcactctctttctttttttttttttttttccagacagtctcactctgtcgccaggctggattgcagtggcgcgatctcagctcactgcaacctccacctcctgggttcaagcgattctcctgcctcagcctcccgagtagctggcactacaggagcgcgccaccacacccagctaatttttttgtatttttagtagggatgggatttcaccatgttggccaggatggtctggatctcttgaccttgtgatctgcccgcctcccaaagtgctgggattataggcgtcagccaccacgcccagcctgtttttcacgttcagtacagcattcaataaatcacatgggatattcactactttattataagtataggctttgtgttagatgattgtaatcaactataggtaggctaacataagtgttctgagcatgtttaaggtaggctaggctaagtaagctatgatgttctttaggttaggtgtactaaatgcgttttcagcttgtgatagtttcaacttagatgggtttatcggaatgtaaccccatcataaactgaggaccatctgtatcaggtgaagtatgatctatttatctgctaggtaaagtgagcaatatctgctaggtgaagtaagcaatgttttagacctaaatgaacaaatgagctattacattggaataagatgggcatctacccaagagttttgaaagcatttatataacattatgaaactgttgtcaaatatatgcatcgtgtcattacaaagggtactgtgccagaggactggccagatgccagtgtgatgtcaacaaacctgggaaccctgagaaatgcatatggggactgggccctctatagtaggcaagctagaggaggctctcataaaggatacgttctttttttttttttgagacagagtcttgctctgtcgccaagactggagtgcagtggcgcgatctcacttcactgcaacctccaactcctgggatcaagcgattctcccacctcagcctcctgagtacctgggattacaggtgcccaccaccacacccggctaacttttgtatttttagcagagacagggtttcaccatgttggtcatgctggtctaaaactcctgacctcaggtgatccacccacctcggcctccaaaagtgctgggattacatgcgtgagcaaccacacccagcctgaagttttatttatttatttatttatttgagacggagtttctttcctgtcgcccaggctacagtgcaatggcacgatcttggctcgctgcaacctctgccccctgggttcaatcgattctcctgcctcagcctcccaagtagctggggttacaggcgtatgccaccatacctagctgatttttgtatttttaatagagacggggtttgatcatgttggccaggctggtcttaaactcatgatctcaggtgatctgcccacctcagcctcttgaagtgctgggattacagacatgagccaccatgtccagcctgaagttatttttcaaaagtggtactttggaggaaactgatgtaatgatagaatgtcacacttttgaagggaggagtataacctatagagtctggggttctattttattttatttttcaagacaaggttttgccctgccacctagcctggagtgcagtgatacgatcacggctcgctgcagccttgacctcccaggttcaagtgatctcccaagtagctgggactacaggtgtgtgccaccatgcccagcttttttttttttttttttttttttaatttttagtagagacaagatcttactatgttgcctaggcttgtctcaaactactaggctcaagtggtcctcccgactcagcctcccacagtgttaggattataaccgggtgtggtggctcacgcctgtaatctcagcactttgggaggctgaggaggcaggtgggtcacctgaggtcaggagttcgtgaccagcctggccaacacagtcgaaccccgtctctactaaaaatacaaaaattagccaggtgtggtggcacaaacctatagtcccagctactttggaggctgaggcacaagaattgcttgaacccgggaggtggaggttgcaatgagccgagactgctccattgcactccagcctgagtgacagagcgagactcagtctcaaaaaaaaaaaaaaaaaaaaaccagataggattttgctatgttgcccaggctggtctcaaactcctgggctcaggcagtcctcccacctggtccttccaaactgctgggattacaggtatgagccaccatgcctggcctagaatcttctataaagacatttattcagtttgcttctatggttaaaaaataataaatgacttgactgggcacagtggctcacgcctgtaatcccagcaatttgggaggccgaggcaggcggatcacatggtcaggagtttgagatcatcctgaccaacatggtgaaaccccgtctctactaaaaatacaaaaattagccaggtgtggtggcgcaggcctgtagtcccagctacttgggaggctgaggcaggagaaaggtgtgaacctgggaggcggagcttgcagtgagccgagatcgcgccactgcactccagcctgggctacagagcaagactccatctcagaaaaaattaggggccaagcgtggtggctcacgcctgtaatcccaatactttgggaggccgaggcgggcggatcatgaggtcaagagatcgagaccatcctggccaactggtgaaaccccgtctctactaaaaatacaaaaattagctgggcgtggtggtgtgcacctgtaatcccagctactctggaggctgaggcaggagaatcacttgaacccaggaggcggaggttgcagtgagctgagatcgtgccactgcactccagcctggcgacagagcaagactccatctcaaaataaataaataaataataataataaatgactttattgagaagaaatgagaaagccacagaacctgggatggtgcatacagttttgtggcccattgcagagatcataatgggaaggcagagagccaagggagggagcctcaaacgatcaggtgtgaggctgtcacatgagggagggaggaataaaaggaagatgagagcaaattcagcccatgtccctgataattagaagaggttgaacattttgtttgttgacaatgaacatttttaaatgaattaatgaatgaaataatctgtccactatgtgtaacggcttgctataagtagatgtggtcgctgctctcaatgagctcacagcgttgatggaaaactatacattgcatatgtttgcatacatttaagtagtaaactgtaaaatgcggattctggccaggcatagtggctcatgcctgtaatcccagcactttgggaggccgaggcgggtggatcacctgaggtcagggttcaagaccagcctgaccaatatggtgaaaccctgtctctactcaaaatgcaaaaattagccaggcgtggtggtgtgtgcctgtagtcccagctacttgggaggctgagacaggagaatcgcttgaacccaggaggcagaggtcacagtgagccgacatcatgccactgtactccagcctgggcgacagagtaagactccatctcaaaaagaagaggccgggcacggtggctcacgcctgtaatcccagcactttgggaggccgaggcgggtggatcacggggtcaggagatggagaccatcctggctaaaacggtgaaaccctgtctctactaaaaatacaaaaaattaggtgggcgtggtggcgggcgcctgtagtcctagctactcgggagactgaggcaggagaatggcgtgaacccgggaggcagagcttacagtgagccgagattgcgccactgcactccagcctgggcgacagagcgagactccgtctcaaaaaaaaaaaaaaaaggatgtggattctgcattccagaagcattcgtgaagaattagatcagtgttgtctgggatagtctaagaccatggagatggtgatgttcaaagcggatccttttttttttttttttgatacagagtctctctctgttgcccaggctggagtgcactggtgtgatcttgcttcactgcaacctctgcctcctggcttcaagtgattctcttgcctcagcctcctgagtagctgagattacaggcgcatgccaccatgtccagctaatttttgtatttttttttttttagtagagacggggtttcaccacgttgaccaggctggtctggaactcctgtcctggtgatctacctgccttggcctcccaaagtgctgggattacaggcatgagccactgtgccgggcccaaagcggatcttttaaggaagggtgaatattacatgggaatgttagagaagggggagaatctgcccatcaagcaggcaattgtggacaaaggcatggaatttttttttttttttttttttgagacggagttgcccaggctggagtgcagtgacatgatctcggctcactgcaagctccgcctcccgggttcacgccattctcctgcctcagcctcccaagtagctgggactacaggcgcccaccaccatgcccggctaatttttttgtattttcagtagagacagggtttcaccgtgttagccaggatggtctcgatctcctgacctcgtcttctgcccgccttggcctcccaaagtgctgggattacaggcatgagccaccgcgcccggccggcatggatttattcacaaggttttagtattaaattctgtggatccctttggaggagattaatttttttttttttgaattggggtctcattctgtcaccccggttggagtgcagtggcccaatcatagctcattgtagcctcaaactcctgggctcaagggatcctcctgcctcagtctcctcagcagctgggactgcaggcatgcaccaccacacctggctaagagaactacctattttaatgtttcaaagaatttgggccaagggcagtggctcacacctataatcccagcactttgggaggccaaggcaggattgcttgagtccagcagttcaagaccagcttgggcagcataatgagaccccgtctctacaaaaaaataaaaaattagctgggtgtggtggcacatgcctgtagttccagctactcaggaggctgagacaggaagatcaactgagcccaggaggttgaggctgccagtgagctgtgatcacaccactgcactccagcctttgtgacaagagcgagatcctgtctcaaaaaagaaaagaattaatagactgggggagtggttgaggggaagaattcataggctgagcaacaaagcaagacccctgtctctccaaaaaaaaaaaaaaatttttagttgcatgtggttgcatgtgcctatagtccgtatggtcccaggtacttaggaggcagaggcaggaggatagattgagcccagaagttccaggcacagtgagccatgatcatgccactgccttccagcctgtgacagagcaagaccttgtcttcacacacacacacacacacacacacacacacacacaaaaagtttatgaagcaactggcttaatcatctggtctctacaggggggtaggaatctcatggaatccattttcttcaacagatagttttttgtttgtttgtttgttttgagacagaatcttgctctgtcacccaggctggagtacagtggcacgatctcagctcactgcaacctccgcctctcgggttcaagcagctctctgcctcagcctccgagtagctgggattacaggtgcctgctaccacgcccggctaatttttgtatttttagtagagacagagtttcaccatcttggccaggctggtcttgaactcctgaccttgtgatacactcgtctcagcctcccaaagtgctgggattacaggcatgagccaaccgcgcccggccaacagatagttctgactggcaataagaaggtacaaaaaaaggcccctatcagtggtgtcaaccattgccttagtgataaacactgaaagcacccccaaggctgaacccatgttttggaggtagttcactatgggctagggaagtattttccaaattttttttttttttttttttttttttgagacagagttttgctcttgtcgcccaggctgcagtgcaatggcgcgctctcggctcactgcaatctccgcctccagggttcaagcgattctcctgcctcagcctcctgagtggctgagattacaggcgcctgccaccacgtccatctaatttttgtatttttaatagagacggggggtttcaccatgttggccaggctggtctcgaactcctgacctcaggtgatccacctgccttggcctcccaaagtgctgggattacaggcatgactcactgcacctggctaacctttttttttttttttaacccattacctctaattaaaaacagaagctttaccctcactcctgtaatttttatatactactgtgtgagggccttgttgccagatatgaatcaataaaggggctagaagaatatagaaataggaaaaactagggcatgtttggtggcaaatctttaacctcttgagggtgatccctgtgaagaagccttcctactctgagtcttggggcgctaccaacagaaccaagataagggagctagaatggcagaaagatgatctggagcgaggatgactgtgccagagctggctggggagactgagcactgtcagtgcttgattaataatgtaagagaggacaggctgggccctggggacagcctcaccggcccataggccagagtcacagatggcccagttggcaacatgatggttcacttttggaaaagaaacgtgacacagcctttctgggaacagggtagccagtctgattcaggagagccaacaggatacccaagaactggacgtgagaggctctagccaccagaagggggcagcaccttcttgggatgacagattgaaaggcccgttaggctacccaagagggttgtggctgagtatttgggggcgttgcgggggctaagagggaggtggaatagagacagtacattttcaacaaaaaaatttttttaaagaaatgggggctacctttggcaggtgctgatgtatggacagccgtatcgtgttaccttagagcttgagctgccagagtcccctgtgaatcaagatttgggcatgttcttggtcaccatttcctgctacaccagaggtggccgaatcatctccacttcttcgcgttcggtaagtgttggtggcctgtctgagaaggtgtggaggaagatggcaggatcagaatgggtagggggctgggtgtggtggctcaggcctataatcccagcagtttgggaggccgaggcaggcagatcgcttgagcccaggagttcaagactggcctgggcaacatggcgaaacccatctctacaaaaaaagtttaaaaattagctgggcatggtggctcacggctgtagtcccagctactctagaggctgaggtgggaggatcgcttaaacctgggaggttgagtctgcagtgagccaagattgcaccactattctccctcctgggcaacagagcaagaccgtctcaaaaaaaaaaaaaaaaaaaaaaaaaaaagaatgtctgggtagggattcctcaggagaaaacacccttggctttgaggtcacagacacatcaggataaagaagggaatcaaggccgggcgcagtggctcatgcctgtaatcccaacactctgggaggccgaggcgggtagatcacctgaggtccagagttcgagaccagcctgaccaacatggagaaatcccgtctctactaaaaatacaaaattagctgggcatggtggtgcatgcttgtaatcccagctactcaggaagctgaggcaggagaattgcttgaacccgggaggcagaggttgtggtgagccaagatcgcgccactgcattccagcctgggcaacaagagcaaaactttgtcttaaaaaaaaaaagagaaaggaatcagcttacaaaaccagaaacctttgccaaacaccccaggtatctcaggagcatgcccagctttgggattaagtgtgaagagtggagctgctctcagaaaacaggctttagagccagacagacatgggcttgtgtcctcattcagaaacaagctagctgtgggtcacctgacttaacttcttgggtataaagtattataaaatgggtataatagtaactgcctcatagcaagactagtttggattcaatggggaaatgtaaaattcccattagcacatagtggctgtttaatgtttttttgtttttgagacagagatcttgctctgtcgcccaggctggagtgcagtggcgtgatctcggctcacggcaaccttcacctcctgggttcaagcgattctcctgcctcagcctctcaagtagctgggactacaggtgctcaccactacacctggctaatttttgtatttttagtggaggtggggtttcaccatgttgaccaggctggtctcgaactcctgatctcaaatgattcaaccacctcagcctcccaaagtgctgcaagtacaggtgtgagccaccgcacctggcctaataaatgttaagactccattcttccattaattaacaacacaacctcatcatgaagcaaattcctgatgtctacatgatggggggaaagcttcagatggtgcgctgggacttagatcccatactggattacagaactacttggtgggaaaggcagatcagagacagtgctgggcttaatctaggaaggtttcttggaaggaggggcctttgctctataatagagtgtggttgagtagggagagaggtactcaagcaattggaatggttggatgagactgaggggtaggaagtgaaggtgaggcagccagggacctgaggaagaagccgccttcatagactactcaggggtggttgaggggtaggtagggtcatggggatctggcatgcacctgtccccagcctctgcttcccacccccggcaggtgatgctgcattaccgctcagacctgctccagatgctggacacactggtcttctctagcctcctgctatttggctttgcagagcagaagcagctgctggaggtggaactctacgcagactatagagagaactcggtgagtgaggtgaacgagctttatgactaggagatagcccctcccattagcccaggattctacctgagaagcctccaggctcaggagacatcacaactgagagactggaagtggggctcagatgaggcgggtaagagtgctagcggcaggaccctggcctgacgccaccctcaccccaccccctcacagtacgtgccgaccactggagcgatcattgagatccacagcaagcgcatccagctgtatggagcctacctccgcatccacgcgcacttcactgggctcaggtgaggggccaactggagtgaaccttgggcaactcttcacgggggctaaccttccaccaagagggtcccaagcagaggtaatgggtttacagagcagagctgacttgggtttcacataggccagagggtctcaaagctgccacatattggcctatcagctccctggggaacttttatctaggcatcagaatttttaatttttgttttttgtccccccaaccctccatcatccctgttcatcttccctccgccctgccatccacccagaaatccagggctaagggctccacccaagtgagcagaattttttttttttttttttttaagagggggtcttgctctgttgcctaggctggagtgcagtggcgcgacctcggctcactgcaagctctgcctcccgggttcacaccattctcctgcctcagcttcctgagtagctgggactacaggcgcctgcgaccacacctggctaattttttttgtatttttggtagagatggggtttcaccgtgttagccaggatggtctcaatctcctgaccttgtgatccgtccaccttggcctcccaaagtgctaggattacaggcgtgagccaccgcgtccggcctgtgagcagaatgttttaaagcaccccgaggaaggctgatgggctgacagaattaagagctactcttttgaaccgttggcttagcaagagttggggtgactcttgagagacagacaacattgaggctgtggcatatggccctcccacaggagacttcccaaggtccttggcctttgttagaacctggtatagcaggttctggaaactgtgtgggtgggaggcaggggtgacagagccagctgtaaccaaactcacttgaattgccctagaaattatttctgggacatgagaaggctggtgctctctggtaactgctactaccttcttgtccctacttcctgcctcagatacctgctatacaacttcccgatgacctgcgccttcataggtgttgccagcaacttcaccttcctcagcgtcatcgtgctcttcagctacatgcagtgggtgtgggggggcatctggccccgacaccgcttctctttgcaggtcgaaaggggcaaggacccccttttgtccctttgatctttgtgggtggtcaggggtatgttggggattaaggcatgagggtcctgattgttagtgagggaagtcagtcctggctgctcagtagtttttcctccacaggttaacatccgaaaaagagacaattcccggaaggaagtccaacgaaggatctctgctcatcagccaggtaaaggtgttggctggggagctgttgaggtgggctaggttgagctgggctgggccagaaggagtccctgcttcatccagtatgaggcttagggtatcaccgactgagacaagggtcaaaggaaaggcgaggaaggtgaggttggcactgccgtccaggcacagccaccctaaccctaacctgtcctctccctctttggaggtgcagggcctgaaggccaggaggagtcaactccgcaatcagatgttacagaggatggtgagagccctgaagatccctcagggacaggtatggcgcagccaccacactctctccttcctgactccttggctttctccttcagtgtctgggtctgacccctgcattccccttttggctgcagagggtcagctgtccgaggaggagaaaccagatcagcagcccctgagcggagaagaggagctagagcctgaggccagtgatggtgaggggcatcctgcagcgtgacctgggtggggcccccggttaatctaatatcccatttgagattttgtttttccctttcctggcccaggttcaggctcctgggaagatgcagctttgctgacggaggccaacctgcctgctcctgctcctgcttctgcttctgcccctgtcctagagactctgggcagctctgaacctgctgggggtgctctccgacagcgccccacctgctctagttcctgaagaaaaggggcagactcctcacattccagcactttcccacctgactcctctcccctcgtttttccttcaataaactattttgtgtcagcttcttccttgactctg

SEQ ID NO: 3 - DNA, 57544 bp, Homo sapiens
gtagtttcccgcccttgggggcgcggggacaaattccttgacccgaggaggatagggatgtggccttcggtctttcctcgcagctccggggcaagctaggagtgggatggaagtcgaggtccctaattttttaaggggagggtgcggggagaaggggtagtatgcggaaacagagcgggtatgaagctggctaacgccgcgcgccccctcccaggacccgctcctgccccgcgccggccggtcctgggggcccgcttttttatggaaatgaggagggggggccggggccgggggcggggagccgggagccgtccccgctcgctcactgcggctttctctctcgctcccctctccccgctccctgccgcgctcactctccgcttccccctcccgctctcccagagtcgatcccggagcccggccgcggggagaggttctcggcagagaagacaaagcccgcagcagcgatggggggagagctgggctctgcgtgttgtgggggccaggaaagggtgccaggctggggctggaaccccctggcaaaggatggggtcccctcatccctaaacagcaagccatctcccctcgcccgccccccgcccccccagtctcggagatctcagaggcaccgactgggaggtgagttagttcacgtccttctgctcggtggagggggtcgggagggcggtggagtgatgaagtgcagaggttgaaggagtgcagggacagaactgggggtcggacggagagtgggcaggcaggaaaagtcttaaaggcctggggtgacttaaccgtttggggtgagggccttgagctttggatatgacaggagggaggagagaccaggatggactgggaacccaggcttccttcaggcctgggctgtgaatacagcttggggtgatctgtcctttagcactcatcgtaatttccactctgtttcctccacctctctcccattgaaaacctattcagggccgggcgcggtggctcacgcctgtaatcccagcacttcgggaggcagaggcgggtggatcacctgaggtcaggagttcgagaccagcctgaccaacaaggtgaaacctcgtctctactaaaaatacaaaaattagccgagcatggtggcagacgcctgtagtcccagctactcaggaggctgagacaggagaattgcttgaacccgggaggcggaagttgcagtgagtctagatcgcagagtgagactccgtcccccccacaaaaaaacaaaaacaaaacaaacaaacaaaaaaacccattcagtcccgtttgactccggaaattcggtgagaatgaaaataagtttttaaggcttggagagttaggcacttttagagtttcaggaaggtggagtgtaatgcaattttttcttctccctttaccccatattttctgcatttattttatatcaccctttttatgtctggatctttcccaagggaagaagggatggcactgcagcttgagggctcgaggtcagacttgaagaagaacttccataaagagttttataatagtgggtgggtgagccacagtggtaaggtgtccttttctggagtgacttgaaaaacaaaaatctgaactagtttgtaagataaagtcctgctgagaggcaaggaaacagatgaattgacttcacagacttagcaatgtggggccttgagggctgagctgtttagtccagttccctaaactgtttcttggaaagagtgaatttgcacactaatgggagtgctggaacctgagctgaggatctcagaaggtcggggggtggtgggggcagttggcacactgcagggatttttttcctttgctaggcagaagatccagggaaggagaaccttgactgaggaacccgggagggtcctggagttggggaagtttgccccctggactggggatttaacataagatcaacagtaaaccttattgtgggatttgatgctactgccaccaagcaagagtgaaagaggaagaattagagagacagggccctggagagtgggctatttctgcctcttgtcccctccgtctgccccaggaacctggattgcagagactgtggtactggggaccatggggtctctttgggagaaagtttttgctttgtggaccaggtgactatctttcacataaatagaaatagcaaacagttgcataggtcttattgtgtgctaggcatttttgtggggccaccagctggctgagccttcctgtccccactgaaatgagattccccccagactcccaccctgaacttgcgctgcctctgctttcctcctcactaagctaattaaggcaggaggttaatgaccaaagaagccttggggatttggccagggaggaaaacaaagggcagaaacaagtatgtgtcagtggggagggtccagctgctgaatggggaaggggctggctgaaaggtggggtgggcgaggcagggtccctactggacccacttcgtccctgacccaccaacaagaatgaatcagccctctgggtaagttaatgtccaccctcagggtcccactgggactccagacaaatttccttcacagcagatgtgagcctggcacctggagggcaaaggatatgtgtgcgaatgcagttaggtgtgtggcatggtgtacatgtgagagtgtgtgctgcagatcatgtaggagagtgcccaggtgcagaggaggcccagggtgggcaccttgaggagctggattctcagagtgttttttacgaatccttcgctctctcccggaggctggtggcctcctttgttcaggagaccccctccctcaactgagaaggtagatttctgtatacaggctctccttttccctgcttgcctgatccagaattatggcaatgagagcagaaggtcttacttaaacaatactccccactctctgccttatgaccagaaaggtgtagaggtggcatacttctccctttcaggactttggaggacagcactatgtctctcatcttcatcatggtccccaaccctagctgcccagaacctagcatgcagccctgcccagagcagaggctcaataggggtatgttgagtgtaactgcatttttaaaacttcatggtgtcaggaggaaagagcaggtctccaaagataaacatgctgcttattctgccactttggggagtcatttattgctaagcctcagtttccttatctctcaaatggaaataataagccaaggtctggcagggcatggtggctcgttcctgtaatcccagcactttgggaggccaaggtgggtgaatcacttgaggccacgagttccagaccagcctgggcaacatggcaaaaccctgtctctactaaaaatacaaaaattagctgggcatggtggtgcacacctgtaatcgcagctacttgggaggctgaggcatgagagttgcttgaaccaggaagcagaggttgcagccaagattgtgccattgcactccagcctgggcaacagagggagactccgtctcaaaaaataaaataaaataagccaaggtcctaacacagtgcctggctatcttaggtgttcaatcagttttagtttcctggccctggttgttctccccttgtggttgactttagccagattgtggtcttcatgaattttcattcattcgttcattcattcagccagccacttcctattgagctcctgggatgtgccaggcactggaggagatttattctgacccatcataggtcacttgaagtcacttctaggtctagactcgctgaaataaggtcattctaaacaaacagcaaataattctaaaaataattcctgttaagccctggaatgcaggtgacctctgactctctctctctctctctctctctctctctcttttgtctttcttacacaagccaggcttctggcctcatgcctggggaacaaaggaccctgttgagttgtttagtacatacacacacacaagccaggctgcagggcaggaaaggtgacacagtggagagtccaggattcttgtggaggttgggggaggagtggggtggatgatccagactgtgtgcttgtctgtgtctggagttgtatcccttcttcttccttttttttccttttgagacagagtctcgctctgttgccaggctggagtgcagtggcacgatcttggcccactgcaacctccgcctcctgggttcaagctattctcctgcctcagcctcctgagtagctggcattacagcatgcaccaccactcctggctaattcttgtacttttagtagagacggggtttcaccatcttgaccaggctggtctcaaactcctgacctcaggtgatccgcccccaccttggcctcccaaagtactgggattacaggcgtgagccaccgcgcccggccaggaattatatcccttctttcctctgagcacttctgggcacctgttatcactgtctgaacactcagtaccaagggatgggggtgcacacctaacacttgcccctccttagccctattctcagaaccctgagggagggagacacaggaggctgccaccaaggctgccttaccaagcagactggcccttaaagaggtcatctcccccatcttcatacttctctcaggatgccttctctatctgtcctagttagctgagccttctctcccatgtataatgagattttcccattttcttttctttcctttttttttttttgtggggggacagagtcttgctctgtcacccaggctggagtgcagtggcgccatctcggctcactgcaacctccgcctcccaggttcaagcagttctctgcctcagcctcctgagtagctgggattacagatgcctgccaccacgcccggctaatttttgtatttttagtagagacagggtttcatcatgttgcccaggctgatcttgagctcctgaccttgtgatccacccacctcagcctcccaaagtgctggaattacagtcgtgagccatcgcgcccggctcccattttcttttcttttcttttctttttttgagacaaggtcttgctctgtcacccagactggaatgcagtggcttgatcatggctcactgaagccttgatctcccaggataaagtgatcctcccacctcagcctctccaatagctgaaccacaggcacatgctgccatacccagccaatttttaaattttttgtagagatgagatctcgctatgttgcccagtctggtcttgaactcctggcctcaggtgatcctcccacttcagcctcccaaagtgctgcgattataggaatgagccaccatgcccagcctttctttccttttttttctttctttcttttttttttttgagatggtgtctcgctcggtcaccaggctggggcgcagtggcaccatctcagctcactgcaacctccgcctcctgggttcaagtgattctcctgcctcagcctcctgagtagctgggactacaggcgcgcaccaccacacccagctaatttttgtatttttagtagagacagggtttcaacatgttggccaggatggtctgtatctcttgacctcgtgatccgcccgcctcggcctcccaaagtgctgggattacaggtgtgagccaccagcccggccccagcctttttttccttaataaccctttcttgtgggaagtctgtgttgctatctaaccaccgtcctttctgctaagagtccagacagttttctctcctttgatagaatgcactcaacacattaattaatttaatttaatttttttgttttcttttttttaaccttataccagcagtccaaacctcactagcattaattaattttaatcaatgagaatatggaataaaattctgtaggccacacaaaagaaatgtgagacttgatccccaccctcggtaagacacagaaactggcctgcatgacccagagaacatgggtaagcaggattaaatgtaaggacaaaatggtctttggggtagagcacggtggctcctgcctataatcccagcaccttggggccaaggtggaaggattgcttgagcccaggagtttgagaccagcttgggcaacaaagtgagacctcatctaatttatttttattttattttatatattttttgagacggagtctcgctctgttgcccaggctggagtgcagtagcacaatctcagctcactggagcctctgcctcctggattcaagcaattctcctgcctcagcctcccaagtagctgggattacaggcatgtgccaccacaactggctacttgtttttgttatttttagtagagatggggtttcaccgtgttagccaggatggtctcaatctcctgacctcatgatccacctgcctcagtctcccaaagtgctgatattataggcgtgagccaccgcacccagcctattttattttattgattaatttatttgtttgtttgtttattttgagccagggtcttgctctgtcgtccaggctataatactgcggcatgatcttgactcactgaaacttcgaactcccaggctcaagtgagcctcccacctcagcttcctgagttgtttggtctacaggcatgcgcaccctgcctggctaatttttgtattttttgtagagatggggttttgccatgttggccaggctggtgtcaaactcctgagctcaagtgatccgcctacctcagcctctcaaagtgctgggattgcaggcatgtaccaccatacccaacccttgtttatttatttatttatttatttatttatttttgagatgaagtcttgctctgttgctcaggctggaatgcagtggtgtgatcttggctcactgcaacctctgactcctgggttcaagcgattctcctgcctcagcctcctgagtacctgggattacaggcgcgcacccccacgcccagctaatttttgtatttttagtagagacggggttttaccatgttggtcaggctggtctcgaactcctgacttcatgatctgcccgccttggcctccattgcactccagcctgggcagcaagagccaaactccgtctaaaaaaaaaaaagaaagaaagaaagaaagaaaggtctttgggactcggagggctacagaggttgggggagagaggtcactgtgagtccttctgttgttgtggatctgatctccaagggctttgcaggctcagcctaggtttggagatggacaaacgtctctgcttctcccctgggactacactggcacttcctctgccaggtcccctgcattgtctgatttccccagggagagtagctcagaggggctggggtggagtctgggccccttaggacagctggttgatgaggcccctctgggacttagggctggacccaggcgtccaggaggcgggtgatactggagctgtgacagttggtccccagattccacacccagtggcgccagctctggtttcgagctgcggtgagggagggggaggggcacctggtgccagccaggggctggatgctgtcttggggcagggagggggaggcagtaaacatgtccttcctaggttgttgaggattttctaattgccctctctgtgccaggcactgtgcaaggtactaacgagattctgcaatgcaagatgcagtctggcacagatgaattgacagcatacagtatagaattagaatgtcagagctggactagaagttgaatgaatgaatgagtaggggaatgagtgaatgaatagttcatcagttacctaatgcactcattttacagaggagactgaggcccagagaacccgagtcctgtgactcccttctgctttctgatctgtcatgcttcctttttcaggaaagggaagcaaaaggcagagagaggggtgggaagggacttgacgtttcctgagtgcatgtttggtgccaggctctgttctggtcttttcagacttgatgtaattgtcttcacaattctgtgaagatggacttggtgtctctgcttcacatgaagggacctgaggttcagagaggctaagtaactttcctaggtcacagagctggtaagtagagactttagtgttcaaaaccagagcagtctgaccccagagcctgggaccccagaggtagacagtgatactcagtgagtgcttggagcccacagagggtggagggagtggttcctggatgaagtagatctgaagggtgggtgaggttcatttgaaaggcgagaattggaggcatgtctggaagttggtgctggaggtgcccagggagacttgctggttgagcaggtggccggcttgcatggatcttggggagcaggggacaggttcaaagaaacacatctttaaacattggccaccaggtggcgccgcaggccaattcctagaagcggctccagcctcagcgccctcactagggcccaggtatgacttcgctgctcagccagggctccatgctcggcttgcttcctcaggtgtcatctccagcttccgcttgcccctggcttctggcatccaacacatgggggataatgatgctaccagtcgtttcctgactcgggacgaacacccaggtagctgagacctgggaatccccgtgggtctgagcagctcccccggtcacccggagacccctccagtcgcctaaacctggcttcatgtccagcgcccttctgagtttctgggagacactgttcagaggacaggctgaggaaacaggcatttgagtccaactgacctgggtctgaagggaaataataatatctaccttgtaaggatttaaaacatttcatctatggccaggtgcggtggctcacccctgtaatcccagcactttgggaggccgaggcgggcagatcacctgaggtggggagttcgagaccagcctgaccaacatggagaaacccggtctctactaaaaatacaaaattagccgggcatggtggcgcatacctgtaatcccagctactcaggaggctgaggcaggagaatctcttgaacctgggagacagaggttgcggtaagccaagatcgcgccactgcactccagcctgggcaataacagcaaaactccgtctcaaaaacaaacaaacaaacaaacaaacctttcatccaataaatatctatcaaatacatgctgtgtcaggaactattctaggccctgaatacatcagagaacaagatagacaaggtccctacttgcacggagctgatactctactggggaggaaggcagacagtagtcaagtagataatgtactttctggtagtgatcatcactgcaaagaaagataagccaggatatgggggcagaaaggtttgaggtggctaatttagaaaggatggtcatgctatctggccaggacaacatagtgagaccccatctctacaaaaaaaaattttttttttttgagatggagttttgctcttgttgcccaggctggagtgcagtggcatgattttggctcactgcaacctccgcctcctgggttcaagccattctcctgcctcggcctccctagtagctgggattacaggtatgcatcaccacaccgggcaaatttttgtatttttagtagagatggggtttcgccatcttggtcagactggtctcaaactcctgacctcaggtaatccactcaccttggcctcccaaagtgctgggattacaggcgtgagccacagtgcccagccaaaaaaaattttttttaattagccaggtgtggtgagttgctcttgtagtcttagctacttaggaggctgagataggaggagcacttgagttcaaggttacagtgagctgtgatcgtgccactgcactccagattgggtgacagagcgagatcccatcactattcaaaaatttacaaaaaggctgggcacagtggctcctgtctgtaatccaagcactttgggaggccgaggcaggtggatcacttgaggtcgggagtttgagaccagcctgggcaatatggcaaaaccccgtctctacaaaaaatacaaaaattagctgggcgtggtggtgggtacctgtaatcccagctacttgggaggctgaggcaggagaattgcttgaagttgagaggtggaggttgcagtaagctgtgattgcaccactgcactccggctctagacaacagagcgagactccatctcaaaaaaaaaaaaaaaatgtgatcatgaaatgcccctgataaaagcaccatcttagttccccgttgccatcagattcacactccttagtgtggtgatgatgtccctcttgtcctggccagctcctgcccacctctccagcccatcctgctcctccttcccttactccagccacatggactttctcctttctttcttttcttttctttttttcttttttttttttttttgagacagaggagtcttgctgtgtttatgctggagtgcagtggtgcgatctcggctcactgcaacttccgcctcctgggttcaagcaattctcctgcctcagcctcccaagtagctaggattacagatgcctgccaccacgctcagctaattttttgtattttttgtagagacagtgtttcaccatgttggccaggctggttcgaactcctgacttcaggtgatctgcccaccttggcccccccaaagtgctgggattacaggtgtgagccaccactcccagcccacatggactttctttcagggctttggcctgtgctatcatcatctgctattccctgacccttcacctggctaatgtttgttcatcttgcaggactcaatttatatgtcaattttttttttttttttttttttttttagataggttctggctctgtcacccaggctagagtgcagtggcacgatcttggcttactgcaccctctgcctcctggctcaagccatcctctcacctcagcctcccaagtagctgggattacaggcgtgcgccaccatgctcagctaatttttgtaatttttttagagacagggtttcaccatattgcccaggctgatctcaaactcctgaactcaagtgatctgcccgccttggcctcccaaagtgctgggattactggtgtgagccactgcgtccaactacatgtcactttttcctagaaatctcccctggtcacttctggatatcccttacaattcatcatgctctattgtaactgtatgtttacacttctgtttccccagtacacgggaagctctttgagaacagggattggatctagtctactcctgtattctcagcacccagcacaaagtaggtgcttaataaatgtttgctgttcggaaattgtgtatgctcatccatccttccctcttgaacaccctctcacccatatattgccaaatattccacttctccctctgcttttctggcatacctttcactcccacatccagctccatctctcctctccccttccatccccactcctacacctgggccctcgcagcctgccctcatctgatctccaaggggctactgtctgcatgaggtttagtatatcctgtcactgggttcctccactctccagcacctggtctggggccgtacacagcaggaactccatagtgttgctaactcactgacatgacttggtggatggctcagaagggatccctaagcatgcatacacctcctctcagtcaccgctagtaaaatgagtctatcacgagaagtatacatgggtcacatgctcctggccaggccctgtctatcctctagggctttgcacacactgttccctctgcctcgaaccctctctctcattttggctatctcccactcatccttcagacctgagctgaagtggaggagtcatgtcttctctttttttgagacagtcttgctgtgtcacccaggctggagtgcagtggtgcagtcttggctcactgcaacctctgcctcccgggttcaagtgattcttctgcctcagcctcccgagtagctgggactacagaggcgtgccaccatgcctggctaatttttgtatttttagtagagacagggttttaccatattggccagggctttggcctgtgctatctcaaactcctgacctcgtgatctgcccaccttggcctcccaaagtgctgggattacaggcgtgagccaccgcacctggccaaggaggagttgtgtcttctgagaggccttcctgaccatgctgatggtcttacatatacccccaagtctgctgtggcactcagtatttacccatttgttagcacttagctcctagtgctttctttttttttgatggggtcttgtgctgtcacccaggctggagtgcagtggcatgatctcggctcactgcaacctccgcctcctgggttcaagtgattctcctgcctcagcctcccaagtagctgagattacaggcgtttgtcaccacaaccagctaatttttttttttttttttgagaacggagtcttgctctgtcgcccaggctggagtacagtggcgtgcactcactgcaacttccgccttgctggttcaagtgattctcctgcctcagcctcccaagtagctgggactacaggcgcacgccaccatgcccggctaccggctaatttttgtatttttattagagatgtggttgcaccatgttggccaggctggtcttgaactcctgacctcaggtgatctgtcctcctcagcctcccaaagtgatgggattacaggcgttagccactgcacccggccaactcccagtgctttcaaagccagtttgtgtgttcatgttactcggactgatcgctctccatgaataagggatgtgtctctccaaaacctagcatataggaggcattcaataaaaatttattgagtaaatgattgattgaatgaatgaaaggaataaaatgaccatgtgatactgaatgagtcactccccatctcagtttcctcttctcaagtgattctcctgccttagcctcctgagtagctaggattacaggcacctgccaccacacccagctaatgctttttttttttttcttttttgtatttttagtagagacggggtttcaccaggttgggcaggctggtctcgaactcctgacctcaggtgatccacccgcctcggcctctcaaagtgctgggattacaggcgtgagccactgcgcccggtgtccaagacacttctaacgccaacatccttgtactaggccctcttccagctcccccaggagtctgggggttagaggtcaggctgctccccactctattacctccccacacttctaattaattcttgtgttcagtcccagaacctggagagttacactcaggctgcctggtcctgcttattgcccatatcctagaggcacttgcatgcccttccaggcgggtaactgtacatgtctctcccaggcagccttcacagactactcaggggtatggattggctccagctctgacactcagacctgagcagaggcgtgatctactctggcctcctgtgtttctgtgccccaccttcccacactgcccagggctccctgagagtgatgctgagtctagtctcagcttttatgttgctttgcttttgacctctgacccaacacatgcctcctttccctctgaagtcttgattttctccctggcttttctccaccttctgggtgttcctttttggccttctttgcccatctctcctcctctgtctacacagccaggggctcccccttccgtttcctctgcctggaacacacttgcttctccctttctgactggctattcaagcaggatcctgtaggtgggtggcagtgcactgtgtattacctgtgagccctatgcaaattaccgcacatctctgaacctgttttcccatctataaagtgaagctaatagtagcaattacctcatgggattgttgtgcaattagtgggaagaggcatgtaaagtactgacatactgtctagtacacagaaagtgcttaataaatgttggtgattattatccaggactcagcttaatcatcccttcctcaggcaggcctgccacacaaccgcagaccgggttaggtgccgggttttctgctccccagcgtttggaggcactcatcacacattaattactttatccatgaagatgaaagctgtctttggcacccagatggtactgagtaaatatctgtgaatgaatgaatgatgtggaatgtctcctctggaaggacacagtcagggcctcctgccactcaggactagcctggatgcacccacagggaaggagaaggtggggctggtctccgctgggccagcaggtggcgccagggcctgtcggtctctccccttggccacctcccttgcgcttgctcatctcatgcccaggcgctgagggttctcagctggggcaggagagatggggtcctgaggcctgggtgaagttccaagccctggggctcgggggcagactctcgctcacccctcccagaaagatgttttcctaggggatgtccagcctaacacggcagggaaggaagtggtggtcacctgggaaaggggccaacctgagaggttgggggtcctgatgaggctgctattctcttgaggacatgaaatgagaggcggtgaagtcagtcagtgagtcaaaagctgagatctaggaaccagacacaatgtgggagaagctgcggtgaaatcaatcagtgagagaggaaggggaggcccggtttccatgacaacggtagtgtttgtggcaagctggggactcctgggtccccagggcccgcaagcagtgattcaccacaggaaagggaaggctgcagagtgacctggtgctggggaccggatgccttgtggctccttctcactgctgagcatgcattcccccagcccaggccttggatctctgctctgccagctactggcagggaaactgagggatggcagaactgagccccaataaggggcaggcgctttgctccacgccgtacatgcacatatcacttatatcacttattgctcacaacagtcctatggaattaccgttatccctgttttatcaaagaggaaactctcagcctggcccacggtgccatgggcaggacatgagtggcagagccaggattggaacccaggctagcctgactctttaccatgcttgggaaggcccagaaagcagaaaaggaagagcaggagcatgagagttagaggatggttcagaccccagaggagatggaccccaaggcccctagcctccctgcctgccctgccctatgcgagcccaacactgatacggaggaaagctggccaactgcatggcctaatgtgggccagaacctgggcagccagcctacctctcactctcctccaagcccaagagagtaggccccagttccagtccatccaactggatggtagctcccccaccgttttctctctgccctggtcctaggggcaggggtgcgtgtgcgtgtgtgcgtgcgtgtgtgtgtgtgtgtgatcatcatgtctacaccacccaccaccatcacccctatcctccagcttctgctatagcctgggagttcccagccttttagtccctgtcaggatagtttcattcttgttcagctttggggcccacacaatttcagtacaaaaggggtatgcccatctcaccctgggccttgatagcatgcaagagcatagttcaggccctcatcatctattcccttgtagtagaagggatggaggttggaaagaccttagaaatcttccagtctggttctcttgttttgaaggtgtgaaagctgaggctcatagaaggggagcaatttttctgaggtcacagagcaagttaatggaagagtgaactagattccctgtccacagacttcaggttctgtaatcccagaaaggacttcttgaatttggcattgagttcttaggatgggagtcaggtagatgaacagatggagtgagacagatgaaattggataacctagcataaaatgccatgtgaaaagaaggtacgcaactcatggcccgactgtctaccatgttacatcaggaataactgaggtgagatcatgggaaagacttctcacatatgtatgttaggggtacaagatctgtggtcaaaatgggctgtgtgatcccagacaaatcacttaccttttctgagccccagtggtaactcaggataccctacctgcaggcttgtggagaggatggagggggtggtgggagagtacttgtgaactgtggggtgacatgaatgttgtcagttgtcagggaaggcaagtcatgtctagtgggactgagatagagctaccctgcagtgcaaaggtaaggggtaagccatgatgctcacccctcccacccccatccttgggagagaaagggaggaacataaacttccagtatgcttgatacaagcaacagttctccctgggggtgggcagcaggttcctgtagacccctggggtgcccagaggctgtgggaagctgagcactaattagctctggctttctaatcgggcttaattgcctcctcatgatgggcaattaggaagctgcatcaattagcacagggaggtgacaactcagctggggttggtcagggctccccgttggcagggagggaggcaggcaggcaaaggctgccccactgtcagccccttccttccctttgaaggagagggccctggagccctctccttagcaattaggccttagttccttagctcctaggagctactagccttctaggcctggattgtggcttttctggccttggtttattcaccttcactggctctctgacttttctgcccagaggagacttgcattctgggcctagctccaccactggctgctttgcaactttggggttgacagctatggagtcaaggttgagaatgtggcctttgaagctaggtctctagggtttgaatcccagctccaccgtttattaattgtgcaacattggaccctttgtgctttgttttctcacctgtaaaatggaactgataataaaagttctgtcctagctacacatgaggctggggcaggaggattgcttgagcccaggagttttaaggctgcagtgagggacaatagagtaagacaccatctctaaaaaataatttaggccaggcatagtggctcacaccgtaatcctagcactttaggaggccgaggtggtggatcatctgagcttgggagttcaagaccagcctggccaacatggcgaaaaccctacagagtctactaaaaatacaaaaattagctgggcgtgctggtgtgcacctgtaatctcagctactcaggaggctgaggcaaaagaattgcttgagctcaggaggcagaggctgcagtgagccgagatggtgccactgcactccagcctgggtgacaaaaaaaaaaaaaaaaaaaagaaaaatgcccatgtgtcaccccagatcaattactaatcaaaatatccaagactttgggcatttgcagttttgaaagcccccttcctccaagcaatgttgatgcccagtgagggttgagatcactagatgaatggtctggagggctccatccagcctggcatagcacctccctgtcacataccactcaggacccctctgcatagccctggaggtgctgcactttggaagagagctcattaccaccagtgtggctggaagcaggttcctctccactcacttagagggagttctcctctataaaatgagaataatgaccttgccatgctcacctcccagggctgcccagacataatgttttactagtgataggacctgctttataggtaggcaattatattgttccttcccttgaaaaacttttatcgaggtctagtgcacacccatctcctttacacatggtgactatggagttaataatctcctggagggacctgtcctgaaccatcagacaagttaggtcatagggccaacccccttttttagctcctgctggttatgaaatgagccaaggaatggaaagagcctagaacaggtcctgacacatggtgagtgctctctaaacagtagctctccttactattgttattgtgaccgtgccccaggctaggttggagggaggcctggttgcagtgcacacagggagctcagatgaagggaggcagtgaaggtggggcctgcaggaggaaggggagtttagaatgaggtgctgaggagtaaggtgtgtgggaagagtagaggaaggagtagagcacatcgaggttgctggttggtgagggggctatgatgaggatactctactcaaacatgtattgagaaccttttgtgcaccaggcactgtgccaggtgctggcagagcaccagatccacccagtctctacccttgaggagtccctctaatgggggaagacacacatacacagacaatgtcacagacagtgtgacaagggatactggtggcgagtggggacatttaatccagacttagtggcacttttagctgagcctggatgatgagtaggagttgatcagagggtaaagtaaatgccatgtctggaaaaattagtcagaaagcaatgagagaggttgagattgctggaatttagataaggctagagatgcaagcagagcctaggtctcggagggcattacaagtcaggtcaaggaatttggacttatcctaaatacaatgtggagccactgttagtttaaagtcttctggaaacaaaatgaggctttcattttgaaaagatcgctgtgggctgggcgtggcacctcacgcctataatcccaacacttttaggaggtcaaggtggaaggatctctttgagccccgcagttcgagaatagcctgggcaacaaagtgagaccccagctctacaaataaaaataaaaataattagttggttgtggttgcatgaacctggtcccagctactcaggaggctgaggcaggaggatcacttgagcccatgaattttggattgcagtgagctatgattgcaccaatgcactccagcctgctggatgacaaagcaagatcctgtctcaaaaggagagagggaaggaaggaagggagggagggaatcactgtgggaaggagaaacaatgggagcagaggcaagggtggaggcagagagactggttgggaggctgctgcagttacccaggaaggaaggacggaaggaaggaagggagggagggagggagggagggagggagggagcgagggaatcactatggtttatagaggagaaaggatgggagcagaggcaagggtggaggcagagagactggttgtgaggccgctgcagttatccaggtgggggttggtggtgagcagaccagagagctggaggtagaacatagaacagataacaattctggaggtaggaaccacaaggacttgggggctcattggccaagggagatgaaggaggagaggctgaggaaggaggagcagagtgaagctgtgtgttgggagacatgaactccatctgagatatccaaggagtgatgatgagttatggatgcttaggaatcactcctggtctgggaatacttggggtcgtcttctaagaattctcaagagtggaaggccactcacccttggcccagtgccaggccctcgccttccctagtctctctgggacaggctgggaattcctggctcagaaataccctgttggggcaggccggccttgatgctccccagcccaaagggaggaatgtgcctgtgcacctgctccgtgatacacgggggcgggacagtggagtggaggtgagtgaattcaggggtgttcctcacttcccccatcctggacttctaggctcagttgtggccccaggaatggattctgagtaagaggccagaaagcaagttctctgggcctggggtacagagggcagcaaggtacagttgacataccgcagacacccaggcagggcaagggcacccaggtttcagtcctggttctgtcacctctggttaggagctgtgagcacccaagggctcattcttgtcctaaaactgggtcttcaagatggtttggtttaggtggtgtgaatgaacccttccatctccaccctcgagagagctgaattttcccccacccgaggcgttccacagtgggcagaacgccgcagcccagtctgccctggcccacccagtgctcatctccatttccagcccctcctgggcgcctctggcctcctgcagccggctctttgcgctccttcattaagctttggtttctgtgggaatgtgacttgaattaactcttgggagaaaaatcacagaaatcaggaaaggggggccatggggagggggcagtgagtcacgtttccatgtgtatttttcaaactttccgatgggagtttggaaaacaacagtcttttggttgccagcttgggcccagctctccaagggtcccatatgctgtgtctataggtcttcccgtaactgctgcgtcccggcccacccttcgggtctgccttatttcctgggatcccttcccacttctccctggggacttgtgctgttgcagaggcactgagaagtccatcccgtggtgcagcttccgtctctcccctggtttcatttcacagccaagccctcatctatccttgcctcaaggtggacccatgggggctgcagctgagctggggactttgtccctgcagctgctctggccaaccctccaaccctggagaacctccataggcaccaggaagtcctgggctctaggaacctccttcctgcccccacagcttcctctttgtgtcctgcctaccagtccttcccgcccatctctgcatcccagtttccagagggcctgtggctgagggtgagaagccagcgatcgacccccagggagaagtcttgcagaggagggtcctcatcctaatgaggccgccaaggaagagggcccccagtatgcccctcttgtgtgctgggccctcgcacttcctctgtctgggactcctctaacagctgtggggagggtgccaggatccccattttgcatacagtagctgaggctcagccaggaaaatgggtgggaaccttaaggatggtgggcagatggtggcccccttgaggaggagatgggagggcagcgcatacccctagccaacccctccttgcaagccctcattcgggcggggagaaggaaggggtcctggactagctctcctgtgtcccctctccgggggctgccccctcccagtttctgactaatcctttccatcggcagtggcatgtcctgcccctgctgtgcctgtgccaacaacagcccacatcacagggccacatctggctccatttgaacataccccaccctccctgctctttatccctcaacgccctcccctgcccaaggaccagagtagggagtggccctctggacccccttctctggctccaccagccctgctcccctgccctttcgaagcctctagtggagtcactcctttccttccccgaacccggcctcagttcctgggacatcctggctccattcttcagcacaccctccctcatcatatccacactccttggctccccccttcacagcccccactgaaggagggattgggaaggggacattttgcaaggtctgagccccaagagatgtcccagagtagagggaaggcctggcagcccagggattttcccttagcccagctctctggcatattgccagcttgggcgtgttgggcggcaggggtcggggtgatcccaagaggtgcgtgtatggaggggtatagctcagcctcccagctcgggtggggagcggtggctcaggcctgtgtaggctggcttttgttggggaggagcctggaagggcctgcagctactggcctccctcctccttcctccttgcttagcaactgttgtcgtctggtaaatatttgccccaacaggatctggggctggagcactggcgtcagccgaggtagttgccccctcctcaatttatgagtctcccctctgttcagttccctattccagagccccctggactggattctaaatgtggtcctcaccccctccctcatggttgatttcctccccaacctcttccctgtctcccttttactctccttcatccccactcatcctctgtgcccaggtctctctcgtccatcctccactcctggatccattcaccaaggggcctgagtgaggtcccaccccctaagccacacaggtcctctgcttctcccccgctacatctcactgggaccccagcctggcaacggctagtgcgtcgtcggtgctcagttagccccaacatctctttcctttcctccaacaggaaacacaccttccacctctaaaccttagctccaccaagctccaaggggagagaagagagggcatatgggaatgttttgctggaccccatactgcactcccaggccaggaagctctgcatcaggaagccagcaccattttcacctcctctggggtaggactgaggggaccatggccaagaggaaacagatgcccccttagctcctccctgggtagcctgagcgggccagggcctgagagcatgccagttttagcctgtcttcctgtccttcccagccagacccctctcatcctcctcccccagtggttttctcattacctgtcactgacggagagcccctcagaggtcaaggccaaagtgaggtgggctcttggcatgctgtgcaagcttggagctcacccggcaaagagggccaatggggtgctcctctggggcgggcctgtgggctggcttagggatagcagacaggggaatctggggagttggactaatgtggatctagaagggaggtggttgggctgcacgatgccttaagactcctcccagctctgaagttctacttctaggcaatgaatgatgaaacgctggcccaagtgcagtcccttccccatcccacagggcggagaccccagccatccctactctacttggcagcccccctccccactcctcctgagagtgccccttgctccaccccagcggccaggaaatctcctgcagattcaccaccaccctcctggctgggagttcactttcctagttgacctcctggcctgagggccagaggagagctttcaacggggaccttgaggagtgtgagggctgcaggggctgtggactggagtggattcaccagggagcaaaggaaagtgaagtttcagggcctctcagttgcacgggccctttcaagacccgaagaggagccctgccaaactttccacatgatcccatgtccataacatttgcaaaaagatcattttgtatttcttaaagagggcctccccacaaaattgtgtgcactttaggtaggatgaacaactgtccctattttagcattaaaagtcccttatctggccaggcacggtggctcacgcctgtaatcccagcactttgggaggccgaggcaggcggatcacgaggccaggagaccgagaccatcctggctaacacggtgaaaccccgtctctacttaaaaaaaaacagaaaacaaaaaacaaaaaaatagcggggcgtggtggctggctcctgtagtcccaggtactagggaggctgaggccggagaatggcgtgaacccgggaggcggggcttgcagtgagccgagatcgcgccactacactccagcctgggagacacagcgagactccgtctcaaaaaaaaaaaaaaattcccttatcctaggagactcctcagtccccggcaaacccagggcaattgcccaccctagtttcaggctccacaaacatttggcccacactgggttgagggtgagggagaaagggaatccttttctgcctcacttcctggaaatgtgtaggggcgtgagctatggggcgagggggccctctcctccagctccagtcctggagttgtggcgcttctgtttgtttccttggcaacagggaaaggaaacccccatttaggaggccggggaggccccagcaaccacacacacacagagacacacagactcacacgcggccgcccacacgcagtcgcctgcgtcagcaccagccgtcaggccctcgctgcccgcctgcggggtgtggagtggggagaacagccccgcctgaatccagtccgactacccgcccctactgatttgcctgggacgccgccggcccacatggccccacccacgcctctgtccccacccccccacgcctctgtccccacccccccaccgcccccggcagcgttccccccttaacaccttcccccaggtggtgaggggccaaagctcggggatggacaagagaaaggaaggagatgtcactgctgaatttctgctgaatttccttctgcctgctgcccgatttctatttgactggcaatagtctggtgtgtattgtgacagttcagggagcctgagactcacaagtgaggtggcgatttgggcctggttgggtgggggaggtggtttggaatgccgggaatggagctcctggggagactgtaggattttgctgttggattgggttccttgggatctgggtcgaagagggaaagagtatatttaacctggaggcagaaacctggggtttgggcctgcttctgtcacttgtcagctgtgtggcctggagacaatcagttagcctgcctaagcctctgtctcctcagctgtaatatgcagataatggtgcttgcctgtgcacctcaaagaaatttgagaaggctaaggtagtaaccttgattgtgaaaagcttgtcaccggggggactggggcaggtgggagggcttggttactacttatccagtcgcccctccagccccgctggctcagtgagcccagcagccacttcccgactttgtaggaacccatgccagtccctaagtggacctgctctttgacatctgtccagtgtggctgccctgtctcccatggcagcgtggcactgagctgcagtgctgtcttggaggttttgaccctgcctggctgtgtgactttgggcaaatctttcctcctctggcctcagaattccacttataaagaaaggggcttattcctcagagcccaggggcctcctggaagtgggctggggggatcaaccaggcaggggcctcaagctccctaagtagagaagctctgcctctgtcatttagtagattgtgctttttgggtgagatttcatttgagtgagggggatctctgactttacaaatagctgtcattcacttgctccatctaagggcccgcttctcacccggcaccacctatgagcctgcctcattcccccttgctgagcctgagaagaaatctactcagctgtgctacctcaggtcaattacaacctcgtggagtttgggttgttgttttttttttcttttttttctttttttttgagatggagtctcactctgtcacccaggctggagtgcagtgacatgatctcctgcctcagctccctgagtagctgggattacaggtgcatgccaccacgcccagctgattttttgtatttttggtagagatggagttttaccatgttggccaggctggtcttgaactcctgacctcaagtaatccacccacctcagcctcccaaaatgctgagattacaggtgtgagccactgctcccagccggaatttgggttcttgacacataaaatgcagataataatacctacctcataagattgaggtgaagattaaatgggatcacctccatatagcccctagcctggttgctggcacatggcagccccttaaagagtaggcacggtcttccactgtcaaggttataactatcacctgcacatgagtgactcagcacttcaatcattgtttaataccctgtagtcaaaggatgaataagaccctccctcaagaactcactataaagagattcccaaaccatgtcccagaatctgtacatcacctccacctcctcagaccctcctccaaagagtcgcctgtctctgactcctacctgtccagggttctagccacctgatggtaagatcttggctttccaattcaggcaaactcgtattaaaatttcagttctggctgggcgtggtggctcatgcctgtaatcccagcactttcggaggctgaggtgaccagatcacctgaggtcaggagttacatggtgaaatcccgtctctactaaaaatacaaaaaaattagctgggctaattttttagtggcgcatggttgtagtcccagctactcgggaggctgaggcaggagaatcgcttgaattcaggaggtgcaggttgcagtgagctgagatcgtgccattgcactccagcccaggcaacgggcgaaactccatctcaaaaaaaaaaaaatccagttctgttactaaccatatgaccttagacaagtgattctcaataaggggtaattttatcccccaggggatgttaggcaatgcctgaagatatttttgattgtcataactgggaatggaagtgctactggcctaaagtcgggagaggctagggatgttacaaaccatctcagagtacaaagaaccttctctgacaacagagaattatctggcccaaaattcagtagtgccaaggttgagaaacctgaccttaaatgaataatttaacctttctgggcttcagtttcctcatctgtaaaatagggctattaatagtatctaccttgtagagctattgtattggtccaacaaaataatgcaagtacctagtgcctaataataaaggtagtccataaatattccctacacgaatggttcttagcatgcctctggaatccctcctggcttcatcactgggctgaacccaccggaccctagtttggcttatttcttgccaaggccagtgctcctgactctttcttcactggtcttgatgctttccttctctccttgacctcaaggcctatcaagcagctggaagcctctccctcaggccccatcccagcaccaggcccctgcctccacctcccacttctcccttcccgtctctcttccccctctttccagccttacctctagtggctccccatgccccttggcaggtcagagcacactcagctttttcccacttcagcacctctgcacagacttcctccagccagagtggcagaagacagagaagcatgtccaggatttggaccagactgcctggtcccagttctgcactgggcctgttacttacggcctagttttaggaaagtcagcctcatctgcaaagcagggaaataatttccaacccatgaggttgttgtatggacccagttgagaatgtatgaaaacatgcaacaactataaagattgttaaaatgtaagttattcactgtgtgagcattattctgctaaatgtatgcatgtatgtatgtgtgtgtgtgtgtgtgtgcatatatatatatatatacacacacacatatatatgtaattttttttttgagacagggtttctctcttgctcaggctggagtgcagtagtgcaatcacagctcactgcagccttgacctcccaagctcaggtgatcctcccatcttagcctcctgggtagctgggactagaggcatgtgccaccacccccagctaattttttttttaagagatggggtttcactatgttggccaggttggtcttgaactcctgggctcaagcaatctgcaatctgcctgtctcggactccccaagtattgggattatgggtgtgagccaccatgctcagcctctgctttatatttttatttccatagctcctaccgtgttctaacaaactataactttgtctttggtttattgactgtctccctccactagaatgtcagctccataagggtagggatttttgtctgttttattcacttctgcatccctggtgtctagaatagttcctggtatatagtagatgttcaataaatatttgttgaatgaatgaattctctgtgtataaggcagttctgtaaagcccctaaatcttccttctccaagaagccctccttcaaatgtatgtaaccttatttagttattgcacatccccattttacagatgatgaaactaacgctggaaaaacttgtccagagtcactgacctgaaaagtggcagggccagatgcatttgtttatccaagaaaccctgagagggctgggcatggtggctggcacccgtgatcccagcactttgggaggctgaggtgggaggatcacttgactccaggagttcaagaccaacttggcaacatagtgagacccccatctcttaaaaaaataaagaggctgggtgcggtggctcatgcctgtaatcccagcactttgagaggccaaggcaggcagatcatctgaggtcaggagttcgagaccagcctggccaacatggggaaaccctgtgtctactaaaaatataaaaattagccgggtgtggtggtgggtgcctataatcccagcaacccttgaggctgaggcaggcaaatcgcttgaacatggaaggcagaggttgcagtgagccgagattgtgccactgcactccagcctgggcaacagagcgagactccaactcaaaaaaaattagaaacaccgagtgcctgctgtgtgccaggtgttagggagacagatgaccaagacttggtcctttctgtccctcacagcactttcaggaatgggggtcacagaggaaacactaggatgctataagaacacagaggagcccccccacccaatcaaggtcattggtcttcaagataaagtgcctaggcctggtgcagtggctcatgcctgtaatctcacactttgggaggctaaggtgagggggcacttgaggtcaggagttcgagaccagcctggccaacatggtgaaacctcatctctactcaaaatacaaaaattagccgggcgtggtggcacacgcctgtaatcccagctactcaggaggctaaggcaggagaagcgcttgaacctgggaggcagaggttgcagtgagctgagattgtactgttgcacttcagcctgggtgacaagagcaaaactctgtctcaaaaaaaaaaaaaaaaaattctcaaaccgtccgttggacccaaccactgcatgctgatttcactcatgcatgctccagctcccagacttctgggatccctttggaattcatccaattcagagtcctctactgctctttaatcatttgtgcttgttggttttgcttctccctctgtctttcccactggtgggaggctcccttgggagcaagcatagtattgctatcaccctgtggcatagtgtgcagaactcttgcacacccaggagctcagtccccgtctttgatagccactctacccatactacaggtaagaaaactgaggtccagagaattcagtagcagatctagcatcactcagcagttaagaggtaggattagaatgtggtcaccctctagccaccactcccatcacctctcacctccagcaggctcagctcctctggacctggctgctgcttctgggttggtgatttttgccctggcaaagggagagcagacttatgacatctggagtccagagttaggattgaatattcaggataaagtttcactttgaagttcagatttaggattgaggccattttgatggtggtcgcagggaacctttagaaagagcttggagtgaaatttggggtgaaagccctccttctttctcctgcaccttttccacaagatggggaggtctggccagacacagtggctcacacctataatcccagcgctttggaaggctgaagtgaggatcatttggggccaggagttttagaccaaattgggcaacatagcaaggctccgtctctatcatgatagcctatagtctcagctactcaggaggctgagataaaagaatcacttgaacccaggaattcaaggttgcagtgagctatgatcctgccaccgcactccagcctgggcgacagaccaagattctcaaaaaaagagcggggaggtctaggacaggaagggttaagtgtggtttagctttcccagcctggaaaggaagccaggccaagcagctgggtaggggaaaagggggcactgagtgccagggaggggctgaggcagtgggaacagcatgaacccttccctcacgtttcactggcttacccctcctgcctctgcttctggtgtggagaagaacaagactttttattgtctgggaagctgagggtgggtgccacgccctgggggagagaagccatattctgggccccctcatccgggtgcatccctagtgctcaccatgctgcccaggcaccctcactgagatgagaactttccttgttcccctcccctaataaccaaagttcctgccttaatcccactccagtccctcatccaccccgaggccaggcttcagaacccagaactgagggcctgtccagccctgctttccttgtgtttgagggaagccctgatatcttggagctgtacaaggtagcaacataatttgatttccctggttgctcatcggaggataagctgtgggaggcagttgggcagggcatgcaggcagatgggacccaggcctcaatgctgtcacctcttagagaggataaggtgggacgggcatggtggctcacatctgtaatcccagaactttgggaagctgaggcaggcagattacctgaggtcgggagtttgagaccagtttggccaacatggtgaaaccccatctctactaaaaataaaaataaaaaaaaaattagctgggcttggtggcaggtgcctataatcccagctactcaggaggctgaggcaggagaatctcttgaacccgggaggtggagattgcaatgagctgagatcatgccactgcactccagcctgggtgagagagcaagacttaatctcaaaaaaacaaacaaacaaacaaaaaaaacaaacaaacagaggatgaggtggatggtgggaccaggataggggccaggatgaggggaggggagtggaaggcttatcaaaagggtccttggtgaggcctgaggatggaggctccagagcctgaggtagcgaaccctggggacctgagtgatctcgttttgtgagagagcctggcccctccccagagccagctgcagaatggacctggccagagaggaaagtagagatgagtatggtgctggcctttgcagccagcaaggctgtggggtctttgctgttcctgtcccccactaccttcttgccccccactaccttctttctggctgaaaccaggatagagacccaatattggctgtccagcccccagccctgcttcccctttcaggcccctctgggaaccacagaaatctgggacctagtgtcttggcaacgtaatgaatgcatgcacagctctggtatctgttttaaattatccattaaaataagtacagttctgggggaaaaaaataagttgactgggtgcggtaagcagtagaaagggactgaagggggaaggaacccaggcagtgcttgggtccaaaggaaggggacaggagatggaaggggcagtgcctggctcctattcttggctttctttaggggacttctttaggggactgtggcttgttgcttgggtctaaaaacgaatgcttggctttgaagagagatagattggggcaaaagaaagaaaaaaagggaccccccaaactccttgatccctggccccaaactgggggcataaaggaactcaggttccagaactttgctccccccagggaacccaggcattccttctccaccccactcctggcacactgagatgcagctctgaatgggctgcccacgtgtggaggggggttggggtgactcactattactactgggaggacagggggagccagtggtggaagaagggtgagtcacactgatgggcaccagcctcagccctccccccactttcctggctcccagccctgcctacctgaccctctcccttgctttgcgcccacttccctctctttctccccgacccttttgcccacccactctccctccttggctctgccctctagcccagaaggtctgaggcaatgggggcaagcttggagccgacagtgctgagcaggcaggagccaagagaggggaagcttgagcctcacgcagttaggggtgcgctggagagggtggggcccgactccgccacaccccaacggtccttccccctcctcaccactcccgcccccacccccaatggatctgggactgcccctttaagagtagtggcccctcctcccttcagaggaggacctattagagcctttgccccggcgtcggtgactcagtgttcgcgggagcgccgcacctacaccagccaacccagatcccgaggtccgacagcgcccggcccagatccccacgcctgccaggagcaagccgagagccagccggccggcgcactccgactccgagcagtctctgtccttcgacccgagccccgcgccctttccgggacccctgccccgcgggcagcgctgccaacctgccggccatggagaccccgtcccagcggcgcgccacccgcagcggggcgcaggccagctccactccgctgtcgcccacccgcatcacccggctgcaggagaaggaggacctgcaggagctcaatgatcgcttggcggtctacatcgaccgtgtgcgctcgctggaaacggagaacgcagggctgcgccttcgcatcaccgagtctgaagaggtggtcagccgcgaggtgtccggcatcaaggccgcctacgaggccgagctcggggatgcccgcaagacccttgactcagtagccaaggagcgcgcccgcctgcagctggagctgagcaaagtgcgtgaggagtttaaggagctgaaagcgcggtgagttcgcccaggtggctgcgtgcctggcggggagtggagagggcggcgggccggcgcccctggccggccgcaggaagggagtgagagggcctggaggccgataactttgccatagtctcctccctccccggaactgcccccagcgggtgactggcagtgtcaaggggaattgtcaagacaggacagagagggaagtggtggtctctgggagagggtcggggaggatataaggaatggtgggggtatcagggacaagttggggctggggccggcctgaattcggtcagattgggatttgccaactatttggagccggggggaggggcttgagcaaaacagaactagccctgccagctcgaagaactctgggcacccaggacacatcggagtggcagaaagggtcctgttagaactttgttagcgggcttggcactgtgctagctttgcccaagctggctctgaacacatgatgcccactaagacataactctcaagttggcatctgtccagcgtgttggagcgaggtcaggaaggcagggcaatcccccttttccctcccaagggcttggcggtggccccccctcagcatgaccttgtcctgggttctaagggttgggaagttctccctcactctgccactctgcgtgtctgggaccttccttgggctctgacaggcccaccaaaagagctccgggagatgagagatcggctcccccgcagctcccacagcccttggcctgcttggcccaggaatgcaagggagggagggaggcagagggcagaggctcccagctcaggaagttgtgttatgcccaggtctggccgcactcctcccttggccctctgcctagtgtcttcgagggttgggggcactgtccttccctccttggggtgagccactttcattttcccagcggggccaggcagtctttgctcgggcccatcctcttagctgctgacgttttgatctttgtcttattgaagtgctggaatacagtgacatttttgaaatccagccgttggaagattcaggccactcccactttacccacccctgccccaccctaccccaccctactcaactgcaccttcttcttttctaaaaaagcctttgggagcttggaagtataggccctctcttccagccccatcaaaatttgtttcccttcttcctgccttccctttctctatgcagacccaggccaagagcactaagggtgcttggagatccgtaaagggctgttggctttgacttcttctctctcttttatcatctactccaaacttctgctcttcctagaaccctttgctaggtgtggttttgttgcccaggctggagtgcaatggcacaatctcggctcactgcaacctccgcctcccaggttcaagtgattctcctgcctcagcctcccgaatagctgagattacaggcatgtgccaccatgccgggctaattttgtatttctagtagagatggggtttctccatgttcgtcaggctagtcttgaactcccaacctcaggtgatccacccgcctcagcctcccaaagtgctaggattacaggcatgagccaccacgctgggcccatcacccttctttctgaagagtcaatggaagttgtgtgtaggaagacaggcttaacggtttttttttgagacagggtcttactctgtcacccagactggagtgaagtggtgcgatcttggctcaccacaacctctgcctcccaggctcaaaagattctcctgcctcagcctcctgagtagctgggattataagtgtgtgccaccacacatggctatttttttttttttttttttttttaatttttagtagagatggggtttcaccatgttggctaggctggtctcaaactcctgacttcaaatgatccacctgcctcggcctcccaaagtgctgggattacaggtgtgagctaccatgcccggccatcaacctttattttgtttttttgagacggagtcttgctttgttgcccaggctggagtacagtagtgtgacctcaggtcactgcaacctctgcctcccaggttcaagccatgctcctgcctcagcctcccaagtagctgggactataggtgcctgccaccacgcccggctactttttatatttttagtagagacggggtttcaccatgttggccagggtgatctcgaactcctgacctcaagtgatctgcctgcctcagcctcccaaagtgttgggattagagacgggagccactgcgcctggcttctttttttcttgagatagggtttcactctgttacccaggctggagtgcagtggcaaggtcatggctcactgcagcctctacctctctggctcaagccatcctcccgcctcagcctcctgagtagctgggaccacaggcaggcaccaccacccacagctaatgtttttgtattattttgtagagatggggttttgccatgttgcccacagtcttgaactcctgggttcattctgctgaaagagaccacacctgtccttttctttatttttattatatttttcagagacagggccttgccctgttgctcaggctagagtgcaatggtacaatcataacttgctgcagcctggaactcctcctgggctcaagcgatcctaccgtctcaccttccggaatagctgagactaagggcaggcaccaccacgcttggctaatttttttttttttttttttttttttgctttttgtttgtaaagatggaaacttgctatgttgctcagctggttccgaagttttggcctcaagcaatcctcctgcctcggcctccggaagcactgggattacaggcataagccaccaggcctgacgccaggcctgtcttttttctactagtgatatgaacaatttagttagcaagacagataggaagcaaggaaggggagacccagagaattcgttgcattctaaactagtccactcatctaccaaagccctgtgaaggacatttttagcagttttagcagttttctggtcaaaactttgatcgagaaacagattgagtggattcgatattctcttgctcacccagccacgccagtttgtctcctctgcctcctagtgcagctgtccaggcctgggacaccaggcgggtatgtgcgcatgtggggcagggcggaggtggtgtgtgtacttgttatatttagccacctccctctgttctcccccactgatcctggctggaaaggctgggcttccggaaaagagaggtggatttgcacacctggatcccaagctgatagaaagtggggtgaagacaaaggggactcagactggggtgtctgtcctcttctatgcccacagtaggaggagccaggattggttactccctgctgggtctgctgtgctcagagtgaggtagagaagtgggtagagtaaagaatttgggagaggaaaaaaggcattttcccaacccctcccaccaaagcctagagagaaggtgttgtctggtttaatgtttaattagagctcagagttcagggccagatttggagttgggatggaaagttgtttttaagaccctgtagcaatttttgacccagcctgggtacctcaaccacactcaggagtttgggggaccttctgttgggctggattataggctccaagaagaaacccctttcgccaatactctctctctcttctttttttgagacagggtcttgttctgttgcccaggctggggtgcagtggcatgatcacagctcactgcaacgtcagcctcacaggctctggtgattctcccacctcagcctcctgagtagctgggattacaagtgtgtgccaccatgcccagctaattttttttttctttttttttttttgagacggagtcttgttctgttgccaggctggagtgcagtggtgcgatctcggctcattgcaacctccacctcccaggttcaagcgattctcctgcctcagcctcccgagtagctgggactacaggcacatgccatcacgcccagttaatttttgtatttttagtagagttggggtttcaccatgttggccaggatggtcttgatctcttgacctcgtgatccgtccaccttggcctcccaaagtgctgggattacaggtgtgagccaccgtacccggccactaatttttatatattttgtagagatggggtttcaccgtgttgcccaagctggtctcgaactcctaggctcaagtaatccacctgccttggccttggcctcccaaagtgctgggatgtataggcatgagctaccgcacctggtaccccctgccccttctctgtctctttctagtctgtagcccaagggatttggatacccaagtgcaggcagaatgggaaggttgtaagcaccagggaagcctgtctggagtccaggcttgcagctgggccccaccccaggcaaggcagctgggtggatgactcagatgctgcccccctccctcccaccctggtggctttacagaagacagcaggagacagggtggagacagcagttgtcttaaagggaggagtggtggtctgaatgtctacctcttctgcccccctccccattgcatcctggagtcccttgcctggctccttcctgagaccctctggtggtgtctggacacatagctctctctggacaggtaacatgcacaagtaattagaatccagagttgagttcagagttatggattgggctgcaggatagtgccagggtctgtgccttcccatgtgaaactgatggaggaaggctgagtcagaagtggggagatccgaggcccacaaagcagaagcgctacttccactccaaaaaggccctggtgcttgacaacttcctggattgcccactgttgcagccccagtgtggacaggcagggagatgcaggctccagttcatgtaggctctgatcaagacaagaacagcaaaggccacagaggcacagatgcttgtcccatgtcacacaataaaggggtcagcacttgatcacaggccttatgacttccagctgggtgtgctcttaccattaagcctcacttctctagcttgggggacaggttggagggaggatctagagggtgaggtaaggtgaagtcaggtagctgaggctcacttctgcagcctggaaactctgctctggggccagtgacaccttagtgctctatggccatacttcgtggctcatgcctgtaatcccagtgctttgggaggctaaggcaggaggatcacttgaggccaggagtttgagaccagtctgggcaacatagcaagacccccttctgtacaaaaaaattagccggtcaacacctgtagtccagctgcttgggaagctgaggcgggaggatcacctgaagccaggagtttgaggctattgtgagctatgactgcactactgcactctagcctgggagagagaaagaccctgtctctgaaaaagaaaaaaacaaaacaaaactctgctgtcctgcagggcctgttagcatatgatcgatagcctttgctccagcctatacctggacccaggacccctgccagcccctcaatcgtgagacggtcagagctctgggaggctggtgattcttgtcttgagactatcttgagacttgtcatgggaattgtccacccggattgaaaggaagctgtgccttttggcagacccattaggttaatggggttggagacctttgaggatgcatgggccctgggctttatctgagggtatctcctggtgttacctctccaaccctccaccaccaaatccattctttttttttttttttttttttttttgacagtctcgctccctggcccaggctggagtgcagtggcatgatcttggcttactgcaatctccacctcccaggctcaagtgatcctcccacctcagcctcccaagaagctgggactataggcacgtgccacatgctcggctaatttttctatttttagtagagaccaggtttcaccatgttactcaggctggtcttgaactctggggcttaagcagtccacccaccttgacctcccaaagtgctgagagccactgagcctagcccaaatccacgttctgattcaaagggaaagaagaagggtgcagctaaacctggggggtgagaagtacttaaaaagcccaagagaaacaaaagagagaataattcctcactaggaccccctattgccttcccactattggtgcccttgcttggcacttcccctggcctccaggagtctgagacttactcttccatggatgtgcccattgcccccacttccaggtccaccccccagtgattcggtagcttagtgtctgcgctgaagcccaggacagctggatggacaactggtagatcccttcacctaccaactgtgctttctgctcccctcccccttgcttccctcctccccagcccctcgccacccctagcagctgcagcagccaagaccaagtcttcagagacccagacacaagggcagggttcattccattctcacctccttggggtcccagtgtactgataggccgaactctaatattataggagatctctggaagattgcagggtctcttatccctcaataaggggcaaggcaagccgggcgcagtggctcacgcctgtaatcccagcactttgagaagccgaggggaacagatcacttcaggtcaggagttaagagaccagcctggccaacatggtgaaaccctgtctctactaaaaatacaaaaattaaccagaaatcgcttgaacccaggaggcagatgttgcagtgagccgagatcacgccactgcactccagccagggcgacagagcaagattccgtctcaaaaaaataatactaataataaataaataaataaggggcaaggtagtccaccaacaaaatgacaggcagtgtgatatagtggacaccctagccctcggtgcccttagttctgtgtgtggccctttcactaaattgctgtgtgaccttgagcaaatcgcctcccctttctggctttccttagctgtaaaagaaagggattggagcggaaagtctccagagaccttttaggttccaaagtagtacagtgacccacaaagtgagaaaacagtcttctaaaataccaagttattaatagtaaaatcaaatataaataatgtgaatatagttaatagctaatgttgttctcaatagaaatgtttcccacaagctgtggaattaaacatactaccacatttctctatttccccgtgaaagtttgttagaaatggttaaattgtgacattaccctcttggcaaatgttttgttttcattgctactaggaaagggcaactcgttttcgatgcctctcccttctggacggtggaaagggctgtgtcatagagtaggaacgggagatgcggcacaggaatggctcccattgacccgggttgggggctagggcgaaggcctaggagaggcagaactgttaccttagagctggccaggattagagaacagtgcctggaaccggggggaggggcacggtgaccttgggctgcccaccttctacccttccagcacccatactggctcccccaacctgcggctgggctgggaggaggtcttggcccctaccaatcccttaaggaaggggaaagagtttgggaaggggagtcctcccttcacccctgcctcccccaagttgtgagagaggaagccggaatcctgcctgctgaagccaggaataattctggctgagatcccaggcccggcaggggcgctgagtcatggtagagggcagagtggagagtggacaggagaccctaagcttgtccagtcagaaaagcagaggctgaggggtggccttttcttgagaactacattcaagttgcagcaagaaggacagtggtctgaatttgacggggacaaatggaagggagataggacacatgagttcctttaggtctggctcaggggagctagacttcatttcaaggggtctaggttctgggcagttgagaaggaggctatttggggtcaccaaggctcccctttcttcccaaagctctaacactgccaccttctgctggctaggagagagctgtgtcttctgaggctagagctggaatgcagtgagaccagactgcctaggtcctccctcacttcttctcctgaccttggggtgtggctcccactctctcccagtgtcctcagggttaataactatgtgccaccagatagagagttaaggggctgctgaattggcttcttgtgaagggaatcccctaaatgtccctcgttttggtcactggcctccctcccgcccccttcaggacattctactatcttcttaggccatccctccctcctccaggcactacttcttttgctctatccccaagccccacccctgcatttttgtgacaacaccggaatgatttctagagagagaggccaggaagaaggaaagtggcacttggcaggagaccttgcagggggcggctggtgaggaagccagccgcccattgtccaggaccccagtgccctggcctccggcctcaggcttctcctgcctctgtacaatgccacgttgatacgcccagcagctgtgactcaggcctggccccctgccaggcccagcacttctactggagttgcgtctgaacatgtcaacaggcttcctatccctctctcagcaccagttctccccacttcagcccctccctctgcctggaattaaaacctggctttgtcttagggaaggacagctgggagcctagtggctctggtaggggatctgagaggcctcagaccctaggcatatttggctgtttggcaggtgtcacgcccaagggaagcgtgtggaagcagagccatgcctgctgtgggtgcacatgcccgcgtgagggagtcggggtgtttcatcctggggcacctgtgggcttttgaggtgtatgatattcagaacttcacaggttggggtttggggaaggctcaaggggcttctaagtccctggaacagctgcccccctcagttcctctctctctctctctttttttttgagatggagtctcgctctgttgcccaggctagaatgcagtggcgcgatcttggctcactgcaaactccgcctcctgggttcaagtgattctcctgcctcagcctcccaagtagctgggactataggtgcccgccaccatgcctggctaatttttgtatttttagtagaaatggggtttcaccatgctggccaggatggtctcaaactcctgacctcgtgatccacccaccttggcctcccaaagtgctgggattacaggcgtgagccactgcgcccagcctcagttcctctctttaaggtctcctttccagagagggatagcacctcaaatgccagggaggggaattctccacatcctgcccttacccgagttgtggcagacccacagactagccaagaaaccaagcagtggttactttgccgggttgggggggaggtaggggctatcaaacctcatgattggccgcacacaaaggtgtgagtatgtgtatatttgagggtgggtgggagtggcactttcactaggcctccgtatcactctctgactggggtatctcccagcaagcgagacagaggcagacacgcttcccagactgtcttactgggtctctctgtgttattctctgcagtgtctgtgtgtatcgtgccattttctatgttttgcaccaatctgctgtgagtgtcctcaggtgacctgggggcaggtttttagtgcctgagcctacccgtctccaggctttagtttccccctgtaaaagtataggagttggttcaagagaaggttcctctagaagccttgagcctgtgaaccgtctagtctccgggtatttgtgggacacacagaaaaagccccacgacccaacaggtagaacactggctgaaatcagcagggcagagctgagacaggctcaagtaggctgaggggtagggaggttttgggtgaatgggagggagggacagagagaaggaggatatattgcagtaggaggagttgctggaacaaaaggaggggtggtaggagtggcttggggtggcagcagaagacgccctgtcacatggcgggaagtcagcctgggcagaggtctaggtgtccaggaggggctgggtgtggtggctcacgcctgtaatcccaggactttgggaggctgatgcaggaggatcacgtgaggtcaggagttcaagaccagcctggccaacatggcgaaaccctatctctactaaaaatgccaaaaattagctgggtgtggtggcaggcgcctgtaatcccagctactctggaggctgaggcacaagaattgcttgaacctgggaggtggaggttgcagggagccgagatcgcgccactctactctagcctgggcaacacagtgagactctgtctcaaaaataataataataggggctgggcgcggtggctcatgactgtaatcccagcattttgggaggtggaggcgggtggatcacctgaggtcaggagtccgagaccagcctggccaacatggcaaaactccgtctctactaaaaatagaaaaattagctaggcatggtggtgcaggcctgtaatccagctactcgggaggctgagaagcaggagaatcacttgaacctgggaggtgaaggttgcagtgagatcacctgggcgacagaatgagactccacctcaaaataataataatagtaataataataaatgaaaaattttaaaattaaacaattaaaaattttaaattaaaattaaacaaattagatgcccaggaggatacaggagagcatttgccaccaggcggactccctgtacccacccggccacagggggcgatgttcctgggagacaggaaatgcccaggggctgggagaccctctgctcttctgctcccttcctgtgtgctgcctggcaatggggaactctgagggctggtgagcagggctgctgaggagtgggtctaaggagtccctgcagggctgggccagctcctccacctcccctttgtcttcccctcccacttgttatttttagctacagtgtctgtccctcttgcttctcccccagattgggagaggaaacggaggcctctccctccgggcctagcctgttgcccccagcaaccgggcccaaacaggcctgtggccggccctggcttccatatctggcatcagagttgggctgagcagggtgactcagagggtgggtcagcgcctggcccggtgcccacctagcccctttgctgtgctggtgcctttcttccccaaacagccccaagggcccgggcctgctgcagctggggagccggacttccttgtcccaccaggcacagctcttcagacccctgccttgggtcacatttgcaagtgccaactctcatttctaccttattcttttcctctctgttcccctccccaccccctctcttccctctttctgagatcagatttgccagtgatgggaagagttagaaacaggatgcccagcccttctcgcctcaagaggccactgggatgcagccactcctgtgcttggggaacctggaggatgcaagggaaaggactggcactctgctggcacagcacccggcctggggcaggacacgggcgaagccagggtctcccctgtgagcactagaggatttcccgacccctgcccgggtattgtgtgcctgagcatgagtcacctgaggggcccaggttcccacccttcccagctcctctggcctgccccaccctgtcctccctgccaacccagcacggggacggcactcagcgtgtgctcagctttcctgatgccaacccccagtggagtgggctgcaccaccaccctgggaccgaatgcctggctagggtctactttggtccctgctaggtctgaggacccctcctaggaaggaaatggcacttgggggcgggggcagggaggagggaggagagacactgggctctactgtacccctagtcatctcttggggtgtgcgtgtggctccctggccacagagctcccaaggtctgagtcatgagcccatgggtgatagtggcttcttccccgcagatgggagctccccgtgcctaagaaaaccacaaaggttcttcctcacttccctctctgctcgtggtttttctcatctgcagggtgtgtcttagtcctttaatctcctctctttgcagtgctagtcaaaacctccaccagggaaagacaaataacccccttactgtttttttttttttttttttttttttttgagatggagtctcgctctgtcacccatgctgtagtgcagtggcacaatctcggctcactgcaacctccgcctcccaagttcaagtgatcctcctacctcagcctcctcagtagctgggactacaggtgcacaccaccgtacccagctaaattttttttttttttttttgagatagagtctcactctgtcacccaggctggagtacagtggtacaatctcaactcactacaatctccgcctcccaggctcaagcaattctcgtgtctcagcctcccaagttgctgggactatggacgtgcaccaccttgcccgactaatttttgtatttttgatagagtcagagtttcaccatgttggcaggctggtctcgaactcctggcctcaagtgatccacctgccttggcctcccaaagtgctgggattacaggtgtgagccaccacactcagccagcccccttactttccttggagaccatatactgtggcttgtgccaaagtggtacagcatggatttccagctcccctatctacttgctgcgggaccctagatatagctttctgtgcctatttcctcaattgcataggaatagcacctatcgcatagggtagctgtgaagatgacgtgagttaacataatatttagagcagtgcttggtacctaataagctctatataagtgtttgctattatattattattatcactgccaccaccgcttttgcaagcagcagaaggtgaagaggttagactgaagaaaaaacttctgtgctcatcagcccataagctcgcagagcacagggatcatgcatctatgttttcctcagtcagtgtctgccaggcactggcaaggaaaggctgttaccagggggaactccaggaattcctcctggcacctaaggaggctggggagacaggactagggaaaaggtgcccttgagacaccttctgaaatcatcccattgccttccagcttctttcagctcaggctggctggtcagggaaacgctttgtgccatagtgtctgccctcttcctcctcctggcttctccattctctctggaacttgtggcttaggaaagcagtgaggtggaggaggaggaaccctagatcagcagctagaattgactggaatgctgctgctggctttcggtaattgacactgggccattcaccttcctcctttgcacctcagtttcctcatctataaaagggagagggttgagctgaatcaactctaagctccttctagttctctaaattctgagagcctcctagtacagccagcagcagccattagccttcagggtagagaggcctcttctgggaagccccagccagcctgggggtcagcccaaggagctcggaatctaagttgccccagttgcttcactttaccagcggtttttcttcattttccctcctccccctgcagctgcttcagcttcggaaaagttctgaagtcatggaaagttggggctgtgctcccagccaggggctaggccggatggcagccaaaacctgagctgggttttgactttatttttagcttttctgactgagacagaggagggaatacattctccggttctggaaggggctcttttttgcaggagacagacacttacattaaacaacttgttctgaggtgtggccagaggcctggactgagcaagtgtgcaggctgggggagcttcctctggcttctcatgtccttcccctgcccctctgagtgtcactctatcctcctccctgcctggtggggggaggtgggggtgactccttttttggactctcctaagcagaacactgcctgggtctcgtcctccagagcttctgcaaatctagccttccctatccctcttcacagtgaattgctgggcctcttggagtttaggacttttgtggtagaagaaaaatgttggcagggctgcttttctcctttccaggatagatttttccttctgcccacgcttggttttccttttttccatctgctgtggtgggctcatgcttaagcactgatgagttacagatggcagctggaaccaggtcctctggatctttccctccgctccctgggtctgctgctttctctcaccctatatttgtgaagcaattgtaacatctagaaagttcttgggttctctggaggtttttaagaaaataggacctttctatttctccagtccactagcaaaaataatcaggggcccagaaaaggtgagggaggtggcagaggcagcgctgttcgactggttatagctaaagctttacccactttgaggagcagggaggcttaaagctggggcccagatggacctggaggcctgggatccacatctggaaccagatgctgaggctatggtagatgggtagggctcagccttctcccagggcacggatgaggcaggagggagggaggcagggacccctctgttcagtgcagatcagggcacccagactgggtcctgagaaaggaaagggtcaatattgtgcctggtcatccttgtctgaggtccctctgagctctaaccagactttccttccccacagtcccacatgtgtaaaagggactaggagaggtgaccagtacctttggggctcagatcgagaagtgctagggacatgtgggccatgagcttagttgtcaggctcctcagagggagggaagcttggccaaagggaagtgagtagagtccagggagaaggctaagtaaggccctgtgtgggaaggggcaggagacaaaggtacccctgtctctttgggaaagaatgggaggagagagagggaaaagcattcatatcacggggtagagctctgcccttggccccaggcacgttcctgagccctgagtcatgggaagggtggagaagcaggaagggggttttcaaggaccttggggaggtgggagcccagccccagaggcaagcagatgcaaaccaacctaatgcaaggatgccctctcctggtaattgcaggcatagcagcgccagcccccatggctgacctcctgggagcctggcactgtctaggcacacagactccttctcttaaatctactctcccctctcttctttagcaataccaagaaggagggtgacctgatagctgctcaggctcggctgaaggacctggaggctctgctgaactccaaggaggccgcactgagcactgctctcagtgagaagcgcacgctggagggcgagctgcatgatctgcggggccaggtggccaaggtgaggccaccctgcagggcccacccatggccccacctaacacatgtacactcactcttctacctaggccctcccccatgtggtgcctggtctgacctgtcacctgatttcagagccattcacctgtcctagagtcattttacccactgaggtcacatcttatcctaatttggctgccaatgggatctaccacagtgaatttaaaataatccaggaggccgggcatggtggttcacgcctgtaatcccagcactttaggaggccgaggtgggccgatcacgaggtcaggagatcgagatcatcctgactaacatggtgaaaccccgtctctactaaaaatacaaaaaattagcctggcatggtggcgggcgcctgtagtcccaactactcgggaggctgaggcaggagaatggcgtgagcctgcgaggcagagcttgcagtgagctgagatcatgccactgcactccagcctgggcaacagagtgagactccgtctcaaaaaaataataataataataataaaaataatccaggccatgtgtggtggctcatgcctgtaatcccagcattttgggaggccaaggaggcaggattgcttgagtccaggagtttgagaccagcctgggcaacacagaccccatctctagaaaataaaaatttaaagaaattagctgggcatggtggtgtgcacctatagtcccagctacttgggaggctgaggcaggaggatggcttgaacctgagaggtcgaggatacagtgagctgtgattgcaccactgcacttcagcctgggtgacagagggaaaccctgtctctacataaataaatacataaaataaaataatccacaagccatttctacttaactttgcaatgaactgtacctgaccctagatccctcccagtttggccctccggtatacaagggcctcctataggcccttgtgatttctctggggaaaaggaggactggagttgatcatttattgaggccatcagaagcggatggctaattacatatgggacatgtgttaataatgctttgtgtatatagagtggcctttactttcaaaacactcttctccaatttatcatgttaaaagctaggaattgggctgggtgcagtggctcacgcctataatcccagcactttgggaggccaaggcgggtggatcatttgaggtcaggagtttgagaccagtctgaccaacatggttaaactccgtctctactaaaaatacaaaattagccaggcgtggtggcacacacctgtagtcccaacaactacttgtgaggctgaggcaggaaaatcatttgaacccaggatcagaggttgtggtgaactgagattgcaccattgcactccagcctgggcaacaagagcaaaactctatctcaaaaaaaataaaaaatagccaggcacggtggctcatgcctgtaatcctagcactttgggaggcagaggtgggcagatcacctgaggttaggagttcgagactagcctggccaacatggtgaaaccccatctctactacaaatacaaaaattagctaggcatggtggcagccacctgtaatcccagctacttgggaggctgaggcaggagaatcgcttgaacccgggaggtggaggttgcagtgagccaagatcgggtcacagcactccagcctaggcaacagagcgagactccatctcaaaaaaacataaataaataaaaataaaaataaataataaataaaagctaagaatcaaagaagcagtttattcctaatttcacagtctcatctgttcatagtggggccaggattagagtcagtggccaagcttccatcctgggttctttcccttcccaggccctaccatcatagtataccagggaaagacctggagaagccagcaggttgaccaccgaaccaaggctgggccaccttcctcctgggtctggtctccagcctcccagttgtacccttcccccagcccttcctggatgcactgatcagcctgtgcttccttgccctgtttttctttataaatagagccatgttctcctctctctctctctcttttttttttttttttttttgagatggagtcttactctgtcacccaggctggagtgcaatggcacgatctcagctcactgcaacctctgtctcccaggttcaagcaattctcctgcctcagcctcccgagtagctgggattacaggtgcccaccaccatgcccagctactttttggatttttagtagagacagggtttcaccatgttggtcaggctggtcttgaactcctgaccttaggtgttctgcccgcctcagcctcccaaagtgctgggattacaggcgtgagccaccacgcctggcaagacgtgttctctctatgttgttgaggctggtcttgaactcctggctgcaagagatcttcctgcctcagcctcccaatgtgctgggattataggcatgagccaccacacttagcccagcctgtgctttcttaaatgaaaatctaagcatacggctgggtgtggtggctcacgcctgtaatcccagcattttgggaggccaaggtgggcagatcacgaggtcaggagatcgagactatcctggccaacatggtgaaaccctgtctctactaaagatacaaaaattagctgggtgtggtggcccatgtctgtagtcccagctactcgggagactgaggcaggagaatggcatgaacctgggaggcagagcttgcagtgagctgagatcgcgccactgctctccagcctaggtgacagagcgagactccatctcaaaaaaaaaataaaaataaaaaaaagaaaatctaagcgtggtgctcccctgctcaaacatcctcaggttcttttcatggcagataagggcatctcttcatgagccagcccctgcctactgacccagccacctctcccatcccttcccaccccgtacttcaggcttcagcagtactgatctttccaaagaccccagaacacacatgccttcatacctctgtgcctgtacatgcttgtttctgcccttgaaatcatgacagtagctctctgtaggccccgctagcctgtcccttgggtcttagcctcttggaggccttcccagagccccccaaaagtaccccaggcatactttggttccttctctcatgtcccctcagtactttgcacatacctcctttatagcagttgctatgttgtgccagagaagggagtcctgtggctggggggcatatatcttttctttttgagacagagtctagctgtgtcacccaggctggagtgcagtagtgcgatctcggctcactgcaacctccacctcctggattcaagcgattcttgtgcctcagcctcctgagtagctgggactacaggcgtgtgccaccatcatgcctggctacttttttgtattagatatatattttctctcttagcacagtacctaccaagagtgagtgagtagatgtcctgacccctgcaggcatccaaggccctccttccctggacctgtttccacatgtgtgaaggggtgcacaggcagcagcccacctctcagcttccttccagttcttgtgttctgtgaccccttttcctcatctctgcctgcttcctcacagcttgaggcagccctaggtgaggccaagaagcaacttcaggatgagatgctgcggcgggtggatgctgagaacaggctgcagaccatgaaggaggaactggacttccagaagaacatctacagtgaggtggggactgtgctttgcaagccagagggctggggctgggtgatgacagacttgggctgggctaggggggaccagctgtgtgcagagctcgccttcctgagtcccttgccctagtggacagggagttgggggtggccagcactcagctcccaggttaaagtggggctggtagtggctcatggagtagggctgggcagggagccccgcccctgggtcttggcctcccaggaactaattctgattttggtttctgtgtccttcctccaacccttccaggagctgcgtgagaccaagcgccgtcatgagacccgactggtggagattgacaatgggaagcagcgtgagtttgagagccggctggcggatgcgctgcaggaactgcgggcccagcatgaggaccaggtggagcagtataagaaggagctggagaagacttattctgccaaggtgcttgctctcgattggttccctcactgcctctgcccttggcagccctacccttacccacgctgggctatgccttctggggatcaggcagatggtggcagggagctcagggtggcccaggacctggggctgtagcagtgatgcccaactcaggcctgtgcctccacccctcccagtcaccacagtcctaaccctttgtcctcccctccagctggacaatgccaggcagtctgctgagaggaacagcaacctggtgggggctgcccacgaggagctgcagcagtcgcgcatccgcatcgacagcctctctgcccagctcagccagctccagaagcaggtgataccccacctcacccctctctccaggggcctagagtctgggccggatgcaggctggaagcccagggttgggggtgggggtgggggtgggaggttcctgaggaggagagggatgaaaagtgtccccacaaccacagagaagggtcgcaggatgtggagtcagatggcctgtgtgctgtttctgtacactcttacctcaccttcacttctcagggctttggttttcccattcgaaaatggaggctgttcttaatctccctaactcagagttgccacaggactctgcaatgtgaggtgttaaaagcatcagtatttttctagttggctgtgctatttgtgacaggagaaaaagtctagcctcagaacgagaggtttcagttagacaaggggaaggacttcccagttgccagccaagactatgtttagagcttgtgatgttcagagctggctctgatgagggctctggggaagctctgattgcagatcctggagagagtagccaggtgtctcctacaccgacccacgtccctccttccccatacttagggcccttgggagctcaccaaaccctcccaccccccttcagctggcagccaaggaggcgaagcttcgagacctggaggactcactggcccgtgagcgggacaccagccggcggctgctggcggaaaaggagcgggagatggccgagatgcgggcaaggatgcagcagcagctggacgagtaccaggagcttctggacatcaagctggccctggacatggagatccacgcctaccgcaagctcttggagggcgaggaggagaggtgggctggggagacgtcggggaggtgctggcagtgtcctctggccggcaactggccttgactagacccccacttggtctccctctccccaggctacgcctgtcccccagccctacctcgcagcgcagccgtggccgtgcttcctctcactcatcccagacacagggtgggggcagcgtcaccaaaaagcgcaaactggagtccactgagagccgcagcagcttctcacagcacgcacgcactagcgggcgcgtggccgtggaggaggtggatgaggagggcaagtttgtccggctgcgcaacaagtccaatgaggtaggctcctgctcagggtctaaggggatacagctgcatcagggagagagtggcaagacagaaggatggcatgtggagagaggaacatccttgccctcagagggtggaccagggtgagcctgtatatctcctccacactctggttccaggcctggctcctggactctttggctgtgagaccttgagcaggttatttaacctctcagagcatcagtttcctcatctgtaaaatggggatgaatactgatccctaagtctttgagttgtcaggaagatgaaagataaggtatccgtgtgcctggtgctgcgtatgtgtccacagatcatggctattatccccgggggaagggcagtgacaggggtgtgtgtagatggaaggagaggcctcaattgcaggcaggcagagggctgggcctttgagcaagatacacccaagagcctgggtgagcctccccgaccttcctcttccctatcttcccggcaggaccagtccatgggcaattggcagatcaagcgccagaatggagatgatcccttgctgacttaccggttcccaccaaagttcaccctgaaggctgggcaggtggtgacggtgagtggcagggcgcttgggactctggggaggccttgggtggcgatgggagcgctggggtaagtgtccttttctcctctccagatctgggctgcaggagctggggccacccacagcccccctaccgacctggtgtggaaggcacagaacacctggggctgcgggaacagcctgcgtacggctctcatcaactccactggggaagtaagtaggcctgggcctggctgcttgctggacgaggctccccctgatggccaacatcggagccagctgcccccaacccaagtttgccaattcagggcccctttctagagctctctgttgcaggctccagacttctccacccagtaggcaaaccaaaagatgcttcctcaacagcacaaggggtggaagttagacagtgaggattgttaaaggcagagccatactcctacccggagagcttgacagtgtccctctggggtggaaatgagttccttagctccatcaccacagaggacagagtaagcagcaggccggacaaagggcaggccacaagaaaagttgcaggtggtcactggggtagacatgctgtacaacccttccctggccctgacccttggacctggttccatgtccccaccaggaagtggccatgcgcaagctggtgcgctcagtgactgtggttgaggacgacgaggatgaggatggagatgacctgctccatcaccaccacgtgagtggtagccgccgctgaggccgagcctgcactggggccacccagccaggcctgggggcagcctctccccagcctccccgtgccaaaaatcttttcattaaagaatgttttggaactttactcgctggcctggcctttcttctctctcctccctataccttgaacagggaacccaggtgtctgggtgccctactctggtaaggaagggagtgggaactttctgatgccatggaatattcctgtgggagcagtggacaagggtctggatttgtcttctgggaaagggaggggaggacagacgtggggcatgcccgccctgcctctctcccccattcttgttgcatgcatatcctctcatttccctcatttttcctgcaagaatgttctctctcattcctgaccgcccctccactccaattaatagtgcatgcctgctgccctacaagcttgctcccgttctctcttcttttcctcttaagctcagagtagctagaacagagtcagagtcactgctctggttctctgtccccaagtcttcctgagccttctccccttttatgtcttccctctcctcctccgggcccctagcctcccaaacccccattgcccgctggctccttgggcacagaaccacaccttcctgcctggcggctgggagcctgcaggagcctggagcctggttgggcctgagtggtcagtcccagactcgccgtcccgcctgagccttgtctcccttcccagggctcccactgcagcagctcgggggaccccgctgagtacaacctgcgctcgcgcaccgtgctgtgcgggacctgcgggcagcctgccgacaaggcatctgccagcggctcaggagcccaggtgggcggacccatctcctctggctcttctgcctccagtgtcacggtcactcgcagctaccgcagtgtggggggcagtgggggtggcagcttcggggacaatctggtcacccgctcctacctcctgggcaactccagcccccgaacccaggtgagttgtctctgctttgtctccaaatcctgcaggcgggtccctggtcatcgaggggtaggacgaggtggccttgcaggggggagagcctgccttctcttccgcagcccgggggagtgggagcctcctccccacagcctgagtcctagacagcccacctctgcatcctgcccctcttgtctgagccccagactggagggcaggggcagggctggagtgtgagggatgggggagatgctacctcccttctaggggccaggggagggagggtctgggtccaggccctgctgctcacacctctctcctctgttttctctcttagagcccccagaactgcagcatcatgtaatctgggacctgccaggcaggggtgggggtggaggcttcctgcgtcctcctcacctcatgcccaccccctgccctgcacgtcatgggagggggcttgaagccaaagaaaaataaccctttggtttttttcttctgtatttttttttctaagagaagttattttctacagtggttttatactgaaggaaaaacacaagcaaaaaaaaaaaaaagcatctatctcatctatctcaatcctaatttctcctcccttccttttccctgcttccaggaaactccacatctgccttaaaaccaaagagggcttcctctagaagccaagggaaaggggtgcttttatagaggctagcttctgcttttctgccctggctgctgcccccaccccggggaccctgtgacatggtgcctgagaggcaggcatagaggcttctccgccagcctcctctggacggcaggctcactgccaggccagcctccgagagggagagagagagagagaggacagcttgagccgggcccctgggcttggcctgctgtgattccactacacctggctgaggttcctctgcctgccccgcccccagtccccacccctgcccccagccccggggtgagtccattctcccaggtaccagctgcgcttgcttttctgtattttatttagacaagagatgggaatgaggtgggaggtggaagaagggagaagaaaggtgagtttgagctgccttccctagctttagaccctgggtgggctctgtgcagtcactggaggttgaagccaagtggggtgctgggaggagggagagggaggtcactggaaaggggagagcctgctggcacccaccgtggaggaggaaggcaagagggggtggaggggtgtggcagtggttttggcaaacgctaaagagcccttgcctccccatttcccatctgcaccccttctctcctccccaaatcaatacactagttgtttctacccctggc

SEQ ID NO: 4 - DNA, 146507 bp, Homo sapiens
ggcgcccgcgcccgcccccgcgccgggcccggctcggcccgacccggctccgccgcgggcaggcggggcccagcgcactcggagcccgagcccgagccgcagccgccgcctggggcgcttgggtcggcctcgaggacaccggagaggggcgccacgccgccgtggccgcaggtcagagtacgggtgcccgcggcgctcgggaaccggctgctggctgggcggggagtgctcagggagggggcgcggagggctggggccgagggtctgggggctagggccgaggaaacgggaactgacggggtcccagacggatgagagctggggagaagggggtctcggctgaggggtccggggctgaggcagggtcatggtccggcaggacccggactgacgggtcgcgggcgggcggctcacgggtgaccgggtgaaggggtcttgggctgagggcacccgggctgagggtcgcgtctaccggagcgcgcactaggggcggagggcgcgggcacagagagccgcggccggtgccgactcgagtggacgcgagccggtgcgtcccggcttccaggacagcccccctcgggagagcgccgggtgggcgcggagccggggacacggggacctccgcgtccccggcggaggtgcgcaagcgtcgggagccgctccgggggaactttggagcagggtgtctggacccggggaagcgaagatccgcgtgcgtccgtcctgaggccgcgcggcacccctgctgaggaggaggtcccgttcgctgtccagagcccaggggcgaccgccgcttggggaccgggggaccgttcagcgaggggcgcgcttgtagctggccaggccctgtgccctgacgcgttgtgctggccctacgcccggcggaggggctggggacggggactgggtgcctccgtgcctccgcggatggagcgcgcagccaggacccccagccgcaccccggggggctggaaaggcgaagtcgctgctttgtgggctcctgcaagtgccaggcgctgaccactcctcctgggagcctaactgaggggggtagaggcagcagaggttaacagaaatctcaggtcccttggtggaaggtgggtgtgtagtcgtggtactttacgcctcggtgtttagggaggagcctaaggtaaggagtcagaaacggggagtaaccgagctgcggcttttatataaggtcagtggtaggtaaggaaggggccttaacctctgctggtgaccagaagcctgcatttctgcattctgcttaattccctttccttaggtgagttggctcactctcattgcattttgttggaatgtgttggtgataggtccgaaatttgcaaaattattgtattatccttatatcatttttttttgcagtgctcttgttttataagaagaatccaattcacttgggtcataggctaacggttgtaaaaatagtgtcattgttataaaagaaatcaagtcaaaaatagtcgtctgccatgaagaaactaaaatttttcttcttttccttgcaagattagggacaaaatgtaaagtttatttgtgtaaattatcatttggatataaaaccagacaatgataaactgagttgtataatacctattcctggaaaacctctgattttgaagtacattattttaaccccaactgtttctgtcattcgtgagacaaattgttaactggatttattaagagaacaatgaataacttaactctttaaaaggacctgggcaaatcttcgacaggtttcaggtattttatggcagtctttctgtctattgctaagtgatcgattgctgactgagaacagaaccaactgatggtgctagatgatttagaaaacctttctggtatagggactggcatggacattcactgtggcttttctcttaaagaaatacctttcactcctaggggagtttgacagaagacactcatagatgctaccagttagcatttggagtaatatatcaaaacattctgttcaagttttaaagtaagttttgtgtagtatattttcaatcagattcactttggaatcaaatacactttatttgattccaaataaagtgtatttgaatattcactttaatatacgaaactttaaaggtaatgtctcagccaaaaagtgaagtgtattacaaaactgtccatttttgtcttcttgtgtacttcacataatttttctccctgatttaaattatatgatatttgatagttaacttgctaataattatgtcttaagagatctttgaatcaactggtggccatctcagtcttttaaaactcacttgaaggaaaagagaaacactaatgatggttttttttaaaatcaaaaacttttttcatatttacaagtttgtttctagttcttaaacaataactgctgtaaaaccatggcatcagtgaggtataaagttttgtagaatcccaggctgaagactttttggatgtcgttttctcatttctttacattaaaaagaatataatttctttttaaaattaaaactaaaatgtccaccggcctcatattctttattgaacttaagtgctttgaaaatatttttgcgattgatatttttaatgataaaatataatattttctttgaaggactagtttcctaaatgttcatttttttgtatgtttcagaactacttttaaaaaaattctagattatgctttaaattgcaatatttctaaattcctatgacatttaaaatgtgtacaagagaaaaggtgtgacatgaggaatatgaattagaagtggattactaatggtcgtagtgaattgtttttaatcagtgttatataaatataaacaaatacttttttcttctgtaattttctgacaataaggatgttagccactaaccttaattatcaagataattatgtgcatgaaagctgtgtaaaaggcacaaccgtggaacatatacaagtattaataatacatttcatttagttttcaggttataaaatcgttattaataacaagaggctttcatgaagtaggaaaatatgttaaaatgacattttctgaatgtttcacacattctggcccacactttcacttctttcctagcatctatatattcccaatgcataggacagagttggtcattctaattgtgggtgtctttctgctggattcgttgtcacttggacttggaaatacagtatgtcattaggtgtggcaaatgtactggaagaagtttttggtctcataggattccactaaataaatattagctgtagaggatttttaaaaactcactaatagagaaaacttggtgaagtcactagtgtgtaagcgtaaacatatgcaccccccacaactgaatttctaatttgcttatttttcctggtagatctccactttgcttactctctcttaatatcccaaagcaacaaaaagcgcagtgattttaattgcttacaaaggtcctttgaaacggatctaatttgactccacaaaagaatagtaagagttgtgaacaaccatcttgcaagtgtagatatccccagcgtatcaacttcagttagtttcatagcgttctaaatcaaaaatttagatttaagattatttgaaatcttcttggaagttcttacttactgtttgcgttttctcaaaatacagaggtagattgggggagtaatatagtatatgttttaaagctgttatttaactaaaatgtcatgttttaaattttatttaatgcacttaaagtaggttcttgggataaaatctattcagatgcaataggccatgttttggggaaaacttttgtagatgtttagataatatctgtcatcaataatagtatatgtttaaattattttgtaaaaacagatttacatatttttaaaatttctcttcaacaataagtacacatcaatattttaatgccgagaatttcattcagagtatgactgtttacattttcaactgtttcaattattatggcttaggcaagtaccttatcctttctctcaattttgtcatctttgaaacggagaaagtgttgcagttattattgcttgctcttttgctatgattttaaggctttatgagcaaacccatgtactttaaatgatgtgaccatgaccctgaatttaattgagctgatattctgaaaagaaccttcatgaatatctatgtcatagctaactcttttaccaatgttaatagttcactgtagttcaaacctcaataactttgagattgttaagattgaagatgatgcttctcatgtggtgatcaatttataaactgtttctgggaatattttattatcattcaatttaaaattgtattttccattccctgatgactttggacaaaaatcagcatagtaattatttttattccagctttgcgagtcctgtgtagcccctcttatgttaatattatacatccgatgcatctagaaggcacagattgtgcgctttgatttgataaagctctagtctttgcatatctttatggcacaccttgtgaattcttaacgtatattccccaggagcaagttcgttttataatgaggcctaggatccatcagatcagaagacagtttagtgtcacaacttaaaagactaaatagtgacttgtgcttgtcattaaaaatgggtctctttcaacaagcactcaccaaaaaatagtgttttagtggcttgaaaggttcagatatctcctaataacccacctatttgctagtagggagtaagtggaaaaacatataattaactgtgcaccatccaaataataaattttaaattcttcgcgtgaattgttgatacatattgttgtgaagtgtctcctgcttaagaaagtaagggacttggactgaatatgccaaagaggaggcctctgcaagcggtgtcttggtgatgggaagttactggtaacagcacacggggtgatggggtgaagaggcttgtgtgcgtgggccccgggggctgaggattgttggagaggacttctcccaccttctctgggccccaaactgcacactggactggcaagccactctgcccactgcagtgtggagctataaagcccaagtagggacttcactaaaaggatgcagaggcgaaggcaaagaaaggctcatgctggtgcgtgtaggtgggctgagtgtccctgtccgggagtggggagccgtgttatcttccactgccttaatcctacagcgtaggctttctcagagtcattgccataccttcaactgttccagtggccaggctgttctcaacttgaaaatcttttgtgggacagtgccatgaattttttttcctactgcgattttttagatataaaggaattgaatccattaagtggagattttaaaccctccagtcaggatttaaactagattgtgtaagttcagttcagttggattcaatatctgtttaggaagcatttttattccacttcattgattttagaaaataaattcattgcttggttttaactatgtcactattaaaaaaaaaccactttaatttgtggactcatctgggaggattatgtgtggatatgacatattgagtcagattatctgaccttctgagaagagttcagattagcaaaaacgtggggtgagaagtccttcgcagtttgccacaggtaaggagaaggtagatatgagggaggtagagaaatagggaaataccctgagtaggaatgtaccaggtctgtatatgctcaactcccacagtcagcaggttgcatgattactgtgtatagtcttaaaatataaggttgatttctaagaatttaccctgcttcttggtgacattgtaggacatctcagtttgccacttagcactgttattctttctggctctatactaaaggagataatttgggcaagggaatggaaagaggttactttttcatcctgtatgtttaaggaataaattttccttcatatcgagggcagtggagaaatataactgtcaaactgttcgagtgcaatgatgaagttccggaaggaacttcctaaccttgaccacacccctctggctgtcatacacctcttaaaatgtctttgaatttgaatgtctttgaattgaaatttaaaatgtctttgaatttgaaagctttgggtctacttttaaaatgccaaagttcctggcagggttagttcactaaaccactatcagcaaaaatgtgaaaggctagttgcttgtttccttgctacctgaaaacaaggcctttaaggagtagaaaacagatttggagtttgaaagagggggttaggagagggaaaaataaagttaatgcatgtcccagtttcctcataagccagaaactttgaaacaaaataggaaaataaccatttggaatagcagtgataggaaaagatatatttacccatcattttttttctcttagtcaagacaagtcgcagatcttccttgaaccatttttgacctcaaaacagttattctgaaacatcaattaaactaagaaatgtaatcacagttcaagttatgagctagtattatcttaatgtaattaaatatctgaatattatcacttcattatatcttgtatttcctctgtgatgttagacaaattatgtggtctcttcatgagtcaactcttccaggctttaaaagtagcttcctaatagattgaaatgagaggaacttttcaatcacttcagaaaatgatgtgccaaggtagtataaataatgcttaatcctttggcagatccacagatcataaaatatcatacatgcggccagtcgcagtggctcacgcctgtaatcccagcactttgggagacagaggcagaaggatcacctgaggtcaggagtttgagaccaacctaaccaacatggagaaaccccacctctactaaaaatacaaattagctcggcatggtggcacgtgcctttaatcccagctactcaggtggctgaggcaggagaatcggttgaacccgggaggcagaggttgcagtgagccgagatcacgctattgcactccagcctgggcaacaagagtgaaactctgtctcgaaataataataattattattatacatacttaattttatttttgctgaaaatagtatggcaagccagtgcatctctgttagaaatagaaacttagtagccacctcaaaagcaaggtcataattctgtgaatcagctaataggtggtatttagcagttaggtatgggctaccctcgtgcctcatgactaatcacattgcttactcgtcatgcattttttacaaagtcttgctaaagtctgtgaatctaaaacagagaatctcaaagtgttccttactgaatgttatcctttccgtcagttgataattacctgtgtaaacaagcttttgactgcatctagaaaagcacctatataaattcttctgttttatatggtggataaagtttataaaggagaacaaacataagaatgatgtcaatataaatgaagtagaaacttcaaaaatcatttgcatatgtcaagtataatccagaagagtgaagaaaatacccaggcatttggcaatataggattactacatgttttcctaaagaatttggaactgattagaaacatcctgattttatttaaaagcaatataatgtatttttacagtttgttttcaagaaggttcaaaaaaaaattttttttttttttttgagatggagtttcgctcttgttgcccaggttggagtgcaatggcgccatcttggctcactgcaacctccgcctcccaggttcaagtgattctcctgcctcagcctcccgagtagctgggattacaggcgcccaccaccacacctggctaattttttgcatttttattagagacggggtttcaccatgttggccagtctggtctcgaactcctgacctcaggagatccacctgcctcggcctcccaaagtgctgggattataggtgtgagccaccgtgcctggcaaagaaggctcaaatttggtgacttttaggactagagcagtttgatgtctgcaaaaatctagacaccaatttcttgaaggcaggtactgactgaattctattccagcatatagcacaattcttagtaggcacttaaatgttttagaaagaagaaagagctggtcatggtggcacacacctgtaatcccagcactttgggcagctgaggtaggcagatcacttgaggccaggagtttgagaccagcctggccaacgtggcagaaccccgtctctacaataaatacaaaaattaactgggcgtggtggcacacacctgtaatcccagctactcgggtgactgagacaggagaatcacttgaacccggtaggtggaggttgcagtgagccgagaagattgtgccactgcactctagcctgggcaacagagcaacactttgtctcaaaaaaaaaaaaaaaaaaaaaaaaagaataaagaattagtctaaggcagtggttcttaaaatgtggtcctgggatcgtgagcatcctctggcagcttgttagaaatgcagtttcttggacctcatcccagacctgtggtgggcctagcaatctgtgttttagggactctgctttatacgctgtttgggaactcttggtccaagatgattcttgcaccccacctgatagagtggggcccatttgtcagtgtgctttggatgatttctagtaagccttacccttgcttatggttaagttctttgccctatctctctccactcataggatctcttggaaccatcctataaaactacccccattgacctatcatttattacttagaaaagctcagtagtggtgtcaataagcttttgtcaagaggctagccggctcactaattagaccttgcaacactggttctttttagcttagcatggctgcttaggaatccctagtctccgcgtcaaagactacaacttcaaagactggattttataaaatcacagaagatgatcggccctagaggactggttcatgttaaacagatttacaagatatggatttgtaaacctcaattccagtttaataatagtagaaaatattcccttcctagtgattgttgataatagttttttttattttaaaattctataatttcaaggattgaagagatttacactcatatatatctgatgtcactaagatttagttcaggttacttagaaagcattgggcaatgttttgtaacttttgggactttttccaaagggctggagaatataggaaaagccgtatacacgaagagtttcataaaagcattgttataagaacccatatttggaagtaacttaaatatccaacagtagggagatgtttaaataaattatggtataatcaatagttaaaatattgtacagttataatgacaatttagaaactactcacaactctgaagaatatttaaaaagtaggataaaaaatgtgtgtgaactattgattggaaccttacaaaaatgtatgtatatgaaaaagtctggaagagaatatacaaaagtgataatagttatgttacagtagtgagatatggagggctttttgtttgtttttatatttttctctctttcccaaagtttattcagtagtataaaattacttctataatcaaaagcatacatttatagcttttttttctttttggtgtgtctgtgcttctgatagtaaagatcaaggcaatatatgatttttttcctattatacccaaaatttattttatactaaattttatttcacatattggacttccctgtacattttacttgcctagtctctatttggcatggggaaagaaacccatctctgttatggcctcttaatgttctcagccatacgtaaggtttaattttgttgatttgtttgggtccgtgatctacttgatctttagcatatgctggcaaatagagcttttaaaaaatagtttccagagttactttcttttaaagcccttctctctttatttttttctaatttttttctattatgttcctttttttctaaaattgaatgacctcatgatagagttacccagttttcctgctgctgacagaatgttgcacttataaatagcagctgatgcagatgcacagaaaggtaaatttctgttacaaatgttcattatgagcccttcatataatttgtgataataattttaaaaataaaaagaaatgggccgggcgcggtggctcacgcctgtaatcccagcactttgggaggccgaggcgggcggatcacgaggtcaggagatcgagaccatcccggctaaaacggtgaaaccccgtctctactaaaaatacaaaaaattagccgggcgtagtggcgggcgcctgtagtcccagctacttgggaggctgaggcaggagaatggcgtgaacccgggaggcagagcttgcagtgagccgagatcccgccactgcactccagcctgggcgacagagcgagactctgtctcaaaaaaaaaaaaaaaaaaaaaaaaaaaaagaaatgtaaaatagcaagcgagtagaggtttcccaaaatcagtgtgaacattgtgaaagttatatatatgtatagacacacacacacacacacacacagtaatagcctttgaatgaattcttttttcatcttttctgttgcattgtcctagaaatcccaaaggagaaagttaagtggttttgcctttgacagcggttacagattacatttttcttgtcactttctttgggacttcttttgtgaaacttgtggcatgtcctagaataatctttatcttaaagttttaaaaactttttagaaatttcatatcagtgttataaatatttattctgtatgcctccccagtttgggatgatttcatccttaatgataagccaagacaaacttcagctaacaatatttagtttatagatcttagaatgtgtttatgggtaaaagaacctcgatgtgatgataccttagagaatagatctactgagactgatgcgttttattttcatttttgtattttatttatttatttattgagacagggtctcactctgtcacccaggctggagtgcagtggtgccatcatggctcactgcagcctcaacttcttgggtttaagtgatcctcccacctcagcctcccaagtagctgggactacaggtgtgagccactacacccagctaatttttatattttttgtagaaacaggggttcgccgtattgccaggctggtcttgaactcgtaggcccaagcagtcctctggcctctgcctcccaaagtgctgggattataggtgtgagccacgcgcccagcctgaagctttttttaaatgaagatttattcaacaaatgtctattgagccacttatttgtgccacaagctgtttcagataaggaggataaagcaatgaacagacaaattccattcctccttgagtttacaattattgttatctagatttaacgagaacatttttcaatgggaatgacggtgactaagagggggtgaatgggcctgtgtcttgtaactagatggcattgctaggcaccgtggactcagggggctctctctgagattggtatagtgtatgggaaagaaaatctgtatccttgaatattttttcccagctctcttactatcctattatcttaggttatgttgcatgtattctatattgtatattatgcataatatatttataatttctgtctcagtaatgaagtgatttcagaggaggtaagaatgaatcctttgctaataagttattactttccttgaaaatccaaatgacaagaggattattcactttgcaaaaggattatttatttttataatgttttcacagtagatttaagtggttaactattcaagtgaatttaatttataaataaatcaattaatttaaaaaaattatttaaccctaagaaaatgtaaaacaaagttcatggagactgtcccattaacatcaaaaaaactttttttcttgggagataaagttggctactttctctttaaaaggccattacaaacttttacaagcttagaatataatatttgcgtaaaaggagacccagatcctattttaaacgaccaaattattttgatcaaattacttactatagctattttcaatgtctgtattcacaaactgtttactatttcagttctgttttcaagttgacagaaaatatcatattgaccacttagaaataaattctgaaccacatgaattttttgggaaaaaatgataaggaaaatcaattttgatggactttatataaagttaaatagaattaattctttcatatatacagggccgtgaaatgttacagtgtaactcaatacagatgtaaccaaaaccttcaaattttgcaaatttcaccagagttaatttgtttggttctacaaacatttaccaaatgaccactcattaaacctgcccacctatgaacatgaccttataaaatggtgagacatagcagtatctggcactgggcacacagattaccacagctcaataaaagctgagaaaatgactcagtgattgaacacagcgcattgccatggttgtgtctttaagccattcatgtaattcatgctgtgtattcgacttagcctgctgtatgatctttgatcctaaccaagcacaatacagccacagttaatttctttttatcgtgaacattaaatttttaaatgtcaggttgactttctttgtcctataacttgtgtatgaaatatttatcctggtcccagcttttttttttttcattctcaaaggaggtgaaaccagcttttgaattcagaaaatgcagactaatattaatatcagccaaagtaaaacataattagaaggcagatctactctgaaatggaatctgatatattccatgttctgtgttacacatatttgctgattatttgatgtcatgtctgataattttcatgggaaaattccatgtaccaagcaaaattgttagttcctagagggtaagaatgtgtatagtagttctcattctctttaccttgctgtgcactgtataccttccctaattcccagccaaactgggtgcactatatacactttttaatacttttcaacttgattttattttgaggacaagtgtgtgctttttcagtatcacttgtataggattctccacaatcttagggaatttcctaaactactctttaaaatattaattcctattttatgtattgtttttgtgatacataatgatatgaaactaatgccaggagaaatttccattttgttattctaaagcagacaaaatgggatatgaaagggaaaaatgttaattatagacttcaacattctatgttggacctcgtagttgttctggtcagaagaacaggttttttaatggcctaagggccagagcattgttttgcattcttgtttggagactttcaatggcaactgtaacaaactctgcttagtaccacagttggagaaaggtttacaataatgcccagtacaaaatgttattcagacttactactcatgcataaaataaaactggttctcttcttaaacttcgttacatattagataaaacactatcaagttttggcctcatttgttcataatatgtaatttaattcagttgctcagctagttcatatatgaatcacaagtattatcatcacaattatttaacagttaatctccttttatagggtagatatgtgttaataagcaacttcaaaataccaaagaaactgctttacatatacaagcaaagcaaatagatggaccttgtgacatctctggacaaccagtcttcttggtaggccttcatctgtgggtgtagaacgaaggtaggagtctgctcactaaagatcaatgcaaggaacttccatgctctccactgctgggcaacctgccattctcctcctgccaccacacctgttcagctactagggcaaagaagtgccctgctagacacaagcagatgcttctcttaccaagagaagataatataaggaaggataaagaactgaaacaggtttgaggctttctcttattcatcatctagacttcagttttcttgatttattattccttcatgtcttgccagacacaacagttgcacatgtgttcaagttggttctaggcacaaagggagataccagagcttctgtagctacgagattatcatctgcgtgagccaagaaggggtagtgttgaaacaggtaactaacaattcacagcagtatgtgattaagggctaaaataagtaaagggagagaacaaatgctacagaaattcagagaaggctaagatcagtgtggagtggagggtacagccaggtaaagccttgtagagaaagtgggaattgaactaaactttggaagctcagggattgacaggatagcctcacgaaaaatactaaaaacaatacagaagaagaaacaagaagggaattaaaggatgattccatttgtacaaaatatcccgagtaggtacatccatagagataggaagcagatgagttgccaggggctgaagagaaaggaaagggggagtgactgcttaatgggtacaggatttcctttggaggtgatgaacatgttttggaactacacagaagtgatgattgcacagctttaagaattactaaataccattggattgtacacacttcataaatacattttaaattgtgaattttacctcaaattaagaaaaaaaaagttcctggtttgaggtaaccaaaagagaaaataatttccagtataagaaaacaaagcctggaatagcaaataagcatagagttaagggaaggtttgcagagattaaccagtttgaagtggagagtgcgtggatgtgagtagtgagaaacatggttggaagtgtagattttaggccagatgataaaaggtctggaatgcaggagaaggagtgtgggttttatctcaggagcggggagattccatatcgtaagaagaggggtgagatgattgaatcagaagattaatttggcagtagcatgcagggaaagcagtcaggaggctgctgcaaaatccggacataccatgaggagggcctgtaagagtgaagtggaagggcagatgtggacgatgctctgaagcagagaatgatgctcaaagtgtggtccgtggcttgatgctgccccacaagtgttagttacagacctggcacacagtaagggtagatttgagggtaagtctttaaaactttaatattattatgatattcaagcgcatgattttgcatttcatagcaatatccacagtaggtgtaagagacaagaaaacttccttcatgccagagttagaagaccgctgccctaaaggatgaatgagtggagttgatgactgactcctgtagggcagtctttttttttttggagatggagtctcactctgtcacccaggctagagtgcagtggcacgatcttgactcactgaaacctccgcttcctgggttcaagcgattctcctgccttaccctcctgagtagctgggattacaggcatgcaccaccacacccggctagtttttaaaagtatttttagtagagatggggtttcgccatattagctaggctggtttcgaactcctgacctcaagtgatctgcccgcctcagcctcccaaagtgctgggattacaggtgtgagccactgtgcctggccaggcagtcatgttttatggctcctttacagtgttccaccccttgtgctagacactgggaccagagcagtaggcaagatggactggcactttcccttgtggagctcgctgtcacataaatagtccttctttgtcaaaggattgctaggaattaaagggaggtctggttcagctcagcataaggaaagcattctaataactacagctgtcccatagtgggaaaggatgatttgtgagaaagtgagtgcttcgtcattggacatattcatgcagagtctgaatcatctctcaagaagtcttgctttggtgggagaaatggagtgggtgacttatcaggtcttgtgtaactaaggtgccatgattccctattctctaatgtgaacttagtagaacatattttcttcataactggctgtattggaacaccagtacatatactaagtttctgtctgtttctattagcacttggaattacagttacctaaatttacatgtagattcaattttgaacttatctgtacagttagttccacaattctttttaaagcaagtactattctctaatgccttctcagacattcacttatttccaactttaatatttatagtaagttcctctttcacgtgatgtgtgtcacaagtcacgcaaatttctgcagaagtgagttacataaattgcagtcaagctgctgacctgtaaatcattcattcttgcccccgtgatgtcatattggggttgaataagaccaggcgtgactacccacttgaggatgtctttgggatgccattttaattgggtgaaataacttttattattcaacttatgtgaccagttactgcaaacgtcaaactgttacgtttaacagctagaatgatcataggttaattgcactttagaggatgtggaggacgtagactcctataggagtgatagccttactatattttgggggaaattctagaatgtttttaactcaaaggacattattttcatgaggatgattttaatgcttatggtatcaggtgaagaaaaatgttagctgcagaagaaaaaaatttgtttcacttcaagtctagtagaatattttgttaaaatagccaattttcattatgtattattaaaacaggttatacttttcattaagatgtatgtaaaaatttcacatgtgcctggtattacacagtatttacccaataaatatttattgaatattgttgaatgaatgtgaggtgagggcggagaaaatatttcaaatggaaaaatagtatgagcaaaggaaaacgtgaatacagaaagaagcaaagatagtttctttaaagtcgcatttatatgtaataacaatgggaaaactttttggctgagtaattttggtggctccttaatttcttaatatttaacaactaatgacgactctctctgatactgatatccaaagcaaatagctgaccttgaacatggaaaagttactgcctctgactaattggaatgtctgagcctttcttgtcttctatcttctttccttcctttcttcctctctttcctcctcttttcccctcaattgtagcactagctcttgttcttgttccctctacccttctcccctttttcgttcctggtgttccctatgttttcttctttccttcttttctgtctctgtttctctccctctgaatttctgcttcctttcctctctttcccaattgctactgtctctgcctttggttgtctctgagtgtaatatggaagtgaagtatatttttatctactaaacacatgatcaaatagggattttttattctgaatttaacattaacagaaatgttatgttgctctttatgagacgaaataaagttggatgtcacttataaatggattttgattgatttttttaaagtattgactcactaatcttcaaattggtagaaggaggtcagtagactttgactcataaatttttgtaagcgtaatggcttgttgaataactcctaattgtctttaacaaccttggtttctgtcctcttttttccaaatgcagatatagcagatgtcttcagcatctaattttttattacactttcatatgtgcagtcatacaagagctggagtatttgttttgttaattctgtatctttattgtctagaacactgctaacaaatagcagatgctcaacaaataattgttgcttaatatttcaaatctgtagtgtcattctacctcctaatcccaaatagccatcataactttttagactaacattactcattaaggtaacttagtatacaatgaatttataaatgtaaccatacaatttcatgcttagagaacgttttctctccattctctctacctttagaccattctgcagtttaaatcttgaaccaaaatgtcatttaaccatccttctgaacgataactccataattaactcaagcaccaaataagtcatctagctcttgatggtaataattaatatttgttgaatgcttattctatatcaggctctgttttaagcacattatatacattatctcattttattctttcagcagctatgtacaccagcagtcccccacctttttggcaccagggactggtttcatggaagacagtttttcaacagactggtggcaggtggaaatggtttcaggatgaaactgttccaccttagatcatcaggcattagttagattctcataagaagcatgcaacatagatccctcacatgcacagttcacaatagggtttgcactcctttgagaatctaaggctgctactgatctgacaggaggcggagctcaggcaataatgttcgctcgcccaccactcatctcctgccacgtggcccagttcctaacaggccatggactgctactgcttcacggccttggggtgggggactcctaatatacagtattaaagtcagatgaagaaacaggaacagagaggtaaaataacctgccaggtgataaaaactaggatttgaacccagacaatctgaacccagccaccctgtcatgcacaggagcctactttgcaagtatttggccttccagggaatgagatgtccacatcccatcaccaccgccaccactattccacatttaacaattagacctatacaggtgtaaccatattcacatgtaacagtggattaaatttaaacttttagttgtgttaattcactgcaaatcagcttatgtgtcttgtaagtagtaagcaacttgaaagtacctgttgttgcttatgacaagataaattagatcaccaaatctttatatttagtaacttcatcatcacttagaaatcaaccgctgggccgggcgcggtggctcacgcctgtaatcccccagcactttgggaggccgaggctggtggatcacgaggtcaggagatcgagaccatcctggctaacacggtgtaaccccatctctactaaaaatacaaaaaattagccgggcgtggtggtgggcgcctgtagtcccagccattagggaggctgaggcaggagaatggcgtgaacccgggaggcagagattgcagtgagccgagatcgcgccactgcactccagcctgggcgacagtgcaagactccgtctcaaaaaaaaaaaaaaaaaaaatcaaccgctgttaggaaattctttttttttttttgtctctcttaaattcctcttaatctaaataaagacattttattttgtctcaggatacagaggtcaattgaatatagcaaattttgtcagtgttgtaaaaaatctggagaggaatgtaaacggctcatcttgtgtctggcctctaattgaatcagtcagctatggtggggttaggggacagatccgtgtatttacttgcataggacaaaaattagtcatttgtgagtgttcacatacttccacctctcccaacccaccaaagagaattgtaatttgacagtacaataatcccttcccctactcttaaaaaagttgatcttacaaggtcagtcctatgtttttatgaaaaagaattagagaatgagtagaaaggaactagtcaagaaaagcagaagatttgggttgatagacataaagtgctgggaatgagaggaagagagagagatcgtgaaattcctgggctgggaataaagactgtgtgtaggtaggacagcctttggtgaaggagacactttggagagcatggtgtgtgaaaacacttaaaggaaaattaaagggaattaagaggaaattgaagggaaggagtatatgagaagggttgctttgtggttataagctgaattttctttaatgtattttgaaagaccccggtaaagaaaggaatttcttttaattttgcagagaatgaggagttgtccaattaggtgttgaattgttcttccttggaactctcaagagaggagttgtgtttagagatagatttgggagctgtaagcaagtagatgtgttatgagtcacagtgctgagggcaagaatataaagaagagatgaaggccaagaggaaacttagagaacacaaaatagaaaagctcagaaagcaggggtaccaggaaaagggactaaaatagtcatcataaggtaggataataggaggccgtgcacagtgactcacacctgtaatcccagcactttgggaggccaaggcgggttagagatcagcatggacaacataaggagatctcgcctctacaaaaaaatgaaaaattagccaggtgtgatggcgcacacatgtagtcccagctacttgggaggccgaggtgagagcatcacttgagcctgggtggttgaggctacagtgaaccatgatcacaccactgtactctaacctggatgacagagcaagaccctgtctcaaaaaaaaaaaaaaaaaaaaaaaaaaagggagaaactgatttcaaggagccaagaaaagagacagattcaatagtggaggatgggcaaaatatcaaatgctgcagaggcagagaccgtggtgtgctggagccacattgttaaattttcacaattttattgagccaatcgttatgcatgctattattaaaacttaaattgaatacagttaaacaaattatattaaaaataaaagtaataaatacacaaagcataccacttccttattactttacattgctgagatgattcatgcccatggtatctgagtagtggaaatacactgtaatatttgtgctgcttttaaatcagtcatggtgggagtatttacaccacaaaaattggcaaacactaccaatcagggcatttttctctgcaaagagagccagttaaatatttaccagcatgccactgagaaaatagaattgaagtcaagaaaagtctaatggatttagcaagaaggagatgacatatacttaagtttcagtagaaaggtaggtgacaaaaaccacagtaaagtgggttgaggagtgaaataataaaaaattaaaaacatgtgttcaagtaagtttgagaaccattgaattaaaccgagaagcagattgttattgttgatgatggttttgttttttaaattacaaggtttgcagagtacatactatgctaatgtactttattaatctccaaaaatgaagaccatatgcaaatttattttatggtagaaccttttctgttggaaaatcttagggaacaagttatctatagaatacattttgggaaatgctgctcttgtatttcacaattaagatgatactgattgtaaagtttgagattaatattcttgtttttaaagtttcatgctttttaaaagaaattttgtcaagaatggatgttgaattttactgagtactgtttttgtcgtttttgtagtaatcatacggttttttgacctagtgctgtgacacattttaatagattttcaaatattaaatcctctttgcctttctgagataaatgtaacttgattatggtggttcattattttaatagaggatttaatttgttaattaacattttatttattatttaaaatcaatattgataagtgggactgatatttttctctgttgtcaggttttaatatcagtgttatgacactccagaaaagttaattatgtcactatattttttcctactgagaaacagcttatgtagtaagaaaatcatctcttccttgaaagctttaaaaaatcaactataaaactaatagcttttggaggaagtaattctttgataagttttcaattatttccattgttaattatttttctgtatcttaagtcaacttcaccatttatttcatttcagacaagttagtctgagtaaactgcccttggcctgccttattatttacttaggttctggtaaaaatcccatcccagatgtaaaatcattgtgtgagtagagatgaacagcccctgtctaacgtgggctgtgtttaagtgtgaggcaattgtctgctgtcttcacagcccagttctctggcttctagggatgatgggtagggtgtttataatcgaatgaatataaactaatcttgagttaaatgaccagatggcagaggtgaaagaacggcaatccttggtgtctactccccccagaatggatcttgtctctgccctactgtgtttcacatctggggacgattccttctaagatacagtgcagttctgaatgggttcagaaggaagaggactggtagcaggagggggtctgggggaaaggttacctaaatctgcctgactctgacagtagtaagaaccagccaagtgttctgtttgatccgacacttggggctacacactaagaatagataccagtttctagaaatctttactttttgcccatctacttctgaagaaatacctatgtatttcaaaggaaaattggcacaagtatcaaggaaagctggtacaacttaatcttaacctggaatcaactcctttaatttttacatacagatttggttccgttttttagattaaaatttttacctttgttttcacgtatttgagccctttttttttttttttttttttttgagacagagtctcgctctgtcgcccaggctggagtgcagtggcgggatctcggctcactgcaagctccgcctcctgggttcacgccattctcctgcctcagcctcccaagtagctgggactacaggcgcccgccactacgcccggctaattttttgtatttttagtagagacggggtttcaccattttagccgggatggtctcgatctccagacctcgtgatccgcccgcctcggcctcccaaagtgctgggattacaggcgtgagccaccgtgcccggccgagccctttttaatagttgccttaaatgtaaaaaaaaaaaaaaaaaaaaaaaaaaaaaaaaacccaagtggtaatctgtcatagataaagtgtttctcatctctcatgtgttttgcctgttgtgtgtaatgagcggtttcgctgtgggtgctcctttcatttctggctaacacattagagtgtgtttatgtaaattgctgtgatactggtcagcagtcacagccagaaaacataaaacatgtgggcattacacactgacacattagctctttctaaagaagaacatgtcccattctgaaaagaaaattattgctttgtttttgatattgagtctgtattaaaattttttttttatttttgagacagggtctcactctgttgcccaggctggagtgcagtggtgtgatcatagctcactgcagccttgaactctgggactcaagagttcctcctgcctcagcctcttgagtagctgggactacaggcacatgccaccatgcccagctaatttttctgttttttgtagagacaggatttcgctgtggtgctcaggctggtctccaactcctgggctcaagcaatccgcctgcctcagccttccaaagtgaaaaggttttctctcatttttcaaatagaagtactaaacaatgccagagaaataaataaacaggcaaaatacgttggctatagtttatattatttcctgctacagttaacaaaatgggaaaacattttatcttcatggtctactacatttatgccatgtgttaagtaataaaatagcttttgtaaattataaattaaaaggtacagatttaaaagagaaaatactgtagagtttcatgtaggtaagactgtgtagaatgtcgggtctcgatgttggcgctattcaagccctgatgataaggcttttggcattagatgctgttttgtcttcatggaaaatacagctattctaggatccttgagcctttcataagagataaggttgtgaatcctaagaccctaggaccatttacttagatgatctgctctctggttcgtcctctgaaaagtctgcttcgtgaggggtgtgctgcatttgccttgcctaagtggtgtggcacacaactgtactgtcaccttaggcttaataaccatgtgtcatctagaatgaagttatattttactaaggatcgtttttgccatgtataacttctcaaacattaactttcagggttattaatccttttaaggtctagtttttcttaagtctgtgcagtaatagaggtatcgtcattcatgtgacataaaagatggaaaggggcttcattcatgttagtgatggaaataggaaagtaggtgaagtgattctaatagatgtttcttttatgaaataatttttaaaagattctccagccctgcatgatttatgatgaatcattttgtggtctgttagttacttctagagaatagaaagcattgtaggctcagggaaagcaaacattcagaatgaaatccaatagagaaggtaaatttatttgggcatgtacattttggcagcctaggctgtgtacatgtgtacacattctgaacatgtgtgtatattgaaaatcttgtctcttttttattgttaagatttgaaagaagccaacactaaaccacaaatatacaacaaggccattttctcaaacgagagtcagcctttaacggtaagtaaaatcagaatttatactgcatttgtattgaaaagtatcccttttaaagaatatgtaaattatacattgtattttattgtaaaatttcctagagagtgatttttgactattataatactttctgctatataattttccagtcagttggactatgcagtgtaacatatttgtctaacacaaaacaaaggtaagataggaaaatgacctagaagttgagaaataactcaaatccttaaaaatgaatctctaattaaagtcttttatattcatgttatttttctacttaagtaataattacattattttagattcttatgatacttttaattaaattgagataaaaattaagatttctttcatggttccctattggccatgcagtcataatctgtggtcacaaaggagaaagtagcttcgtataagccaaatgtgctccattctaggaggaagaattgctaagttatacccggcattaaaactacttccttcctggaactgactagatgggggtaggatttcagaggctccaggggctcccaaagtttcgggatatccaccatggataatttatccttccgaggcctaactatagggttccaagacagttcttagtccccaacttttttgggaaggaaaaggagacctgtcactttaaggttttatgtagttttaggactattgattttggtaaatcatgagctatcacagaatagctcattttcctgaaatctgcaaataaatgaaatccacaacattgaataaagaatcatcagatagatagatacagaaattaatatagagatgtgtatatatgtgggttattatacatatatatgtttgctaactctgtctgcttctagaggacctagaagcagtgactccccagtagcaaactagatcgtggtttccaaatacaggctgagcatcccaaatctgaaaatctgaacttcaaaatgctttcagattccaaactttttgggcatataggtgatgctcaaataaaatactcttggagcattttggattttggatttttggatttgggatgctcaacaagtaagtgttaagcaaatattccaaaatccaaaaaaattcaaaatccgagatatttctggtcccaaacattttggataagagatactcaatctataccatttttccttaaaggaaaccagagctctttggagaagtggttaattctaaggctggggcagggaaaatacaggctgagcctggaggatgttgtggtcagcctgaaagagctcccagtggccaaagctggaacaatttcatcaataaaataactacgtataatattgaattataatctgatttataaataaaatgtatacacatgagtccaaattgatatacataaatgactgaataagtaaataaatggggggagaagaactctttcttgtagaagactttaataaatgtaaaaggaatgagggaaatagaaaattgttgtcacaaagccacagcagtaattgctgcatgcaacttccactaatactaaaattagtgaggtgggccaggcatggtggctcacgcctgtaatcccagaactttgggatgctgaggtgggcggatcatttgaggtcaggagttcgagaccagactggccaacatggtgaaaccccatctctactaaaaatataaaaattagctgggtgtgatggcggatgcccataatcccagctacttgggaggctgaggcatgagaatcagttgaacccaagaggtggaggttgcagtgagctgagatcgcaccactgtactacagcctgagcaacagcaagactccctctcaaaaaattaattaattaattaattaattaattgagtgaaagtttaaggtgaaacagaatatttgcataacctcaaagtatctgcccccacttactcagtaattacaaaaggaaaaatagtaactttacagtggagaatcctgatggacaccaccttaaccacatgatcaaggtcaatatcaaggatagtaagacatagtgagctcatatgcccctgatagaatgtcctgaaaaggacagatcatgcctttcgaattcttcccaaacatccttaatctcatttgactatgagaaagctttaacctaaatacaaacacattcttcaaaatatctgaccctcgccgttcaacagtgtcagaggcataaaagacacatctttgctaaggaatcactacagattaggaggagaatattgcatgacaattaaatgcaatgtgggatcctggattggatcctagagctgaaaagggacgttagtaggaaaactggtgaaatccaaatagttatagtattgtgccattctcaatttattaggtctttttttgcttttattattatttttaattaacatataattgtacatatttatgtggtacagtgtgacatttcaatacgtgcatacaatgtgtaatgatcaaatcagggtaagtagcatatccatcacctcagacacttatcatttttttgtattgggagcattcaaaatctgctcttctagctattggaaaatatacgataggttgctgttaattatatttaccccacagtgctatagatcattagaactattccttgtatctaactgtagttttgtgtctggtaaccaatctcttgctatccccagcccttcccagcctctagtagcaactattctactgtccacttttatgagatcaactttttaaacttcgcatatgagtgagaacatgtggtatctgtctgcttatttcacttaacataatcaccaccaagctcatccatgttgtcatgaatgacaggatttcattctgttttatggctgaatggtattccatcgtgtatgtgagccacattttctttatccattcatctgttgctggaactcaggtggatgctgtatgttggcttcagtgtatcaacactgataattgtgctgggactgcactgtggtaactggggggaagctggctgaaaggtatgtaggaactccctccactatctttgcaacctttctgtaagcctcagattatttcaagttaagtagttttttaaggtcacaatggagtcactaaataatcattctcattagattttcgctaatttatagaggaagattctgcatttgcaaatcttagaacagagtaatgcaggggcattcttttcatcaagactatttcttgagctattttctaatccttctcatacacactcccatccctacctctacaagtctggtctctatttagtgtacttaatactttagccagttgcttacagccctctagaaacctgtgagtgggaataactccacaaagctctgcctcctcgggaaccacatcttagcagtgttttttgttggtgacagtgagggtctaatttgaatgttcacaccagaactgtcaaagtatttcatgcaatactttgaaaaaaatctcttgatccaccaatcatcattacccaagacaggatttgaacaagaatttagaactcgtagtcttatatttgtttgaatgggaagaggagagaagcagaccgtattgtttctaggcaaaatgtctgggaattcagtgaatacaagtagtactacgggaactttcattcttaaaggaaatcaatgacagcattaagttgtctagaaaatcagtttaactttagaaaagcataaagccgtttaaaatcaaaagagcaaactcttaaaagacaaagttaggagcaggtacgtactttggcgtctgaaaatgagcggtctaaattcaagcattttgacatgaaaaatgaaatgtgaagtatttggaattcaaccctgaatttttttgccggtaactcttgtgtatgttcaactaatttagcaaactgcactagttcacattttttaggtgaaacaagaaataacagaagttgcacctaaagtagtcaaatttcgtagagacagaaagtggaatggtgcttaccaggggctgcggcactggaaaggaacggggagttagtgtttaatggggacagtttcagttttgcaagatgaaagcatcctggagaatggttacacaacattgtgagtgtacttaatgccattgaatgcacacttaaaatggttaaaatggtaaattttatattgtgtatattttatacagttttaaaaactaaaaaaaaaaagtaacaagtcttcagcagttatattccacttcattgccagggtgtctggctctaaggggaatctctgcttagaggaccattaatccttaggaacctttttttttttaaaaaatctaaagatggttcctggacatcatttaccaactggcagtttaaatggcctgtgtaccataaattgtgaggttaaaaaaaatctggtacacaggaaacagtagagctgggtggctgggagagttgcttttggggttaatttataactaagacaaatgaatctatgaaaagaagaaaagccccgtaagaaatatgctaactgatggtgagggagaaaagggtttagataaaagatatctgaacttgtttggtctcagtgattttatgactgaggaattcaattatagatgtactggatggagcaaatcacaattctgtaaattactaataggacacaacgatctttatcattttctgtatgtgctcacatgcaaagttcagtcctgccctctgtaaataaactctctgcctagctatacccatgctatgaattcagggcttgagtgttgaaacataggcaatgcttactgccccaaccttaaaggcaggtggaactgagattaggaacgaagacaaattacttttgcttttgaattttggccttcgggtttgtttcatttttgttcatttcttttcatttttattactaaaaagctgattttttaaaataaaaagatcacaaccacacaaaaaaactattcttctgtagtttcttccagttttgttcacatgtgtatgctttttatttactgacagttaccgtatatacatcacttggccctcagcctctttcactgttaggtcctataaatttttcatttctacctggctttcagagttttcataatggcaacataatcttagtgatagcacgtaatttactaagccagtttttaggtggcttctggtgtttttattattgtataattatgttttattcttataccattacataccatgaatatctttatgtaaatatttcactcaattaaaaaaatattttcttcagataaattgccaagagaggaatttctttgttaaagtgtttgagcaattaattaccttactcttgcattattaattttatgacatttcaaattgcttggaggatatattaatagcaaatgaacaaaacatacaaagcataaatatattttggaaatacatttaaatgatttctgttaatttaaatgaattatcatttacttaaggctttgggtgtatgatagtggtgggaggctggagagtgaagcgcttcaatagttgtttagagattagaacgatgtgagtggtttatttttatttttattctttagaggcagggccttcctctgttgctcaggctggagtgcagtggtgcaatcatagctcactgcaacttcaaattcctggactcaagtgaccctcttgcctcagccaccggagtagctgggactacaggcacacaccaccgcacctggctaattgctaaattattttcagagatgaggccttgctatgttgctcagactggtctcaaacgtctggcctcaagcaatcctcctgccttggcctcccaaatggtgtttttttaaaaaacccagcttaatagcaaagaagttacagattatttcctgaggctatgtcaaatttaaataaaatgtaaagactgtgctgtgggtcccttaccacctttaagtctaagttgctagttattaggaagaaatgattgcttttgaaaatgcagatttaacaaagtaggactttcatagagataagttcaaatcagtttttaaaaaatattcatttaaaaccaaatatttttaaatttgtggtttgaatatttgagaaacaaatttaagaataaagatacaataagaattttaagatacaaaactatatgtttacaatatattaaaatgaataggaaaaagattatgggtgaaactctctcctttttattctcaactttctgtttgggggttcagtgtctccttccacagttttctttgcctcaaataaataaagccaattatgagctctacagtctgagttatagtattcttggttctttgggtaccgccaataacttatatatcacaaataatacatatacagtgttacatagtaagtgctcaataattggaagctagttccatggggcaaaaggaaagcaactgtgttctctttcatactgttctagttttttttttcctttaagcttgtgacttcttgttcttcagtccagatcctgtgatctgaaataatttaaattattaaaagttagatatttttaaccttttatactcccactagacaggacataggaaatttagtgacgtaagcatctttaataatccttaaaagtcataagacttggtggctgggacaagaaacaattaggtataaatttcagggaaataccataatggcatgacttttgttttcttttgttaacaagccacatggctttcctctcaagcccaaggaatacatagtgtgctctccttttaagatttcagttttgtctgcatcacagagatgtatgtttccctaagtttgctcactataatttgtgattgctatttttcttatctgcgaaggaagtacagcagctcccactggggaaataccatgttaagttgatcctaaaataagaaatctaagggaaaggtcacatcctaataacttgtgcaaatctaaattcccatggagctttgcaatcattttgtaggagtataaaaaatctattattgaacttcttccagatagcgtcgtaaaattgaagtacttttatatacagaagtaaccactaaattataaatacaattgacaagacctaattttttaagcaatctattatgaatacgtagagaaatctggcatgactaataaaccacaaaatctttcttgtcactttcactggaaatataaagtatattactcatgaagtattgtgataattacatacaatagtaatttcctattactatagagatatttaatactcaatttttactttttttttttttaactgatgctgaccttttagttatactttggaagaaccttaaccatgttgttccagggaaattcactgctttgggagccaggtcactgagttactactgtctttcctcttctgttctttggccatcgtttaagtttccttacctgttaagtaagaaaggtttatgttaaggatcaagtaagatcaaatatataaagggtttagaaataatttacggcgtatatttatggtattactgtaataattgccttgacttttttatcttttccaaatgaataaatcaaaatagaaagaggttgagtaacttactcgagctaacagtcaccggtacacgctaatcagtgccccactcactggtcaaatctaagtaattcttcaagggatggaatgtaaatggaaactgctcccttatgctgcatacagaggtattacattttgcagtaaggattgattgtttcctagcacgtagtaactaacctgttttgaggatgcgttgttagaaatgcatcttggacatttatgtatatatacgtgcatatatatatgtatgtatgtatgcatgtatgtatcgctagaggaatgtgcgtccttgctttcttactacaaattaaagctgaacagaatgtggtgtttgtcttttggtattgtctgaataaataccaatatctactagtatttatttccttgctggtaataatgtgttttcattaatttaagatttataggaacatacttgggtgctctgcctgctagaaactgatctgtagcatgagccatttgttgaaagtacttagatatttctgcatttatttatttctgtttcagaccccacaataactatattactataagaaatataggccagacacagtgggtgggtcacacctgtaatcccaggagtttgggaggccaggtgggtgggttacacctgtaatcccagcagtttgggaggcccaggtgggaggatggcttgaggccaagagttggagaccagcgtaagcaacatagtgagaccctgtctctgcaaaaattaaaataaaaaaattagccaagcatggtggcgcccgccagtaatcccaactactcaggtggctgaagcaggaggatcacttgagcagggaggtcaaggctgcagtgagccataattgcaccactgcactccagcctgagggacagagtgagaccctgtcaaagaaaaagaaaagagaagagaagactaaattgttttttacatgcttatctatgtgatgtgagttcaaagaatctctgcaatgattcaatatctttttaatatttttgtttgggaaaagcattaagctccttcattgtgttaattaattcagttttctcttttatgttatacaccagcatgaaaagtgtgagatgttagaggcccacatcagtaccctaccacatatttaccaggaaaagctgactttatgcttaatggcagcagtcacccctttaaaatgtgtggcctctgtgtccgatggcgtggactcacttcctttaagttcatggccttctgaagatcagctgggctgggatgagtggtggctggcatgtggtggagtgtcttgcttccctctgatacatggaagttataaaaagtctcactaatgaatgttcttaatatatatggggtaactaatttatgcatcagactttggaataaaagtaatagcaacagcaaatgagagtctaagaataattcatttttttgatacacttctgcccccaggtgccaaatgctacagggtggaggcggagggagacagaaaatctttctgtataggaacctactgatcgctgctactataaatttttctaaccattttatttccattgattattgttttagttcaggttttccttattcctcatttgggttattgcagattaactctctgaccaagcaatccatcgtttccatcatcctccagtccatagttatatttcatcccctttcagagagcattacagactctgtggtcggcccagcctacctcttcgatgtcatcacctccacctgctcccacaaatcttatgggtcagacacaccaggctgctcagtgtcctctcacagtgctgagtttatgactgggatggattgattttcatactattgttgattcaacattttttatagtatacttcttgagaacagagaccatgttcagggatccctagtgcttaatacaattcctaccaaatattatgcaagtaataaaattgcagtgtcattgaacattttcagatatgcatttatttctactaattaatcttagtagtattagcagccttgactaaatattatttctaaattgatcaacatgcgatgcaaagcatgctacagaattgaattaagaaaagaatttccatgagagatatgcatttacaattttgtagatttaatctttgcatagacttttttgttgcagatactgaaaaggaatccataaaaatacatagatccctttactagccacttttaatttacggatgaggaatattcagttcacagactggctagcgctttttagctttctttagcgtttgaagcagggcttctaagtcacatgaaagactagactagttattggcatttaaattttttggctcctacttgtatgtattatttattaaacaagtagttattgcactacctttatgtatgcagcactgttttagacaaaatagaattgtgacatattccttgctcatggtcttgtttcaaagacaatacctacaactgaaagaattaaataatagagccaggcaatatctgattgaggaaaagcatgcagtaaaagttcagagaagaagatacacgtgcagaactggaatggtcagtagttgcttcatgagatggttgcactgagctgaacccttaagtaaggatgtggatacatgagaaggaagaagctggaaattttaaacatgaggagcttcatggggaaagaatttcaagctatcaaaagatatcaaagattgaggaatttggacttggtctgggaggtattggggatcttgaagatttgggggtagggaagtgatatgacaaaagctgtgtttggaaggcacaagtgaaacagagttgggtttttttgtttgtttgtttgtttgtttttttcagacagggtctcactctgttgctcaggctggagtgcagtggtaccatcttggctcactgcaacctcaacctcccaggctggagtgatcctcccacctcagcctcccaattagctgggactataggcatacaacaccatgcctagctaatttttgtatttttttttaatagaggtggaatctcgctatgttggccagcctggtcttgaactcctgagctcaagccatcctcccacctcagcctcccaaagtgttgagattatgggcgtgagccactgtgcccagctaaaagagagattttgatagcttatatgatttgatgactgaataaaaaataaagaggatgaaaaaagtgaaaaatgaatttttggctgataaatagagaaatggaggtattaaaagaaattgggaagagagactctgtttcaggaggaagagggattagcttgatttttggatttgatgaagatgtgaagttaggtgaccagacctcgattcagattttagaatcagactctttgatttggtgtcattaacattgattgaagaatgttttgaaagctgaggtattaagaaacaacacaaaggtggagtttaaaagaggaagttgagcgtttggagagagtgccatgccaaaggaggggacttttaagaaaaggaagacaacacttagtacttctgtgtacccagccttgtaggaataactttacctgtgtaatcttattttattctcacagtaccatgtaaagtatgaattatcattgtccctatttgacaggtgaattaagtgaagtttattgtggttaaataacttgcctgaatgtcgtgctgctggtgcaaggttaatctggatttaaactgagatctgtctgtctaacccccaagcccacatatttttttaattgaaatgctgcaaaatggtaatgaaaggtcaagaagaataaaaaaaaaaagagtaaggccattagaatgttcaaggagaagatcattgtttaagattataaaagcgtagtggtaggaatatttcagcatttagaaggtaatagtaaattaatggaggaagtgaccctagaccattgctaatttgggcagcgagtaggacagttctcttctgaggcagctgagtcaagtacagcagagaagagtgctcatcagtggagaaggtgggaatagtgttggagaggtctcagaataaacattaagtaatcccagcactttgagaggctaaagcgtgtgaatcacctgaagtcaggagttcaagagcagcctggccaacatggtgaaatcccgtttccactaaaaatacaaaaattagccacgtgtgttggcactcacctgtaatccccgttacttgggaggctgaggcaggaaaatctcttgaacccaggaggtggaggttgtagtgaaccgagatcgtgccactgcactccaggctgggcaacagagcaagactctgtctcaaaagaaaaaaaaaagaatagacattaaaaagacacagacgatgtgagaaaaaagactgttcccaccaagtttagaagaaaggataatgaggttgatgtgcctgctgtgccggggggataagagaaaggtgtcaggcaagaatagatgtataaaagcactagttacaactatactttgaaagtcatattttacctggaagaaatctcaaaaaacaaagactacatggcagttaagtgacccaaatcaagtcactttccctcttcaggcctcagttttcccatatgttagacaggttaaattaggtgagacaaactgatcttttaggaccattctataatactgtgcttaactttctaagcattctggaatggagcagccttgatcttttcacagatacgtcaaccatatagctttatttataaaacgaacatgttgagataactaaaattagagttaatatcttcataggtacaaagaaacttttagaacttaagtccacaagtcagaatttctggatgaattcctttttcctcttattaaaaattgttaagatgtctgattagactagttgtaaattcccttgaaagcaaggatgatatcctgctctctactgtgtcctcagcatcaaggacagtgcctaacacagaacaactaccaatgaatatttatgaaatgaatgaaagtttgaattgcaaaattaaatgccaccaataaagaggctttgtgagcgagcgtggtggctcaagcctgtaattccagcactttgggaggctgaggcgggcggatcacttgagctcaggaggtcaagaccagcctggccaatatggtgagaccccgtctctactaaaaatacaaaacattgtctgggcgcagtggctcacacctgtaatcccaacactgggaggctgaggtgggcagatcgcctgaggtcaggagttcaagaccagcctggccaacatggtaaaaccccgtctctactaaagatacaaaaattagccaggtgtggtggcaggcacctataatcctagctacttgggaggctgaggcaggagaatcacttcaacccaagaggcggaggttgcagtgagccaagatcacgccgttgcactctagcctgggcgacaagagcgagactttgtctctaaataaataaataaataaaagtataaaaaacgagccagtgtcgtggtgtgcacctgtagtcccagatactcgggaggctgaggcaggagaatcgcttgaacccaggaggtggaggttgcagtgagccgagattgcaccactgcactccagcctgggggacagagcaagactctgtctcaaaaaaaaataaataaataaataaaggcttagtatagaactcttcccagtcttttttctccactttgattttttttttttttctcaatctcttccatgacaattcagggacacattaattgtgcaaaaggcagtcacttgtctgtattgtctatttttatatcacttaaattggtttttcatttggttgctatgttgacattttatgaaaagggaactaaacttgtgaattgactgacagtggaaatcataggattttcaaaaataagttttgtagttacttaaaaattagtattccagttttcggtcgttcattagtttaaatgttaccaataatattttctttataaaaagaaattatgttttatattgtagaggaaaaatgatttagaaaaacagaaatctatttctgaacaaatttagtgatgctgatgtaagaaaaagaaaaccacaagaagtcatcccaaatttagtaatgcttcctatcttaaacacatgatttcacttgtcttctggctagctaaaggttacaaaatgtcaccataataccttttacttccttatgagttcttgctactccaccccctacccccaaatatggtagatcgagttgtaaaaaccctgaagtatgacaatatagtttattgcacttatagttagaattattctgccaagatgagctgagaaatggaggaaatggatttaaatttacagcagtccagtgatacacaatatgcgttttccagaccactgaaacagcaagccaacttttaactttaatacttcatacagtggaaaagttacagctgtaataaaatcaacagatacatctctggttaatgttctcaaatgagaaaattgactgcaagaacatttgtccaaatacatgtttttatatattttatgctactttccagaatattaacagttttacatagcgctgttttggaagtcactcattgttttcatctttctgattaagaaatagggccaaagttgtacattgcactagtagtagacttttaaagatgtctttgtctctttttcagtactaagcttcaagttcatagattatccttgatctttcctttccctaggtagagatttagagtatttgtttatgtgagatatataaaaaaaaaaaggagattttaacaaatgactgtttgattcttgggtagaaactaatactgatcaagaccaaccaagtattcagacaagatcgggcgtgttcagggtggtacggccatagacgacaaccaagtattcaaacattgagtgttcagtggaggctgtacagatatcctgggttttgattcaagatagtgagatagccatcctatgagaatgtgaacttgaaataagattttgattacttttttttcctcctttaaaggtttgttcaattttaactattttttttcaaaatctgcattatcacagtatattttaaaagaatgactaatttactgtgatatatttgggagttaccttgaaagagattatcttgtaatttaaaataccagaggtagagtgaagagtcaggtggtaaaagagaaggagactggatgtttgtacccggtttgcttcttcccctttgttctgctgctggttttattttagctgttctgtttgcctcaccgcaggcagcgtcaccactccggttccaatcttctggacgtagagttttcaaaccatctataaattagcagtctaagtggaactttggcaaatgaggttgccaacaagcattttctttatatttagataatttgagctcatggcctctgggcatttggtaagaagtccagtaaagagaagcctaaagaacagaatctttattatgagtagtcgcctttttccactcagcagtttgattccaagcaccctgagaaaaactagagacatagtctagttggcaggttttttgaagagaatgagaagcccaaaccaaaagataatgaagaccacagcagagaactaccgaggcagctggcgcagatgcttttttggcactgttgagaaaggcttcagacccacagtctgaagagatggagaacccctcctccaggagcaaggaataagaatgacaattgttctgtaggacagttgacttagattcaggaaagttcttcagaaaatatattcagatcacaaaaaaggtgttaaaatcccaggatttttcagccaaggatggcacttgcttctttcagcatcactcatcaagttgaaatttatgagtgtcctttgttatcacaaacaaagcaagcaaactgaacaaatgagaatgcttctttagtatctacatagcactttaaaagctctgttttctttacaaaatgtgtgtatggaaaggtaaaatgggaataaatagtattaaattctgttcttagaacctactgtatagtgattgaacagctaaacagaacgaaaaagcatgcacatcttacagatacagagatcatgactttggtagatgagactaacatgtatgaaggtgtaggaagaatgtttatttttcagtccaaggaagcaattcacaatcaactgttagagaagcagcaaatagcgaagaaaaaattaaagaactagaacagaaaaagtcctacctggagcggagcgttaaggaagctgagaacaacatccgggagatgctgatggcacgaagggcccagtagggagcctctctgggaagctcttcctcctgcccctctcattcctggtgggggcagaggagtctctgcagggaaacagcttctcctctgccctgatggatgctttatttggatggcctggcaacgtcacattttctgcatcaccctgagccccatttgcttcccagccctggagtttttaccctggctttgcctgccacctctgcccaggacactcttccctcttggggtgtgtgatgaactcccaggagaggaaagatgggagccagggcaagataggaagctctgcctgagctttccactaggcacaccagccagaccaataaaaagcatctgtcccactctgctaagcctgcttttcttgagcagagggtgagagaggcagtggccatctccacctcagctcctgctccctctgcatcagagcccttcctttcttgggggatgggctcttgccctcttctcttttcccttcctgtatctttgactaacgcctcagctttcaggcctacatgtagcagacagaagaggaagaaagaacagacgttcacagctgaatctcagtgaacagaatagcagtccctggatggcagtctgcctaaagattcctttccctgccttctcccatacattccaaaaggaagttcaacagtcagcagcacctccaagactgtctccttttggccaatatcataagatggacgccgtaatcctgaggcctcctagaggctgagggggcaatggtgtgctccagctggctcatcccagcccaggtgggccaattattcaattttcaagaattttgttgcaagccagttgttaaacacagccattataattatgtaaatttgcaaattatgttaaaaacaaggacaataaatactcaaaatgcaaaaaaaaaaaaaaaaacctactgcatagtgtaatagtgctctcacttttctctccttgattctgatgtcaatcactcattgtcacctccactagagggggataaaaaggataataggaaatcagaatattttgatttgtagttcaactgttgatcaattatctttgagacttttaacattcatgactaaggaggattaataattaacatgagctgtagaattaaggtttgtatggcatgataagtataaaccagttttgggaccgctataattctaaaaaagcaggtagactagatgattagttgtacacttattactgctaattcttgattgtagaacaaattttcctatgaaaaccatgttgtgtattttatatctctattagttcgttaaaagtttagcagttttagatgtcgaaccagtaaaaaacaagttgcccattctatcatttttttttattgtggtaaaatatatttaagataaaatttacgattttaaccatcttaagtgtacattggtacagtggcattggttacgttcacaatgttgtacaactgtcatccctatctatttccaaagctttttcatcacccaaacagctctatacccactaaacaacaactccacatcacccactccccagccctggttatctctgttctactttctgtctctatgaattcggatattccagttgtttcatataagtggactcatataatatttgtcctttttgtatggcttatttcatttagcacagtgtcttcaaggtgcatccatgttgtagcatgtatcagaatgccattcctttttatggctgagtaatattcccttgcatggatatataccacattgtgtttctccattcatctactgatggatacttgggttgtttttgccttttgctattgcagataatgcttcagtaaacattggtgttcaagtatctgttttagtccccacttttagttcttttgggtatatacccaggagtgaaatgctgggtcacatggtaattctgttttgctttttaagcaactgccaaactgcttttcacagctgctataccattctgcattcccactgcaatatatatgggttctaatatctctacattgctacaacacctttttttaaaaaaaaaaaaaaaaaaaaagctgtcctattaggtgtcaagtgttatctcattgtggttttgatttgcattttcctaatggctgctgatgttaagcatcttttcatgtgcttattggtcatttgtatgggttctttggagaaatgtgtattcaagacttttgtccatttttaaattgggttgtttgccttttgcctttctattcttctctcacttttaaattgattttctcccatatttctccacaaagtcaaaaagatgatttcatttgaacacagataaatgctgtcagccttccattgccatatacattgaattaaggtcaaatgctttttgtgctgttgctaaaatcacttgagaatctgtctctcttcaactgaaagaattgttctggatgcgttgatgctgtggaattccttgtactctctcccaaatctcatttaacaagctcacatttagggtgtgattcactgtgaagtattccagaaaataaaatccattttctcagaatgatgagaaattttacaccttgaagagaccatctcttttccaaactgagatccacagattcttggagatccacaaatatattcttgggctctgagagttttagaattttaaaataatctgtttttatatcaataatctaaaaggtcattagaaagcccacatttcacaatacaaggacattttcctggaatgagactgttttcttacattagtaagatggaagtgacttgcctaagataacacagcaagttaaaaccaacactaaaactcatctttggaagcttttctcagtgttctttgttactagtgtggagatcttgcaatgattctgtcttcattaagtctacaggtagtggggaaagcagaaaaagaacaagtattaactaaaatgatgagtgctgtaagggagagaaatggcagtatgagttgtgtagaatgagagcagttgcataggatgagttaagttactgtgagagtttgaggaggtggtttttatgccgatagttgaagagtgaattggagttaccttaagggggttgtgagaagaaccatgttacagatggaggaaatgagatgtgaaggctgtgatgtgggaaacagcttagtttactccctgatttaaatggagaaaaggaatccagtgaggcttcagagagaagcaagaactaggttattcagggtttaagacaatcaaaagtcaaaatagagtttaaacaggggagtgacataacctgatttgcatcatgctggctgtagtatgcagaattgattggagaggcaaggatggcagacaggacgccaggtgggaagctatggaaaggtgcatatggaagaagagagtgtctttggattccagttggtaagtggaagagactgaagcatgtggctgaatgtaaatgtttaataagtagaattaaaaggacatggcgattggtttgatggggatgtctgggccttcaattatgactccctgtttttggcaatgatcagctgggagattggtggcagcatttactaagacagggctcattgaaggaggaccagttttagggaatagaggatgaatggagtttttaacctgttgagtttcagttgcctgtgagatatttcaatggagttaccaaatagatatttctatgtagtgtcaaggttcagactagaggactgaggcagagatataaatttggtcttaatatatccatagtgcttgaaactatatgtgtggagatagttatatactaatggttgtcaaattaagcattttttccaattcctttcccagaatctagtcatgtgaaatgatgaggaactggaatgtacagtgtatacattgttcttggcaaaactaaatttaaatttctgcttaacctaatcaaccaaaaagccctttttatttctgcctgtgtaaaatctttcttcaacatattaagccatttttctgtcttggtaaatatgatagggtgaaataaaatttaaattgaatttagatagccctgaattgatgattcaagaacttttagaatcttagttttatttttgagtagcaaataacattatactgtagatataaaaaatgttgaagattatcccaggaagtgctctggaagctgcttttaaaaataaataacttttaaactgccttttctcccttagcatttgcccatttgttctaaaggtagaagaacaactttcagtgtgttaagggtttttaatagttaatcacagttatatggcatttgtttttgtttaatatgtggatttttatgtaatgccacccagaggcctaggtatatcagtacattcctcagcattattaatgtcatccaaatagattgtctttgattactgatcactgactccatatatagggcagtgatttcaaaattattctactaccattgcaagtagacatgtatacagtaaagaaccatccaaaggccaggagcagtgactcaggcctatagtcccagcactttgggagaccaaggtgggaggatcgtttgagcccaggagttcaagactagcctgggaaacatagtgagaccccatctctacaaaaaattttaaaaattggctgggcatggtgggatgcatacttgtagtcccagctacttgggaggctgagggaggatcgcttgagcccgggaggtccaggctccagtgagccatgttcacaccactgcactccaacctaggcaacagagtgagatcctgtctctaaaaataaaaattctgaaaagttacatctaaattaggtttgagtaaatgaagtcatgagccaactgcataggtgatctttctagtggtagcaataccaaagtgggccttgtaaaaacaaacaaacaaacaaacaaaaacacccttttaaatgtagttcagggagaaggaagctagtatttcaacttactgtatgccaagcacttctaagtgctttacattattctctttaatattatttcttatattcatcaccacaatcctcatttacaactgaggaaacagaaggaggttaaatagctttgcccaaagttacaccactggcatgtggtaatgtagggcttccagcttgagtatatctaattttatagccatcattctttctctttccatctctgtatctctctgggagtcaggagacctatattcctgtgtttttgttgttgtcgttttgagacagagtctcactctgttgctcaggctggagtgcagtggtgccatcgtggctcactgcaacctcaacctaccgggctcgagtgatcctcagctgtgccatctcgctatggaaccttggctaagccacgttctgagctatggtaaagcacagtaataggttcacattcactctcccccatcatgcttatgccccctacctcgtctagaaggctccacacttcctttctgtgtgctcgttcattcagtcgtcagtcatttcgtcagttagtcagacaacagactttgttcatacgacattagtgactgaggttctgttgagtgacgcagataggaaataacctagctgcatggttgctgtctttcaaaaggccgtactcttaggatccacatacaggcccccatcttctgtgaagccattgtccccagccatcccattttgacgagtttttttgagtccctgtgaaactttggcttttttacatttatctggcagttaactaggtgctgctttgtgacagttcttctattattggatgcaattcttaattcaaatcatgtatcattacttaacttttcacctctttgcagcttctttctcccagaagattacaagttccttaagagcaggcttatacttctttgtataatatttcccacagtgtctcttaaatagcaaagttttttgacctgatttactaaaaaaaagtttgttgacttggcttaaattttattttcttttctttttttctttttttttttctttgagacagagtcttgctctgtgaccctggctggagtgcagtggtacgattttggctcactgcaacctctgcctcccgggttcaaacaattctcgtgcctcagtctcccaagtagccaggattacaggtgcccaccaccatgcccaaccaatttttatatttttagtagagatgggttttcgccatgttgtccaggctggtctcgaactcctgacctcaagtggtccaccctcctcggcctcccaaagtgttgggattacaggtgtggaagccaccgtgcctggctgacttgggttaaatttttaagtttagagtttactccctggttgaatgatttgggccaagttacatagcttctgtgaacctcctcagttttcaacaaaatgggaataattcctacttctcaagatgaaatatataatatatgcaaaagtgctctgtgaactctaaaagactatacaaatattactgattattttatactttttagtcaaagcccccctgtcctgcattgttccagtagacactttatgtaccaacatgtacttcagctgttcacattatatacttttatctctgttatcctaccatctaggtcaatacattttaacatctttatttgctgtaagtttatttctaattttttattctttattttgtttattcccatacttaacttttacttacgactacggatttaagtagcatgaactgtcttcagactggcctcctgtgatgagtcacaaggcccatcataatggatccagattgcaaaattgggagtaatagaaattactgttaattttcttgtaaggtagtttaatgcatttacttgcagtttcagatgggtagaaatggtacgttgttaggaattcactattgagatgatttttagtctgtattagagctctgaatactctgcaaataatatgtgagcactggtaccatcaaagcatgtgttgccttaggtggtgtttacagaccttgtcagagttggtagtaattccagaatataatcatttcaatagctaaggcttatggttaagacttccaaagtgttcagaatttactaaattgaattatttgaggttttgttactgtgttacttgtagtcatcaaatcaagctttattttactgccttcattgaaataagcagttttgaccatttaaaaatatttttaataggatacttattttctctagcatttttagagtgtggttcctgtgtggtgaatgtctgtcacctcttggaagccttgaaatacacaaattgactattgttgtttatgttgaaagatattaaaatgagatttgtaattgaaacgggcattacttatttcacatctggttgcttgaaaagtggccagatgcaacaattcttaatgcaagagagcctacggcaaaaactttgcaattctcaccctctacaaaacagctgccctgtgtgaaccttcagtggaaaaagaagaagaaaagcaaagcaaagaaaattcaccccaccacgttctaattacagatttccaataaaagtattaatatgattcctacatcaatgagctaatcattagaagtagaaggcaatgtggggaaaaaaaaaaaaaacaagaatggcagcactgacctagtttctagttgccaggaccaaagacgtcgtgacaccactgtcctttctaaatctccttaccttgcttaatcttaaagtgatggtatgtcctggtttgcctgacagtcctggtttatgcctgttgtcctagtgtgattttttgatagtgccctctttcactcttaaaagtgtcccagtttgggtaaggaggtatatgcccatcttaatttaatctctttatctcagagacattaattaggaagggtaagaagtgggagttatgagaatgtatcaaagggaaaatgatactccagttgagacagtgctggaaccaaaatgctgctgatctgaattctccgtaataagatgagaattggtttgcgaacacattctaaacatcacaagatacatcagattaatgctggttgctcttggggagttgtggggaggaggataaccaggtcaggagtttgtggtatcttttatgcaacactaacgttaagcttcctgtctgaggttttccatgttgtgagttttcacctcttgatactctttgaggactagtgatgctgacatctactagaaacacttcagagaaaaaaaaagatggaagataatagttctttagaaaaatggaaacatttacattattcttgataactttgttcttctttgaaaaagattagctgaacatccctgctgattaccttaataatctactctgaaattaattttatctactctagaaggtttgctgtggatagattctcctttagagtcctccagatttgatagctattattttttaataatagttaagtgcctttattttgtttaaagtactgaaaactgaatcaggtctcaggaactagttatgagcaactgattatttagctgggaaaagaaggtaaggaagaagaatatttgaacaaaggcctttaggtttttataaagacattatgcaaagagtgataaccatctttcttacaacctcttctcaaagtaaagcaagggggctgagcttcttgtggacatttaagtttgaatagggtactctcctgaaggcctttaaaaatacagtagcttgtctgtttggagtttagcaatcaatcaatcaagtcttgaacacctaatgctgcgctaagaattataaatgaactagaaggcatactcttttcatgtcttcaaatgacttaataatatacttggggaaataaaacaaatatacgtgaaacagatcaggtctaaattatgtggtattgactctatgagttactaggatgaattggcattcaaagagcatttaatttgtgccaaccaatagaaatatctcattgtgttacagcataagaaagatcaggaagccagaaacaagtcttagtggatggcagaaaataagaatatatacattcatttctacactaatgcatctattcagtatttcttgaggacttactgtatactataggcacataggagtgagtaggtacattgttctgagtagaacttatgtgttaggaaatagggaacaaaaaagtgggaggacaggtgggtgtgtgggcagagtatagcaataatattgttaacctatgcaaaggttaggctgattctgaatagagcaagagtttataatggaagattccttttaacacttaaaattcatgaaacgaagagctcaaagaaaatcttagaagccatctaatctactctcctttccctatctccacttgaaaaatggggacttaagagcagagctgttgcgacttgcctgaattcataaaccgagttagtggcggagctgaaagtcgagcctggctcttctgtttttaccatttgcagttgctcccacccctgcccccattcatgggttccacatccatggattcaaccaactatggatcaaaagtattttttaaaaataaataaaaatagcaatacaataattaaaaatagaaaatgtaaaatacggtataaacaactatttacatagcattacattgtattagttattatacataatctagagatgatttaaagtatatgggagaatgtgcataggttatgttcaaatactatgctattttagattagggacttgagcatcttggatttttgtatccacaacgtcctggaaccaatccttcatgaataaggaaggacaaataaatatatccttctgcctcttcagtcttaaagttagctaaaacatttcacttccccatgccaactctgtgaataccagatcttagatcctaaactaaaggtttggaaggcaatcattattcaggcataggtaggaaagtatgacttagaaaatgaatctgtttcgtgtctcaattttaataaaagtcttgtttataaaatgatgaaaggctcaatgagattaacaacatacaacttgtaataaatgttttgtaaccatgacactgatgagctgtgttttagacctagctgtcactaattggctgtgtgatttttgcacaaattatgtctcctcttttgtctttatttatattatgacagaatttaaattgtttcagttattttattactatatagatatctctctcttaaaattgcttgagtacagcacagacttcaaggaaaaaatttcttatgattctcagtgttcacataatttgtattaaatgtaacttaaatatgtgtaaaacaagttgtaaactatgtttatagctcttatatatttacacaaccaaaataaattttaaaataagttgtaaagcccttaacattaaaggtacaaatatggaaattactgctatagcatatacttttggggattcaacaccttttgctgcttaatgccatatctgattaaatttttccaagacttttttctttttcaactcttctgtgaaacattcctttctacagtattactggatctagtttgaacttcagttaactcagatgaatcatttacatattaaaagtttacctttgttcttctttcactaaccttagacaactaaagttgtgatattgggcaccagtgaaatcacaggtagaaactgtggcactgaaactgcttgtctgtattcagttataaaatcatacatataatctaaaatatgaattcttatacattttatgtcattaaaattaaggtaaaattgaaaacagagttaaaaacttttcctcagaaatcggaaacaatctaaatgtccatcagctgaatggatagacaaaatgtgctgtatcaatacacagcctaatctaggcagttcagcagtaaacaggcatgaactgctgacacatgccacaacctgaacctccagactcaacctcggaacctccaaaacattatgctaagtgaaaaaaaaccaaacacatgagaccatatatcatatgattccatttatctgaaatgtctaataaaagaagcaaatctatagaggcagaaaagtataatagattaacagttgtctgggattgggggtagtaattggcgtttgattgtaaatcagcatgacagatcttactaggtgatgaaaatgttctaaaactgatttatggtgatggttgcaaagcttggtaaatttactaaaattcgttgaattgcacattcaaaatggtgaactttatgctatgtaaaatatgcatcaataacgctgttttaaaagctttttatgaagacttctagatctcctaagaatatttaggtatcagatattttcgcccttgacccttccaaggtcagaagacacagctgaaaaaatacttttagtggaaccaaagaatggatccagtaactacagtatcgtggattctagctttgactttgtcactgactgtacttgggcaaggcctgggcaagtcacttaacctctttttgctttggtttccccatctgtaaagagggtgttagtttaaatgttctccaggatttcttgcacacatgagagtcgcatcctcacatttctgttatctgtgaatctctacctcctaatgattccccctccctcaaatactctaacaaagcccctttattcagcatggcatatgtggcatattgcttgtctgactaattcttgctcacgcaagcttcctgaccttactgcataggtctttcaggtacacagttctcaaagtgttgatctgaaagttccgtacaagactggctccccatcactcattaatctcccagaggctaccttctcatcctatgtaactctcttatggaagctctcccatagataacaatatacttccccttcaggcagtacatggaagaaacaatgggctatacaatggaaccatcctatgagattacacagaatgagaacctggcccattgttttctcctgctgcctaaggaaggagttgtgctctttggatccctgtacagattttggcttgtccacctccctgaattcccaagtaacaattaagcaatctgcctccttccgtcttctcatttcaatgtgtaggatatttgtcaacttgcccagagttgctgtaggcattttgagctattcatctgactgtgtgcaatagataacaaaaatataattaggaggaggactgaattaagtctgaattttatttttatttttattgcgggggagaggtgcttactttagagtggtaaaagttacttcttgtccaagttttcatcatcccctccttccctctgtgtttcaataaaatgtttccaaagtttcaaataaaaagtttattaaaatagaggccgtgtttttaaaaataaaaatgtaacatcttacttcctagaggtatctgaagaaaagcactccaggagtataaagaataaactagatggttggaatgtggtttattagtccacaaacctgtcaggaatcttggactcttttcctaaagctatgcagacatacaacataactttggaagaaccagttaagtttgtattctaactatgccctgtaatttaggaataactacctgctaaacactgaaaatctgttggtggaaatcatcgggtaaaaccaaagcataagttatattattaggggttggtaagtggttttgtatatgcaataaaaaggattagatggctgggcgtggtggctcacacctataatcccagcactttgggaggccaaggcgggcggatcacaaggtcaggagttcaagaccagtctggccaatatggtgaaacctagtgtctactaaaaatgcaaaaattagccaggcatggtggcacatgcctgtagtcccagctacttgggaggctgaggcagaagaatcgcttgaacctgggaggtggaggttgcagtgagctgagatcacgccactgcactccagcctgggcaacagagcgagactccgtctcaaaaaaaaaaaaaagattagatatttgaagaaccatgggatgggtgtcccgttaactgtccatagggccaaggaaaatggagaaagatgaatttgaaataaaattctctaaaaatagctgaaaaagaaaaacttagatatgtaagtgttttatgatgtgaacattcaatgaacagaggaaagagatgtgcctaggaatgtttggtgttgaagatatttacctttttaataaaagagaacaaattaaacaagcagtcttcctttcttgtaggaactgtatttcagggtaattacatggctttagttgctgaaggaaagatcttccttatagaaaaatgcctgctgtctgggtacagtggctcatgcctgtaatcccagaactttgggaggccaaggtggacagatcacttgatcccagaaattcaacaccagccttggcaacatggtgaaaccccatctctacaaaaacatacaaaagttagttgggcattgtggcacgcgcctgtggtctcagctactcaggaggctgaggtaggaggatcgcttgagcctgggagtcagaggttgcggtgagctatgatcgcgctgctgcactccagcctgggtaacagagcaagaccctgtctcaaaaaaagaaaaaaaatttcctactaataaatgtggaaagagtggtggaattagaaaaacatttcataaactccaagtattgattcaggaaaagatgataaagtttgttacacctttaggtgaatgattaatgaaactgaacatccttgtaataccaaagtaacattgcactgatgactgtctaattgaaggagaaaaagtaactgtacaatggagagatgagacatatcaccattctaacttagtggtagattttaccatggctgatggtcagtcatatattttgccttctgatgtgatggaataggaagtacacatcccctatggtatattcaagcctaaaatgtttaacttgaatccagaaatccttggatatagtgtccagtttataagaaacacaggggatggagaatcaagctaaatgatacaacaagaaaacaaccagacaaattcagaggttgagacattctatctgataactggcctgacctcttcaattagtcagtggccttaaaattaaggggaaggagtagtcatgctagatttaaaacagcttaggaacatacagccaaatgcatttcatcatactggattagattagggttggagcaaaccaactataaagaacatttggggtcagctggggaaatgtgactatgggctgggtattgtttgaaatgaaaatgtaattaaatgaaaagatggagattattgacttgaaaaagcagaggactaccaggaagtaccatatctcattttgattaaaaaatacacatgctgtatgtatgtgtatatgtgtatgtgcttatgtatctgtatatacatatgatcatacatatgcccagaaaaagacccagatgcataaactttaagagttcaacattggttatatctggtagtggaagtataatcattttgtttttaccagtctgtattttctaatttttaacaatgaacgcggattggttctataataatgaaaggtggttttttttttttaaacaaaaaaacccctaccttttcagatagaaaagcagcagtgtattctttccttcaaggaaaatgaaaatgtttccttggaggaaagaagatataaagactagaaaacactctagtatctttaaaaaccaccaagctttcaaatacaaagatttaacatatgtaaactgtattgtacttcatttgaaattcagtatctttactaaagagatttcttttatcctatcctacacacgtacagtattatttaatgatgtgcacacagaactctgccagcctgaaacagtaggtctaagacacgtttgttgtttttggctcccctgtgttgatctaaatcactgcctagtagcccttggaaatttctatcccaaaacgtcccacaggtgcccaataccatggattctcattccccaagttcattctcactcttctctcttggttgagtcttgcctttcttcactctgttccctttccttgttcctgccatccaaagcttctctgtctttcaggctcatctcagatttcctctctttcctgaaggctttttggattccttccatctccatgcacagtcagaagggctctgttcatctctgaatatctttcgcactccattgcatctggtttaaatcccttctctgcctcttattagctatgtgatctcaaaactcaatttctttatctgtaaaatggtgataataaatgacttcgcagggttgagggcctcaagtcagtggcgagtactgcagtagaggcagaatacatagctcaacagaaattactctagtaatggtcatgattgtttgcacttctctcaaaccacctactcgtatattagagtgcattgtgcttgtcttatcctctttactaaaagtaggaaaatggcttgagaattgatgctcagtcttatttatattattctatccctagttatgtgatacttaactgtattgctcggtatttagcagcccattatctaatcataacatcaccctcattctttctgagcttgtttcctttttgtgaaatggggctaccattcaatctcaccgaatttttttttttttttttgagacagaatcttgctctatcacccaggctggagtgcagtggggcaaactcggctcactacaacctctgcctcccaagttcaagtacttatcgtgcctcagcctcctgagtacctgggaatacaagcatgtgccatcacacccagctcatttttgtatttttggtagagacagggtttcgccatgttggccaggctggtctcaaactcctgacctcaggtgatccacccacctcggcttcccaaagtcctgggattacaggtgtgagccaccacgcccagccaatctcacagaatttttataaagtctagacaaatttttattttaaaagacttagcacagcccctgacagcgtaggcactcaataaatgtggaatctgaatcttacttaaagctagtttatattttcaagtcttcttacttttactaatgctgctttaagatatattctataagttcaaatgtaaagttctagattgggaaagaatttataatgtgtctaataaagattgttagagacaaacttttagactgaaccatctactattaatggactgatgaatcttaccggaaaataaacataaaatatactctgtgttgcctgtggtttaattgccatcaaaattaagtttattatgtactgatttcacagttacattttattttcagtcaaatatctatttgttggtgtgaagaaaatatgtctacttccgctaagtgaactttaaaaatttgtacttgcttttgaagattccatactctatataccgtctcttcctgaagcgtatttatgtagctcctgttaacataagagcgataagagtgttgcttgatgactgctcttattgctttattaaagtgtcatgtaagatttaaatatttgattataagaattcataatataattttgaaagcctactctgttccagacgctgatttatttaaatcatctctaattctcacaactccgaaaagataagaaaacagagatgtgacaaggcttgaatgcaaacccaggtctgtttgactctttgttcattttgaataaagacaaattactcaaagctttactgccaaaaactattatctggttccacattgaaaccctattcaatagttgttattattttcttcttttgctttgaataacctcccagcattgttgtgagtttcaaacgaaataaaaacactatacagatccaaggtgtttttttttagagtattgttacctcaaagtagaaattttcaaatagtaatgttatcattttcttaagagcaaggaactttgtcaatagaaacaaaaacatcataccagaagaaccccagaatgctgggatgactcatgaagaatctgtgaatgaggcaagagaacattcgtcagcaaaatgactccgtgaactgatgttcaaatattatcaaattttaattaccgagtcataattgagtacttactttgtagagagcaccatatctaaaccctgtggtgaatgatgacattttcataggtaattcctccacattaagtgattcatcatagaaaaatgctttcatgctgatgacttccaccatgactgttggcattaaggatcattgacctgaagaactgatttgaacgtcttactgcatcattttaatgagatagagcatcgtttggagctttggactgggaatcaagaaacctggtttctagctcaaactctgctgctaatttatctaggtcatgctttgggcaaatcatttcattcctctggaacccattttccttatatcaaaaattaagagttggaatatgctaatctccctcctattaagtttcgtgagcccgtgatttcaacagtacgtaaatttcctttagtacagtcacagaactggtaagtgacaccagctgaaactgagtgcagctccgcggctctacgcttgctgtagaacctttgtgaattcccactgtgcttcaccagtgacaggatgtgctacccaataaatctccatataactgatcttgaaaaattcattgataaaatctgaaaactgaaaaagatacatgttttagaggtttaggtttcccacaacatgaaaatgtgtcagttgataatatagttcatggtattttactatgctagaaaacagcttactagagaatttcagatattacatgcaacgacaagttaacatagtttaggtgggacgtctttggttcaaagttacagtaatttcaaaagtgtttagttgatccctagaaagtggttaaacaggttagtctctttttcaggtattagagggaagttaacttttgaagctgggcagaaaaggaagttttcagttggttttttccttttgagacttttaatttatcatcagacactattttttcagatgaaagtgccttttatactgtcacctaggtatgagtcaagtatgaaaaggaaatagtaactcaggatcagaacatgaataagtatgcaatatggagaatttccattaaattttaaataaatgaattacagtcatattattcagtatctcaacattaaaactcaaaaagaccttaaggattattctgttttgtggaaagggctttttctgattctagagatgagaaatgtggacccaaagagttgacacagtaggtttgtggctgagccaagacaaaaacgtaaaacctaggctttctggctccaatgtcagtgttcttttctactgcttctgagttaagaagacctttttttttttgataccaaagatacatttactggagttattggcttctttttttttaagacagggtctcactctgtcacccaggctagagtgcagtgatgcgattacagctcactgcagcctcggtctcctgggctcaagcatccctcctgcctcagcctctcaagtagctgacactacaggtacatgccaccatgcctggccaattattttatttttttatagaggcagggtctcactctgttgcccaggctggtctccaactcctgggctcaagggatcctcttgccttagccacccaaagtgctgggattgcaggtgtgagccattgcacctagcagcattattttcctttaaatcaaaataatataattgtttaatataattatgtccataaaatatattacttttgaaaatctgaacaaaattacagtttttggttaaagaagaatggatttgcctttcaaaaaaatgatacacgagagactgaggcaggagaatttcttgaatctgggaggtggaggtaggttgcaatgagcctagattgtgccactgcactccagcctgagggacagagtgagaccctgtctcaaaaaaaaaaagacacagctgagtgtggtggcgagcaactgaaatcctagctgctctggaggctgaggcaggggggtcacttgagcccaggagttggagactagcctgggcaacatagcaagaccccggtcaaaaatttttttaatttaaaaaattttccaaaaggggcacagatacattgaacattgaccttcctttgtctgcttttatgggactatccggcttgttgtgatggttgtggtgaaatttacatccaggatacaggattatttgttcttctcaaaggagtgtcccattggcccagcatttctctcatgttgtctctcccctgggactatgaagaaaccctgaactgatgcaaccaccacttgaaaataaagttccaagccaatctgaattgcactaggaaaaggcagccattcttttgggtagggggagaagtgagcttggctggtcattctggttgttagcaattatctggcttccgtgggagcatcatccagaaatttacatcttattgaaacaagaagaaaatggatgctattatcagttcgttaggtctctaaactccttcttgaatgatacatgtgatatggatgtgctgttctttcattactctcatcatcaacaaaaacaatatgtgtgttttaatgccaaccagggactctgctaggttctagggatgcaatgagatctaaatgcttaaccttttagaagctagcataaatgttagtagcacttacagcattaaatgaagcagatttttaaatgaggttatagcaaaagaaaaaaatactgtacatacaattttataccaatactaacatagcttatttcacacaaaagaaaaagctatttaagcaattttaaaaattcatattcattctagagaataaattccaaatttatagcaaaaaaagagtgaagttttcattagctttcaagaataatgcaggctccaaagtatttaatattctatattatgggaatattttatgtaagtcatcccaacaccaagtaatgctaaaaataactttatataggttcaataccatgattttattaactatagatttttagaccttttcttactttttgttattgaatcattcacagcttttattggttgaattgccttctcctaaaagtaccaaagtgtgaagagtttttatggctgagtaatattcagattcttgtagccaaagacaggttctgtgaagtaatattgaagactaaatgttgaagagtagctttctctaataccaagcggtcaattttttcaaatatcatgaactggggtagagttaagttttcatcctcctcattatagaatcattcctcattatccccattctttctcctgctgaggatgtggtggtgtactcaaagtcacatccatacccttctgtctccaaagtcctaggagagatggccctcttctcaacctctacattgcacagaaccttacatccctcaacagaaggagtacatagtgcataaggaaagtggtcttttaccactcctcctcactctgtaacattaccaaggactatcctgcaaggggaccatccataggatgaatagcaatagactgctgcattgacgtgcaaagtgctgacaacccattggtgtacaaagcaccctaagcaagatctaaagttctaagatcttatcatgtaaatcaccagcatgctgttttatgtatcttattgtaatatccttccaatcagtattataaagggcaggacactgggggaatcttaaggctttgaagatagggatagtcacgtgcatgtcttataaattctctgatgatcatatactgtttactggtagtaatgactgtacttaaacaaatatattgtttctcatatcattgcatgccaattgaccttcaagtttaagccaatgaaaagatcactcttttaagtatatttggataattttgatatagttacatgtgaactcattaagtatagaaaccttggcttattatttaccacctaagcctaggtcagtacctggtgcttagtaggtatttgatgaatgatgggatgaataaatgtggaaataagtcttttcagtatctaaagacaaaacagatacataataagatacaaatgagattggctcatctatctttattttcattcatttgaatgtccatgtaagcaagttatttcttaagtgacccagcactgcagtgcaactatcattaacataaaatatgattaaataatccacattgtataactgttttgttagtaatgtagggatgctttgcgtgttgcttggtttgtgtatgctgccaaaagtgtaagcaacatttactgagctgctatgtggcaggccctgtgtttattaaaattgtagtttttgctaattgactgttttttgacacatccgtcttgttcatcattgtctacctagcacctagctttaggcttgccacatattatattggcaatgaatatttgttgaacaaacaaatgcatgaaatgtaatgagtaagacaggttttcgctttcaagtaaaacatagtctgggggaagacggaatgtcagaatgtggtgggtgctataggagtaggtgttatcagattctgatgctatggaataataaattgttaaaatatgtaatcttatgtttcagaagctaaagcacagtatgggaactattagaggcttagaaggcctcactacccactagactcagaagttgaatagtgtctgagaaaaaagagaatagaagtaacagtagatgaagagtccagaagtgagaccctttgaggacatgaggacaggtggaaggactgcatctgtgtgttgccaaagagtaacagagactggtataggacttgagttcaattcagtcatatccttaagtattgtgccatcgtgtctggattactttgtagtataagtaaatttccaagcatggggaggaaaggagaagctcctttattgagaattcaactgaatatagagaaaactaattttacacaactgtaatcactgtagtcatttggacaaattagcaaacccaagttttgctttaacttggattgccttaataaagatgttttggggcttaatggcacagttgctcaactcccccactttattccgtgatgttcagacccagccagcatttccccatcaggctcttgcaccatgattgacagggacacttttactagtccccttgaagaatgaatagttactcaatggagattaaccagatatatatttattttactcagaatatcacgataagtataattcagagaattattgccttctaatatactgccctgtgtgggggcgtctttgaaagtccgcaaagtcactgcaattctaataggccactcatgtgacaagacctgctcccacatcggtaatttggcacagctagtatttctccttgccaaaaagggcaaaggccttgagcaagaagccagctttttcctgattacaaaactgaccacaattcctcgccaacctaacagcgtaagtctatttttttctggtggtgtgttattcttctcatagagaactccattttttcattatgacatagcacttatcgtttaaacatcaattgatgttcaaacatcagctggtgtaacattgctgcagttgctattgatggataagctgaagtttttaagaaagcaaacccgatgtataaaattgaaaccatatcaaacccttcttcattctctcagctatttaattttacagaatttagatagcagtcagtatcattttgggcttcacaaatcagtagagtaagtaccttaggaatataacatttcagtagcatgctgataccaacgtttaaactatggatacatatttgaattccaaatttttcttcaaataatgtgattagagattcaaccaggaatagacaccgaaagaaaactttgcccaaataagctttctggtatttcataagcaagagatttaagttttccatttaagaagcaattgtgaattttacaacaataaaaaatgcaagtggatattgaacagtctctgctctgataattctaaatacagtacagttcacgcccctcacaagacactgaacatgtgggtcaccggcgagacagtgtggcaatattttccctgtaatgtaccaagtcttgccaaagcagtgaacattatgacacaactttttgtcacagctggctcctaataggacagtgccagccaattcaagcccagtcctttctgtgtttattcccatctctcccaaatatttggaaactgatgtcttgactcatgggtgtattcacaaattctgttacttcaagtctttttcttttaacggattgatcttttgctagatagagacaaaatatcagtgtgaattacagcaaacccctattccatgctgttatgggtgaaactctgggagattctcctattgacccagaaagcgattccttcactgatacactgtctgcaaacatatcacaaggtaaagttccttccagatacggctattggggacgtgggggcatttatgtaagggtaaaattgctcttgtagtttgtcttccaggttgtgtttgttttaatactatcatgtgtacactccagtattttaatgcttagctcgttgctatcgcgttcatttaaaaacatgttcagaaccttaaaaaaggaaacctaacctaatctattttatctctgtgcatggctcccatttcctgaattttaagcattaaaggtatagttatatccaaaaacaatcctgttcatttttatttcctgagtttgcatagatttcccaagaatacataagggctttttagacttgaagggtcactttttcctcttttcatctcatatgttagagatctctcataactgtttttatccctcttgcagcacttttattcctcttgaagtactctcagctcttttctgttctattttgaaatctaggtattgtgtgtgcacttcagctctcccaaagaattgtaaattcccagagtgtaggaccaagtggtgctctttattagatttcttaggatacttcctagcatagtgcctaatgcattgcagaatagattgtcggaaccttgaaaaatatctgttccaagcatcacattttatgcataaagaaaatgaggtccctaaaacagtaaatgcctattgcaagtgactacctattaacttctcttcagatgtgtcagaatcagatacctccttttcaatatttctttcttggccattgcaaaatataagttctccatttctatagctccttctactggagtttcttagcacagccttttctgttttgtattgtctcgtattgatgtctatatttctattcattttatttttctcttctcagattctgtgtaaaaatagattgttagctagtgtgattcatgttttataatataagtgctaaagcattaaaattaagtcacttaattgctgaaatggatggctttacattttaaccttttaattttcctggaagcctgaagtttgaacatattagtcaccgtttttaaaatataaagctttttaaaaatgagtaagaaaatgtttcctgaagtactgttttattttattacagtaatttgattcaacatgtatttctatcatctaagcctcttaactaggagttttgttttattccataaattgttgttattctgtttttctgcaagccataaaaattgttgctatctttcttcagttattaatttagcctgcctcagggcagaatgtcctggctatactgtgaaatagttcttgagtcaaaatttgatagttttgtctattttgaaagaacatgtctagaatgcagtgcaacaaatatcaacaaataggttaactgagaggcaagtactgtttgtggtaagggatggttatgtgattcaagttagttttcattctgtgctaatctctaattttatgctttgaatttactctgagtttaatttttttgagtctcaatttcctctaaaagaatgatccagtacttttcttcctaattaacatagtgcagaatgaaattattagcaattaaaacattatattttgtaccaataatttaacccttgtcttatagggtctctggctactacttcttcataagcactgtttctctgttaaaaatgtgagctcctaaaggcccaagacctatgattgttactcctattgcattttgtaaatgacctttaaataattactccacctcttccaaaacagacagacttaaaaataaattagctatatgatcatcattagccgaggccagcagagttgaacactgtggagttgaggtatttggcatgggtacagtagtcctcttaggtgctccttcttcctcatcataggcaaagtgaaattgggggtgttgcaaatggttgggtactgctatttaagaaaaagtggcacatttagtgtgaatgactcagatatccaggtagattggtcatattcatgagattaaaaatctcgactacacttagccactatgcccaaagaggacttccatactttttccaaggctccatcattgctgtgacccctttatattattcccctttctctttttatcaagagctcctcaagcaaccccaagtaacctcatgctggctctgaaaccatttccactagtaaaaattggcaagattgaagggaagcttccacaagctcaagctgaatatgcctcctgcttttagtgccagacttagtcctgtgtgaattctacaattttaagtcgttgcaccagagccttgaaagatgatgacacacttgtcctttggagctgattgagcaatggaaatatgccttagagacatgataaatgctgcaatagacaatccttcaaaacgagtctctcctgagataggagtgagtgattgattgcatgccatttttattgagggatacttaatctgtggttacatatctccaggcaactgtatattcagaaacatttagataactgaccaaagttttcacctttccagaagtttaaagggacccatgtagtaccagggaatataaggaccagtcctttgttggccacatatttgctgtgtgaatttaagcacgccatttttcttctctggccttctgtttccacatatattaacagaatacacaggattaatgggaggattaaatgacacaatacttgtgcaagtagatctaaagccctatatggaatttattgctttagccttgtagcgttaatcacttgtggctagaatagaaggctccgtttaaaaaaagaaaaggctgggtacagtggctcacatctgtaatcccacaactttgggaggccgaggcgggcagatcacgaggtcaggaggtcaagaggtcaagaccatcctggctaacacgatgaaaccctgtctctactaaaaatacaaaaaattagccaggcgtggtggtgggcgcctgtagtcccagctactcaggaggctgaggtaggagaatggcgtgaacccaggaggcagaggttgcagtgaggctgagattgcgccactgcactccagcctgggcgacagagcgagactccatctcaaaaaaaaaaaaaaaaaagaaaagaaacctacattttaaacactttatggatatggaaaaacaaaaaccgcagaactctttattgcacaagagtaagtggcagttatatgaatgaagcggggcatccgcatgatctcctgtgagtgttttgagaagatgatcaaacttagagaaagcatctttgtgtctaatttttttaaatgatgaaattatttcccactttcattaatattttcctgtgaagaaattgaagttattctttgggcctaaataaaagtgacaattacatgttttcttctcagaggcattgagaaacttggattataatgtttttcgtacactgaagtagaagtccagaaaagatttgagttattgacacagatcttcttttttttttgagacagagcctcactctgtcacccaggctagagtgcagaggcctgatatcatatttcaccacagcctcaacctccctgggcttcggtgatcctcccacctcagcctcactagtagctgggactacaggcgtaccttaccacacccagcaaatttttgtaatttttatagagacaggatttcaccatgttgtccaagctggtcttgaactcctgggctcaaggaattctcccgcctcgacctcccaaagtgctgggattacaggcgtgagccatcgcagcctgcccctgaattccataatttcacgtgtaattagaattcaagacctttttcttgaatattactcttgtccatattaatttttttctgagtatggctctagctttatcatttttttctgacttaactctgattaacctcttttttcatcaccagtacttggcagattttgtagatttctaaactctgaaaatcactgtttttctgaaaactgtcatttgtctcatactttcctccaaaattatgaattataggaaacaccccaaagaagtatcatttacattagctgtttacaagaaaatacagggtttcatttttaattttattttcccctactatctctaaggaatacaacttgtcttgaaagctccctgacagcagctatccatttaatctgccaattgcagtataaaaattggttattaaatgaaaaacaattgtatacctctcacagaggttgtcatatattactatttttgtcagcattctcacttataactctgttaggataatcctgggaaaatttctaaaatcttcatagctctttcgttaggtacatattccttgagacagaggaaatctgagcaaagtaaatttcttcattatcttagatcaacaaaagtgatacagagcttctgtcctgttagctccctttggacgcttaaaataggcactagaggttgtccagtaaatgcttcatatccatctactagtgctgaatatttgtgaaaatataggtgataatgaattagactcgtttgaatggaattaagtttctgtaaagttttaccagattgtcttttttttgttttttgttttttgttttggtaaaagcagttttgcacctgtaactaatttaaagacgaagaaattgcacatgtaactgatagcaactgccatccaactcttataacctcagaatcaagatcacgtggctatttgagtacacagtgttttaaaccgtgtaatttttatttgagcaagggattgccaacacaagatcgaggttcccacaagtggtgaatggcacttgtggttaacactgtaaagcagatttaaaacagtcaactgtcaattgcctacaagggaattattcctcatcacctcatgccaggtttttatatggccttttagctgaccttctaaatgagaaactttgccatccagtgcaaactgccaatagtcctaaaatgcctatctttattatcagtctaataatatttattgagtgccgattttcaaggcaccatagtgaatgctgtgaaatctagacactttttccctggctttcaaagctctttataatccaggcatattttatatattttaccttatttctcaccacattccaggcattggcctgtcttgctctgttcacactaatctctttgtcccaagagtaaggccgacccacccttgctccaagcctagtatgtcttgaaatgcctttcctaattcttccatcttttcaaatcgtccctgtccttcaaggtcagcttcaagtaacctctactccgtgaagcattctctgaattcattgctgttcattgctccctctctcctctaaactgaaaatgtgttcagaatatctctcttctgccgcttccactgccttcagcccagtccaggacaccgtctgtgcctggctgctagtctcttgatggggttttcagcttccatcctaacctcacaacagcctagtctcaaccggtcacccacagagatgatgctcaaatgtgagtcagatccgtcactccacccatagccctgcagtggttccccatttcacttgtaacaaaagtccaagtctggcccctacctcctgccctaccccagctgacccctctggtcttctctctctcttccacccactctgctccaagcacgccggcctccttgctgttccttccacattccagatgctctcctgccttcgtgcacctgccccagctcttgttcctgtccaggaccctcttcctctggcattctctgggccacctctgtcaattccctataatcttggctcaaatcttaccttagagtgagggtgaccttggccttccctatcctaggacaggggaattacatatcctccccattgcccttcctcctctactctacaatttcctatttaaaaagcatttatcgtcttctaacatgccatgtaatttattggtggattctatgtattgcttactgtctgcttcacctccctggaatgttaaccgcatgaaggcagagatctttgtctagtttgttcacccactgatgtattccaaatacctgaatagtattgctcattaatacttgctaaatgaaagaatagaatcctctaacacttacggcttctatcacacaatttagccctgagttcaaactgtctggtattatttcctgttttatgtattccctaagccaaatcatatagtcactgaagaaaagatccatgttttattctatattccacaaatgccaaacacataataagtataatcaggtcttgttgacttattaataccaagctgatctcctaaggtcagagactggaatatatttagcaaataaatacaaaattttagtgtatgttaaatattattctaaaaccaatgctaatataaacaaaccaagtactggatatgctacaaaccaggcacaaatatgttttgaattatctgttagttaatttgcaccgtgtccctccattagtttgagtagcattcattgaatatcatttgaatattaatttaaacatcatttgtttgtgagttacccaaatccctggcttcttctgcaaatctacccaccttttgatacttccattatttatttgaacatccaaaacacttatttgctagcaatgtttaaaaaagagtagcttatttaattagctaatatctaatatatgaagcacttaaagtattaattgcctgatatgtaataggtgccaaacacgtcagttctgtcttcttctcatctttaaaatggaagttaatacagacataatgtctccccttgggtcttttaagtcagatctttaggaaaaagtaaaatattctcattattcttattattttgcttgcattagcaataaagtatgcctgatttaagttcactggacattatcttactattatctatactttacctaaagccctgcctttagttttgtaaataaaattatagacctattagatagattgagcaagtttgcttagtggattaaaatatgatgctaataagagccaaagtgagtttgataatgggttggccagatttacacagaagtctttgccatttacaaagagttctgtttgccattgatacagtcacatttctagccacaattacccaagatatgtaaacagttaactcaggagaaaccaactcaattttcaggtatatgccatatacaaaatctcatcacctttgctattatatacacttagtattatttcttaagcaatatcatgtaatataaggggcacgttgatgacagggacatgtacctttttcttgggatttcaatatgaatgaccatccaaatcttgatcatcccaatggctccacataatttggacaccatgtctaccagaaagctatccctgattatttcaggacatgttagtatctttcttttccacatttctaaagtacttactatctctatcacgagtctggtctctgtacttccttctgctactaaacatctctcaccttggtaagcttctcaagaaccacaatactatgtgtttcttgtaaggcctgaaaaacaccttgcacgtagtattaggtgattagctatcttgatttaaggttttagattactgattgaacatttactgagctgaagatatagagaaagaaaaatgccaaagaaaataaaatgaggaagtttggaaaagagaaacacactagtaaaatatgatggtaacttctaagaagcaaggtttataaaatagattgttgatacaaagaaaacattataaaaaaatgggtagcttgaatggatttaacccccaactttatttaatttaaatggaatgcctcaaaagactactcagctgatccaaacatagtaacttcccaaacacaagactcttgacgtaggcctcagaatttggggaaaaaaattcctgcattgtttaaaaatacagtgtccttgtaaagcagaataggccttatatacaagccaaatatatcagttctaaagcaaaaacaaaacttttcctacttactcttttatttcttttttttttttttttttgagacagagtctcactctgtcacccaggctgaagtgcagtggcgccgtctcagctcactgcaacctctgccccctgggttcacacaatcctcctgcctcagcctcccgagtaactgggattagaggcacccaccaccacgcctggctaattttgtatttttagtagagacggggtttcaccatgttggcagggtggtctcaaactcctgatctcaagtgatccgactgcctgcacttttcaaagtgctggattgcagatgtgagccactatgcccagcctgcttcactcttttagataaaatgtacaccctttgaatttttacttccgccatttacactgatttgaagtccttggttgaatatgtatttagccttcactgtgcaaggaagctggttacaaaaacagatagtttattaaaatacttgtgttggtttttaaaaaagaataattttaatacagtttttctcttctttctttttgctctttttgttattttttaatagagacacggtctggctatgttgttcagactggtttcgaactctggcctcaagtggtcctcccatcttggcttcccaaagtgctgggattacagatgtgagccaccatggctggccttaatactgttttatgagttctgaattgaataatttttaagtgaatgtcctaaaattctatgacaatccttttaacacaactggaagagtttttgttggcttacttggtttgtctgcatgtttccttctgtcatcccccactcctaggacccatatggaacattctctcttaattaactcttttattgaagtataatatacttagaggaaaaatgtccaaatcattttcacaagtgaacatacttgcgaaattagcacccagacaaaaaaaaaaaaaaacagaacaataccagcactccagaagccctctgactattctaatttctaactcattgttaagttttgcccagttttgaattttatataaataaaattacacattgtttactcttttgtgtctggattcttttgcttagcatttattcgtgagattaatcacactattgaatgtaattgtacttcattctcattactgtatagtgtttcattgtataaatatatcacaacatatttatcctactgttgatgggcattcagatggttttcagtattgcaaatagtactgctatgaacattgtcctgtaggtctcttagtgaaggagcgtatgcatttctggtagctatatgcttcagccaataatagagtatgttgagcttcaggagacgttgccaacagttctgaacaggactctagaaatctatacttctactagaagtgcatgagtgttctagctcttctacaccatcactggtacttggcattctctgtctttttcacgttagccattctgatgggtgtgttggtatcatattctaattttaatctccccttttctgatgtctaaaaagttaaacactttaaaaagcgtttgttgtccatttggctatcctttcttgtaaagtacctgttcaagtctttcacccatttttatattgagtactttaggtaaacgttctaaagcataccttgtcagatacatgtattgtaaatatctcctcttactctgtgtgctttttcgtttacttaataaacagatatttctaatcaatatagtctgatatactagatttttcttttacagtaagtgctttttatatcctttggaagaaagctttgcctactccaaggttgataaagtgtgtttttattttaaaagttttctggttttatatttcacatttagatccataatccacacagaattgacttttgtgtatgttgtaaggcaagggtttatctctttcatggggatatccagttgacctgcttcgtttattgaaaagatcatcctttgcccactccccagcagtgacatttctgtcataaaccaggtgaccatataggtctgggatttattctggacttactcttctattgcattgtatttttgcaccaattaagattgccttaagtactgtaactttataaatagtttgatatctggtagtataaatcctccagcgttgctcttccccttcaagatttccttgccacttggcctcttgaatttccttataaattttagactcagcatatcaattaccacacaaaagtgccgagattttgattggaattgttctgaatctatagataaattgggagataattgaaagctttacaatattgagtccttcaatccatgaacatgaactatcctttcacttatttattctttaatttttttaaatagtatatttgagttttcattatggagatcttacacatattttgttagattttttcccctgggtatttgattgttttaatgctgttgcaaaaataatttttaaatttcattttctacttgttgctggcttatagaaatataattggtttttgaatattgaccttgtacccagcaactttgctaaattattttattaactataatgagtttttttgtggatttttaaaaacacagtctgcctaacatggtggctcatgcctgtaatcccagaactttgggaggctgaggtgggtggatcactttgagctcaggagttcgagaccagcctggccagcatagtgaaaccctgtctctactaaaaatacgaaaaattagccaggcatggtggcatgcccctgtaatcccagctacttgggaggctgaggcaggagaattgccctaatgccagaggtggacgttggagtgagctaagataatgccactgtactccagcctgggcaacagagtgagacttcgtctcaaaaataaataaataaataaataaataaataaataaataaataaataaataaaacgaacaacaacaacaaaatacagtcatgtcatctgtaagtaatgttagttttagttttatttcttcttttccaattattctttgtctttttcctttgttgcattggatggatttccagtacagtggtgagtagaagtaatgatagtatcattaccttgttgctgatctcaagaggaaggctttaaatatttcaccatcaggtatgatggctactctaggatttttgtttgtttatttgtttgttttttatagattttctttagcagactaatgaagttccctttccttcctaagaggttttttatttaaaaaaaaaattatgagtgaatgttgaattatatcaaatgctttcatagtttgttgtggtggtggttgtttttgagacagggtctcactttgtctccaagctggatcatggtgcattgcagcctcaacctcctgggctcaagtgatcctcccacctcagcctcttccagtagctgggaccacagcacatcaccacacccagctaatttttaaaaaattgttatagagacagggtctcactgtgttgtctaggctggtcttgaactcttaggctcaagtaatcctcccaccttggcctcccaaagtgctgggattacaggtgtgagccactgtatctagcctagaatattttttatagtgctcaggagaaagattaaaagtatgaaaagtgtctactatgtatactttaatatcatattgtaatacatttgaagatactatatggtataactaatgaaaatcttcaaatcagctcataaccactagagatctggctgtatctgttttgacacttttgttctcagtatctgtttagcttaagacccttgtcttctcattaagagtgcttgacacagtaagttgaaaacataatttcaaaagagcatagagtgtcacatggtttgccagactaaacaactctgagtaagaaagtggctttccccctatggtaatgccaagctactagaggaccttcagtggtaaagtatgatttaaaaccctgaccccatctgtcttttcccatttgcagtaagtcctttatagtgtttgaaggtgggcacagcagggaaacagtactctggcagatctgcggtggctccctcctttctgagtcattacacactcttctggatcattcctttcatttttccttttcctctcctctcccctattttcattcccctatcactgcaaagccaaaaccaaaacgtggaaggggacctagccgaggaaaacattccagcttcttttttctgatctaaacaccgttcttcttcttttttattttttaattaataactttatttttagggcagttttagtttcagagcagaattgagtaggaagtacaaagagttcccatatacttcctgtcccccaccacgcacaatctcccccactatcaacatcctgcacccaagtggtacacttgttaacagtcaataaacccccactgacacatcattatcactcaaagtccatagtctacattaagattcactcttcgtgttatattctctgggtttgtacaaatgtataaggacacatatccaccattgtagtgtcatacagaatattttcactgacctaaaaatcctctgggatccacctgttcattccgttctcccccaaacttctgactaccactgatctttttcctgttttgatagttttgcgcttcccagaatatcatatagttggaaccatacagtatgtggccttttcagtttgacttctttcacttaccaatatgcacttaaggttcttccatgtctttccatggcttgatagctcatttctttgcagaactgagtaatatttcattgtttggatgagccatagtttatccactcagctactgaagggtgtcttggtgcttccaagttttggcaattatgaatagagatgctataaacacctgtgtgtgggttttttgtggacttaagttttcaattgatttgtgtacctaccaaggagtgtgattactggattgtatggtaagaatatgtttagttttataagaaatggccacactgtcttccaaagtggctgtaccattcatcagcaacgaatgagagtttctgttgctccacatcctcatcagcatttggtggtgttagtgttttagattttagccattctatttgtagtggtatcttcttgttttagtttgcagtgctctcgtgacatatgatgttgaagatcttttcacatgttcacatgccatctatatatcttcttggatgaagtgtctattcagatattttgctcatttttaaaacagggttattcactttcttattattgagttttaagcgttctttgtatattttggataacagtcctttatcacagtagtccctgacctttttggcaccagggaccagtttcatggaagacagtttttccatggactggggtgggcaggaatggtttcaggtgaaactggtccacctcagatcatcaggtattagattctcatacggagtgtgcaacctagatcccttgcgtgcacagttcacaatagggtttgtggtcctatgagaatctaatgcagccactgatctgacaggaggtggagctcaggctataatgcttgcccatcagcagttcacctgctgtgcagcctgattcctcccaggccatggactggtactggtctggacctggaggttgggaacccctgctttatcagatgtgttttgtacagatacattctccccatctgtggcttgtttactcattctcttgacactgttttgcacagagcagactttttttattttaatgaagtccagataatcagttatttcatggttcatgtctctggtggtgtatctaaaaagtcctatccgtgctcaaggtcatctaggttttctcctatattgtcttcagggagttttatagttttgtactttacatttagatctatgttccattttgagttaatttttatgaagagaataagctctgtgtctaaattattattattattattgttattattatatttttttgcatgtggatgtccagttgctccagtcccagttgttgaaaagactgtctttgctccagtgtattaccctggtttcttcatcaaagatcagctgactacatttatgtggtcctatttctgggctctgtattctgttcctttgatctatttgtctattcttttgccagtaccgtactatcttaattactttaactttctagtaaggcttgaagtcaggtaatgtcagtccttcgactttattcttcttctttaatattgagttggctattacgggtcttttgcttctccatataaactgcagaaccagtttgccaatatccacaaaataacttgctgggattgttattgtgattgcattgaatctatagatcaagttgggatgaatgacatcttgacaatactgagtcttcctatccatgagcatgcagtgtctctccatttatttcattctttgatatctttcatcagagttttgtagttttcctatataggtttcatatatattttattaaatttatacttaagtattttacttttcggggtgctaatgtaaaacaaaacagacattatgaggaaaatgtaatgtctgtttgggggtaatgtgtttttaatattaaatttcgcttgttcattgctggtatataggaaagcattatgacttttgtatattaaccttgtatcctgcaaccttgctataatcacttactaggtccaggagattttttttttttctgttcttttggatttttttatgtagaagtcatgtcatttgcaaacaaagacagttttatttttaccttcccaatctgtatcccttttatttccttttcttgtcttgttacgttagctaagacttctagtatgatgttgaaaagcagtagtgagagaagatatccttgccttattcctaatcttagtgggaaagcttgagtttctcaccattacttatgatgttaactatatgttttgtttgtagatgttcctaagtttaggaagttttcttattttgtcaaattctttttctgcatttattgatatgatcatgtgatttttcttctttaggctatgggtgtgatggattacattaatttgttttctaatgttgaaccaaccccacataccttagctaaatctcacttggccatggtgtataattcttttcatacattatcggattcaatttgctgatattttgttaaagatttctgcatgtctgtccatgagagattggtctatagggtttattttccttgtaatgtctgttttgttttagtattagggtaattctagcctcattgaatgagttaggaagtattccctctgcttttatattctgaaagagatttcagagaattggtataatttcttccttaaatgtttggtagaattcaccagtgaacccagtccggtgccttctgtttcagaaggttattagttattgattcaatttcattaataaatgtaggcctattccatttctctatttcatcttgtatgagttttggaaaattatgtctttcaaggaatgagttcatttcacctagattatcaaatttgtgagcatagagcctattatcctttaatgtcaatgagatctgtaatgatacccctctttcatttctgaataactttcattcattcttcacgtactagacaaagtagaatttcaactaaaccaaagcaactgttcaacagatttagccttagtcctaatcagattattagtaatatctccctttagaaatatatgaaggtacttttagcagaggaaccaagcttgagctagtttgaccataatgaatttttgctgaggaagcagaagaaatgtttatattggtgagggcataatcctgtaatcttttctgtgtcctgatacctcaggaccagcgtaagcaaactccctagacaagatgtggctcatgcaagaggaaatatgcttttgaaaatgaacatgggccaagcatagtgactcatgcctgtaatcccaacactttgggaggctgaggcgggaggattgcttgagtccgggagtttgagaatagcctgggcaacataatgataccttgcctctacaaaaaaaaaaataaaaataaaaaaataaaaataaaaaaattagatgaatgtggtggtgaatgcctgtggtcccagctacttggaaggctgaagtggaaggatcacttcagcacagggaggtcgaggcttcagtgaaccataattgtgccactgcactccagcctgcatgacagagtgagaccctgcctcaaaaaataatagtaaaattaaataaaatataataaaaaacatggtctttctaataccctttgttgatctgaatttgaaaatttcctgtgagaccttctggcttattataaagaaatttgcatctaaagatacatattggaatgtttcaatctcaaagctatcaatgggaaatcaaacttcagaaacattgaatttggttttgaatatagtctaagtagacaataaaatgaaaatacatttgtttatattgaagaagcaaatgacacaccaaatagttaatttctaatagttttcatttttttctttttcttttttgtttttttttcagacagtgtctctctctgttgcccaggctggagtgcactggcatgaactcggctcactgcaacctccatctcccgagttcaagtgattctcctgcctcagtctcccaagtagctgggactacaggcctgcgccaccacgcccggctaatttttgtatttttggtagagacagggtttcaccatgtaggccaggctggtctcgaactcctgacctcaggtgatcctcccaccttgtcttcccaaagtgctgggattacaaacgtgagccaccacgcccagcctctaatcattttcttaattcatggtgcccaaaataaaagattctatattcagagaaaccatgacagaatttaaactctagccccaatacctatttaacattgggcaagtcacttaacctttttatgtctaatatttttaatctgttaggtggtaataatacttgtgtataacaacataggactgtttacaacaagcacttaataattgttagctattgttactaaattgtatttctgctgatgggttccagagtcttttcctccttcactaaacaaaactcaggatactatgtgaaggaaaagaagtgagttggtagaatttattttttaaatgcttgagtcaaaaggagagccatttgtggctgaagcatcaaaaccattttctgattttctttcattctacttgaaactacattaaaattttctccaattttccagatgattgcatgataattccttttaagaggactttaacacatgtaagaaatagagttactccctctgttaggagaaatcatggaaagatattataattaaaagatagtgaattttagcttaatttttacctttatctaaataacactcgtacacagctacagctttcagtgaagaagagatactcattcccacagctaaatcccgctcctcataggcagtgattttacctctttcacatatttcttctggcatttaccttcctatctctagaataacatacttacattaatggccaggcatagtgactcacgcctgtaatcccaacactttggtaggtcaagatgggcacatcgcttgagcccaggagtttgagatcagcctgggcaacatggtgaaacctcatcagtacagaaaatacaaaaattagccagcagtggtggtgcatgcctgtggtcccagctacttgggaggctgaagttggaggatttctcgagcccgggagtttgaggctacagtgagctgtgattgcgttacagcacgacagcctgggccacagagagagaccttgtctcaaaaataattataataataataacttatactattatttttaaaattgtattcttatccattgacttcttactgtgggagataataatttagtccttttactaccatattcccttttccaaaacacatacttctccctcccctccatcttcacaatgtagttacttgtaatttggggttaaatcagttgtctgtgcttacattgtagtaactgaaagtgctgcttaaggttgagccaagcagtgtgctatgattctgtttcctctcctgcttagctatttacttttctggagttaatttttgtcacttagtcattgctcattttgttgtcatttcttgctatgtactcatcactaattcatcctcaaactctctgacagagtaataaaaactcctctgaaagtggtcagacatatcgggcattctggtttctcaggtgatcccacctggccttcttcctcctgcattttctctaggctgctctgtatctgtagcctgggactaccttccacctctctcctgtctttcagatcctttgtttcctggtcccatcttttctcttcttggttctggtggagaactctgggaaagagtacgtgggaggtaacttttctggatgccttatcattctgaaaatgtctttactctcatacttgatggacgatttggatgagtctaaaattctaggttggaaatttttttttctcttgaattttggcaacattgttctattgattttctagcttccagtgttactgttgagaagttcagtgtcattctgattcctgatcctttctatggtactttttttttttcctgtcaggaagtacttatgatcatctctatagaaggggtattaatcttgacagtgttcctttctgtgtggtttaagaaatcctttctctcttgggttttgttactgttttataaatccttgatcttagtcattctaaggttaaagaaaaactgaggaacaactcaggaatacgtttcaagtacatgtgataaaaggcaaatatgcagaggatgataactatatgtttaaaaaatgggaaaaaagagatagtcgcaaacttcaacataggttagatcatcatttctcaaaccatattccatcacatattaagctcctcaaatattgcacactgtgcttttttctctcttttgtgtattctcgtgaacattagcctgttagtttaagaagtctcacagtaaacctatttaaccttgcttaacctagcattgcctaaatttacttgatcaccagattttttttgttttgtttttaattatttttaaaggaacattattaacatatgtataacacagttggggaaatgccagataagacatagactttcctggctgctaaagagcaattaagcattggaatgagtaaccaagaggagctgtggactctccttttccagaggcctttaaaacaggacatctgttttaggcgtagtcctgtttaaacaatagggctttgctggcggttcttcctcttcctgtcccctagtggtagtgttggaggcttttagcccaccttttatatgactgtagtactctggtgactaatgtgtacacaaattactgttatactattaatattaattatatttattgagaacttattaagtatccagccctatggtaagtgctctacatacattatctaatttaatcctcacaacaactctaggagggagacaatattaatattctacagatggagattagatatgaagagatacgaacttccagcaatagggttggattggaacctcgggcatctgacatggagctgtgcccatccttcttcaccgttaatgttcaaaacagcaaacggcacaaagcaagcaaacaacatttgtatatctgtacttactatgtaaaaacaaaccaagataatgcttactttattagactattcaagactattccctacgccttttgattaattcagagttcatgacttttgttagaaacaaattctgttacagatttagttcttcgcattttattctgtgataatttgccatccaggaaatggttccttacatgagactttttccctcataaatttgatgacattcatatacattcactaaagacccccaaaaaataaatctggatctgtgaaaagtggcaaaggttaaaagtcccattttcagcatatttacatgtagtaacaacttgataacacagcgctgagaatgccaggctcagaggagccaagctgggctcccgtccagtcagcctctagctcaccaggaaaccctgagcaggtcactttgcctcattttctttatctgtcaaatgagaactcagatgatctcaaaacagccttttctgctgtagtgtttcaggatgattcttgtctctggatgtcgctgtcatgatgggagaggaattagtaaattcccaggtcagtttacactgcagagtgatcttgatctgcatgaatcagtaaggatgggctggataaacaagttggaaccacagttatgagtaccatgacattaactgaaacaaatgagaagggattgaaaaatcggcggttaagtgtcttttaaggtcatttccaacaaaactacaaaggacctatgaaaggcttttcaggcacgtactcttgttgcctagctgaacacatttctccaataaagctttcatttgagagttactcttcatagtacatcccctgcccccctgcctgcctggcacattataggtattcatgaagatttgttggaactgttaaataattgtttaatatagaactggaagatcaggaagaaataatgtttttctaaccagtatccagtgtaaagtcacaagtgtgggctatggagcttcagaccatctggattcaatgccagttctactgcttagttactgcgtacttttaggcaagttacttacttccaggtccatgcctcagttttcccatctttaaagtaaattataaatgcacctacttcctcgggctggttgtgaggattaaatgacacaatccgggcacaatatttggcgtagtactttgcacagatttagtgtcctgttagtattagctaatgtaaatatgaagaagatgtaacattttaccagctattctaatcatagcttttcttttcttttcgtaagggacaacataaaaccccaaaggtaaagcatatatttctggtgttcaattttacaatttagcatcttttccaacccacttccatactcccaaataataatgcctatcagattaaagaagttactttatttttgcatgaaattggttgtatttttgttgcactacttcatcctttgagtattgtgaaaagtctgcgcattgactataatagcatttaaagccaagtttgaaggtctaagataatggcacacatccatgtcctttgctcacaagttctgcctaaagactttagcctgcttgaaacactctaaaggttgtgaaattaatggatataccactgatgcaacacaactcagtgtcaccagctagggagttaagacatgttcttgttactgagaagggctgttgttcatgcaaatattttgttcatttgttctttcatgggtaattgcacggagtaacaaaaaacacctcctcaacttacagcttggcagaaacagtagcttatgttttctagagacaaaattagaagcaggctttctggacccatctctaagtcaaaataaaataaaatgtctagggaaccactgtgcctcccagtaactatggtccatggaataatattgtttaggtcaaggtgtcccagagtaaaagggaagggagaagggcaggggggaaggggaaaagtgtgtttgggcaatgttttcatagagatgaagaggcaagctgtttttctttcttactttgtcctttcaacttagaaaaataagaaaaccatctaggaccacaaccttcctcttgtaatcagcaaaagagaaaaaactgaatatacatcgttaaggggaatggttatggcttaagttgtattttattgataatgttgaagattaacattttttggagagaacttaagtcttaacaaaactaaactattgagaaagtattgttatttttccatctttagtagcaaaatgaaaatttactctactcttctgcataaaatatgatcatcaattggcaagaatttggttaaaaaaaaaaactgtgcatagaatatgcacatgggaagatagaataataagatgctgtccttttactccagactggaagtctaatggtggagatagttatccagataattatatctgagttaaaggaggaggtacaaagtgctgtgggaacacagagaatggagtgtttttaagttggaggattcatagatggttttacagagaaagtagcatttggactgggttttgaaagacaaacaggcttgtggcatgaagaggtgaggagtaggaagaagatacagcagcctcagtaacctgtatgactaaaagaggcagggcatgtttaacctgggcagatagtatgatatgcccagccaccagatatgccaagaatgggagcgaggtctgggataaccagtggaaataagactgaaaaagtaagttagggctgagacatagagggccttgaatgccaagctaaaaggccagtgtttctcctgcttttccaccagtatgcccttactagtagaggacagcaaaaccataccattcttgggtggaggaagggaggaatgaataggtggagtatggggtatgtttggggcagtgaaactattctgtgtgatcctctaatgaatgcatgatattatgtctttgtcaaaacccatagaacttggctgggcatggtggctcacacccgtaatcccagcactttgggaggcagaggcaggtagatccattgaggtcaggagttcaagaccagcctggccaacatagtgaaaccccttctctactaaaaatacaaaaattagcagggcatggtggcacacgcctgtaatcccagctactcaggaggctggggcaggagaatcatatgaacctgggaggtggaggttgcagtgagccaagatcgtgccactgcattccagcctgggtgacagagcgagactccaccaaaaacaaaaacaaaacaaaacaaaaatgtagaactatgcaacagaaagagcaaaccttgatgtaaatcatggactttaggtggtaataatgtgtcaatattggttcatcagttatagcaggtatactacactgtgcaaggtgttagtaataggggaaaaccatgctttgtagagaggggacaggaggctggggaagggctattttaagagctctgtactttctgctcaatttttatgtaaccctaaaactgctctcacaaaagtaaaggctactatgaaaagcaaaacaaaacgaaaacagaaaacagcatccttttagccatgagatgtactacaagtacagagttttctaggaatttcatataatatttttaaaaattacatgaacttttccttctttccttaacacatagatggctattttcacattaaaaattttcccaaaatttccacaatgtaatacacgacccctagggatatgacttttgtaataagaagcatgactttggctgggcacagtggctcacgcctatattcccggaagtttgggaggctgagccaggaggaccacttgaacgcaggagtttgagaccagcctgggcaacacagtgagatgctgtctctttaaaagtgaggttggggaggtgggagagagagagagagagagagagaggaaagaaagcagcatggctgtgattgataggaagtcactgaaattgtatgttaaaaatgaaacaaagcaaaattaaaccagtaaaatcatgagcaaccaatttctctcccaccccagatatttgtgcatccttattttcatttcccagtattcatacaaaagcatatccactacctttaactcactttgtcagcctttattctgtatgcagaaaactttgcaatagcatagtgagaaataggtgataaacccacctttgtggatattacagtgtagacaagggaaatatactttaatatgaaaataagttatactaagaaaagtagatacagaaatgaaaattaagcctgccaaggagacagacaaagtaaatggaatgacctgggttagacaggtttggacgaattttgaaacaaaggacacagttggatgggtgccctgtccttaggagcacagacttttttctggatgtcattgagacctagaatgacctttaactatcaccagtacacattcccctttttggtgtgcagtgttgacactgaaacacagataacttaaggttgtttgggtacttatttatgtagatagacatgtgaacccatacggtacctaattagtcctcttgaaaccacaaacgcgcattcacaggaccagtcttgtttatattttcaaggctttttttttttttcttttgagttggagtttcactcttgttgcccaggctggagtgcagtggcgtgatctcagctcaccgcaacctctctctcctgggttcaagtgattctcctgcctcagcttcccgagtagctgggatcacaggcatgcaccaccatgaccagctaattttgtatttttagcagagatggggtttctccatgttgatcaggctggtctcgaactcctgacctcaggtgatccaccctctttggcctcccaaagtgctggaattacaggtgtgagccaccgcacccggcctcagggcattttttcttatgaagatgacagccgaaatcttcaatcttcaactcatgcaaaagtttgcctttggagtgaatttcttggcaatctgcttggtaagaaaaagcaagtggtgttaaagtgagagcattaaaataaaattgctccaaaaaaatgataaataaataaaagaaaagaaaaaggaaaagcaagaggcccatgcacagatacacagaggagagtggcccctcctccataagcctgtttcatcaatgagagaatcaagagagtatgtatatacagaagagagtttcagggtcagtcttgagtttgcatcttctacttcctagctagaggaccttgaacaattcactttacttctttggtcctagcttgcttatctataaagtgggaataatatttcagattatgcagaagaaaaggtaggatgtaccgggcacatgggaagtgcttgatcaataccaatttgcttccctccttccctgccccttctgctgaaacttcacagctatctcattaagtcatcaatgtatttagtatttaataaataaatatttaacgagcacctactatgagacaagccctttgttagccagtggggatatgactgcaaacaagaaagatacgcctatgatttgatgaatcctggagtttatgagcagctcatctctgtctcccgtgtgagcagtcctaaatttacacagctctccactggtgtttttgaccacagagttaacatttaaaatacctatgactgcttttttggcaccactttgaataatgaactggtgtcaggtaaacagagtccaggtacctattgcagaaagttttccagtctgcaccctcggtggctcacaagttcacatgaagtgaaccaaaagagttttgttgcacttactgtgacaataaatcatgtttagaaaaatgcatgtggcatacatgatattgggcaaatgattcatgaaacttgctattctgtggccttagaatccatgtaagcatttttcctttgtcttttatgtgttttcttgtttctatctctgttttgttttgtttttgttttggggatggtttaaaattaatgctgggctagagtctgattttgacactctcgggtgtgatcaaatgtttccacatatctctctaacctttctattctcaatgtggggaaaactttctccgctttaagaaaaggtgtatccacagaaaagattattgagtaatttttcatagtacagaagtaataattgagtgatttttccctttaaaatttttttatttttgtattattttaagaacaaaattattttaggatgcaattaaagaagaagttcatttacctaaatttaatttgccaacttttctattacacaaaagcattataaatacaagtagaatagcaaaaatttcatcctaaagagaaaaatattttgggcttctaatttcaagagagtaaaattgtcctggaaccctgtgcacactttaaaaacaatcattaacagattgcatcaaactagaaacttcttcaccacaaaggaaataatcaataaagtgaaaagccagcctacagaataggggaatctgtacatctgatatgaagttatggattgcaaatcgtacatctgatatggaattaatattccaaaatatgtaagaaactcaacaaactcaattgcaagaaaacaaataacccagttaaaaaatgataaaggaatatccatttctagaaagaagacatacaagtggccaacagatatatgaaaaggtactcaccatcactaatcatcagggaaatgcaaatcaaaaccacggtgagatatcatctcacacccattagaatggctgttatcaaaaacacagaagataagaagtgttggcaaggatgtggataagagggaacccttgtacactgttggcaggaatgtaaattggtatagtcattatggaaaacagaatggactgcctcaaaaaattaaaaatagaactagcatatgatctagcaatcctacttctgggtatatatccaagggaaatggaatcagtatgttgaagagctatctgctctcccatgttcactgcagcattattcgcaatagccaagatatgaaatcaacctaagtgtcaatggatgaatggatttttgaaaacacagtatatatctgtatatatatacacacatacatatatacacatacagatgtatatgtacacacccaatagaatattattcagccttaaaaagagaaggaaatcctgtcatttgcgacaacagagatgaacctagaggacgttatgctaagagaattaaactgggtggtatttgacacagaaagacaaataccacatgatccacgtggaatctaaaaaagtcgaactcataaaagcagtgagtaaaatggtggttgccaggagctggagggtgagagcaatggggagatatcagccagagtttcagttatgcaggataagctctggtcaccttctaaggtgatcatctgctaagtataggtcaccttcagcatggtgactatagttagtgatactgtatggtatatttgaaatttgctaagacagtagatgttaagtgttctcactacacatattcatactcaaaaaggtaactgtgaagtgatgaatatgttcattaccttgattatgataatcatttcatagtaaatacgtatattaaaacatcacattgaataccttaagtgttttgtttgttttttggttttttgggtttttttgttttgttttggtttaggttttttttgaggtggagtcttgctctgtcacccaggctggagtgcagtggtgtgatctcaccttactgctacctctgcctcccaggttcaagtgattctcctgtctcagtctcccaagtagctgggattacaggcgtgctaccacacccggccaatttttgtatttttagtagagatgaggtttcaccatgttggccaggctggtctcgaactcctgacctcaggtgatcctcctgcctccgcctcccaaagtgctgggattacaggtgtgagccactgtgcctggcctgtataccttaagtttaaaaatttttgtctattatactcaataaagctggacaaaattttaaataaataacagcagtcattaacagactcaattgatgacctaatgtagaagttaatgagagcaggcctgttggcaaaaaggcatttatatggatacactgtatgtatctgcactgtttcaggatcctctattatgatacctgggtaaagggtgacttcctttctatcataaaacagcctagacagcactaagaaggtggttatgttcttttctgttgttgtgagcgcccagatgagattactttgccaaagactcttttcatttctctttctgaaactctgtgagattgctgtgttctctaggacttaacttcacagctagtctatttttcctttcagaaatgaccatggttgacacagagatgccattctggcccaccaactttgggatcagctccgtggatctctccgtaatggaagaccactcccactcctttgatatcaagcccttcactactgttgacttctccagcatttctactccacattacgaagacattccattcacaagaacagatccagtggttgcagattacaagtatgacctgaaacttcaagagtaccaaagtatgatgtttattttcacttttcagactactaggactagaattggactcatctctcagtaaccctgtaataaataatgctccagagactagaaatctaatagagaaggcattcaccattcatttattcacccatttatccatgaaatattaggtatctatgaaatgccaggaagggagctggcacgaggtcaatcttgccttccccgccccctccttccttcctctcttctttcctttcttgcttacttccttcttagggacattttctaccttgattacactgtcagttgtatctcaggctaagagtccatcatccaaactttgaattattttggccccttataagttaataagtaacaattccaagtcactttgtattatataacttatcttcactatattactgctactacagtacattttttctagtatgtcctttcaggagtttatactgaaacctacttttaagcacaaatttgttttaacattgtagcctattgataaattttcatgccattaactaaatcaacatcaaaacatcagactcaagaaaaactagaactgcactttttgagataaaatgtatttctgtgatgctatttccttgatttttatcatgtgtgggctgacatgctggttttccaaagccaccactatgtaattggcaatcattacttgacaagtaagaacttcctgtatttctagtggcttttaaagataaataatatgcttagttatatttatctatgcggttttcagttagagcctaatatggaaatttagtagccctgagcccaactagtaaaaggtagggcttacttccataagttccgccacgctctgtcaaagattatttcacaaaccacctcctttgactgaacactaatggtaacctgggctcatatttttcatgcacctcaagctaggcataccatgaccttttcacagtggtgttccttctgtggttcttttgcaagttgtttcaaagtacttataaaatggaatatttaaataaaatgtataatttaatattgtgatgaataccaaagacagaacatttcctagcaaaataagcttccttgtctcaaatcctctatgttgtctgttacagctgtagtattgcaggctactgggggtcatagaaccacagacttaattctcattcccattttatatttacaggtgaggcttagagatgttataacttgcctgtgattacaaacctttctttttcttgccttaactccagtgttttttcatgttccactgtgcttttacactgaaacaatacaaatacagcatgaaggtgtactatggtggcttgccctgttgcctttttaggactgttttcatgggataattatcctctcacatgtctccatacacaggtgcaatcaaagtggagcctgcatctccaccttattattctgagaagactcagctctacaataagcctcatgaagagccttccaactccctcatggcaattgaatgtcgtgtctgtggagataaagcttctggatttcactatggagttcatgcttgtgaaggatgcaaggtaattaaaaaaaaagtcttcaaagaaattgttgaaactttattatttcatttcagcagaaccccttttttaggtgatacaatatatgaattttttttcttcatggaagagtatcttgctctgtcacccagaccagagtgcagtggtgctatcctagcacactgcagccttgagcttctgggctcaagtgatcctcccacctcagcctcccaaaggatgatataagatttgaaagcctaagaaagtggataaaagttgggctgctttgccccacatttgtttctccaaattgattctttgagacactgatctaatttaatccttgcatcttgcagatttttacaaaaaggtgacttctccaaggtgtcatggcaagacagtgatgacctggaaccaggatgcagatttcctgcattctctagttgtttatttatttatttatttatttattttgctataccgtgtaatattgtatagacagaagattttacagattatttatatatgcatatctttggatccctagagcaatcctataagattggacttctttatggccacattgcagatgtagaaattacaggttagtaaaacttgccaaggtcatcccttagtaagtggcagaccgcagtctagaccctgactccttaggtggtgttggctctcaaagcagtgcctcatgtttccttattccagcatcaatgtgagccacatacacggacaggtaaggaggtataacagtgtgggaaaggagatactatttatcctcatctctttcctttgtgaaaagggtattttggaaaattcagacgtttagtaatttattgcttaattattaagaaaatatgtgaatccactcatattttgatatgcagtcttttaagataattttaatatacacaatgcaaaaatagtactaatttattatatacgcattgcacagaataaactgttcataaatattcactttataaaataaatacctgcttattgggctttttattcaacgtaaagatacagaacaacagttaggaaagttttaggacatcttggttcctaaatggatggtgggccaaaaaaggaaactgatttttattgaattcttactacatgccaaatactgtatgaggtaacgttttcatacatcagtatttaaccacatcctacccaggttttctcatctataaaatgattgtggctgagtgtggtggctcacgcctctaatcccaacactttgggaggccaaggcaggaggatcacttgagcccaggggttcaagaccagtctgggcagcatagtgagaccccatccctacaaaatttttaaaaaattaaccaggcatggtggcacatgcctgtagtcctagcttcttgggaggtttaggtaagaggatcccttgatcccagaagttcaaggctgcagtgagctatgatcataccactgcactccagcctgggtcacaaaagcaagaccctaactccaaaaataaataaataaataagtaaataaataaaatgacactgaattacatgatctctaaagtcccttcaattgaaacagtttttactctaaaggcaacctaatacagtgaaaagggacaaaccagaggtcagctctgccactttccaaagtcattagccagtcacatacacattgtatccttcatcaggccccccatatgtaaaatgacttggttagattggctacacaaggtcttattcatctctaagttctgtaatttcaaaatgacactttgaaacactctggcaagagatgctttcatttaaaacactaaaacccgttcgttctgtaatttaaaccaagttaaaatatttatgaccttatatgaaatcattgtattaagtcagttatctaagatcttgatgagtagtaagaacatgtcattttattgacttagattttttattgaatgttttgtggttttattgagttttattttagatttaatgagttagagtttatttttactgagttggataccatttttgagtgagtaaactgttgctaaattctgagccatatttatctgaagaaaccattctgttttggtcatgttgataatttacaactataaataaataaaaacagcatggttggtttaaggttccacaataagttattgttttttatgggctagtaaagcacttagaaaactaaaacttttaagcaaatagttccaccataatcctattagtcacaaatttgtggaaagctaacattactgaaaacagctgaaaatggaaagacaagaacattgtcagtagttggttatagtgataaatctgagttctgagggcactaaaacttaaaagcaagcagggaagaagagtagcaaaaagagaagagagaggaggaaatccaagtgccttcaacttaggattagaaagggtagaaagcagggagggagaaaaaatacttaataaaacctgaagactctaggggagtccttaacctatcatagattaaaaagcaaacatagaagaaaactaagcgtattgtatactaacctgcagtaatttgcaaaccatccaaaatgtggattccagaattcctagactaaaatgtaaggaaggttttactcccttggaaagggaaaatagacatctacacgttttaaaggtaagaataagaaaaatattattcttggaaaatttaaacatttagtaatatactacttaatataaagaaaatatgtgaatccacttatattttgatatgcagtctttcaaaataattttaatatactcaatgaataaaatagcatttattgtatactgattgcacagaataaatttttcataaatatttaccttataaaataaaccctgcttcggctggggtttttattcggcgtaaagatatagaacagcagttaggaaagaattaggacatcttgattcctaaatggatgatgggtcgaaaaaggaaactttttttgaattcttattctgatttttattgaattcttagtatgtaccagatactatgtgaggtacatttgcatatatcattatttaaccacatcctactcaggttttctcatctagaaaatgattcagatgatattggtaggatgtggttacataatgaaacattatcatttggcttcttcaagtatacatcagcagttcccaaagtgtggtctggggcttggaccctttcaaggcatccatgaagttgaaactatattcataataatgatttttattacttttttcactttcattctctcaggagtgtatgtggagtgttctggaagctacatgatgtaggatgatgacatagctctggtagctaatggaatgtttgcttatgtgttcttgtatataataatttctcagttgtaatttctagtgtgataaatgtcagtaaagatagcccacataaacaaaatctctttggggtcttctataattttaaaaagagtaaagggatcttgcaaccataaagccttcagtctcattctgaagaaatgcagttactcattctttgctactttgaaactagacaaaattgctgccacattctgaatactgctgcattagaccagtggctgtcagtaaagacatctccttagcttataaccaatacgcaataaaaagccaggaaaatggaattagcaaatgtgtttaactcatctgcatgtattttctgagaggtgttttggaagcttcacatttgctgttgtagtaaaatgtgggttatattcataattaaaatacagttgaccacaaagctgcaggcttatctatggattgaaaagaataaacacagcataaaacaacttttggtagatcttcctaaacaattttacaatgtaaatggaacaggtttatattagtgattcttgccaaaaattcagtcagacagatgagaatgagtagattgggtttgaattaaggtgaaatagttacagattcttgggtatgttaatattgcaaacttatgtaaaataaaacctttagcaactgaaagatatatataatttagagcttggaaagctatcatttatttgttgtttacatgttggagatatatttttttctttggtaagttccatcctattttttatctttagtgaaagatcattttggaaccatagccgtacctaaagatataagtaaatacagtctaccatacaatacaatgtaatctaagtttgtatttctgtatacaaaagaatgccgtatgcttgttagccacttgtatgtctattcatccctacttcagaaagagttttaattcttctcacctgccagtcgtagcttattaatccaacttccaggggccagactgcttcatgggaaagccaactgcaagcccagcacagggctgttaatttcattgtaaaatcaacaacaccattctgaggtctcattgtccctgatgttttgttaaaattaaagcaattaaggagagatcaaagaaaatttttcaaatgatatgaaaaatttgtctgttactttctagcagacaaatgtgcagacattagaataaatatatacacaaaacaattttctcagatggtgactgatgcatctctaacacaccacatcacagacttcctgatcatcagaagagtaagttgttgaaaatacattgttctatggactctaaaataataaaaaagtgttattagagtgtttcttcatcagtaatatgaagaaaatttggagaagccagccttgttctgtaaatcgataaataattttacagaaaagcactcgataatatccttacatcttcctctctagattccttgagtcacataacaagaaaacacttggttatcatgaactcggtgtaaaaaaagtaaaagcaagttccaagatgcatgctaggatctgtgtattcagttcaagattttatttacatatacagtgtataatgttcagaatgattattcattgaaagaccttggatcatcacaaaaaatgtgatacacttttgaaagtcatcagctgagtcatgtcagaaatttccattataagagttaaacttaattctctttttttttttttgaacatgtgtcaaagatttatttaaaacccattaacgagggaaaagcaggatggtaaatctagttcaaagaagaacttgaaggaccaagaatatatagtcactgaaagaaagaatcctgagataagattcagaatggttaatgttctacagggcaataaaaccataaccttgaggttatcttttcttttttttattattcttatactttaagttctagggtacatgtgcacaacgtgcaggtttgttacatagttacacatgtgtcatgttgatttgctgaacccatcaacttgtcatttacattaggtatttctcctaatgctatccctcccccaaccctccaccccctgataggccccactgtgtgatgttccccgccctgtgtccatgcattctcattgttccactcccacctgtgagtgagaacgtgcagtgtttggttttctgtccttgtgatagtttgcttaggatgatggtttccagctccatccatgcccctgcaaaggatatggactcatccttttttatggctgcatagtattctatgatgtatatgtgccacattttcttaatccagtcaatcattgatggacatttgggtcagttccaagtctctgctgttgtgaatagtgccataataaacatatgtgtgcatgtgtctttatagtagcatgacttataatcctttggatatatacccagtaatgggattgctgggtcaaatggtatttctagttctagatccttgaggaattgccacactgtcttccacaatggttgaactaatttacacttccaccaacagtgtaaaagcattcctatttctccacatcctctccagcgtctgtttttttcctgactttttaatgatcatcattctaagtggcgtgagatggtatctcattatggttttgatttgcatttctctgatggccagtgatgatgagcattttttcatatgtctgttggctgcataaatgtcttcttttgagaagtgtctgttcatatcccttacccactttttgatggggttgtttttttcttgtaaatttgtttaagttctttgtagattctggatattagccctttgtcagatgggtagattgcaaaaattttctcccattctgtaggttgcctgttcactctgatgatagtttcttttgctgtgcagaaactctttagtttaattagatcccatttgtcaattttggcttttgttgctattgcttttggtgttttagacatgaagtctttgcccatgcctatatcctgaatggtattgcctaggttttcttctagggtttttgtggtgttaggtcttacatttaagtctttaatccatcttgagttaattttgtatatggtgtaaggaagggatcaataattctcttatcaaaaaaaccttcctctcctagccatgccaaataactgagcgctctgtgagccatccagctgtcagctagccaagtagcattttcacccactaatattagtctttaaaaagcaagaatcaaaagagaagtcaaggaatatcataaaatatctatgatcacaatggcaaacatcaggattttcaccaacatttatcaatgagaacagttctatacattagcattctgttcatctgtgaaaaaaaaactttttgatatctgaaggtcctcagaaccctttgagaattaaagagacttcaactgccagcaaattctacatcgggcatattctttaagacattgctgatctcacaagaggtagtgaaaactctccaaaacaacatcaatgaaacaaaatgaaacaaacataccctcagttccaagctgtgagctgcaggcacacttagcaggtcagggttttccccatgatagatgcccagagaggtacagtggtgagcattgccccaaaggcagatggctgtagcagttttctgagcaataagtgagaaggagctaagtgtagggcttctgaattaagccaggattctcaatagtgtctgaaatagtcataagtcagtaacacccctggctactgaattcctaattcagactttgtaggagtcccccagtttaaggtcaagaaactagttccaaataatatcagcaagcatgtgagaagctaaccatcttgaatgacattcatcatcaacaagaaaactgatacagactccctgctcttcccctacctggggaaagcagaaattgggattgcctgatacagaatataaaaaagctattatgaattatttaaaggaagagggcataggcttgcaacaataaactattaggaataaccaggatgatttgaaaaccaagtgaaaaaaaggtgaagcttctaaaagttaaaagtataattattgaaataaaaattcagtggaagggttagatagcttactggacacagcagaagaaaaacctgaaactgaaagagatatgcaaagaaattaccagaatgaaagcaacagtgtcttaaagaatatgccctatccaggatctgctggaatttgaaggcttttagttcttgaaggattcataggatctttgaatattaaagaggtatgtcttcttcactgatcaatggaagagtaaagtgcagaactgttctcactaatgaactttagtgagaaatgtttgtttttacttttgccagtgtaattatatattttcatgttattttgcttctgttctgatttttactttggcaatcaatattagtgagtaagtagtgagaagaagatggaaaataaaaaagaggcattcagaaatgtgaaggctagaataagaaagtctagtacttatctaactggagtccagtactgatgaacatgtgaagccgtaatgcataaacaacatatccaagaaggtaaatgaaaagaaattcatgcccaaacacactgcggttaaactgccgaatactaaagacaaatagagagtataaaagcaaccaaagagaaaaggtagatcaccttagaagtaaattgacaagagacttctcaacagcaaaatggaagcagaaggataatggaattcaaagtgttaagagaaaatgactgagtacttggtaatctctcaagaatgagagtgagccaggcgtagtggcacatgtgtgtaatctcaggcctttgggaggccaaggtgggtggatcacttgaggctgggagttcaagaccagcctggccaacttggtgaaaacccatctctaccaaaaatacaacaaaaaaaaagagccaggtatggtggtgcatgcctataatcccagctacccagaaggctgaggcccaagaatcgcttgaacctgagaggtgaaggttgcagtgagccgagatagcaccactgcactccagcctgagcaagagagcgagactctgtctcaaaacaaaaaacaaacaaaacaaaaagagtgaaatgaaatatctattggttgaataaacaaaaatactgaaaaagtacatttcaaggatagtaagccagagaagatatttgaacatctgtaacagacaacagattaatatctagcatattttttaaaaaagtctaaaattcagttttaaggaaaagacagttagaaactgggaagtgaaaagccagggacatacaaatcaagatttaaattcaattttatacatttcagattgtcaaaattaagaagtctgacagcaccaaatgtggaagagactacaattttcataaagcgcttgtggggagtgtacattggtagccactttggaaaacaatttggcattatcttgtaaaattttaatactcacacattgtacaacccagaaatgccacttaaacatatttacaggaatatttagagaaactcttctcataatagcaaaaaattgatatttcaaacgtccatctgcatacaaataggttaataaattatagtatagtcatacaatggaatactatataattatgaaaatgagtaaatcatagtctttcactaacaagaacttaaatgttagaaacctcataccaaattttaatagtgtcccagaaaactacatatagtataataccatttaataaagcctccaaacaagcaaaattaaatagtatattattttgttttacatagtatgtgataaagctgtattttcttcctttttttttttttttttttttgagacaatgtctcactctgtcacccagactggagtgcagtggtgcaatcataacttggcttactgcagttagacctcctgggctcatgtgatccttccatctcagccgccagagtagtagctgtgactacaggcgcatgccaccacacctgcctaaattttttgtatttttttatagagacaaggtttcaccttgttgcccaggctagtctcgaactcctggactcaagcgatcaatccgcctcagtctgtcaaagtgttgggattacaggtgtgagccaccacacccagcctatattttcttaaagatcaaggaaatgacaaacccaaaattgagaataatgattactttgtcaaacctatcgcctttgtaacatctcagtcacaattgggaatcacaaactaccagagatggaagtgctttctcattttatagttgaaaaaggtgaggcccagaaaggaagttacgtgacatctgtattaattccacaaattttaatttagcactatcataggcatttggattccatccaggggtataagagactattcctgccgctgaggaacttataattttaattgagcagtgagggaggggcagaaaataaacactaaaacaataataagtaaattctgtggtgtgttagaaagtcataagtgctattggaaaggagaaaaactggagcagagtaaagaagatcaagagttccagaagagaggtggggcagggacaggccacagtattaaataggttggcttagccccattgagaaggtgagatgcaaaacttgaaggcgatgaaggaaataggcaagtgtgtatttagagaaagctttcttcttgaggaaagaaagaagtcaagagtgatttcaaggattttggcttgagaaactggaaggatggcgttgcgtctgctgagatggagaaagccgcaagtgaagtgagtttgtaggggagggtcaggacttcaatttggacaggctggcttagagatggctattaattatttaaatgaagctgtcaagtaatcagaacatactcatccagagttctaaatggacgtttggagaaagtcaaaatgcatgtcaatgaccaaagcaatactaatgcagtgctttttgcaccaaacagggccagatacatttgaaagaattagcaattaagttttatgaagatgaacacaatagaaatgaataggaatatgatgaacccaaaaaatggagtcagaaaacacgtccagttcgatttttttcagctctttccaaagatgtaaatatagcagaatattcagttggcctcaaattgacatatttgccattttgagatagcagtcgattttgtgaggatttatctgattacttctcatgttattcccatagtctctttctgtctgtaagccacaacaatgatggggcacactttaaacaacataaataagtgaaacaaattctgcaggaaattatgcaacatagagaattccaaaaaaattatccacatgtggatttttattggccctgatcatgttttagaatgaaatgattataactttgccaggctgcttagcacaattacaaaggctggggcatgctgttggaatgatttgcagagagacttaactgattacacggtgttttatttgcattacattaaatccagagcacttcttagagcatgccgaaaaccaaatgtagagccaagatccaatgaaagccgcagctggatgtggtcagtgtctaagagctaaaaaagggacctggagatcctctggttctttattttatagcagagaacatttagttccagagaggttaagtgacttgtctaaagtcaccagtgacagaatcgtgtcaagaacctgccttttctgattcccaggccagtatacctttcgctgtaggttgctgcttccatgtgtcataaagacttaaaatttgcttcttttttatccctttgcagggtttcttccggagaacaatcagattgaagcttatctatgacagatgtgatcttaactgtcggatccacaaaaaaagtagaaataaatgtcagtactgtcggtttcagaaatgccttgcagtggggatgtctcataatggtaagtaaacagtcatcaccatatactttattattctcattatagctgccagaccagtggacactaaagccattgccaaaaatgtgtacagtttttccaccaaatgccagaatttagaatattgcatggcgataaaacatttctcttttaggtcagtgtttttaaagttttattatagaacctttctctctgtggttgggcatctgccatgaggagaaaagagacttgaaaaatctgggggattatgggaaaaactttagtgttagaacaaatataatgatcatcatgattaatttttatattgcctcatgcattaaaaacagaattaaaatttacttttgtgtatcagtagtgtcgttctgctttaaatgtaagaaaatcctgaagatgtctgggtctgaaactttaaatggcatatgagaatttagattcatttaattttttttttttttttttttttttgctgtttattatgcaaagacgagtattcgtttccttcacttcaaggaacgtccatgagttgtggaaagggtaaaacaaattatttttatagttctgcaaaacttcaaagccatagaaacataagagaataaaactttctacttttgaaacctgtgtctaaagtcaagcttatgactttgggtgtcaacttaagggaaatgatgtagaagacctgagaaaagacccatgttttcatgatctgtggaactaactcagtaaggtcctggaagacaaacgtgtttgaaaaaataattcttggtcactatttggttctgattggttgcctggctgacaaaataaattttaggtgtaacttgatagtattatatgtgtctagtattaatcagttattttcaatgcactcaaggaataaaaaatgccaacatgatcaaattaaatttgaagtttcatttcatcagctatcctattttttctttcttttgttttcatctctctcaaaactgatctagtaagaaggcagtcttcatctatatgtactctaaagcatctgagaaaattaaatttttgctctatcatgcagatacttggttttcacagtggaaaccatcaagatatttgagagtcactcattgattaataacctaccctttaaaaatagttcacaaaattctatctgtatgattactaatgggtcagttcctctaaaggcaagaatgtattttgaatagagctatcagcagtactgaacaggatggaaatttggtccaactttcctcaacaattgactacatctgctgtatacaactgtagtcatggattgcaaaataatttgctggttcagttccatcagtttcctttttatttcagacctcataaaacataagggcaagcttccaagtcaagggttgtattttaggtgatgcaatcaagttgcaataaacttatttcatcttcatgaagaaatcattgactttgaggtcattatcactgagacagcctaggaaagcatgccagttccgcttttaacactaatttttgcctattaaaaaattatcacttcacacattcatgtaaaatataattcctgttacttacaggcagtgcatacattgatgtaaaatatatttcctgttgcttacaggcattgcatacgtttacttaaaatcaatctgcaattgcttattgtttataggcaaatattatttaaaccccaacctatgtagtatggatggtataattttctcctcccataaatgtcaaagaaagctatcataaacttgataggtcatattttgacttcatatgacaatttattttatttctagagagggtcaatcttgatagggagtgtttgttcagttctagcaaaagcaagaaaatatatgaattaagtgtcatgacatcagagacttaggcagttcaacacttaaaaaattgcaaacataactaagaaaacctcagaggtttttatgggagtttgtttgctgcacagaaaataaacatcagactaacaacaaggttttgattagttttgcctgttagctgtatgaagaaaacatttctaaatacaaagttttctaggaagttgagtcactatttaaaatccattgctttgaccaatgtgaagccaacagggttttcattcatatagaatgcctttttatcaaaagaggctctgagtgttataccaaaatcagcacccagctgttttcagtatgcgcaagaaaacatgaacgtcccccaaagcaaatgtactccattccgtgttaaccttatttctgctccctggtctctgattagtctggttgatgaatgtagcttcccctttggccatgcactcacagctttgaaggaggcttctgaaggaactggtagggaaccctaggggctcttgctaatgttgttggatagtcactgaaatcagtgatcaccatgtcctttcactttgaaaattctatgattctgtataatcaaacaacctaactcactggatttttcacttttcaaccagctattccgtgtcttacagtgctctggtcttcacagataggtaggcacctggcatgtcagttgcctgaatttgaaagttttcacctgtatgttttggtacgataaaaataaaaatgtaatttatatatctgaatcaggtctgtatgttatgatcaattgctcagcaatttcgggcagttggtttgatggttatgtagtaatgtagcctgagagcagaaatacagagcctctgggctagagaaagtataaatggcatcctaggctatgtagggttacagctcttcagaaggaactttcattttcattgtgacacatcgtctacatgttgtagaagaacatagtttcagaattcttccagttagaaacatagtttctcaaatattcactttcagcattgggtagaaaaagtacccaggtgatggactgattgatatgcttcagaaatatgttttttcctgcaagtaatgaaaataaacacacgttcttaattattttggtcatttaacaacccatcatttctggaccatgggttaattccaccgattgctcagaagccttcctgagtcatctcaagcacaatcaattcactactgtgtctagacactgcccttagaggaaagctcaaaatgaaaatgaagcagaaatacagacccacttcctaggccactcagcggtgtatgtaacttaccaagtgatcattgcatcaactactctttgttagtgaagctggtgtctgcagaatcttgataaaaagactcatggataatgttgcttctttagtcaaatgggctcttgcatgcatttttatagattttcaaagtagtcatttttgttttaaaatcctgctgccaagtcctttatagtaattgcattttaaaaatatgtcttctttattttttattattatttttttttgagatggagtctcgctctgtcgcccagtctggagtacaatggtacaatcttggctcactgcaacctccgcctcccaggttcaagcaattctcctgccttagcctcctgagtagctgggattacaggtgcccgaaaccatacccagctgatttttgtatttttagtaaagatggggcttcaccatgttggccaagatggtctcgaactcctgacctcaggtgatccacccacctcagcctcccaaagtgctaggattacaggcgtgagctaccacactggccttgtcctctttatttttaataaaattatttagtcattaagaattttggcttgtgggccaggcatggtggctcatgcctataatcccagcactttgggaggccgaggtcagaggaccacttgagctgaggagttcgagaccagcctgggaaacatgacaagactccatatctactaaaactacaaaaaattagtcgggcgtgatggcttgtgcctgtggtttcagctactcgggagtctggagtgggaagatcgctttagcccagggggtggaagttgcagtgagccatgattgcaccactgcactccagtctgggtaacagagcaagacccagtctcaaaaaaaaaaaaaaaaaaagaattgtgactagtgaataattcatattcagagctctttttaatgtatgaactaaataataaaatagtaagtttaggctaagtgccctttttatttttaaaattttctgtatttattattactatttttagagatggagtctccctgtgttgcttaggctgggctcacctcagcatctggagttgctgaaactataggcatgtgccactgcacctgacctatttaagaaaataatctagagaagaattgacatgttatttatgaatcattcagttaggttttctctataacattgagatttggttaaaaatatgtttttcataaatttaaaaactattgaaatcttagctttgctgactaggatgtcttattatataaatgagagagagtttgtaattttttaaaaaaaactgacaactttcaaaatagtttctcagtggcttcataaaaatccagaaaaaaagatttcacaaatttttgtgacactcaacatttttaaggtagttctctcagtgccttgcaaacagcagctctagtgtatattgagaaaaggaaaaattctgaatggcaaatactagatttcaactttaacctattcctttttattattattattattattattattattattattattattattattattgagacaaagtctcgctctgttgcccaggctggagtgcagtggcgcgatctaggctcactgcaagctccacctcccggattcacaccattctcctgcctcaccctcctgagtagctgggactacaggcgcctgccaccatgcccggctaattttttgtatttttagtagagacggggtttcaccgtgttagccaggatggtcgcgatctcctgacctcatgatctgcccacctcagcctctcaaagtgctgggattacaggcgtgagccaccgcgtctggctaacctattccttttttttctttagtcttcttggttgtgtaaagaaaaattacccaaatcttaactttcagaaaaggtaacaaactcctaatgcatgtggcagtacatgtttgctccagctgccctgtccctctaccctgcgtggactccagaaccgcccatgttggagcccacctcctctccccacccctgcccactttccactcacacctcaggcaaaccctgcattttgctgttcctggaaaatggcttatgcttggatacctctgggcctctgttgatgcgtctgtctgttccttgtggaaattcttcctgacattcaaggctgcattcaaatgccttccttcccatgagacttttctgattccctgctcttccttttctatgtgtttcataccttcctgtgatatcacattctgccttaaaaagcaagagagtgatcagatacatattaccaaaaaaaaaaaaataccgatagcagtaaaaatgatagagaaaagactggaattgggaaatccatctgaagaccaatgcaatattaggaaataagacattgagaagggtgagaagaaaggaaattgagagaaatatcagagcagtgaccaatgaataaaagcgcctgaagggagagaagttgtcaaagattatttaggatatctaatttgggtgatagaatgaattaaccaagagaggaacacaggaataagagccagtttggaggagaaaattttaaattcagttttatattgtgttagagatgcctatagagtatccagcaactacgtatagaggaatataggaggatgtgaaaaataaggctccaaatttcagtgaagagagagcaggaatcatcagcacccattgtaggttagagaggtcacactagggaccacagaatacaagctaagggagaggcaccatcagagagatctggagagcagctggcagaatgttagaggacccagaacagcaggaaccaggacaagaaattttggaaataagagggcggagatggtcaccagaattaaaggctgggagaatactgaagagctcaagtctattgagaacttagaagaggccattagttctggccgagagaaaagcattcaggatttttacaaagttttggtaaatcccagtgagcgcaaagctagactgcagtagatcgagaagtgaatagaaagtgacaaacacagacggagtgaaaacaactctttcagtaagttcagtggtggaggaaagatagcttaaagaggaggtaatagtagagtcagaaccttcaacctgggcagaaaggccagagccagtacagagaggcacactagagaaagactcatctgtgtaatatacccagtctcccctaccagacatgtcctttccatcctttcccccagccccatctgtgcagcacagtgttgcatcaacgaagtgttcagtgaatgaattaaaggttgtcaggaagacttcagggaatcagagattgagttagaaggtgagctggtggtgttttgctcatcttcatttttgaaagaaacctaagaaaatgtttaaatgtcctctacaaatagtgtgaaaaatggtacctctgcataaaaataatttaatattagtgctaaagttgctccttgttttcctttagaggaactacaaaaggatagttattagattgccaaatcctgcagaacctccagcaggcacaagttggtttttctccctcaagaaagagaagtgagccaagcgtggtggtgcatgcctgtggtcccagctactcaggaggctgaggtaggagcttcccttgaacccaggtgttcaaggctgcagtgagccaagatcacactactgtgctccagcccaggcaagagaatgagaccctgtctctttaaaaaaaaaagaaagaaagaaagaaaagaaagagaaaggatggaagggaggaaggaaggaaggaagggagggagggagggaaaaaaggaaaaaggaagggaggaaaagaaggaaggaaagaaagagaggtgagccaggtgtagtggctcatacctgcaattccagcactttgggagactgaggcaggaggatcacttgggccaggagttttggcgagatcccatgtctaccaaaaaaaaaaaaaaaaaaaattagctgggcatggtcatgtgtgcctgtgggcccagccacttgggaggctgaggcagaatgattgcttgagcccagaaggttgcagtgagctacgatcatgccactacactcccacctgcgtgatcgagaaagaccctgtctctaaaaaaaaaaaaaaaattttaaagagagatgggaaaacaacttattggcaatagcacatgttcttgtttgtatgcaacaaacatataaaaagtagaaaaattgaccataagacatagccttactatttttcaaaagcctgataatgattgcaaattcatcccttagatgtggaaagacaaaaataagacttgtcttatttcattctgttattttctattgatttattattttgaaaacctgctttaattaaccttagtaattaagtagcatttgtctttaatgcttctacaattaggtagatgttttaaaaacatgccgattaattgtttcttagcgttatacccagatccaaaaagcaggacatctaactattccatgtgcttggtgtttgtgtgttcagcaatatcagtactaaatacacaatacaaatctataacatgctagtagtagctacattcagctcaatagaagtgtgttgtataaaggaatgccaatatgctttcccagaagttaatgagtctctatcaaacccagtcttcctgaagggagcctaccagctctgatatggataattttttttaccaagataccagatttctctgtaggtgatgaattattccaaataggaaactgcgtgccagtgttctggaagttgctctgtgtgacaggtgccagatctgtagaacagacagaatgtaattttttttgagcctcaaaaactagacaaatactgcttttaaaaagagagcagagcattttcttgagtactatgtagacatttgtagaactttagagctaaatttagagctagaggaaggaaactttagaatcttcttttctaagttcctcattcagtaaataaatcagttgaagaccagagaagttcagtaattttcttaaagccacatagctagtttgtcactgagctgaaactagaaattaagcttcctgactgcattcagtgcagtattcattcccttgctttccagagaggccaaaagttcttgctcagtgactcacagtaagacatgaagcattttccttttaagattaatcagcagtggtagaagatttggaaaaatacacttgtatatgagtacactcagcctttatagttctgacccccaatgcagaatgttcatcttctatttctaaggtctgacttcagtctttcaggaaacacctcccaacctccttcgctttcctcctcttggttgtcagattctccacatgaagggtagaacctaaggctcaagaattacttaactaaaaaacaccatctcacaaacaatttaaagatggaaagatagaaatagagataggcagacagatggacacatagattaggttttcagattctgttctttctctgtgggagaggtcagcgtgccatgactaccagcctggtctagaacccacagcagccatagttaccaagaccacagcaataaaagctgtgtttgttaagcattgccatgcacctagcactgtgttaaacacttcctgtgcctcctctcatcacctcacgtgaggtagataggtacaattatgtgggactcaaaagcaacgaaataataaaactggcttttttcccccttaaaaaaggtatgcgtgcctcagctgaaagacagaaaaaaaaaaaaaaagtaggcaaataaaaatccttgttttctgtacaatttcagatttgtgtatgttttatgggaatttagtctcaccaaatgggatcgatttttcattccttttatgaacttctggtgtataaaaatgttaaaatcacctatataattggttgcactggtagttaaatggaacctaatttgtagcatgggcatttaacaatacagataatttattcattagagttagcctttggggggaacttcaacttacaaaaatacgttttagtgagattatagcagagggatctcactttaatatatcctaattctaaaatggtttttcggggtattttttggttatatttgttaggtgatagaaaagcaagactagtaaatgtagtaagtggctatctttggctgtttgtccatttaaattaacttttttgattacccattaggaaaaaattgcttttctaggtgtgtaaagttttgcatatgattatagcctcttacctgtaaaaactgaatcagagcttagaaccatgcctggcgcacaatagtcattcaacaattattccttgaataaagagacagtattgtgtggtctgtgaaataacaaaagttcaggagcaacagacacttggtgattctagtcacctctattttgttataggataactggttttctctgagttttacaccagaatgccttcttcttttaatacgccttcttacttagtgcttttctcatgatcattttcccaaacttcctaatggacatagtgtctcattttctgtctgtttttaaacctgatactttggagtttaatccatcaaacatttattgttaacctactatgtgtattttgttacacctaggaacaaagatgagaaaacatgttactcatataatttatattcgagtagaataactgatataaaccaacatcatagtatagtaacagtttaatacatgagctaatgtatgcatacaacaggagcccagcatttaggaagctttttataaactgtcccaaaccccaggttttttatatactcctaattgagtggatggatggcccatcaaaatgtcccgtggaagacatataatgtcagaagttaccgagtgactctgataggctctgcctcccctaccctccgcactcctcaattaggaactactggtgaacaagaaatgaatgtagtacagccatccctccttattccctgggattggctccaggacccactcagataccaaaattcctaaatgtgcaagtctctgatataaaatggcatagtatttgcatgtaacctacacataccctcctgtatactttagatcatctctagatgacttataatatctaatacagtataaatactgtataaatagttcctatggccaggtgtcatggctcatgcctgtaatcccatctctttgggaggtggaggcaggaggatcccttgagcccaggagttcgagaccagcctgggcaacatggtgaaaccctgtctctacaaaaaaattaaaaattagccaggcgtggtggcacacacctgtagtcctagctacgcaggaggctgaggtgggagaatcgcttgaacccaggaggtcgaggctgcagtgagccatgattgtgccactgcactccagcctgggtgacagaacgagaccctgtctcaaaaaaaaaaaaaaagttgttatactgtatttttttatttgtcttattttttatcattgttttatttatttattttagagacagagtcttgctgtcacccaggctaaagtgtagtggctctatcacgactcactgcagcctcaaactcctgggctcaagggatccttccacctcagcctcctgagtagctgggattagaggcgcacaccaccacgcccagctatttatttttatttttatttttgtacagacagggtctctctatgttgcccaggctgctctcaaattcctggcctcaggcaatcctttcactttggcctcccaaagttctgggattacaggtgtgagctaccacacccagcccatattattttttattggttcttttttccaaatattttcaatccacagttggttgaatctgcagatgtggaacctgcgggtatggagggccaactgcataaggaattctatctgagcaaagtggtcaggaggtcacagaggattttgaaggaatatgctatctggggttttctttccattgtatttcaaaatacagtagacaaggacttaatatgtgccagaaattgtactaagtgctgaggatacaataatgaacaagattcctgcctttgtggagattatatggcaagggaggaaagaaaacattatagattaattcctgtgatcagtgctgtgagataaggtcatatcctgagggacccagacaagcctagcaccaggggctggggtcaccagtagataaaaatatgaacaaccataccccagccccatggaacaacacagaaagaaaatggggaagagtgagtagggaacaggatttttgaccaaaacctcctagtttaaaggtttctatatgaagaggccatgtaccaaggatatattcactaaaagtatctaaatataggctaattcaatttaaaggaatcccaactgagaggtaagattttcttcccctctaacacccaagtctggtctctaaggaggcccctaccaaacaatagttgtattagtcagcgataaaatttatttagggtcaatggaaatgaatttatgaatttgagactcaagggtacatgtcagtttcttcttgggaaaatccaaggtaatattcacagagtactcagatgggaaagttgtcgtgtgctgtcatgtctcatggtagaaatgctacccttagcataataacatataaaataactatctctggctgggcacagtggctcatgcctgtaaccccaacactttgggaggctgaggtgggtgaatcacttgaggtcaggagtttgagacccacctggacaacgtggtaaaaccctgtctctactaaaaatacaaaactgagccaggcatggtggcccaccccttgtaatcccagctactcagaggctgaggcaggagaatcgcttgaacccaggaggtggaggttgcagtgagctgggattgtgccactgcactccagcctgggcgacagagcaagactccatctcaaaatataaaataaaataaaataaaaatctctttgttctgggattcagcagagacagagtcttgctagagacataaacaaatatgacagtctctgcgctcgaggagcatatagtctagtgaagccgatcaccataaaaacgcatggctgtgataccacatcgtgagtgtaagaacagagctatgtatgcatggggaacagtggacatgatggccaggtacccttattatgggaaacatagaacccaaagtgtgttcccaaaggaacatgattcaaaataatattttgctgaacatattggtaagaaggaaagaaaacatatctgagttgaaaagttacagtttattttttccccaaactagtccccactaacccctcttacattttccttgttgcttttctttctttcattatcttctttgacacttcctcaccctacaggcagccagtttgtctttctgacaaacatcattaagtggtaactctggagagtcgggagataactatggcttctctccgctacatgtaaaagtgctctctggactagtaaaatgtgataaagcaccatccatatttctttttaacagagacaaacaaatcttataaactccctgagaacaaagcctatgtctgtacaactttgaattctgcacagtttcgtattttaattcgtgaaacgtgttgatccttctagtgcctgaccttaggtcaagtgctggggatacaaagaaggtgacctttgaattgggtcttgagggatgagtaggagttggttctcaattatttcacgtttaagtcgacatacttccctccctttgctaaactcgaattctttcactttctcagcaggagtatgcattaacttttaaaaatgaaagttaacggtttaatttttactgatggtctgtgctacttttgtgaaataaaaacatgagcaaagtggtagacagaaaccaggactcaagagcagtggaggaggagggcttctactgtgtgggaacgagggctgggagagcacagtgtgtgttcagagcagtagtaatccaatgattcatcctgtcattcctcttcctctatagccatcaggtttgggcggatgccacaggccgagaaggagaagctgttggcggagatctccagtgatatcgaccagctgaatccagagtccgctgacctccgggccctggcaaaacatttgtatgactcatacataaagtccttcccgctgaccaaagcaaaggcgagggcgatcttgacaggaaagacaacagacaaatcagttagttctcttctgctgtcttcattgggggaggcgggaagttgttttgggtttttgtttctttgagtaaatggtttactgcgctacaaatgcacacacagaatattgttcaactgttggctgttaacatattgcaggctgagtctgaaacgcaggaagtgttttcagatagcacaagtcaacattattttgaaaagggagaaaagtccataaagtttttgagaatagtgtaacaatgtattggtcatcctaactttaatgaatgaacttaagccccctgagcctatcaccctcttttaggatccatctcctttctcggcatcaaaataactggccacagcaatattcattcaagcagccttgctttcctctgggaagaacccgacctaggtttagaaaaggtattgaatcccactatttcacctttcaggagaactaccagagttacctctttttttttttttttttttttttttgagatgtcgtctcactttgtcgcccaggctggagtgcagtggcacaatctcggctcaccgcaacctccgcctcccaggttaaagcgattatcatgcctcagcatcccgagtagctgggattacaggtgtgcacgaccatgcctggctaatctttgtatttttagtagagacggggtttcaccttgttgatcaggctggtctagagttacctcttaagaagttcaacatgaggcataaaaatagattctagatattatgttacctttctttccttgtagcattcttgattattccggggcaactagaagctctatccttttattacatatgtgaattttatcttctgcaaggctctgagctgcctggactacttcagtggactctccaacatctgttcactcattgagcagcactgtttagaaaacccagaatgggttgtaattgttactgagttaggttttccaagcagcagagctcttctctgactggaaactgagaaaacaacatggaattatagaataaaattgtacctgaattctagtcccaattctgccatcagcttggatgacctggatcatgtctctgtacctcagtttgttgttgttgttgttgttgttgttttgagacagagtcttgctctgttgccaggctggaatgcagtggcgtgatctcggctcactgcaacctccacctccctggttcaagcaattctcctgcgtcagcctcccgagtagctgggattgcaggcgcatgccaccatgcccagctaatttttttgtatttttagtagagatgggttttcaccatgttggccaggatggtcttgatctcctgacctcgtgatctgcccgcctctgcctcccaaagtgctgggattacaggcatgagccaccgcgcccggctttgtagctcagtttttaatgacagggtttggctttgattctgatgtctgcacaacgctcccagggctgtaagaactgcagaacacactgagaagacaccgtcccagccatggaaagcccttctatgcagtcatttattttcacatgaacacctcccagagagctatgcaatgacttgatagatatgtgtaacttttcccctattcagatatattgttaggaaaatctcattaacctttcatgtattttgatgactatctcaagatggtagttcttctgtttagctaaactagtaagagtagaatcagttaatagaaaaaggaaacttatggcataaagccatatggtaataaggacaatgagttttctgatttgaaaataggtctttgatacctcaacaataatatacctggattcaattctttacagtataaaaaaaaccttgcacatttgttaggttcgagccacatgcacctgactactgctattattatcctctttattttagagatgaggaaaataaagtccagagaggttaaacattttgccctaagaccccacagcaagtgggtagaagacctgggactataatgcaggttttctggcctctagttcagagcattcgtagcatgctagcccctcagtagcatagtactttaggaagctagtctttcccacttgctggtagtaagccagggagacataatacactaagaaagcttcggtctgagaaaactttccatgattgttttttctcaatttttagaacagtaggaattcttcacctcatctgctatcccaacatattcacaattggaaaatttttgtgctgttctgttcttttcaaattacaaaagtccttgactaggactgctggcttttacatgcacaggactgcacaacactggtgtttataaagctcattcgttcaatgataaaattggccatagcaaatcaaaaataaatgaagacctctgcgtagctgattggactacagaaaataagccattcatatccaggaagaaaaaattaaaaggattgctgaaaaggaaagagccctaaagcaaactatctacagttttggttagttttcttttcttttcttttctttttttttttttttttttgaggtggggtcttgctttgttgcccaggtgggagtgtcttcttatattggtcaggctggccttgaactcggggctcaagccgtcctcccacctcagcctcccaaagagctggtattacaggtgtgagccaccagccatggttagttttcatcaactgctttctcaccttcttttcatcctctaattcatacctgcaatccttattagaaatcctgtcaacttaatagtaccagaaagtcaggcaaaagactgataaaaaaaaagcctcggtggtcaagaacttaaaattacactactggtgtgcaacgtgctcttatctgtgttactggaggacaccctcaagatacctcactccagttgactccttggctctaaatctttgagttaaaaaataaggaggttctttatggaataaacaggtattaagctatttaaaggagagggctgcgaaatgcttgattatgggcttttcaagttcagaggccaagcgtttgtcattttcttccagtaactaggactgtgtcttgcccctaatagttactcagactatatttgcttaattgaattaaacacagttgcctatgccctttgaaattctggactttcaacagagggcctctagcccaatatttgcttaccaaactggacatcattgatgatctggattcaggcagggtctggaaaaagagagactgggccaaattaaaataatccattcactgatgacacaaaactaaactacaattgtttggcaccctctcttctccttatcttgcaaaatcaaattaagcactagtggaaagaaacagttcagagaggaatatgggaaaggaaaaaaaaaaaccaaaatgtgatttccaacgagactagagatttgttctttatctacatggtcatgttactcatttgatagcatctatctcaggggtattatgttatctcttggccaggactcatgaaagttaagatttgcattgataggaaaagttttgcagaaatatggactcttgagagggtgggaggtatataaaagtggctatggctgtgcagcagagcaatttgcatttcttatacaccctgcttgagactgatgtcattagtgttggttaggccaaggctgggggaggctactcagaatagtgggtgaccaattacccagaacctttggaaaaggaaatgacttgattgaagaggccattctttaaatgtatcttactgctcaatttcaccattgcctattgtagaatgcgccctgcccgctcagcatttgcctctaggcagaccaggactccttcccctccccaactaatcttccttctcctgtatcttattccctagggccacttgcacctttcaaaatggataaattaaattcccctgctccccttctaccaactttaacatcatatccttggtgtatgatgccctacttttcattgttttgcctaaataagagtatgcttgggaattttaggcttagggctgggaaaagatgggatatggaatcgtcagctatagattgccggacaagaaattttaggagcagtagacctttcaaccgccctcaataggatggaactgactgtccccacaacctatttccctttcagtggcttgctccagccaaggccccttatttaagagatctttgtgcgctctgaaaaccatgcatatgccagaactaggtgctctgtttcatgaaggcagtttgataactgaatggactttggaagtgcccagtgtttgactattaccctggtgcgatgatcacactgggcgtatcattgtcacatgatcccatgtttgttgatgaaaataaacagtggttttcaaaaaccattttcagtgccatggtttgtcataaaacaataaatagaaaagcgcttgatgtacctgccaacttttcacccctatctaagagatggcgaagctccggctgcactctgtctgcaaataaggaataaaactttttatttgtgctatattggactttgggttccaaagcatcacattgtttatggtaggtacatgtggagtcactgtatttataattataagagcaaactggtgccattggaaaaaatggtctttctaaatagtgtgtgaaatatgaaactagtgaagaataaaatcaatatggaaatgtaaacttctatgaaaacatttccttacccatttaagtttgtgcattctgaaatttacaatggccatcaaacccagtgactcaaagctattggaaagatatctgtccttttaaatcctacacttacagctaaacaacgtatgggttcttttccctttatctctggaaaagcccgactgagctggaatttgctccccaaaggaaagacatcaggcagaatcctcagagaaaggggcttagggaccccagtagaaataaaatcaggatagtagtgccaattcatttctggggctgctggagaagttaggcttctgggatcattcatccatgtatcaatattaataaaataaattcttctattgccaggactgtcaggaatagctaaagatgtaaaagatctaaaccagtgtgttctttcagagaataagttaaaaataatattttatagagttaaaaagaaaagcacttggattttttttttccaatggctggactcagttggccaatacccttgtttgcctgctgagtaactctttggctggcaattgatttttttaagagctgtggttatgtgctagttcattagcatgtgaataattcttcatattaagttttcatttgtttgtcgtaaatctctaaaccctgcaggaaagcagaatatctttgacagctttcaaacttcacttctatcacacacagcactccaccagcacctctaggaaacagtggttcaaaaggccctgccccaagaccccagcatgttaagagcgttagtttaaaatctccagttcatcctagaacaatcattttaaaaggaaaaaaagaagtggggaagtcctatagaaagcaatggaaagggaaaagttctcaacccagaatcggggcttaggttctagttcctggtttgctataaatggcactttgactttggaatgtctctggcttccgtttatccattagtcagagaatgatgggacagattacttctgaagaccccttatgtcctataaatctcatagttctcaacaagagggaaacgggaacaacaggggcagaaaccaacgagaggctgaggaaaccaaaccaatgagttttggattgtagctaaccgaggagaaccaggtgtgtcaaaggtgatgaaagtcagatgtgtaaaaagcaagaaggaaacaaaatgaagaacatgagccctaagaattcagtgtctttccctggtaaaactaagaacatcaggaaagccacactcgatgaggcacacatttttccaagccagttttcactctgacagggttacctcaagtgattgtttgagagagggtagaggggttgactacactcattcaatctgccaaattagaaaagtcacccagaataccctgtgctttttgagtgattttcatgtttatctcccgtagcttgctattttgttattcaaaaggagtgtaaacttagggcattcatgtataatacctttcataactcatgttaaaatgctgatgttattggatttctcaggaacactactgttctatttctccattcaaaaaggtaaaaaaaaaattaattactctattttcatcattaatcccatttcccccattgtctttgtattctacataggaagcatgccatagatctataattgaatttactcatctatataaaacagtacttttaatcccatcctgaaaatcaaaatatatttttgaaaatcaaaatatgtcaaaattgaaaatcaaaatgtcaaaatattgaaaattgaaatatatcaaaattaattatatttgatgtatccctcaaaaaattgatttctccttacagaaatcattttgctttcctggaaaaacactgggttcaagtgcagctttcttcattccgttctggtctgtccagtatatgaaaagcacaagcacacaatgaccagtgacagattcaccatgaagctaatgaagcttcatggggcccttcccttgtgcagaagtccttccagtgcttggggaagggccgaagcaattttattctcataatttgtaatattttccttagagagactccactactgtgtatattttatgccccacaaaacctgaattcaccccatcatgtcccatttgtaattaagctttctttcctgttaaggagggacgcttacccagccctttgatgtacggtctgaagataaatacactgttagcccccaactagaagtgtaaaatcattaagacaaaatttgagaatgatacggctcctataatggcaatctttggatgatactgtccaagctgatggaaagtgtaaggactccactttgctttgtaaaaaatacaaaaaaacagccatggaagtgcagtacattttgtatggaacattgtaccacattgtaccaaatgtcatgttaaattaatgattcaagataaaatgcagaccaaatttgaccagggttttaagaggttctctctccataaagcaggctggccacgatcttctcactaggtttgagggtaaggaggtcccctgtgaaatatgaaagtgaactctctgctatctaagaattggtgaattggttgcaaagaaaggcagaccaggcccacccagggatcactcttgcagtttgacagatctacctggaggctgaaagaatctatgcagccttcggaaatgcagtgaaaacagtataatcagatatgactagggaaaaaataaaacaactggtgttcatttgcatgtcaacacccagaattctctgctagttcacctcttcttcagaatgcagaaaggattgaaaaagctggaatggggccgggcatggtggctcatgcctgtaatcccatcactttgtgaggcagaggtgggtggatcacctgaggccaggagttcaagaccagcctggccaacatggtgaaaccccgtctatactaaaaaataaaaaaatttaaaaaaaattgccgggcgtggtggtgggtgcctgtaatcccagctactctggaggctgaggagaatcacttgaacccaggaggtggaggttgcagtgagcctagattgcgccattgcactccagcctgggcaacaggagcaaaactccatctcaaaaaaaaaaaaaaaaaaaaatgaaatggtttggcatgatggtggggtgctgattcctctctctgtccttgagtatttactgccaggaaatcctgaccaacatcaattgccaaggggttaaagtcacattgttgttaaattgggagagaaaattagcaggattttgtgccctatgttcaactattacagagtttcaaaaacaggaacctggagctttatatgaggggacaggggaagtaaaggggaagagtgacaagggtgatttatcagggcatagttccagataaatcacttacatggaggtgaaagaatttcatgcatatgttctgtgccaagaagccaaatggttgagcagaagctctgaaagttgaagtgggcagttctgggtacagtctttccacatgtgaaaaggtgatgattttatattcatgttatttgtactcctgcatgtgcagttggtcttttcaggtgtccagatcatcattttctcatgacatgaaagaaagccaataaaggctagttgtggtggctcacacctgtaatcctagcacgcacttagggaggccaaagcaggacgatcacttgagcccaggagttcaagaccagcccaggcaacatacatagcaagacctcatctcaaaaaatgaaataatttttttttagaaaaagaaagtaaatatttcattaatataaattataatacactgtaaaatgcctggaggacaagaaacacgttttctctaggttctgtgaaagcatcattatccattcacatttttctcataattagaacctgacgttctggaaaaatacagtaaattttcatcgatgttacttgctctgccccccttgtattcctcccattaataatattaatcctgaaaagaaaaaaagaaaaaggaaagaaaccacccaagcaaaatgtttaagaaatacggtaaggacagtttggtcccaatttccttaaaaattagagccaccaagtaaacctgtttgggggtcctcacatctgtcctcttttggagtttgctaagtagtacagttgaagtgaattttttggtaacaaaagtaaaaacttccagaataactccagtaaagggtgttgatgtgacgtacaagtttggaaatatatttaatatctgagactcctcaatgttggtgttatttcctccagtttagaacaaacttaatcttttgatgaaaatcatagagttccatagatatgaatagaatctccaaccagtgcatcttgaagccggctgccacggccactcatgcaccttctctcctttcaggagagaattgtcagcatctctgctcaaaacaatctttacttttagtccatcagagacttcatgtttgtcgtgcatgcacactcacacctctccacacatatgcccactccccacacacatcctgcttctgagaaatgcaactagaatttcctccggagaagagatggaaacccctgattgagaaatttaaggcctaggaaaggtaaatgacttttcttaagtcacatggctcaccacagaaccaaagctggaacacacataccctcagctcttcttccagtgctttcaggtatacaagtatttgaacaggattcaaggtttaactccttatgtgtcattcagtgtatccagtgtaagtaaaatgttaccccttagtcagaacagccttgagaatttctcaatgactacaaaccttgcttagttctatacaagggaagtaaaagagagagaatgtcatgaccaaatgacttaacttttgaaggcagcctattgcaaacttgaataattggggcatgtattttattgattgtttgacctataaggacagatattttaaaatataaattcaaaattgcttttttgcagcattcacctgtaggccttctatatctgctctttgtgaaatagatggcttccagagcattaaaaaaagaaatagacattttcttggttcaagagctgtaatctgctttccgagtgatttttcatagttaagcatattttatgtgcacatagtttcatttgagatctgagaggatttcatctaaaacggaaaagttattacctgtgaaggagaacaaaggaaaaggaaaaagaaattttaagaaaaaaaaatttttttaaacctgtagttactttatgttcaagtgggtattttcaacgctgagttgctgaaatggttttttattgaaagaaggttaaaagtgtatttcattgaaagccaagataaacagtagattttgtatcattttaaaaatcaacaatcggccgggtgtggtggctcatgcctgtaatcccagccctttgggaggccaaggcaggcagatcacttgaggccaggagttcaagaccagcctggccaacatggtgaaaccccatctctactaaaaatacaaaaatcagctgggtgtggcagtgcatgccagtaatcccagctacccaggaggctgaggcacaagaattgcttgaacctgggaggtggaggttgcagtgagccctgattgtgccactgtaattcagtctgggcgcagagtgagactctgtctcaaaaacaaacaaacaaataatataaaaatcaacaatgaagactccccaaacaaaagaaatcaggattcttagccagtcataggtaaagtgttacagtgtaaattgaagggaaagaagaccaaaattggtgaaatatgtttggtcccagaagataattaagatgaataaaagaacttgagagtattttctcattattaagcatcttcagctttaaagattttagttagcaaagcaagtttacataaacagttttctgaacctgggatggcattcactgtgagttagaaatctccaagtcatccacgttttccctgttttatttgcagccattcgttatctatgacatgaattccttaatgatgggagaagataaaatcaagttcaaacacatcacccccctgcaggagcagagcaaagaggtggccatccgcatctttcagggctgccagtttcgctccgtggaggctgtgcaggagatcacagagtatgccaaaagcattcctggttttgtaaatcttgacttgaacgaccaagtaactctcctcaaatatggagtccacgagatcatttacacaatgctggcctccttgatgaataaagatggggttctcatatccgagggccaaggcttcatgacaagggagtttctaaagagcctgcgaaagccttttggtgactttatggagcccaagtttgagtttgctgtgaagttcaatgcactggaattagatgacagcgacttggcaatatttattgctgtcattattctcagtggaggtaagatttgtcttttgatcttctatgaaagagggtgggatgatggtggggtggccaaaagaattttggatttccttagtgttagtctgcattgtcactttaagtgccagtggcatgatgccatgaatcatcactacacgtgcaaaacactgtaccaagaaagagtgtcataggtttgcggtgtgaaggttataagtaacccaggatcttctctttcatccctcaacaatatattttttccagtcaaatgccctcttctcctttttctttttcttttcttttctttgtctctttcttttccttccagaaaggaagattagaggccagtctaatctttggttcctgtaagtaatggcatgtataactttgcttaagacaatgctcacttgatgtagagaagggaccactaagtatttctggtcaaatcatgctcctgccccaccatcagtaagatactactttgaattgcttttgtgatatcaggaattaaggaatacctactgacctccacccactctcttgtgtggattcctctgatggcttcccttaaagtacctttgtgaggtagttggttgtgattaagaatgcaatgtgaaccgaacacacaacatagaaataaatttagttatttagcaatttgttgtatttttggccctgcaagtagtacatgatctgactaaccaattccaaaagccagattttcttctgagtgtgacttttttcttttttttttcttttttttttttcctttttttttttttttttttttttgtgagacagggcctcactctgtcacccagactgaagtgcagttgcacagtcatagctcactgcagcttctaactgcagttagtaagctatgatcatgctacttcataaaagtaatacgtggccattttacagttgggctcaagtgattctcccacctcagcctcccaagtagacaggacaacagctgcacaccaacatgccctgctaattttaaaaatttttttgtagagatgaggtctcattatgttgcccaggctgatctcagattcctgacctcaagcaatcttctcaccttggtctcccaaagcgctgggattataggcataagccaccacgcacaatctaaacattactttcattagcctctcatgagtagaagaaagttttactttcatctctctgtataaaaattgtttttaagtggcaagttttcctattgaagtggactttaggtgagctttttgtatgtactctacagtgtatcctctgtgaaaaacccgtcgggggatagtggtaaactgtatataactgcacaatgtattataaagagcacgatccctttgtcatcttttgttgtccagttgaaaggagaatgtttctgaaatcaggtgcatagtgcagtagtcataagaatagcaaacatttatttaatacttgctgtgtgccaggcagtgttctaagcacttcatgcatatttattcaatcctcacaacaattcagggaagtaaggaccattttacaaatgaggaaaccaaagcactgagaggttttctcatttgcgcaaggttacgtggctagcaaaagatggagacaggattgtgagccaggcatgctggctcagactctacactctctaccatcacatcatgcggaataccggcctttaaatgcctagggtgcaaatagatcacagtatggcatagtttacctttggagctgtgaagtccagggtcaagtcgtgattcactttaaaataactacttgatattgtgggtttttgttgttgttgttgtttgttttttgtttttttgtttttgaggcgaagtctcactcttgttgcccaggctggagtgcaatggcacaatctcggctcactgcaatctccacctcccgggttcaagggattctcctgtctcagcctcccgagtagctgggattacaggcacatgccaccacactcggctaatttttgtatttttagtagagacggggtttcatcatattggtcaggctggtctcgaactcctgacctcaggtgatccgcctgcctcggcctcccaaagtgctgggattataagcatgagccaccatgcccggccgacgttatgtttaatagaacaaaaatatactacctggcatggtttaatatcttgaccaaatttacttcctaaattaatccctggctgtctggtatctttgattaattcagcaaaaaaggatgagattccagtatataagaggtttgccagaagaaaaatattaaattttgaaaaaccagattagaaaaacagttgatattatttatctagtattttattttacttcattttattttattttattttttgagatggagtctcgttctgtcttgcagtggtgcgatctcggctcactacaaccgggttttccgcctcccgggttcaagtgactctcctgcctcagcctccctagtagctgggattacaggaatgtaccactacacccagctaatttttgcatttttactagagacagggtttcacaatgttggccagtctggtctcaaactcctgacctcatgatccacctgcctcagcctcacaaagtgctgggattacaggcatgagccactgcacccagactatttatctactattttaaggcacattttatttttaataaaatttgaaaatatattaatatttaatttatttcaaatatttttcctaagcatactatatatcccatgtctattcttttcacttagtcaatttcccatgtcattacatactctttgttaatagtttttgttaactttttaaataaaagtattacatggtcattttataattgagagtacagaaaggtataagtgaaaagtaaagggctttgccctcaggataaaatagaattaacaaagtcctatttttcctatcaagaagtttacatgcacactcatgtagatagatacatacatacacatatgtgtacgttcatagcagtttgtattcctacccacatatacgaagggaagcatttgaaatgagccattcaaaggtacttagtttctactaggcaaggtctagacaagaaacagtcatgagaattctgggctttatgaagtgagatctgaatctcaggcatctatcgaggatgagatccacctccccgattgtgcttccttagagtaagacaaaatttctcaattagtttttcctcctattccagattttccattcagctaagcaggaggctttcatccactaaacaaatttctgatcctacccagtcttaacaagagtgattgttttctcctctgcaatttattcgaaagtctatcagttactttggtggtttcagaaagaagtgcttttgttggggatccagttggcctcattctgagctggctgtggattcacagaagaacttcacttcagcaaaggctcttagggccccagactaaaagaaagatgagcaagatgtgcgtcatctaagagtgggcgggggtaaggaattggggattaatgggggtgtccctagaggacccaaagaggagatttttagatgaaagtaactgatttgacccaactcagtatttgctggggaaactggagttgctacctaagattgctgccattgaacttcagccttaaaaactgctgataccatttgattggtacttagatttgcctgggcttagcgtctctgctgttcactcagctctcaaaccaagctgctgagaaagatgacctagttcttctcgagtctttctcctctctctctttctcccctgagcttgggggcaagaattagactaaatgaaaggaacagcaacctattctagaaccaaagttcaggacttggtacctttttaagggcatttcatgttcattctctcattccatcctgacagcgctcctgggcttttaattctcatttcacagaaagaaaagtgagggtcgtaaacttaagtgatttgtctgagatctgacaaactagtgccaccgtggaactaacactgaaacctgggtgtcctaactgtcccctctggccctggccagtggactgtggtcagcacactcactcttgtgttggtggctgcaacctgttctctattcactatatccctaaggtaggcccatacctagaagccaggaacagcgcttccttggtcatcatggtaaacaataacttcccttgcactccctagtcttactgacaggctgggcggtgtcaagagtaataaaactgcctccagcaaagccatgtttgcaactggcgccgcagcatcatgccagccaccctagcaaactcccaaaacctacccagcagccaaagccaagcgtaaagctcagtgaagctgacaagccccaggacactctggtggatgttttgcccccttgagtgtttgctgttttgtgaagaaaaagtgtaggagagttccagctggaactctgcagcaggcaaaagctctttttgttaattcaaaacagtttggaatccatttcagttcttcctaaacctccaagatacgggggaggaaattcactggattttacaatatatttttcaaggcaaattgccatcgccgtcctaatgacagagaagctgccgatatcactacaacggctgcagatggcaagtcatccagcctcctcctgtgccctcctgagtctggcttggtgacgcaaaccacctcaagggcagctccttctttttccagcagtcccagcactgttccttctctgtgaagccactttggatggctattcttggcatgatcatagacttttctgctgttgagtgggcaaggagaatagggtagaagcaggggaattgttctcattgctatgcatttatagatcaaagaggagcagatattttatgttaattggataacacctagttctatcaggtcagtaacctggggtccctggggctcccccatgggtcataccagatagatagattttttaaaaatttgttggaattaaagtagaatcaaagcctagtgctagcccagaataagttattcaataaatgcttgttgagtgaataatcatccgaaatgattataatactgcctaccattcgttgaaagcttcagtatgtgggagaatctgtcctgggcactttatatacacccataatctgtttggatttcttggtaggaagactacatggctgcatacatggtctttcattacctccattttatagatggagaaaatggaaatttaaagaggttaagcagtttgccgaaggtcacagagctactaatcggtgaggtcaaggttcagacccaggtctctcagactttaaaactggtgcacttaaccattagactctactttttgcgtattgagatgtggaagcatctcaattaattgcttcagtcttggagttctttggcggttatcaaaaactgcctatttccatattgcctccagatattgtaattaagaatttccaaggccaggcacagtggcttatatctgtaatcccaacattttgggaaaccaaagttggaagatcccttgagcccagaagtttgagaccagcctgggcaacatagggaggcttcgtctctacaaaaaatacaaaaattagccaggtgtggtggcgcatgcatgtgtagtccaagctactctggaggctgaggcaggaggattgcttaagcccaggaatttgaggctccagtgagctgtgattgtgccactgcactccagcctgggcaacagagtgagatcctgtctcaaaaaaaaaaaagaaaagaaaaagaaaaaagaatctccagattatagttcagatattacgtattaatatccaaagccctccttaagacaccattttaatctgtctaacaaaattaacaccctaggttttgacaattgcttgaggagggagtagggcctggtgatagagctaaagactagtgttctggttctttaatttgttcagccagcaggtacctggaaataatgatactgtttgcagaactccccacaaaaatggacagtcaactttaaataactaagtaaacacatctctgcttaatcagataccatcttatactaggttctaatatagataagtggttgcagaaacatttatgagaggaatgttaaattgtcgaaataccatccagtcccttcatttcttcctgcccttcagaaacaaacagaataaccctgatgcagctgtcacggcagaacactcattccagctcacagaacaccagtgttcaatcagcactgagtttgaggagcaccgggtttgggagtattcatctctcttccctgtcccatagctgcccttcaaactcttgagactgttcccagctctaaaatggaaagggtcgtcttctctctggaacaaaagacaggatgtacttgcttgaataggccagtgctttcctggaggaaactccggcttatggagcctctgcttctgcacagcccagtgccttccaccccctcttcatctgctagcatctacattactagttaactggagaggagaccaaacccaaaatattacaaaacatagatattaaaaatactgtgaacaacaatcagcccgctttctcccacctctcctgtaccagctaaatatttacctccctcatccgtagagggtttttggacaagacaggctggtgcctacatggtgaaagttgtttttctctcttttcttgtaatattgcaaataaacaaggatatctgctttctcttgagcaggacaacagccaaagagtattattgcatagttcctgtcccctatctgggctacgattgtgtctgagagtaatagcacttatttattaactggaagaaattttacaggcacctatgttgtttctgcctagcttctgaggctcctctcttaggccactcctctcaccatgcccccacagctagttggtaaaatggctttgaataatgactcgctttgagttatctagaaatgtcccattagttcactattttcataaccccatagacttggtttgacaagggtgaggtttatttatgagttctcaactggcacccaatttttaaaaaccacagagctgggggcggggatagaatgatggggaggagatgaaacatcctagaatcatctgatatactttaaacatatacatatacaggcttttgttgtttgtaaaatatagaagcatctttatctgtgactggttgagaaataaaatcccttccccagcccaggcatggtaggttgggatcatcccagtgagcgagcaagcaattggatttgccctcagagactgactatggggtcgaatgctgagtcattcctgtccccacaccagcagtcctggaagaggctcagtcgatcagtcagtgggctgctttctggagcactttatcattttgggctctcacaatgtgtgtttaaactctgagtgaccccaggcagccttccagactggcacagttcaccaagacgttcagacagatcatctgcaagtgaggaagaatgaaaacccagcactggttcaaatcactcctagcagaggtagtgaaggcatcactcagtcctggagaaatcttcggagggctcaccagcgtcacaagtaggtagaccagaagaggcaggctggcccattgcaaagccctggaatcagagggacaggagttccaaatccccactctctctcttcctagatgtgtgcttgcggcaattatttatctctctgagccttaatttccttatcaggaccctgagcaaaccaaataacatgatgtgaagcctctggcacaatgcctgggacatgttcagcacacaataaatgctaattttcccttttgccctatatgatatgatttgaagcagcgccactcaatcctgccagtcacattgctggaaatgccaccaacagtcattcttaagatcgcttattacaatataaatacccctaggggatatcagctaatcgctttatcaactatcgctttaaggggattaggtgtggattgagaacaaaacccagaaggaaagagatcctggaggaaggggagggcttggaagtggggaggattcggacgtcctgtggcccccgaaggagacggggaatggcaggtctgctggattcccagccttcaggaattcccatgaacttcttggggacttcatgtgtttagaccatatttagagagcgtgcatgtgttttgagatcacaggtttttctgagagacattgggaaggaggacagcgggggagtggagtggagagaagtcatacctggcagcttttaacttcacttactgcccttcatgtgctgttgccatttccagaagttgagggagtaaggaagggtaggagttcttggtattcccttatctcttcctttccctccccttatcctgcccagagcatcgcagggtagagccaccagacggattcagggcatggagatggcaggttagtggtagacttggaagggatcgttcttcctatttcagcaccacccttcttcctctacctcccaccctgcatttccagccccagtcagattccttcttggacagagaaggagcagaaatgtacgtacatcactaggcttaaggggggactgcagatggctcccctggtcctgacaaaaaggtggggagcaaaccgcatggctggacgtaaattgagtgacctggaatgactagaacgacagggggcctctaaccccagggtgcacagaggtagggacccaagggctttccggggtcggacagaaatctcgggttccattttcactctgagagacccagaaatgacagagcccatgctcagtttccactggtggtgctgttgagtggaggggctttgaacatggcacacaggaaagggttgcccaccctgtcaccagatgggcttgcactgttttgtggtttgggagttgcatattggaggtgggagtggagtgttgagatgaggcaagttgagatgtaatcgctatttaatgggcagatcaaagaaaacattggctggtttgatttttattatggttatcccgctccaccatcctggcccagtcatatatattattgttatcaggcatagataggggaagcaattaaatgtgaaactgttatttaaaccggcaagtgtcagaatgttaacccacttcagagcgaaggctctaccagcaacatgtgtgttcactgcagaatgtgctggttacagatgatctttatctgtttgtcaaagcatttgcaagatctctattttatgtcatcccctttccctgaaagcagcagcagtaaacgacattgctcctcaaatcatccatggctcagacctctgattaatttaagctggccctgctagcgttcttataaatccccagcaagatgcaggcccaggagtggaaaggggaaaacatccggaaagcagcggggggcagggagggggaaggctgaagggaaaagaaaaaggctgaagggagcaataattgaggaagaaagaataggaaaaatgtagaaaggacccaagaaaatcagagaaatgagaggaaaagaaaggtaagaagaaaatgaggcaagtttacagagcagtaatgaaaatagtaatagctaatttactgtacaacagacactgtgctaagcactttccgtatgtccctcagtcctctgaccaacactatgagttaaattaccgctacagctccaattgttaaagtgaggaaactgaagtatagaaaggttaaggaacttgcccaaggttacctagcttgcaaagttgtaattccaactgcgtctcactcagctggcagtgtgcacccttggccacacattagaatcacccaagagctattaaaaatcctgaaggtcataccccaggcaagtgaatcagaatctctagggatgggtcccaggcattcgtttgcttttttagtctccccagataattttaatatgtagccaaaattgataatccctgcactagaccaagctccgattcaaatttaaatattaagaggacacatgtataactttgctgaataattttttcctatcggtaggatggtgggaaggaattgctataggccacaccatgatactttgctgaattggatcagaaaagtaccctcaaattcccagcatcatttcttcaaggaagtagtgaattggggcaccgtgggttttttgttttgttattgttcttaaaccaccttgcctatgtgcactcaactacaggaacgagaacttgcctgaaaccccaaacgtgtctgcatcaggcattatttttcttgagctcctgaatttctgctctaaccaagaacaactcaaaaaccttcaatttcataactgttgatatgccacccactcctacttacactcctgaccccaaccccatggaaactgcctttattcgaacaaacacaagaattataatatgttctaaagttcctctacctatttcattctcaccatacgcttatgaaagaaatgtcattgtcttgaaaactaagcctaaacccagaaagattagatgatttgtccacaggcacacagaaactaagtggaagagaaacaggactcaagtcttcagtttcccaatccactgcctcctagaacatgcctgtgaactgagggagcagtgcaataaaggaaacttgagaggaagcgcaaaaggaacttaaattgcaaagtactgggttccctaagtagccctgcctcccctccagttttcagcccacacctgaactaaggtctttcattgtctgtggatcaatttacagctgccctgcaatcacatttgttttgtttgtttgtttgttttggttttgaacgccctagggacagtcggtttcagattagtctctatcagagagccaatagggaaagatgcagaaagccaggagagcagctcctagaatagagacaaaaccacctgcattttttctgcctcttcttcaggagactccactgtggtcatctggttccctaattgagacacctcctgagaaaatctgtgctagggaaacacccagcccaggtgggcagatctcaagaggaccagaacgtttgaatctgcttcagataaaccctccacttaacagtaccagatctggacgagatggaagctttgtagatttgcccataatccacagtgctctgtaatcaattccaggaccgtgtggatggagaccggaaaggtttctttcccaattcttcacagcctggggagtagggaactagtaattgactccagcctcctcgagtgctaagtcctggagtctgagtcacacacacttcccaaaagggccttctaagcagcctaccccagcagagaggacccttggccctggccctactccagactcaactgtaacctcagcaaggtgcagggagaggatctcaggtgggcctcctggcaacagtgccttttcaggcactaggctgttgggaatggagagagaagagaaaggccctggcactgtctgggtatttggatggatttctgtggcacaactgtggcacctggcttagctcagtaaaggactgagtgtgcgtggagcagaaaggggaaagagactagtggggagctggtgaccagttggagggtggagggtagggaacaaccctggcagatccattttgcccttgactagagcttaaagaccaaaggaagcgttcttgtttttaagtaagtcaggtgttctaagtttaatgttaatatgctttttcaattagatgtttgagtatatttccccagttgaattatgtagagggaattacaaatgagaatacatcttacaatttttgaattagaatctttgagattatctatttagtccggtctgttttgtttagacaagaaaattgagcccattgatcttaatcagtttgcccaaggtcacagagctggtgagtgaaagctttagatgtagaatatgcaacttttctctgctgccctccttccctaaaccatcttgccttaacggaggctcagtcagggggacaatgggactgatctgtaaatgatctaacttgtcattgcagaaaatatacttactccgtggccacaaaatttcagcccttcagccaagcttcatccatcacatcataaagaataacttgccaaagaggggtttgaaaatccaaaccagatccctctccactcctagagaggccagttccatgacactgcaccggagccaagtgtttccagcaagccctgatcagttgcatgttagtccttgtttgtatgataaacgggacctagtagattaacatcatctcctcagcccaagggctgaccccagtggtgctgctcaagaggaaggaggaggctgggagcggtggctcccgcttgtcatcccagcactttgaggggctgaggagggcaaatctcttgagcccagcattcaagagcagcctggaaaatgtagagattctgtctctaccaaaaaaaaaaaaaaaaaaaaagaagcaggtggaggaaggaggagctgcagaaaggctccgggagccagtgtctagtaagcagaggcctagcccaggctgagtggtcccagcccaggagggcctctctcggcacctgtctttagatgcacgtctgcctctggcctttgagatgcacacattcatcaagcaggaggatctgtagtcattaaaccaaggtgccagtctggggcccagccttcaatgaagcatcagtggctttttcattcccatagagcaactgcgaggaagaccccatgctcccaggggcccaattctccaggccaggaccagatcatttgttccttctcagaatctgagatcttttgctgatggttccaaaattgaatgctgaaatctaaaaagaaaattatacttactctggtcaatgggatatgttggagacctgagagtgaaaaaatcaaactgaagaccagtttccttctttaatggaagaaaagtaagaaagttaacttagaagaatatttagagttctgaaaatcccactctgagaccactctgccatctcacattgttctcttgagccttatctcaatggataaaggtcactagagtttattagacacaggaaaacatcgtttacagacttcttcattgtgtagggattctgccttcaggcctgttcaccagccaggagaattgtgctttttgatccagggctcttggccagaacagccttctcgctcattaactcagtcaaccaaatgcttcctgtgtttccactggtggttcagaacgtagtctctgccctctgatacctcacggtctaacgggggagaaacaagaaagtcagaacttacaatacaagataataaggaataagagcagagcctttggaaccagacagacttagatccaaatgtcagctctgcatttactagcaagtggtctatcttggtaagatgcaactcttttctctaagtcttaatttctccctctgtaaagtatgacctcatggggttattgtgaagattaaatggcatatcgtatataaggcccttggcacgatctttggaaaataataaccattcagtgaatgtaatctactagtatagtagcgattgaggtgtttatagggtgctatgagggcccagagaaaggagatgagatgaacaggagagaggaagggatactgttgaggaaggatttcaggatggaatcccaccacaggctgtattttgaagaaatggttgaagtcaataagaagagtgaagggaaggtgagaatgtttcaggcaggaagaataaaatctgaataagtgcagagtcctgacagaacatagggtgtttgaggaacttggtatggaaatagattaatatggcttaacatggagcaaaatgaaacagaactgagactagaggatgaccaggaccaggtcatgaaggacattaaaggtgggtaaaactcataccatgaaggagagaagagttacagtttttaaaagaaaataaatagccttcaggagctcaaatttgaaatcaatacgagatagatgatcaagtgaatgttctgtcttcttgatgagcctgactgagaaaaccttataatctaacccaaggcaacaaactgagaaattagaaaataaaacttagagattagaaaacaaaaactgaaacccaaactttttacttggaaatttaaaaacatgcttctatataactcttaggttaaaaatgaaatcaaaattgtaagacaagctgtttagaaaagaactatgagagtaccgtttatcaaagcccatgggatgaaaccaaagtggcactaagaagaaaatgtggaggcttcaatgcatttattagaatacaaagaataccaaatcaaatgaattaagcattcaactcaagaaaactagaaaaagagaagaaagtaaacctaaagaaagttttaaaaaggaattcaataaattaaaaacaaaacacacaatttttaaaaacagacttgattaataaaaccaaaagctatgtcttttttaaaaaagcaaaaaatagataaacctttgacaaatcagatttttttaatgagagaatgcacaaataaagagctctaagagctgggcatggtgactcatgcctatattcccagcactttgggaggctgaggcaggaggattggttgaggccaggagtttaagaccagcctgggctacgtagcaagaccccacctctacaaaaaatttcgaaattagttgggtgtggcggctcatgcctatagtcccagctacttgggaggctaaagcaggaggatcacctgaacccaggagttggaggctgcagtgagccatgatcatgccactgcactgggcaacagattaagactctgtcacaaaaaaaaaagcgttaagttaaggagcgtgaataaaaataatacatttcatattctaagacaatatcatctacaactttattccaatataaattactaaaactttaccatataagtagtagaaagcctgaatagataaaagctatggaagaaaaattaaagatagtgatagatttatcaccaagctcagatggttctgcaaacaaattctaagaaaccgtcagggaacagatcattactgcgttgacacagaaggcaaacctctcaacttggtgtataaggctagttaacattgttactaaaacaggcaaagaatggcccccaaaataactctaaaccagtatcacctacaaatacaaaaactctatataaaatattagcaagtgagtacaagagtataaggaactctattaatgtaattcactgtgctaggttaagataaaaccataactcttcagatatcatagctctttagatagaaaaatgtaaaagtatttgataaaaattcaacaacattcctgactaaaacaaattaaaattgtaaaacccctggaaagccaggagtaaaacatgttctccataatctatagcaaacatcatagttgctgataaaccattagaagcatttccttcaaagttaggaataaaacaagaattgctactattgctactattatttaatcccatattggaggttttagccaacacactatggcaagaagaggaaatgagaaaggttaatgctgcaaaagaagaaataaataaagctaccattgctgatataattatctatccaaaaaaagtagaaatccatgattaaaagctatttgaactgataaaagttcagaaaattggaattgtttcaatatactaaaatcaattagttttctgcacatcagtggggatatattagatgtaatgaaaatgaagggtccattcacaatagaagctataaagtatttgagaataaatctaacaagatctacataaagaaaaaagtaaactgcttaaaagtaatttaagttcaccatttgcaaattgaactaaagaaaaatctaaagaagaactagtgaaaattgcttgcaatcttaccactgaaaagaaacctctgttaaaatgtatcagtattttaaaactgtgttttgtcctttggaaatatgccatattaagcaaacatagcataagcaaattaagcaaaaagacaaacctttttttaaaagcatatttgtgaaaaaagcatatttgaagctatgataagaaactgagtagaagctataaaacgtagattcatcaggaaggagcagaagttgaaaaggagatttctggctgggcacagtggctcatgcctgtaatcccagcactttgggaggccaaggcgggtggatcacttgaggtcaggagtttgagacaagcctggccaacatggtgaaaccctatctctactaaaaatacaaaaattagaaaggcgtggtggtgagcacctatcatcccagctactcaggaggctgaggcaggagaatagcttgatcccgggaggcataggttgtagtgagccgacgtcatgccactgcactccagcctggatgacagagcaagactccatctcaaaaaaaaaaaaaaagaaaagaaaagaaaaggagatttctgagtagaaacacgttagcagaggaaacttcatttggggaacaggattgttttgatgccatgctccctcagctgaccacacagattacatcctggccagaaaaaggccctgcaaagagctctgatcattttggttctgattccatccttagttcttacccattatctgcttacccttcctccccacctatttaagatacaaagcaaaacaaaccaaaaatacagatgagttgcttggtagagctgcctaggcctccaaggcggggcccagaggattttttgactgaaccccctgttgtgttttccatatgtgcttccccagaccgcccaggtttgctgaatgtgaagcccattgaagacattcaagacaacctgctacaagccctggagctccagctgaagctgaaccaccctgagtcctcacagctgtttgccaagctgctccagaaaatgacagacctcagacagattgtcacggaacacgtgcagctactgcaggtgatcaagaagacggagacagacatgagtcttcacccgctcctgcaggagatctacaaggacttgtactagcagagagtcctgagccactgccaacatttcccttcttccagttgcactattctgagggaaaatctgacacctaagaaatttactgtgaaaaagcattttaaaaagaaaaggttttagaatatgatctattttatgcatattgtttataaagacacatttacaatttacttttaatattaaaaattaccatattatgaaattgctgatagta

SEQ ID NO: 5 - DNA, 15051 bp, Homo sapiens
attgcccaagcctcccaggagtgacaggaattgtttctgcctgaggagacactctgcagcctgggctctgtgagactgaggtggcggtcagccggagtgagtgttggggtcctggggcacctgccttacatggcttgtttatgaacattaaagggaagaagttgaaggtaagggcctcttggggattttgctggggatgaaaaactctgcagggaaactactgagggagagattctggtatagataccagaatctagaaatagaggaattggataggcacagtggatcatgcctttaatcccagcactgcaggtggccaaggcaggtggatcacttgagcccaggagttcaagaacaacctgggcaacacagtgggatcctgtctctataaaaaaagagaaaaaattagccctgcattgtggtgcatgcctgtagtcccagctacatgagaggctgaggtgggagggtaccttgatcccaggaggtcaaggctgcattgagccatgatcgtgccaccacattccatcctgggcaacagattgagaccctgtctcaaaaaacagatagggagagggggagagggacagacagagagaaagagagaaatagaggaattaacccagagccttttcaaagatgaaacctttaacttgtttatttatttttttagagatgggcatctcactgtattgccgaggctgatctcaaactcctgggctcaagcaattctcccatgtcagcctcagtagctgggattacaggtgcttgccagtgcacccagcttgaaacttcttgttttagtctcttttggaccctcatttaagagccttaagattttatctaaaactttttatctcttatggctacctgcattttttatttcctaacctgaactcctttttttttttgagacagagtctccctctgtcacccaggctggagtgcagtggcaggatctcagctcactgcaacctccacctctcaggctcaagcgatcctcctgcctcagcctcccaagtagctgggattacagctgcccaccaccacgtctggctaatttttgtatttttagcagagacggagtttcaccatgttgcccaggctggtcttgaactcctgaactcaggtgatctgcctgccttggactcccaaagtgccgggattacaggcctgagccaccgcgcctggcctcttaacctgaactcttataggaaacattattcttggccgggcatggtggctcttgcctgtaatcacagcactttgggaggccaaggtgggcagatcacttgaggccaggagttcgagaccagcctggccaacatgatgaaaccccatctctactaaaaatacaaaaattagccagtgtggtggttcatgcctgtaatcccagcactttgggaggtctaggcaggtggatcacttgaggtcaggagttcgacaccagcctagccaacatggtgaaaccccatctctactaaacagaaaaaaaaaaaaaaactagccgggcgtggtggtgggcgcctgtaatcccagctacttgggaggctgaggcaggagaatcccttgaacccgggaggcggaggttgtagtgagccgagattgtgcaactacactccagcctgggcaacagagcaagaatccatctcaaaaaaaaaaaaaaagaaagaaagaaaagaaaaagaaagaaacatcagtcttgacaagtaagctatggagcaactcattgctatgggaccctaaggagtggggctattgtgtgtgatattcaccctccaacctaatgccttctgttcagggcaaatgggcccccaggcagcctgggcctagttggtggccatgagaggtagggaagtgacccagtggagctcaagcctgagggcttctgatgggacctgggactctgttttgcagcttgaggagcgaggatggcagtcaacaaaggcctcaccttgctggatggagacctccctgtaagtaacttgggctcatctgtgacaggggatggacaactgagggaggaggaagaagcagggaggggagatgcaggggacttagagcaagatattcccagattataatcctagttcattgactaccatggcttagttattcttgccctcaccaccttttgtagggggcagttggaggatctgggaggggaaaaaaagatgcctattaaaaattaagcaccttgaaaaaaagggacagaaatcctggcttagggacggggggagtggagaggaaggaaactactgtttaaaccctgacgcagaagcccatgctctgtccaccacccagctggatgtgcattcagtgcagagccactgtattctgtgtctccagcatcattcataacggcaaatgtcacctgtcggagttagacggtgcacagcctgtgctgctgtgtggggggaagggtgggatattccatgtgtctggtgttctatctctgacctggttttcactgtgacttgaagtgatttgatacttttagggatgcttgatggctcctggcaaggcatctcagatttgggcacagttcggatagtgaaacttaaaaattatgaacctgtgcaatatccaccttgcccaagttacgcttttactaactcctgcactaggaatcactttgttttgtattgaatcatagaacagagagaggcgtaaaatggtcaagtacatggttaagccattctaacagacggtggtttattcccttcccaggatctctttcctccttcggttttgtgtaaaacaacctctgaagcctcgtccaaaatgttaaaaagaatgttttcttgtttatagtcatagtctatttctagttcaaatttcaaattcacctgcaggataaaaaactaagatattggccaggtgcagtggctcacgcctgtaatcccagcactttgggaggccaaggcgggcggttcacaaggtcaggagatcaagaccatcctggctaacacagtgaaaccccatctctactaaaaatacaaaaaatcagcggggtatggtggcgggcgcctgtagtcccagctacttgggagactgaggcaggagaatggcatgaacccaggaggcagagcttgcagtgagctgagatcgcgccactgcactccagcctgggagacagcgagattccatctcaaaaaaacaaacaaacaaacaacaaaaaaaaactaagatattgtgggctgctctgtcttagtgtacatgtaggtgtcattgatgggggtctttgaactctgcctcctactaacagaacaggtcaggtcttcactttgcgagcaaaaccatgccacagtgtcatattcaaagctacagagagcctgagcatcggaatctgacctgggtttttttttctttttacttttgatatggagaattctcaacattcataagaatagacagaaaagaacataggaagccccacgtaccctttattagccccgaagaccatgaattcgtggccaatcccgcctcgtcctcacttccattaattcccccacttcatgttattttgaaggtaatcccaggtaggccatttccaacttgtgttggaatcctggctctgccacttatcagctgtgggatcttgggtaaatcacttcctgtttctgattgtcaatctcctcatcgactcagtgttggtgacagtgctgaccccagaggatggctgttagaattaagtgagctcatgtcaccaaggcacccagaccagtgcctgcagcataatggcacctggcaaatgttctttccctctgcatgcctagaatttgttcttttaattttatttatttattttttagatatagagtcttgctctgtcaccccggctggagtgcagtggcattgtcatagttcactgcagcctcggactcctggtctcagacagtcctctcacctcagcctccccagcagctgggactgtgtagtccacatgccatcaaatattagcatggccagctttttttttttttttttttttttttgtggctcagagtggtctcaaactcctggcttcaaatgatcctcccgcctcagcctcccaaagtgttgggattacaggcatgagcttctacacttagccctagaattcattcttttactcaaagaccagtgttttttcactaaggttcctaggtgacctaaaacttaattatgtgactgctgccaaaggtgcattactaacagcacagcaccccggaaatgataaacgattgttccactacacccagcacgtgcagatcgcacccagagtgctgtctttgagtgttgtttgtttgttttttgttttttgagacagggtgtcattctgtcatcgaggctgtggtacagtggcgtgattatggctcactacagcctccaactcccggactcatgcgatcttcccgcctcaggttcccgagtagccgggaccacaggcacacagcaccatgccctgctaattatgttattattatttattatttttttttgtagagacggggtctccctgtgttgcccaggctggccttaaactcctgggctcaagcgagggtagtatatttttaagaaagataatgtaaaactgtagcatgtccaggagagggggtcaccagaccacagtgtgaaccatttgtaaacgaagttaggaactggagccatttaacatgatggcggcacttctaaagggagacatatttgacttcaaatatgtgaagggccaacccatggatgaggaaataggtttgctcagttttgttccagagggcagagtgtggaacaggggggtggaggcaaatttcagctcagcaaagctgtccaacaagggacacaggccactttgtgagggggagagctctcattcagggagacattcaaatataagtcttggctgggcgcaatggcttaggcctataatctcagtgatttgggaggctgaggcagaaggatcacttgagtccaggagttcaagaccggcaagacccctcctctacaaaataaaaatattagcaaggcatggtgtcacaggcctgtagtcccagctacccaggaggcacaagtgtggggactgtttgagcccacgagttggaggttacagtgaactatgatggcactaatgcacttcagtttgagcaacagagtgaaaccctgtctctataaaaaataataaataaataaaaacaaatacaagtcgaccagaggaaggatggaaaggattcttactacaaagtcccctaagattctgtgattctgtgcagttgggcagatctaaacctacacattctactaggcacagccctgactgtggttcacgtattcagctaatattactgtgccatgtttggcctacactctggggatgcggagatgacagtctacggggagtgagatgaacacacataaccacagcacggtgtgacgaatgtggagagagccagggagggagtggtttccgacgtgatggaggagagtctggaagcttcacagggggagtgctggtcaagcagagtcttgaaggaatggcattctgccaggcagcaagggctgtgggagggaaggtgagcgttctaggcagtggggtcgggatacacaggcagggaggcagtaccccagaaagaccagaaaaatgtttggagaagccaccaatcaaaggtggactaaaactctagacattaacaacacttgctctctaccagtaggtatcaggggagtgagctgaggtgggggtgggagtgatgggatctgacttggtccctggagaacagagattgccttggcagctctccagcaccccggatggtgaccaggaggacatggccggctgactcttgttttccccatgggctctcccacgctcccatgattggggcatctagagacctctttggccttggttcctcccaggtcgcttggactcaggctagttgcaccattggtccccatgaccccggggtcagcttcccgggaggctggagagggaggtgggacaacttgaatgtgttccttccgcaccaggagcaggagaatgtgctgcagcgggtcctgcagctgccggtggtgagtggcacctgcgaatgcttccagaagacctacaccagcactaaggaagcccaccccctggtggcctctgtgtgcaatgcctatgagaagggcgtgcagagcgccagtagcttggctgcctggagcatggagccggtggtccgcaggctgtccacccagtgtgagtccttgcggcgctcagcagctgcctgcttgttcactgcctaccttccagtctgtctgtccgcctctcagtctgtctgactgtctgataacttgcttgtctgtctgcttaactgcccacatcccattgcatggtcttcttatctatggctataaaggtatacatacagatctcttttcttctatagatgcctatctatggcccagctggcattttcctaggataacaatgatggccagagaatctgcaggggaagggaaggcagagctgtgcaggcagtggtgagggtagggcccatggcagaccagacactcactgtcgcagcacaggcagcagcagcagcaagaaccagcagagtctggcagaggcagaggcagccggcactagggggcctggcagggccggagggcacggagggaggggaaagctggctgagcccagcagctgatggtagcggctcatgacgggtgggggtggcagagagcctccagccagggccctagaggcagaagatggctctcctactggccttaccagatgtgcaccactcaaattgttctgccccatctgaaaatgtggtctccaaggcatacacagggctgagggaaatcgcagcaatgccaaaaagtctggtaccttccaccatcctaattacagaattatgggtagctgaggactccacacagcaaacaggagaggaccgatgtgaagtaggcccaggccctgtccctggctacaatctccttaaatagtttccttgagtctcattctgcccaactggaagacagccaagtcatctgggctctctgctgcttgagaattttgtgaagaaaaataaggtatctggcaaaagaatatatgaaagagtatgaagaactctccttgaaagctgtggcccccattggccatggctgcagagccgatgtcccggccaatccaggcgggatccccttgaagcaggtgcatctctgatgcatgcccactctccgggggcaactcttttcccttccctgtgaccctcttcggacagttgaccatctcaacacctagtggttaaaaagaagagcatggacggcctggggcctgcactggctgtgctgggagtttgtcatgttgatagctaagcaggcccagccccaaggcctcactgccatctgcttccctctcacacctctcttctccctcctcatgctcactcagagcccccttgcaggtcaggaaggaagagaaggagggaaagaacggtacttgttggtgattcatatagctcccatcattcttcttcacaaaaatcctgccaggcaggcaccattaattcaccttttacaaggtaggaagcagaggctcagacaggctgtgactagctgaggccacaggcttcttagatgtggtcacttggccttttgagtgcaggtctgagcgactccagagttcccacacagacagaagaaactggcccgggtcttgaggagtccctgcagacaagggcgaaagagggcaaagggcctggggcccagagatgctggggaggcagcaggccttgggatttgagggctccaagtcacttccttggcacagaagccactgctagattctcatggggaggggcagggctcacccaaacttggccctttccttccagtcacagctgccaatgagctggcctgccgaggcttggaccacctggaggaaaagatccccgccctccagtacccccctgaaaaggtgagcccttcccacctctcgaagcctccctgcactctgtcgtcaccgcctctcagggaggagagggcaggggctgctgatgccgtgactggaagtctgtgtcccatggtgagcaccctccaaggctggcggagagtgcctgctcccaaggccacacaacaggctcctcttctccctgtgaagctaaacggaggtggggtttctgggaccagcaggcacgagcccagaggcaaacccaaacaggagcttgaggcgggtcccactgcctcccgataccgcaccctgggatctgaggttccatagtctgccctggaccacttggcaactacagctgcctaagatcatgagtcacactcattcagatcccaggagtgtcatcccatatcctcaagatccaaccttttatggaccatgagattgaaggtcactttacctggctacatatttattgggtagttgctatgataaggactcatttttggtgcttctggggaataagcttcagtctctgacctcatctatctcactgaccaactatctacccttcccacaaacaggtactgagccctaccctatgcccggggccatcacctatgctttatcaagcgctcttctttaatccacttgtcaaccctctgggataggggttgttatctccattttacagagaggaaaccagtgtctcccccacggccacacagccttaagtggggacaatgagagcaggtctattcagctgtgagacctgtgctctttgctccccatttttccagataaaaggcaacatttttgtcacaaaagagctcccagaataatgaagaaaaagacaaattatttcagtgcagaacttgtaggaagatcaagcacacctataaatcaacagaaatgctgtgttctgaggaaaggatggaggagattccttgtgagcccaagggtatcaggacaggtggcaccaaggacgagcctttggggtcatcctcacaggatgggagggatcttgacaggctgtgatgcggggtgaagaagccaggagatccaaacagttggcacagggaaggttggagccctctcctctaaagcccagcttaattccaggtacctaaatatcagtaccagttccctgggcctttaggtgatgccttcctgggagctgggctcaggccaccagaccccggaagggcagcccttgtagggcctgcaagaccacatgcctaacacccccaccaacttcccccagattgcttctgagctgaaggacaccatctccacccgcctccgcagtgccagaaacagcatcagcgttcccatcgcgagcacttcagacaaggtcctgggggccgctttggccgggtgcgagcttgcctggggggtggccagagacactgcggaatttgctgccaacactcgagctggccgactggcttctggaggggccgacttggccttgggcagcattgagaaggtggtggagtacctcctccctccagacaaggaagagtcaggtacctgccattcggaggctcggcctgggagtgagttgtcacacacactgcctgggaatcagcaggaggtggctcaaccagccactgctctggaatagggaaggccagtgggattttcagatggattcataatcatttaccccccccacccccaccccatctgtaaagagggaaagtgagttgtggaactcaccagtgagtttgtggtggacctagaacacctactttccagtcctagacatgacccagatctctccggcccctctctgacctgttttctcttctttccccccaaatccctacccagcccctgctcctggacaccagcaagcccagaagtctcccaaggccaagccaagcctcttgagcagggttggggctctgaccaacaccctctctcgatacaccgtgcagaccatggcccgggccctggagcagggccacaccgtggccatgtggatcccaggcgtggtgcccctggtaagtatctgccgtgagctctgactgacccggtgcctgggagccctgtgggcctcctagtccctcccatcccccacccatcaccccttcctgtcctgagaacattctctcttccaacgtgggctggggcttcagttctaagaggacttgagaggggtagagggatggccttccagaccacagggaaagagaagggagaggatgttaagcaagggcaggcctgccttgctagagtgctccttccccaccactcacttctatctcccccatttccctctgagtctcccacaagccccagccttcatacccccaagtggacctctttctatgggtcttcccacctataagacagcaggacctccatctgctatgcaagcactagggactgtcatccttaaataaacaaagtatacaacccattttgtcagggagagtgtacccagcaccaatgcacccaaggtgaagttgccctttctaggcccagggagagttaactccccaccactccccaccactccccacctccacctctctgtatcttagcagaaataggctggatgtttttcataaggaattttagtaagaaactaaggccagaaaagaaaataaaacaatactaaagcataaaggccagcaaacaagatgactacttttcagccatcttttgagcagtacttttaaaagcatcatcaggcgtggatcattcattcaagcaagtgttgcccctgcaggaatacaggagtagtagctcccctctgtcccctgcatcaataacagctgagactgagtcacatgcctggaccccgcctgcagtgggcagaacctcacgtgccaccattcgcatggaatagagtggcatccccaaaagctgtgtaataaggagtctctgtttgtggggctccctagaggtttagaatgtgcctcaggagataccagcccccttgctcagtcaatttcttcccatgggacactctggttcattgaatttttcactcttaaaagatctgggaggagtatctaccaaagctaacatacatatatcctatggcccagcagttccactcttaagtgtatacccaacagaaatgagcacttaagtctcccaagacaagtgaaaaatgctcagatcagcaatatccttagctaagaactggaagcgatgataatgtccatcaacggtagaatgggtaaattacaggaaattcgtatgatggaatattacacagaaatgaaaaagaaggaactacttccatttgcagcaacatgggtgaaacttacaccatcatatttaactaaaggagctattaatagatgaatacacatggtgtgactccacttacatgaagttcgaaaataggcaaaactaaggcatggtgacagagagcaggactgtggtcactcttgggggaatgtggactgagtggggacacagggagtctttgggtgttgaaaatggtctatgtcttgatctgtggtggttacacaggtgtgttcttataaaaattcatagagcttacactaaagatgtgtgcactttagtgtatgtagattaccaactcaataaaaagtttccagaaaagtctgctggggctgagccctgctcaccacctggcccggaccctgggggcgaggggcttgggctgctggggctccctggccttcagcaggggcagttgagccagctatctgctgccatcctccctcagagcagcctggcccagtggggtgcctcagtggccatgcaggcggtgtcccggcggaggagcgaagtgcgggtaccctggctgcacagcctcgcagccgcccaggaggaggatcatgaggaccagacagacacggagggagaggacacggaggaggaggaagaattggagactgaggagaacaagttcagtgaggtgagggggagagtgggagcctcaaggtcccccagcccacagaaggggtgagaagcagaggcatagggtgaactcagggcctctgccccagatgcaggggcacggcatgtgcgtgcaacacccttcaccccacccccaaatgcccagctggcgagggacttccatgtcattctctcagctgacccttgcaacactctgtaaataggcagggcagagattattgtcccattttgcaggagaagaaacagaggttcagagagggaatgtgacctgtccaaggccacactgctagtggcagaataggcctgaagttttgtaaatttggtattctcatgctttcccctctagccctggggctggtggggagggaagggtcaggggagttaccacaggaggcactgaccctgctttggcccccaggtagcagccctgccaggccctcgaggcctcctgggtggtgtggcacataccctgcagaagaccctccagaccaccatctcggctgtgacatgggcacctgcagctgtgctgggcatggcagggagggtgctgcacctcacaccagcccctgctgtctcctcaaccaaggggagggccatgtccctatcagatgccctgaagggcgttactgacaacgtggtggacacagtggtgcattacgtgccggtgagtaccacccctggcaaactgttagtgtcccaagggggcctggacatggcagataaagtagatttgactgaaggggctgcagtccccctcctttcccctactcctctggagaccgccccccactccagtcctcagtgccctggcaacattttaacatactgggcctctcccagccccaggagtatagaggcccatggctctggcctgaggcctctccccacggcccctcccacctgctggtagaggagtctccagccatcagtcccaggatcccagtggctcaccttcacccctttgctctcaactgaagggctgggcgggtttgcatgctgctgcctgggaaggggttggagaggcttccagggcaccctgaggggtgggccatgagttccaggggacagcagcaggctccacccaattctatcacctgtcaccaataggaaaaacccaggggataccaatgccatccctttgggagcccctttccaaagcaggttacagattatgcaggcctggggggcggggctggtcagggcagaaagcacccttagagctggtaagggggtgggttactcagtcactctaccaagcagcatgccagggatcttagcagcccgcctgttcatcctgcgctcgggccagccccaggaaggtgtaccggtccctggccaggtcactgtgtgggctgcaaggaagagttaggaaagctgatgacctcccattgagggttcccctcaggaaggcctaggggatcctgaaactttgggaggcttggcttgcctgagcagcctggtccatggagaagctcagagtgggcaggacctcagggctttccccggtgccccccaaactactgaaccgctccccagcctgtgtcctcctgacggccgctcccgggggaacaatcgaggggcccgggaaggggcggtgggtcagaggcgcagggcccagggccaagccaggactctaaggcggctgccgggccctcagctccccaggctgtcgctgatggagcccgagagcgaattccgggacatcgacaacccaccagccgaggtcgagcgccgggaggcggagcgcagagcgtctggggcgccgtccgccggcccggagcccgccccgcgtctcgcacagccccgccgcagcctgcgcagcgcgcagagccccggcgcgccccccggcccgggcctggaggacgaagtcgccacgcccgcagcgccgcgcccgggcttcccggccgtgccccgcgagaagccaaagcgcagggtcagcgacagcttcttccggcccagcgtcatggagcccatcctgggccgcacgcattacagccagctgcgcaagaagagctgagtcgccgcaccagccgccgcgccccgggccggcgggtttctctaacaaataaacagaacccgcactgcccaggcgagcgttgccactttcaaagtggtcccctggggagctcagcctcatcctgatgatgctgccaaggcgcactttttatttttattttatttttattttttttttagcatccttttggggcttcactctcagagccagtttttaagggacaccagagccgcagcctgctctgattctatggcttggttgttactataagagtaattgcctaacttgatttttcatctctttaaccaaacttgtggccaaaagatatttgaccgtttccaaaattcagattctgcctctgcggataaatatttgccacgaatgagtaactcctgtcaccactctgaaggtccagacagaaggttttgacacattcttagcactgaactcctctgtgatctaggatgatctgttccccctctgatgaacatcctctgatgatctaggctcccagcaggctactttgaagggaacaatcagatgcaaaagctcttgggtgtttatttaaaatactagtgtcactttctgagtacccgccgcttcacaggctgagtccaggcctgtgtgctttgtagagccagctgcttgctcacagccacatttccatttgcatcattactgccttcacctgcatagtcactcttttgatgctggggaaccaaaatggtgatgatatatagactttatgtatagccacagttcatccccaaccctagtcttcgaaatgttaatatttgataaatctagaaaatgcattcatacaattacagaattcaaatattgcaaaaggatgtgtgtctttctccccgagctcccctgttccccttcattgaaaaccaccacggtgccatctcttgtgtatgcagggctatgcacctgcaggcacgtgtgtatgcactccccgcttgtgtttacacaagctgtggggtgttacgcatgcctgcttttttcacttaataatacagcttggagagatttttgtatcacattataaatcccactcgctctttttgatggccacataataactactgcataatatggatacgccttatttgatttaactagttccctaatgatggacttttaagttgtttcctttttttttcttttttgctactgcaaacgatgctataataaatgtccttatcaaaaa

SEQ ID NO: 6 - DNA, 13545 bp, Homo sapiens
ggcagtatttccaggaggctgtgagggggaggtaaggagtttgttattgttttgggtttggggatttttttttctttgctcctggagagcaggttaggtagctcttggcagagttttgctagggatacgaagtgggtgaggctgaggcaagcagaatcctcctgcctcagcctcccacgtagctaggactagagcttctgcagtgctaagtgggaaaggggctttctgcctctgtggggcctggtctggaaagtcccccaaattgttaaaagcaaagagtgattgtccccagctggcaaggggacttttgttttatgtccaccccttgtgccccttgctctgcgaagccctacctgtgccgggagcttcgaataggaagaggacctcagggccaggcatccagggactgaggggctacagcagaaacagacccagaggcacccttggctaggaagagcaggatgaaggtgtaataattttgggagtggtctaagaagactttccttttaaagagtggaaagcaatgaaactgaggcttcaaggctgcaaaaatttcgtcctggatcagagaagggcactacaggtggagggaactatctgtgcaaagggttgggcccttcaagagttggagcacagaggtttaggagaaaaagtggtgacagatgagcccagggctatctgcttagacctgtgatctaatatgggtgccaacagccacatggcaatgtggctatttaaatttaaattagttaaaattcaataaagtaaaaaatacagctcctgtgccaggtgcagtggctcacgcctgtaatccccgcactttgggaggccaagacagggagatcacctgaggtcaggagttcaagaccagcctagccaacatggtgaaacccagtctctactaaaaatccaaacaaaacgaaactaaactaaaaattagctgggcgtggtggcaggtgcctgtaatcccagctacgggggaggctgaggcaggataattgcttgaacccaggaggcggaggttgtagtgagcaagatcgccccattgcactccagcctgggtgacaagagcgaaactcaaaaaaaaaaaaaattcagctcctgaatcacactagttatgtttcaaatactcaatagccacatggggctagtagcaatggtattggagagtatgtacaaggcatgcctctatcatcacagaatgttcttttggccactgtgaagcctcaggaaggggctcggattgctcaaggacccatgggagagaggaggctttgactgggctgcctgcctgtgaggtctctggactagaggtgagggaacggtccccagaagtgtgaggtggtgtgactcacctcctgccagcgtaaagtggactctgtccccatggacaggaacaaatcgggcaaggcttcttcagccctgagttgggttatctgctcagtgtgaccagagccaggggatgagaaacatggagtgagtgggactggggcaggagcagcagctgctgagtgaggctgagcctgagcacccggtggggagaggggtggggtctccagcttggtcttggttggcatgcatggggccggacaggagaagggctgggggaggtatgtgtgagttgggatagaagaatagggaggaaggttgctgaacaggggttgggttgcaccttgcctaacaatgagagagatgggaaccctctcatctcaccctgatgaattgtttctgcctccttcccaggtccaacgcagtccagctgacaaggatggaatacgccatgaagtcccttagccttctctaccccaagtccctctccaggtgaggaaagtcatgggcagaatggaaagagagccacctatgagctgtaagccagaacttctcaaactttaatgggtatacaggtcacaggggaccttgttcaagtgcagattctgattcaggaggtctggagtgggacccgagattttgcactgccaagaagtccctgggtgatattaatgctgctgcttacaccaccaccacaccatgcataaaaaggagtagacactatgcttggtgcttttactcctgatctcatgtaagcctcacaacaactttgggaagtgggggttattataccaaattaataggtgagagaactgaggcacatagtgggtgctctcaatcacccagcaaatccatgagggagccaagattctaaattgggactgcctgactgtaaaatccaggtcctttcctttcctttggttctaaattgagtgcttcagttaaaaaaataccttacattgacttcaggcttcccccacttcatccaactttacaggtgaggagagtgaggccaagacaggtggtaaatggggaatgtggggtgggtcttttattactttgctacttactcctcatggtctgagaaccagcgttatcaacatcacttgggaacttgtgaaacatttggaatctcaggcctcacccaggtctactcagtcagaatctgcatttgaacagaagccctggtgacttatgtgtccttcgaagtttgagatgcactgctctagcaggctttttttaatttttattttattatttttttttaagtcgaggtctccctctgttgcccaggctggagtgcagtggcaccatcacagctcactgcaacctctgccgcctcccagggttcaagcaattcttgtgtcccggcctatggagctgggattacaggcatgtgccaccacactggctaatctttgtatttttagtagagatggtgtttcaccatgttagccagggtggtcttgaactcctgacttcaagtgatctgcccacctcagccttccaaagtgtgggattacagacatgagccaccgtgcctggcttctagcaggcttctgagtaagactagggcaggattagcaggtgatggggggttgactgatcatgagcccagtcaggaggggtggggaggtggctcccctgtgtggtgggcaagggttgggcaaagtgggtggctcaccctgatgcttcaactgccccaggcatgtgtcagtgcgtacctctgtggtgacccagcagctgctgtcggagcccagccccaaggcccccagggcccggccctgccgcgtaagcacggcggatcgaagcgtgaggaagggcatcatggcttacagtcttgaggacctcctcctcaaggtgagagcacctgcgcgttgcacagcccagctgtgagctgggattcagcagagatggaggtcagggcttagaaggtggcacagtttcctttctgatacctaccagcgcctacagatggtaatgcccacctggagagctgtccaactaattagcaatgactgatgtctgtttggtaacaggaggggggcagtacgttaccatcctacactatcagtaggtcagtggtattttcagggcacatttttcttgctggaaaatctcatgcttcccacttccattagtttttttttttttttttttttttttttgagacgaagtctcactcttgtcatccaggctggagtgcaatggcgcgatcttggctcactgcaacctctgcctcccagaatcaagcgattctcctgcctcagcctcctgagtagctgggactacaggcacacaccaccacacccagctaattttttgtactttagtagagacagggtttcactgtgttgcccaggctggtctcgaactcctgagctcaggcaatccacccaccttggcctcccaaagtgttgggattacaggtgtgagctaccacacccagcccaagatggagtcttgctgtgtcacccaagttgtagtgcagtggcatgatctcagctcactgaagtctccgcctcccaggttcaagtgattctcatgcctcagcctcccaagaagctgggactacaggcatgcaccaccacgcccagttaatttttgtatttttagtaaagatggggttttgtcatgttggccaggctggtctcaaactcccgacctcaggtaatccacccacctctgcctcccaaagtgctgggattataggcgtcagccaccatgcctgtcctcactgatcatctctataggggtgggaccctctcatctgcaatcccaggtgcctggtacaggtcaaatgtttggattaggcttccaattattaggctctatttaacatacagaaatcaatcatagctaacctctttattctaagtttcctcccctatttctgtgtattagttctgttcttccggagagccaatttcccctcctttgtaaggcttctggctgcgcagactacatttcttgtgctttcaatcaaaaggacctgttcatactctaacctggacttggtgctgtttttttgccctgaggtctgggcaccccctgcatcaaagtgtccttgttatatgtggcttcccctggaggaagcaattgcaaactaacaaaagttacacagtacaaatacaatacaaaactttgcctgaatcatttgagagtaagttgccaacttgctgcctattactcttggatacttcaatgtgcattttctacaaacaaggatattctcctttctacaaacaaggatagccatcatacaactattacaatcaggaaacagtattacattactgtaattgaatcctgaaactcaagcattgccagctgtcccaataatatcctttatagtgaaaggatccaggtcaggatcaagtgtttatttaattgtatgtctctttagtttccttcagtatggaacagtttttcagtgtttccttgactttcatggccttgaccattttgaagattatgagccagctatttttgtttactgtcccttggtttggaactatgcatttcttttttctttttttttttttttttgagacagagtctcgctttgtcgcccacgctggagtgcagtggtgctatctcggctcactgcaagctctgcctcccggcttcccaccattctcctgcctcagcctcctgagtagctgggactacgggcgcccgccaccgcgcccagctaattttttttgtatttttggtagagacggggtttcaccgtgttagccaggatggcctcaatctcctgacctcgtgatctgcctgcctcgacctcccaaagtgctgggattacaggtgtgagccaccacacctggccctatccatttctttaattcaggtttatatctttggcaggaatatgataaaaataagttgcaggccaggcatagtggctcatgcttgtaatcctaacactttgggaggccgaggcaggaggagtccttgagcccaggagttcaagaccagcctgggcaacatggtgagaccccgtctatacaaaaataatacaggaattagctggacatggtggtgtgcacctgtagtcccagttactcaggaggctgaggcaggatgatcacttgagcctaggaatttgaggctccagtgaactgtgaatgcaccactgcactgcagcctgtgtgacagagcgagaccctgtttaaaaaaaattttaaaaagattgtcactgcaaaattatatgcaatttttgtcaccttactgttttgattatttgaataaagtggtaggtacagtcaagcttctctatgtaacattttcccctctgttatttattttctaggtcagtactttgtactaagtaaacattgaattctgcatctgtcactgtattcacttattattattattattattattattattattattattttagagaaagggtctcgctccattgccccggctagaatgcagtgatgtgatgatggctcactgcagcctcaaactcctaggttcaagtgatcctgccacctcagcctcccgactcgctaggactataggtacacgatctcacacctaatttttaatttttttttaaatagagagggggtctagctatgtttcctagcctggtctcaaactcctggcctcaagtgatcctcccacctcggcctcccaaagtgctgggattacaggcatgagccactgcatctggcctatttatttatttatttatttgtttcaaaagggacacaggtatttctattttaatagcatacatgaatggcaacagattatttattgatgctcaagttgtcccagacttcaacatctttttatcatgtccccagcattctttgcatgctttctttctggtgcaacaaggtattacaggctcatcttgtattttccctgccctaattcttggccatttttttctaaggtctgtttcattttagtggagaatggtattcagaaaccgagaccagggagctagatatgttcattgctattggtgtgttgctgatcccaggcccttactgaaagagttttaagtagcaagatatgttccttagtgaatccaagcaagaaaagtgacgtgataatgccatcattccaagataactaatgatgtggttgtaaatgtcctgaggagcctattcttattatggaccatgtaatctgccaggtgatttgcagcatttcttctgatgagagccttaaggtagttaccactatttccattctccagaggcagaggccaagtgcattcactgggattgaaacccaggcagttaacctaagtgtcatgacactttttttttttgagacggtgagacggagtctcactctgtcgcccaggctggagtgcagtggcgtgatctcagctcactgcaagctccacctcccaggttcacgccattctcctgcctcagcctctggagtagctgggactacaggtacctgccaccacgcctggctaatttttgtatttttagtagagacagggtttcaccgtgttagccaggatggtctcgatctcctgacctcgtgatccacctgccttggccttggcctcccaaagtgctgggattacaggcgtgagccactgcacccggccgtcatgacatttttgcatttaatcactataacaacttataaactgtacttgcctcaactttttttgatgatgaaactagttcagagaagtaagtaaaaatggaccaaagacctaaatgtaagaactaaaactataaaacccttagaagaaaacatagtatgtaagtcttcatggccttgacttcctagatacggcaccaaagcagagcgaccaaagggaaaaacaaacaaacaaaaaaacaaaaccaaaacaaaccaaaacaaacaaaaccaaaacaaaacaaaataagcaaaaacaaaaccaaaaaaactggacttcatcagcaataaaacctttttttttagacagagtcttcctctgtcacccaggctggagtgcagtggtgcaatctaggctcactgcaacctctgccttgcgggctcaagcaattctcatgcctcagccttctgagcagctgggactacaggcgcgcaccaccacgcccagctaattttttgtaattttagtagagacggggtttcaccatgttggctaggctggtctcgaactcttgacctcaggtgatccacctgactttgcctcctaaagtgctaggattacaggcatgagccactacgcccagcctaaaacttttgtatatcaagggacactagcaagaaagtaaaaaacacagcccacaaaatgggagaaaatagttgcaaatcatatagctgataagggtctaatatcaagaatatatagtgtttacagctaagcaagaaaaagacaacccaattaaaaatgcgcaaaggatttgagtagatatttctccaaagaagataacacagtggccaataaacccatgaaaagatgcttggccaagcgtattggctcatgcctctaattttagcacttcgggaggtggaggcaggcggatcgcttgagcccaggagttcaagaccagcctgggtaacatggcaaaaccttacttctacaaaaaaatacaaaaattagctgggaacggtggcacatgcctgtagtcccagctacttgggagactgaggtgggaggatcacttgagcctaggagggtgacactgcagtgagtcatgatggcaccactgcactccagcctgggtgagagagcaagaccctgtttcaaaaaaaaaaggctcagcatcattaatcattaggggaatgcaaattaaaatcacttcatccaccaaaaagtggatatatggctgggtgttgtgcctcacgtctgtaatccaagcactttaggaggccgaggcgggaggactgcctgagcatccaggagttcgagaacagccttggcaacatggtgagatcctatctctattaaaaaaacaaaaaacaaaacaaaaccaaaaacaaaacaaacaaacgaaaaaaagatttttgggcctggtgcagtggctcatgcctataatccctgcactttgggaggccgaggcaggcggatcaactgaggtcgggagtttgagaccagcctgaccaacatagagaaaccttgtatctactacaagtataaaattagctgggtgtggtggcacatgtctgtaatcccagccactcaggaaggctgaggcaggagaatcgcttgaacccaggaggtggggattgcggtgagtcgagatcatgccactgcactccagcctgggaaacaagagcaaaactcagtctcaaaaaaagaaaaaaaaataaggatttttggctaaaatacagacaatgaaagcatcagtgaggatgtaaggagactggagttctcatacgttgctagtggggatgtaaaatgatgcagccactgtggaaaacagtttggcacctcctcaaagttaaacataggcccattgtggtggctcatgcttgtaatcccagcattttgggaggctgagcagggaggattacttgagcccaggagtttgagaccagcctgggaaacaaagcagcaccctgtctccaccaaaaaaaaaaaaaaaaaaaaattagctgggcaacatggcacaaacctgtaatctcagctacttgggaggctaaagtagaaggatcacttgagcccaggaggttgaggctgcagtgagctgtgattataccactgcattccaacctggttaaagagcaaggccttctccccagaaaaaaaaaaaaaaaaaaaaattaaacatagagttaatgtatgactcagcaattctactcctaggtatatatgcccaggaaaattgaaaacatatgttcatataaaaacttgtacacaaaaactcatagcagcattattcataatagtcaagaataaacaatccaagcgttcatccactgataaatagattttaaaatctgttatatctatatagtagaatattatttagccaaaaaaaaaaaagaatgaattatgtccggacgtgatagcacatgcctgtaattccagcactttgggaggccaaggtagaaggatcccttgaggcaaggagtttgagaccagtctgagcaacatagccagacaccatctttaaaaaaacataaacattaggcaggtagtaatggcatgcaactgtagtcccagctactcgggaggctgaggcaggaggattgcttgtgcccaagagttggaggctgcaatgagctacaattatgtcatgtgctctctacttatgcttatgatatgcttatgattactgcattccagcctagtagtcactgcactattaggtagtggtgattgttgcacaactttgcgaatatactaaaaaaccctgaattgtacacttttagagggtaacttttagagtatgtaaactgtatcacaattaaaaattatatgtgacttacattatattttattggacaatattattctagactaggaaccctgaagccaggagccaagaggtaccatgataaagtctctgggtagtgattccacagcagagggcctgggctccagttccacctcatagagtccagcccacttgttctgggatggcagaagcatcactgaccttattccccaggtccgggacactctgatgctggcagacaagcccttcttcctggtgctggaggaagatggcacaactgtagagacagaagagtacttccaagccctggcaggggatacagtgttcatggtcctccagaaggggcagaaatggcagcccccatcagaacaggtgaggtcccacctgttgcacctgatgggggagaaaatcgggactctgggagatgtcttcaaggggtgctaaccatttgcttgtctcaatgcaggggacaaggcacccactgtccctctcccataagcctgccaagaagattgatgtggcccgtgtaacgtttgatctgtacaagctgaacccacaggacttcattggctgcctgaacgtgaaggcgactttttatgatacatactccctttcctatgatctgcactgctgtggggccaagcgcatcatgaagtgagcaaattgggatttcctggggacagggtgggtacaggcaatgggagagcccctatctgtccacatggactcagccttctcagcgcctgggtctggctttggtgctgacaaagcatagcctgtatcagacaacacggagcctgatgggctctcgaagtgctgtggacgacaccggcgtgtgcccaggggtgcacgtgttcctcctggtggaacctggatgcattttatttttatgactactttccactctagtaactacattttttcattgaaataggaagttttcctaagtagtcttctagtcaattttataaaaatgaacatgtgttggccaggcacggtggctcatgcctgtaatcccagcactttgagaggccaaggtgggtggatcacttgaggtcaggagtttgagaccagcctggccaacatggcaaaacctaatctctacaaaaatatgaaaactagctggacatcgtggtgcatgcctgtaatcccaactactcaggaggctgaagcaggagaatcgcttgaagctaggaggcagaggttgcagtgagccaagattctgccactgcactccagcctgggcaaaagagcaagactgtctcggaaaaaaaaacaaaaaaaagcacgtgtcaaagacttaattgaattcattaattaatgagagaactagcaagaagttacaatcagctcaaaggagatttcaaaatcccacatatatacaggtccaggaagaatgatgagataggttgagtgcagcagctctcacctgggatcccagcactttgggaggtcaaggcaggaggatctgttgagcccatgagtttgagaccagcctcggcaacacagtgagaccctgtctttatcacacgcccgtgtgtgcgcacacacacacacacacaaaatgctggcagtggtagtatgtacctataatcccagctaggtgggaggattgcttgagcccaggagtttgaggctgcagtgagatatgaccatactactgtgtactccagcctgggcaacagcatgagaccttgtctctaaaacgaagaaaaagagagataaatgtatacatacaaaattgggctttttcccctgtgggaatgggagggatgaaataatcttccctccatatgacagaattaatttgcaagtcttatggaccagagtcatttgtactatataatgaacttttcttaaaacttatttttgaaagtctaaccaaatgtctcaagaagcatgagaatatgtgaggttagaacccgctcccgcttccccatttcccctactttccaaacatccaaccccatgttaattgctttatgctatttcagccttatgaccctgggtggcaggtgcaatcccattttacccatgaggaagctgaagctcaaagctagagcagtttgctgaagttcacccagctaataagactggattaggacttaggcctgcctgattcaggttctgttctcagtacatgctcatgccaggcatggatgtcttcatgtccagcactctgaacatgagctagactgcaggtggcttggacaggcctcagagtctagaacagagtgtctcaaggtgcagaaccatgttatggagattaatctggttggtccaaagtagtagtagaaaaataaaagccttatgaaatgagctggggttttgcatgagaaagtagggaggttccgggcatctcagttaaggtggaattgcaggtcctcgcactaatcttgatcccatctgcagcatctgttcctatttgcacatcgctctagaattgatgaaatgcttttagtgctaccacctcagtggtattcaaatgttattaatgaagaaaacaaacataatgaaaccaatcccttccaggccagcatgggaataagggggtgcagggagggggtacactcttgtttttatttattatgtatttatttatttttgagacagagactcactctgtcacccaggctggagtgcagtggcacaatctcagctcactacaacctctgtctcccaggttcaagtgattttcgtgcctcagcttcccgagtagctgagattacaggtgtctgccaccacgcccggctaattttttgtattttttagtagagacggggtttcgccatgttggccaggctggtctcaaactcctgacctcaaatgatccacccacctcggccacctaaagttataggattacaggcatgagccaccaagcccagccttgtgttttttgtttgattgttttttgagactgaatcttgctctgtcacccaggctggagtgcagtggtgccacctcggctcactgtgacctccgcctcctgggttcaagcgattctcctgcctcaggctctggagtaactgggattacagtcacccaccactatgcctgcctagtttttgtatttttagtagagacagagattcgccatgttggccatgctagtctcaaactcctgacctcaggttacccgccctccatggcctcccaaagtgctaggattacaggcgtgaacgccatgcccgacctacttcgtggttttgacttgcgtgccctctcattccacccagggaagctttccgctgggccctcttcagcatgcaggccacaggccacgtactgcttggcacctcctgttacctgcagcagctcctcgatgctacggaggaagggcagccccccaagggcaaggcctcatcccttatcccgacctgtctgaagatactgcagtgaaagcccaagtccttggaagctttccccagtgaaggactgactgggggcctcacgcttaactggtagtgcccacaagcctggcagctgtagagccgcgaacctccccacacctccctcaccgcgcaggaccctgagtgaggaggaggagctggaaacctggggtgggttggccaaaggagaacctcaagctcctggcctgatccagctccttcctgcccaaggcagcttagcccatccagactggtcctgaagtctgtccctccattggcatgaagtctgccccttagcaatctggcctcgcaggctgtactttcatggtgctctctaccttctggcccccatcccggaacattcctgagtgaattcgcaagcgcactagcatgtgatattagggagtttgcaataaattattgaggctgatgta

SEQ ID NO: 7 - DNA, 25920 bp, Homo sapiens
cttcttgtaagagagtgctaggcacatagcctcctcctattcctaatcctcccaccaaagaaagaggcacagagttcattacttagtgggggccagctgtgatcggccaactgccagctgccttaaaaaggaagaccagtgatgctaggatggagtgaaacccaagaggaagtgccatcatgaggaatcaatgagagatctgtgaagagagagggctgggtgggagcccagaaggatagaacctggaagatcaatatctcccgtgagggaaataacaatggagccaggttctaagtcagtgtctaggtcagactggcaacctgaaccacaccagaggcctataaccccgctagagcctgggccagaaaagacacccatagcccagccagaatcgaagactctgcagggatccaatacccaacagaagcctgcttcaaaccaaagacccctcacccagcaggagacccctgcacaacatgatgctgaatcccagaaggaacctagagcccaacaaaaatctgcttcacaagaggaatttcttgccccacagaagcccgcaccacagcaatcaccttacatccaaagggtgctgctcactcaacaggaagctgcctcccagcagggacctgggctaggaaaagaatctataactcaacaggagccagcattgagacaaagacatgtagcccagccagggcctgggccaggagagccacctccagctcaacaagaagctgaatcaacacctgcggcccaggctaaacctggagccaaaagggagccatctgccccgactgaatctacgtcccaagagacacctgaacagtcagacaagcaaacaacgccagtccagggagccaaatccaagcagggatctttgacagagctgggatttctaacaaaacttcaggaactatccatacagcgatcagccctagagtggaaggcactttctgagtgggtcacagattctgagtcagaatcagatgtgggatcatcttcagacacagattctccagccacgatgggtggaatggtggcccagggagtgaagctaggcttcaaaggaaaatctggttataaagtgatgtcaggatacagtgggacgtcgccacatgagaaaaccagtgctcggaatcacagacactaccaggatacaggtgagtgtgaatctggaggaattgaagcctctcagggacagtcattcatttacttaaaaaatactaaaaacttacggtgtatcaggttagctctggtcatccttcaacaatcctgagataaagatcaacatccccattattaaaaaaaaaaaaaaaaaaaaaaaaggctgggcgcagtggctcacacctgtaatcccagcactttgggaggccgaggcagatggattgcttgaggccaggagttcaagaccagcctagccaagatggtgaaacaccgtctctactaaaaatacaaaaattagccaggcgtggtggcaggtgcctgtaatcccagctactcgggaggctgaggcagagaaacacttgaacccgggaggcaaaggttgcaatgagctgagatcgtgccactgcactccagcctgggtgacagagtaagactccatctcaaaaaaagaaaaaaacaaagtctcactctgtcgcccaggctggggtgcactggtatgatcatggctcactgcagcctcaaactcctgggctcaagcgatcctcatacctcagcctccggagtaggtgggactacaagtgcaccaccatgccccgctaatttttacattttccgtagagacgtggcctcgctatgttgcccaggctggtctcgaactcctggccccaagcaagcctcccacctcagcttcccaaagtgctgagattacaggcatgagccaccgagcacctggccaacatcctcattttagaaatgaggacatcaggtagaaaataaagaagtaagggttcttgcacagggccatgcagttaggaagtggcaaagtcaggatttgaagccggggtctttctcccctggaccaggctgcctccctatagaggatttggggaaaatcaaatcccagagtgggaaggcactctgagctagggaccaggagaccaggaaagcggccctcttgccagtgttcattcatccatccacagaaactgactgagcaccagctctggggtagacacgggcgtaagtcccagcaatacaatacaaagacggtaccttacccttctggagtttccaggcaggctaggcagaacattggggcatcaacagtctctcagtttaccaggtctgtcctcttctgccaggaagatacctgctccctgccaggctgcctccataggggactcagtccagccaggcggaagggccttgcgggttgtgttctcagcaaggaggagccgggagagaggtgcgggagccgccaaggtctcaggcaaggtcagggactgacggcaggaaagttgggggagcagcatcagaggataacgggcccaaggctctgggctcaccgcaggcactccctcgccttctccgcctcttcccggggcccagtgctgccccctgcctgcttcacaggagccccaggaagctccgatcttccttctctaggctcaaaacccctgccatggagaaggggggaatacaagttttatctatggaactggaggttacttgttgaagtcccctgatgtggggggaaaaaccaacgcagccctaatcctgaagctgagggtatctgtgaggccctggtccagagggcaggcaatggcggcccctccgcccagaggccagtctgtcttgtctctcccacagctgataagagagtaacgcgtttctcagcgccggggctgctgggtatgctttgagagggaaggaagaggggcggcaggcaaaggcagggattgaggcctgctctccccagtgctgaggatagaaagctgtttctccaggcctccagagcacagtctcaagggatgcccgccccacaccaccgcggcgggcgaggttgcctttcaggcggtccagaagcagttggctgtgtgcgtgtgagcaaggcggtccagaagcagttggctgtgtgcgtgtgagcaagcctcgagcctcctctgtttcccgccatttctcaagcatgcgtcgttccctctacctccgctcttgggccggcccgcggcctgaagctagtgcgcaggcgccgagcgttctcctaccaccatcccccaccctcattatcgggagcatggattagggggcggagacagagggagaaagatcccccaggaaaagcgcaataggagaactaggagacaggagagggaaagcggggctccctcctccaacggctctcaggcacagccacgcctccagcgctggcgccccgcctcgtctttgagttggcagaggacctggcccgcccccaccccaggcagaggctcagaggcgccccactttcccccttcaaaaggcgggccgggccgcgtgctagggcatccctccctgggaaggtgagtgggatggcgagactgagccagcacatgggaggcttcccagcacgtgggaagatcgggacttccctgctggggtgctccccgcggggctccgcactagtcctgcccgaaggacgcttttcgccacccccggggttgcctttttgttggccgcgagccctttaatgcgcggtagggggcggagccagatcttaaattctggcggttgggggcggagcacggcagcggagggagaccagaattggttagggagctggacccagagaagagcaagctcgggattgggtaataggggggcccgacagtagtgtgtagcgaggattggccctccgggcagggcctcgcggctggaattggcccccgcgggaggaccgcggggaattgatggagttggccgggggaacggagcccgccgaggccgctatggccccggcctcgaagaatgccaaagagggctcaaggagccacggtcgccggcggtggcgaaaagacaaggccaaaggtgagcgcgcggagaccggcagcagcccggaaggaacctcgtacatcctacggggcagtggggacagcgtggactggcccactgccacagggagttgggggggtccgcggactggcccattgccgcttgcggggacaacgggatgatccattgtgagaagggggtgatgtgacagccgagaggcccaccctggcacagggactagctgacgcccggatcggcttgggagcagagtccggaatgcgagcttcgtggaccagggttcccgctcggattccgtattcgctgtgtgaccaagggcaacggatgccctcagtgtttcccacctggggaatgcttcaagcgccgatgttcagggctccagcgctcttgagaggataagggggaaggaggttagcaggtgttactcaaagaggaagtctagcggtgagtcaaggagaggtaggaggaaggggtgcccatagtggagagtgcagcggggccagtaaagttcatcgcagtcccggcctcccacccggcaagtcagtccatccatccatccattcattgtcagtcaacccgcttcagccggctccactggggcctagccctgacccctaccctttccaggacaacgggccctggcttggcttcagacctccacacctgcctcagcaaaagggaaggcctcatttctctagggtgtcacattattttcaggtctcaaaaggcggaacaaaggctgacagaccaacagaaatgctgcaagcctcaggcaggggcaggtcctcagttacaagcatttatcaatcagcatggggacccgtgggaggttcccatgcaggaaggaaactaacaccacccctcagccccagggaccccaaactcattgcttcccctgccgtagaacacatcattagcccagaatagacctggagttggcttcagacacacgcaggctcagacaggctcagagggcagacggactggagcccaagggcactcgctgtcacagcaagacactgagcctcacaccagtatagcatacccagaggcagtacagactgtagttaagaaacgtgggcgtcggagatccggttcctcaggctgggagctgtgatgttctgggcaggagactccctctttccctgcctcagctttctgagctctaaaatgggtatgatgataggagtacctccttttgtgaggttagaatgaatgtagagcgtttggcacaggcacgcagcaaaccctcaggagaagtgaaggttattgttactgttagtccccaccggacggtgaacatcctaaaagcagggctgtatctgccttgtttaccctgtatttctagagcctggtatgccaggcaaataggcactcaaatatttattggattaataagtgaaggaatgtgtgaactgtaacagagatcataataatcacgcttcccttagggttgtcatgaggagtcagaatccagttgggcagggctccctgcaaaacgagccattcacaaaggctcattcctgcaaactccaagatttgcctgtcatttcctgaagccccgccctcgtgccaggctttagataaattaatcagacctcactcctgcctcaagggcatctgggctggctggagcaatcctcgtcatcagtttcatcttgccagtaactggatgaggctagtgtccttaggatcaccccatttcccagatgacgacactggggctctgagggaccaaggccgcctgggcttccttacttggccagtgagggcaaagagatggctgcctcagcctcctcggggcagggagtttgtggacgcacaactgctgtgggcccaaacttgattctgggcccaagaggttccagtcacacagattcattcagcagacattcactgaacactgagctgtgccaggcctgggctgacagattcacagttgagctggggacacagatattactcagataagactatgattgtaaactgtgataaatacaacagagaaaaacaccctgggaagatagaattgagaggcttcccaaggaggcaatattggacccaagatggacaggcgttaagtgctgtgcagggggcaggcaggggaacagagaggacagtatggagaaagacccgaggcttgaggaaacactgagttacagtgcagagggagctcagccagggccaggaggaagaggaagaggaaacagggcaggcagtttaagagaggaggatggtagctgggtacagcggctcacacctgtaatcctggcactttgggaggccaaggtgggaggaccccttgagtccaggagtttgaggccagcgtgggcaacatagcgagaccccgtctctaccaaaaacacaaacattagccgggcatggtggggtgtacctgtagtcccagctacttgggaggctgaggtgggaggatcaattgagcccaggaggttgaggctgcagtgagctgtgatcgcaccactctactccagcctgggcaacaatgagactttgtctcaaaaagagagagagagaggaggatggtacctggcttgggaggccatgtgcaggattgggggctttatcctgaaagcaagggagggggtgttgaagggttggaagtgtgtgaacagatttgtgtgctaaaaagatcctgtggctgctatgtggacaacaaatcaaagagaacaaaatcagaaatatataaagatctgtcatagtcacccaggtaatgatgctaccgcctggaccagggaggaggcagtggaggtggggagaagggccaggttccagaggcctttagtaggcatagtgggcaggactaggggatggattgcatgggagttggggggtggggaggtggccaggatgacatcctgggttccagcttgagcaaatggggagaggtttgttctcaacacacacacagagaccaagacacgcacaagcatgtatatccattcatacataaacaaacacgcccacacaagctcacaggccaagagaaacagagacacagatacattcacaggcatacaaacattcataccttcaaatgtaccagtgccgacggacacaggccaggacacacgccagccatgacacatacctgcagaggtacatatgtacacgtgaaccccagacacactcacacccgaccagaaagcatatactttgaagcacagacagagggagcagcacacagaaacacagcagagatcagcagctctgccctggagctgccccctgctggccgtgtgaccccgggggttatgtcacctttctgtgcctcaattttgtcatctgccaaaagggatgataatggtctctccctcataggttgtggggcaggcgacatgagtcacttcacataaaacacataaagaaatctcatcacctcctttgagaatgaaaaaaaaaaaaaaaagaaaagaaatcccagcactttgggagaccaaggcaggaggattgcttgagcacaggagttcgagaccagtctaggcaacacagctagactcgatccctatttttaaattaaacacacacacgcacagagaaagagaccaggcacgcagaggctgttggtgcaacgtttgctcttttatgcacacactctcggggatccagccactcattgtccccacctgcatctgattccaggcttcctggcttcagaagcctttgcttcctcccaggccccacacctggcccttcctccatcccccatccacccccaccccacttccttccccaccgccctccttacccctgctcagtccccaggagtcggattcagctgtccagaaactcttcagggcaaagaaaggtccagtgcccagtcagtgggacaccacccaaagccaaatcccctctgccctacctctcccctagtccttacccacggccccagtgggcctacccctgccccagtgccctctgccagggattcctggcgagttgctggcaacctcagcatcccttcctgccagctccacccagcccagtcctcggggagccgcctggaacaggacaaagggcagagctctggagtccctagggcctgggtccagccccttccccactcctttgtgcagtcacttgttttgttggaaatgtcccctctcctcatctttttatttattttattttaatttttttgagacagagtttctgtcttgttgccgaggctggagtgcaatggtgtgatcttggctcaccgcaacctctgcctcccaggttcaagcgattctcctgcctcagcctcccgagtagctgggactacaggcacgcgccaccatgcccggctaattttgtatttttagtagagacagggtttctccatgttggccaggctggtctcgaactcccgatctcaggtgatccaccggcctcagcctcccaaagtggtgggattacaggtgtgagccacggcgcccggcccccactcctcatcttaaaagatccctaccggcatggatgctaccactttaccaaggggcggcagtgcaccaggcactgtccaaaggctgaataaaaaaggcaaaagtccctgccctcagggagtttacaatctagtgagggaatcagagagcaaacaatataaattcaggcatgtctcagagatatttggggttcaattccagaccacttgataaagtgagtcacacaaatttgttggtttcccagtgcatagaaaagttatgtttacactatcctgtagtctattaaagtgtacaacagcatatgtctaaaaataccatgtacatattttaatttttttttttctgtagagacggaatctcactatgttgcccaggctagtctcgaactcctagcctcaagtgagcctcctgctttgacctcccaaagtgccaggattacaagcatgagctaccacgcctggccccatactatgctaatgatcatctgagccttcagcaaatcctcatctttttgctggtggagggtcttgccccagtggtggtggctgctgactcacgagggtggtggttgtagaaagtggctgtggcagtttcttaaaataaggtaacaatgaacttttctgcttcgattcactcacaaaactgttctctgtaacatgtgatgctgtttgatagcatttggtccacagtagaactttcaaaattggggtcaggccaggcacggtggctcacacctataatcccagcactttgggaggccgaggtgggcaaatcacccaaggtcaggagtttgaggccagcctggccaacatggtaaaaccccgtctctactaaaaatactaaaattaaccaggcgcggtggcaggcacctgtagtcccagctactcaggaggctgaggtaggagaatcacttgaacctgggaggcggaggttgcggtgagctgagatcgcaccattgcactccagcctgggcaacaagagcgaaacttgtctcaaaaaaaaaaaaaaatgggggtcagtcctgtcaaagcctgctggtgctctatcagccaagtctatgtcatagtctaaatcctttgttgccattgttcctgctatcaaatgtatttctgtttttttatttttttatttattttttgtagagggggagggtctcactatgttacccaggttgatctcaaactggcctcaagctatcttcctgccttggcctcccaaagtgctgcgattataagtatgagccactgcacccagcctacaaaatgtatttctaaatgtatttctattttttattatttaataagacttagagctgggcatggtggcacatgcctgtaatcccagctattcaagaggctgaggcaggatgatcacttgaggtccagtgttcaagaccaggctaggcaacacagccctgtctcttaaaaaacacaaaaaattagccagatgcagtgactctcccagctactcaagaggccaaggcaggaggatctcttgaggcctggaattcaaaaccagcctaggcaatatagcaagactctgtctctaaaaacctggaaaaaaaaaaatgaaccaggcatagtggtgcccatctgtagtccccactactcaggaggctgaagtaggaggatcacttgagcccagaagttcaaggctgcagtgaggtatgatcgtgccactacactccagcccaggtgacacagcaagaatccatctctaaaactaaaaattttaaaaataagaataacaaataataagacttaaaagttgaaattggccgggcgcagtggctcacgcctgtaatcccagcattttgggaggccgaggcaggcgaatcacgaggtcaggagatcgagaccatcctagctaacatggtgaaaccccgtctctactaaaaatacaaaaaattagccaggtgtggtggcgggcgcctgtcgtcccagctactcaggaggctgaggcaggagaatggcgtgaacccgggaggcggagcttgcagtgagctgagattgcgccactgcactccaacctgggcgacagagcgagactccgtctcaaaaaaatataataataataacaaataacaagacttaaaagtcgaaattactccttggctggtgggctgcagaacagatcatgtgttagcgggcatgaaaacacattaatctactccaccagggccctttggtaaccacatgcattgtcattgagaaatattttgttgttttctgtgcagtccgtctcaacagtgggcttgaaatattcagtaaaccatgctgtaaacagacgtgctgtcatccaggctttgttccatttaagagcataggcagagtacatttggcgtaatcctgagggccctagaatttctggaatgataaatgaacaccggcttcaactgagtcaccagctgcattagcccctaacaagggggtcagcctggccttgaagctttggcgcccaggcactgacctcccctccagctaggaaagtcctggatggcatcttcttccaatacctggtggttttatctacactgaaaatatgttgcttaatgtagccgtcttcatcaatgatcttagctagatcttctgcataacttgctgcggctcctccagcagcacttgctacatcaccttgcacttttttttttgagacaaagtttcactcttgttgctcaggctggagtgcaatggcacgatctcggctcactgcaaccaccgcctcccaggttcaagtgattctcctgcctcagcctcccaagtagctgggattacaggcatgcgccaccacgcccggcttcttttttgtattttaagttgagatggggtttctccatgttggtcaggctggtttcaaactcccgacctcaggtgatccacctgcctcagcctcccaaagtgctgggattacaatcgtgagccacgatgcccggccgtcaccctgcacttttatgttacagagatgccttctttccttaaatctcatgaatccacctctgctagcttccaaccttttttctgcagcttcctcacctctctcacccttcaaagaattgaagagggatagggcttcgcttgggattaggctttggcttaaggaaatgtggctggtttgaccttctatccagaccgttcagactttctccatatccacaataagcctagttcactttttttttttttttccagacggagtctcactctgtcacccaggctggagtgcagtggcgcgatctcggctcactacaagctccgcttcccaggttcacgccattctcctgcctcagcctccctagtagctgggactacaggcacccaccaccacgcccggctgattttttgtatttttagtagagctggggtttcaccgtgttagccaggatggtcccgatctcctgacttcgtgatccgcccgcctcggcctcccaaagtgctgggattacaggcatgagccaccatgcccagctaagactggttcactttcttatcattcatgtgttcactggagtagcacttttaattactttcatgaatttttcctttgcattcacaacttagctaactggcagaagaggcctagtttttagcccgtttcagcactggacatgccttcctcactaagcttaatcacttctggcttttgatttaaaatgagagacataagactcttctttcacttgaacactttgaggtcattgtagggttgttaattggcctgatttctattttattttattctatttttcatctcatttcatttatttgagacagactctcgctctattttccaggctggagtacaatggtgtgatcttggctcactacaacctccacctcccgggttcaagcgattctcctgcctcagcctcccgagtagctgggattacaggcacacaccaccatgcccagctaatttttgtatttttagtagagatggggtttcagcatgttggccaggctggtctcaacctcctgacctcaagtgatccacctgcctcagcctcccaaagtgctgggattacaggcattagccaccgtgcccggcctcaatattgtatgtcttagggaatatggaggctcgagaagagagatgggggaaatggctggtcagtggagcagtcagaacacacacagcattaatcggtaaagttcgccctcttacgtggacatggttggtggtgcccccactcacaatagtaacaccagagatcattgatcacagatcatcataacaaatataataataatgaaaaagtgtgaaacactgtgaaaattaccagaatgtgacacagagacacaaagtgagcaactgctgtcagaaaaatggtgccaacagactggtttgacgcagggttgccacaaaccttcaatttgtaaaaaatgcagtatgtgcgaggcacagtgaagcaaggtattcctgtagtaaactgtatagtatgctagatggtgacaagtgccgcagagaaaaatcacccaatacaccatctcaggaaggccaagtgagcccacaaagcaaaccacatgcagaacatgccggccatggccccacttataaattatgtctcccaccctgagccagcagaacagccaagtgcagtttccagatgaggaaaacagactcatccagggtcgccagcaagggcttgggccatctttctttccttttcttttcttctttcctttctttctttccttcctttcctttctttctttcctttcttttctgacagagtctcgctttatcgccaggctggagtacagtggcgtgatctcagctcactgcaacctctgcctcctgggttcaagtgattctcctgtctcagcctcccaatagtggggactacaggcatccaccaccataccgctaatttttgtatttttagtagagacggggtttcagcatgttggccaggatggtctcgatctcctgacctcgtggcccacccacgtcggcctcccaacgtgctggcattataggcatgagccactgtgcctagcccacatcgtcccattttcacagctgcaagcctaacactgcctcctgaggcctctgtctatccatctgtctgccagccctggggaggggccgtcctctcctgagctaactagcccctctgccctcgctctgcttcttcatgccttccttcctgcttcatatatccactgagcacctgctatgtgccaggtcccatgataggccccagggccacctgggggatgacagtccgggacagaagattcacttccagccaagcaaatgaccctgaggcctcagagcccctcagtcacacaggcccctttcaaggccctaatcttttttgttttgttttaagacagagtctcgctctgtcacccaggctggagtgcagtggcacgatcttggctcagtgcaagctccgcgtcccaggttcacaccattctcctgcctccgcctcccgagtagctgggactacaggtgcccaccaccatgcccggctaatttttttgtatttttagtagagacggggtttcaccgtgttagccaggatggtcttgatctgctgacctcatgatccgcccgcctcggcctcccaaaatgctgggattacaggcgtgagccaccacgcctggcccaaggccctaatcttgtatttgtaattctgtatttcttcttcctaaagggccacaccccggtaccttataagtttcatggctccataaaacctagtcctgtcctgggggagatgaacaaacacgtaaacaaattataaagtgtcaaatggcaggatggaacagaactaaatatcatggcagaaaataacgggggcgtgcatatcttagctggggagaagcaactggggagtacctcttggaagtgacaagctgaggccagacaaggaatctgtggaatgacacgggtgaatgacatgggtgaggaatctctctctctgagcctctgtttccctgtgtgaggtggggatgccagtcacagcagggtgggctgagaggggtaagtggcaggtccaggttacccagtgtggtcacaccacccagtaggaccgacaggcagctcaggcccctagctgagcctggcactgcccttaatcggcttttgtccttccaggagggacaggcagggctggctacctcctctctccccccaacagctggggctggctgtgccagggccagactaggaggcgggagctgagatcactgggcctttggctccctgagtcctctctggggacagagagggcagcctggctgggtgagaggggacggtttgttcaggactggggctaggactcctgggtcctaaaggatgaaggggctgggggcctggactcctgggtctgagggaggaggggctgtgggtgtggactcctgggttctaaggcagaggagcctgtaacaggactaccagttgagctgagtctgggagcaggtggggggcagagcagggagctgagccctctactctgtttacagcacgtggtcctcactgatctttctgggtgggaggtggcttgtgcggctacaccctgggcaggccagccccgcccccgggtttattgccccaggctgctactggcacaagccacagaccagcagtcccagcccagggaagctcggaagatgcctaggaggggtgagtgtccagccagctgagagacgggaatggccgctgccaggccccaggacctagaagggaaaggaaataccttcttctaggggaacaggcaaggtgctgggctttgggagggaagtcactggagcagatggtagcattccaccaagggcagcttgttcaggacctgggtgacagggcatccaagaaacagggcagcctgaatggctgggaaaggatatcagaggaaagatcactggggactggaaagtgtggagatcagacgtagtccagggcaagttttgagatgtggcttccagagtcctagcggggagccccacataagtgtggctgtggacccagagagctgggtttacaaaagctctgccacttgcagtgcaactttaacctcctcacttaaaacctgagcctcagtttcctctcctgtaaaatggggatgctggcaataatagagatttgaacctaggtggtgtgggtcccagagggttagcacgctaaagcagcaaacacacagaaagctctcagcacgggacctgcacggatatgctcaaggtcgacaccagtgatgagtatcgctgttccaggcaggggagcagggccggccatgcaacccaggaccagtggacaagcctgaaggccaggggaccccactgggggagaagcaagactgagttttggggatctcacagagaaacagggagggaactgggatttggagggggttccacagccagacttgaaggatgaggaatgactcttcagtgaagtctagaattgtgaactcctgagttcagggagagctgcgcttggggaccagggtcccgagagatgggaatgggagggctggggctgaactctcccaaatgttgttggaggggaagggcctatgggtagcacttggggcctaagaaggaaagatgggatgggggacccggactcctgggtcctgagaatggggaccaactggaggtttagacttcttggaatctaggagaaggagtcttgggccccaggagaattcatggagacaggtgactagactcttgggttcctggaaggaagaaagaaggaccggcagcctcctggatcacaggagaggtgaatgagttagggaagcagagtcgtgtgggctcagggaatgtccggattcaggaggccagggcagcaagtttctgagtcccaaagaggtgatagcaggggctcctgggtcctgaagaggaagggcttggggcttggactcctgggtctgagggaggagggagctgagggcccaaactcctggctcccgaggagggacaaaggcactgggaactggggccccaaacttctgattcccagagacaagagggtgacctcttatgtctaagggaggaggaacctggggtcctgggcccctggaactgaaagaagacagcactgaggtttgaaggaggagtgggtaagctatggcccagactcctgggccccagctaagcaaggcttgatccagccccacctaacaggcctccccacctgcccacagcctcaaggctcatccacaacatggacctgcgcacaatgacacagtcgctggtgactctggcggaggacaacatagccttcttctcgagccagggtcctggggaaacggcccagcggctgtcaggcgtttttgccggtgtacgggagcaggcgctggggctggagccggccctgggccgcctgctgggtgtggcgcacctctttgacctggacccagagacaccggccaacgggtaccgcagcctagtgcacacagcccgctgctgcctggcgcacctcctgcacaaatcccgctatgtggcctccaaccgccgcagcatcttcttccgcaccagccacaacctggccgagctggaggcctacctggctgccctcacccagctccgcgctctggtctactacgcccagcgcctgctggttaccaatcggccgggggtactcttctttgagggcgacgaggggctcaccgccgacttcctccgggagtatgtcacgctgcataagggatgcttctatggccgctgcctgggcttccaggtgagccccctgccccgtgcccctctgggcccacaatgaaggcacctggtggcccctggcctggcctatagtatggtaaagtaaccagtggtcagcattatcggactcagcaaacagcaggtacagaaaggagaagttggctggagctcagacagagctgcaccaggggtctcaaactcatggtccctaggtcatatctgctaggtggtaagaaatgcaacttgcagagcttttttcaccaggatatccactactctctattgtcctacatgcaagagatgtattcactctttgttacctgtctggcccctgaagtcacttgagtttgagacccctcagggagggatctggagccatgtgaccctatagtcattctggccctctctgggcctccccgcacctcagcctccccctcccaccccacatctccaggcttcccttcaggctagcctatcctcagcaacccagcccaggcattcctggaaacctcccctcaccagggctgaacccacaagtattgatcagcccagctgggccagccagctcctccctgtgtcccagccgtgaccctagagtcatgagaaagagatcccggggctgcccaaaccagcatcccagccacaatagccaggcatcaggtgccctctggcctactgggcgcacagccaggggcagggcccagctctgccttggtgcagggcagtctcccctttaaaatgtccgtccccgggagggcagaggcctcccctgtcttggtcactgctccagcccagtgtctagcatgcagtaggaacttggccagtacagagtgaacagaagaacccgccaatcccttgccttggcctcctatggctccatttcctctaatcctgtgtccccattgtatttttttttttttttttttttgtattttgtttagtagagatggggtttcaccatgttggccaggctggtctcaaactcctgacctcaagtgatccacccaccttggcctcccaaagtactgggtttacaggcatgagccactgcacctggaccccactgtctttccttaactgcctgtctgctctgtcagacgctgtctctccctgcccccgtggtggctggctggtcctatgtctaggcctcagtctccctctctgactgtctttctgcctcaggtccctgtgcctggggggctgtctctggccatctccctgttcctgtcttccagctgcctcctctccccatccatcctctctctgactttcaggaagtccctgcaccatcctactccccacctcacagcctaccagtggcaccaccccgcactgggaatgaaatccaaattcctctgcccaggccctatatgagccccctttgcctctccaatgtcaccacctgcccctctccccactgctcactctgctccagccacactggccccctgactgtcgctacccaggtcaagcaccaagcatgagctcacctttttttttttttagagacagagtctcgctctgtcgcccaggctggagtgcagtggtgtgatctccactcactgcaacctctgcctcctgggttcaagcgattctcctgcctcagcctcctatgagctcacctttaagccttcatgctctctgttccctgtgcagggaacaccctcccctcgcttcagtcagatctctcctccgagacccgctaagtagcctgacccctagtcatcactggtgccctctccacctactgcctttttttttttccaaaccattcgtcattccttgtcatgatactgcgcgtggctttgcttatctgctgatcatttgttttccctttgagaacatcaggtccgtgaaccagccgagagctatgcccagcacacagtaggagctcatgaaacgtttactgaatgaatgtgtcttccccgcacatccctgtgcctcgctcctgccctgtccccatccctctcttgagcggtgggtgacgcagccgcgtctctccacagttcacgcctgccatccggccattcctgcagaccatctccattgggctggtgtccttcggggagcactacaaacgcaacgagacaggcctcagtgagtcccagccaggttctactcgcagcctactctcttggccctcctccccactcctgcccaagcagcctgacccactggggctccctgacaccccttctgacccctaggtgtggccgccagctctctcttcaccagcggccgctttgccatcgaccccgagctgcgtggggctgagtttgagcggatcacacagaacctggacgtgcacttctggaaagccttctggaacatcaccgagatggaagtgctatcggtgagggcctgttgcagatggggacactgaggcccagagactcatctgaggccacagtaggtctctgtggggctccaggcccagggatggcacacaaggggaaagactggggatcagagggagaggtgaagggcacccctgcaggcagaccttccctagcaccccttctgtcccctccctccagtctctggccaacatggcatcggccaccgtgagggtaagccgcctgctcagcctgccacccgaagcctttgagatgccactgactgccgaccccacgctcacggtcaccatctcacccccactggcccacacaggccctgggcccgtcctcgtcaggctcatctcctatgacctgcgtgaaggacaggtagggttccagcctcagccagggacagggacagagccgaggagcgtgggcagcccccaccttggcatccccatggaggggtgcagccctggccagctctccccaacctcacacaccgtctccctcctcacccccaggacagtgaggagctcagcagcctgataaagtccaacggccaacggagcctggagctgtggccgcgcccccagcaggcaccccgctcgcggtccctgatagtgcacttccacggcggtggctttgtggcccagacctccagatcccacgagccctacctcaagagctgggcccaggagctgggcgcccccatcatctccatcgactactccctggcccctgaggcccccttcccccgtgcgctggaggagtgcttcttcgcctactgctgggccatcaagcactgcgccctccttggtgagcctcaggcccccacagccacctgcgcctgctcccatcccacccagcttgctttaagctcctccagcctcagcctcacaccctctacctttctctccccccacacctcccagctcctgctcacccaggtcctgcccaatcttccacctcagccactttatcctctgtccccaactgtggcacccagactctccatctcctccacctccacttcccacccaccgtcctgtctccctgggcctctgtcgtccccctctgatagctccaggccctgctctcctgtcctaccagcctcctggcccacagtccagcttctctgacctccctcttcgaagatcaacatcttgggaggacactgaggcacagagcagagtgtggccatgccccagggcacagagcttgcagcccagcccctctgtactgaccctgaaccctgaccttccattcccgccctctgaggcccccctcctgcaacaggcctgagctcagctcccctttcccaccacctgccctccctgctcccaacctccagccccgacagactttcccagtgcttccctgggcctctccaggtcctgagtcatcctgaccctctgtctaccacatgagcccaaatctgtgcctcagtctgccagtctctaaaactgcagtctgctaggccttggagtgccccacaccccaccttggagagcccagttctcccagcttgagttcctccctgccctcctccccaggcctctgcccctgccaggttgtcaccaaccacccctctcccaaaccaggctcaacaggggaacgaatctgccttgcgggggacagtgcaggcgggaacctctgcttcaccgtggctcttcgggcagcagcctacggggtgcgggtgccagatggcatcatggcagcctacccggccacaatgctgcagcctgccgcctctccctcccgcctgctgagcctcatggaccccttgctgcccctcagtgtgctctccaagtgtgtcagcgcctatgctggtgaggcccacaccctcagcagggaaacaggactgcatggatgtctgactcctgcagggacgggctggggaccaggactcctgtgactgaggaaggagaggccaggcagaggaatctaagactccttgtcccttttttttcgtgtgtgtgtgtgtgtgtgtgtgtgtgtgtgtgtgtgtgtgtgagagagagagagagacagacagacacaaggtgtcatgtcgctcaggctgagtgcagtggctcaaacacacctcactgcagccttgacctcccaggctgaagcaatcatcctgcctcagcttcccaagtagctggaactacaggcgtgcaccaccactaccacgcccagctaatttttgtgtttttcgctatgttgcccaggctggtcttgaactcctgggctcaagccatcctctcgccttggcctcccaaagtactgggactacaggcgtgagccactgcgcccagcctccttatcccttttgaagcgattggatattctcgggtccctgggaccacagatgcctgaagttctgggcgttaaaactgggcccagaaaggggaaaacaactcagctacccacatatattctgcaggtgcaaagacggaggaccactccaactcagaccagaaagccctcggcatgatggggctggtgcggcgggacacagccctgctcctccgagacttccgcctgggtgcctcctcatggctcaactccttcctggagttaagtgggcgcaagtcccagaagatgtcggagcccatagcaggtgagtgattcactcccgtcacatgcccagccagggtgggcaggagggacagcctagcacagcaaaagtcccagggaagatgctgggcatggtggttcagacctgcaatcccagcactttgggaggctgaggtgggcagatcacttgaggtcaggggttcaagatcagcctggtcaacatgacgaaaccctgtctctagtaaaagtacaaaaatattagccaggtgtggtggtgcacacctgtaatcccagcactttgggaggccaaggtgggcagatcacttgaggtcaggagttcgagaccagcctggccaatatggcaaaaccccttctctacaaaaaaataaattagcagggtatggtggagtacacctgtagtcccagctaccagagaggctgaagcatgagaaccgcttgaacctgggtagcagaggttgcagtgagccaagactgagcctctgtactccagcctgggtgacacagcgataccttgtctcaaaaaaaaaaatttaaaaaaagagtacgcttactgggctgtggtgaggtctgcctccaaccatttggccagtacaatatatggcacccagtaggtgctattagaagatgccagcacccctggtgtgtccattctgttccagtgaccctctgggccacaaagtacttaagtagttattctcacattgttttgcacctcaccttcgtagatttataaagacaagtaaggctgggtgtggtggcttacgcccataatcccaacactttgggaagccaaggcaggcggatcacttgaggtcaggagttcaagaccagcttggccaacatagtgaaaccccatctctattaaaaatacaaaaatgggccaggcgcaatggctcatacctgtaatcccagcactttgggaggccgaggcgggcagatcacgaggtcaggagtttgagaccagcctgaccaacatggtgaaaccccatccctactaaaaatacaaaaattagccgggcatggtggcatgcatctgtaatcccagctactcaggaggctgaggcaggagaatcacttgaacccaagagacgaggttgcagtgagccgagatcacgccactgcactcaagcctgggtaacagagtgagactgtctcaaaaaaaaaaaaaatacaaaaagtagctggacatggtggcatgcctgtaatcccagctactcaggaggctgtggcgggagaattgcttgaacccgggaggcagaggttacagtgagccgagatcgcaccactgcactccagcctgggcaacagagcaagactccatctcaaaaaaaaaaaaaaaaagtaaaaaactaaaactgtagcatcttggccctcatggtaggcagtgtgggtctgataaaatgaacagagctgaggccaagctgggcacgccaggtgggctggaagtgtatctttagcatcgtttgggcacttgcttgcctggtcacgtgaacggataaaactgaagaacacaaaggaaatgtggggaagcagatccatgtggctacaggtgccagacccctagaaatgtgatacaccctggagacttgttagctccctgtgaacagagcctcccttcaagccactgggcctgcttactattcaaatcattttgatccatttacaccttgtggtaagtgtaatcccagcactatgggaggccgaggcgagcggatcacttgaggccaggaattcgagaacagcctggccaacatggcgaaaccccatctctgctaaaaaaaaaacacaaaaattagccgggcatggtggcacatgcctatagtcccagctactcaggaagctgaggcacgagaatcacttgaacctgggaggtggaggttgcagtgagccatgttcttgccactgcactccagcctgggtgacagagcgagactttgtctcaaaaaaaaaaaaacaaaaaacgatatattatgatccccagtgggtagcggtacctaatcccacatcttatcccactgagtcctgccacctatgaaccaaggactattttcatttccttgatctagaagaaaaaataggttcctagaggtaaactgcacctaatcttcccagtcacacacacacacacacacacacacacacacacacacacacacacacacacatccttcactagaacacactgccccactgccaagacccccaccatgaccacctggggcatctggaactccacccatcccctaggcaagagtctggggaaacacagcctttcacagcgctccacaggcgactgcccttcccatggcattccctgccagccacacacacaaccaaactaacggagccaggcctatctgctctccctctctcagagccgatgcgccgcagtgtgtctgaagcagcactggcccagccccagggcccactgggcacggattccctcaagaacctgaccctgagggacttgagcctgaggggaaactccgagacgtcgtcggacacccccgagatgtcgctgtcagctgagacacttagcccctccacaccctccgatgtcaacttcttattaccacctgaggatgcaggggaagaggctgaggccaaaaatgagctgagccccatggacagaggcctgggcgtccgtgccgccttccccgagggtttccacccccgacgctccagccagggtgccacacagatgcccctctactcctcacccatagtcaagaaccccttcatgtcgccgctgctggcacccgacagcatgctcaagagcctgccacctgtgcacatcgtggtgagcgacagagggggccggtacaggtgcatttagagaagagcagggcacccggggaggagggggaccgggcacgggtacacctgggaaaaagagctccggagtggggagaaagggacaggcgtgtccccaggggagaacaatgccccgaacgaacgggaggaggacaagagtcagggctgcctccgttttactttgcaattttgagtaccaagagacaggatcagtggggcagccccttgggcttggagaatcaagagaaaaaaaaggtgggtttggcggggtggggagggagaggctggagagaggccggggatcgcagttaaagtcattctcccatccttcccacaactccccccattccctccattctctccccaactcctcacccaccatgtctcctgcctccgtccactccgtctgtatccgtatttcccgctccctttgttcatcaaacccctcctcgctctacccgaccccggtctgtctgtcccccaccctctctccacgtccctccccgctgtcccgtaggcgtgcgcgctggaccccatgctggacgactcggtcatgctcgcgcggcgactgcgcaacctgggccagccggtgacgctgcgcgtggtggaggacctgccgcacggcttcctgaccctagcggcgctgtgccgcgagacgcgccaggccgcagagctgtgcgtggagcgcatccgcctcgtcctcactcctcccgccggagccgggccgagcggggagacgggggctgcgggggtagacgggggctgcggggggcgacactaaaagcctgttgttcccatctgcgccggcctccgtcatgaatgccttccgggccgggcggaaggggacgcgggctgtgccttacttaagtcgggggtggcaagggggcggggcgggggcccgaaagctgagaccctcgccacggggagggggacgcgcacacacaccggtcaccgagacggctggacctgcacgccaccgctgccttttgctgctgctgctgcggcgaccgccgcagggacggggactggccctcccttgcaggtcggtttggtttgttgtaaataaaagtatttaattagtttggc

SEQ ID NO: 8 - DNA, 20872 bp, Homo sapiens
aatcagcgatcagactccggagtttggcccgggagctggggagctcaccgatcccccgcccagcagttctggccgctgtcccggtgcgcacggacgtggctcgagtttcctctgctctccgctctcgcccgctagctctcctcccttccgctcctgcttctctccgggtctcccgctccagctccagccccacccggccggtcccgcacggctccgggtagccatggaggaccccacgctctatattgtcgagcggccgcttcccgggtaccccgacgccgaggccccggagccttcctccgctggggctcaggcagcggaggagccgtcgggggccggctcagaagagctgatcaagtcggaccaggtgaacggcgtgctggtgctgagcctcctggacaaaatcatcggggccgtagaccagatccagctgactcaagcacagctggaggagcggcaggcggagatggagggcgcagtgcagagcatccagggcgagctgagcaagctgggcaaggcgcacgccaccacgagcaatacggtgagcaagctgctggagaaggtgcgcaaggtcagcgtcaacgtgaagaccgtgcgcggcagcctggagcgccaggcggggcagatcaagaagctggaggtcaacgaggccgagctgctgcggcgccgcaactttaaagtcatgatctaccaggtgagcccacagggcggaaggcgcccagagctcccgcgcccggcccgcgtcccctgcctgggagttggggtggggagacctgcccgtggcgctgctcccctcccagcctggcccgggcgcccggcagatccaccctccttccggtgagcgcggctggggtggggttgggtggggtaggatggggtggagggagcggcggactcctggggcgcgtagaggggcgcgctctgcacctcgcacctccccactcagatccttccctcgggtcccaaacttgtagcagaaccgaattagcgggtggactcgaggacgctgcccctcgcagaccgctccctccctaattggctctgtcaaacttcttcctacttgggggaagtttctaggaaagtcaggggtccctcgcgatcgttggggtccgctgtaagcggacactggctgcggggcccgggcctggggcctctgcgcctgcgggcctgatatagcgggtgattcagggctcaggcacgcaccagcgggggctgtgccaagcggggaacgcgaccgccaacctcctccccagaccctgcgcccccgcgcccccacatcggcctcggcggcccacccctgtcccctcgctgctctctgctcctggccaccaggcattttcaggcccttgaggacaccctcacccgcggcgcgctggcaccctgggcctcctggaagcgggcggggctgggcggggaagagtgggcgtcaggagtgcaagaggggctggtggagtgggtggacagcccctgcggggtccggcggtctgggcgcagtctgggcgaggtgaatcaccccgcatgcctgcgggggcgggggccaggcgcagcagcgcagccggcgggaggaggctggcgcccccaccccccggctccgcccggggccacttgttgctttctttgcagctctccccattcttcacacttggataaaaatgtagcttttggatcataacccccaataaacctgggagtctcgggtttctatgctgctcctctccacgccgcttccgcagtgccatcggggatgagattttgggctgggcggcaagtgttccagcctggctcctccccgcctcccaaaggcctgctgatgctcctaaccccagggaggcgggatcgtgtggggcaggggggttagtctatgctcccctctccccttcctctccctgctgaagggaatggaaagaaagggaaggctggatttcccacaccctccaagttagaagacccctttccgggagttgtggctctgctgactgctctggggcctggtcgagccgggttctgagtgaccatctggggtgaggcgagggggtccttggcttggtttccccaggaggagttaagatagatgcaaatcaggacaagtatctgggaagagtagagacagaaaggtgggtcagctggtacctatctgaggtggtgggacagaggcagaagtggcacttgtccccctccccacccttacccacggatgacagactcctcccaggaggcatatacgtggcttttaaaagctttacttcccctacttaagcactatttagatagaaaatgtttgcccagagctatttccacttagtctaactgcttggaggacgcaggatgagaggggggcttttgacgttttttaagagacctgcttagtactggggacattgagaggagcggaacaagacaggggtctgggacagcaagccagctctatatttcctccagagcctggtgtggccgagagccaccatgaagagctcgaggggtgacccttctcacgctaactatcccagaccatagaacctgggaaaagtcttggcctggaggtaaacttccctatgccaaagatggagattctgagagaagagggaaaggtatggagggagaaagacttatctgcagaggccactggcatccctctgttctgccctctccagcaatctgcagggatatgtctatattttagggggtgggagggaatgtgttctaatcacaggtggcttcaaatgaatctgcactaaaccaaggccagtgggtgacagagagttgaggtagtaagagtagctcaaatggagtcccaaacccacagagggaataactaagaactgaaatggtcccaacctggggaagacttcagacgtaaggaaaagattccagcaaatggggtgaggcttgggaaagcagaacttccctgagctgcagagcagatgttccaggtggggacccggctgctggaggaaagcagaggctgctcctgctcctcccagaggtctgcaggcctgagaagggaaggaggtaaaaatagctctgtgccttggcaggcctattagggaagattgggggcagcagcatttatggtaaagtccggctgaagggggaggatgttgggtggccaggcaaagagacagagatttggagaaagtttcctgggatggtgtgaattctctggaggggccataggggaagggatgctgtattccccactacaggggtgggaggtgatgatgtggcagtgggggcggggcagagaacaccaagggatgggaaggcccccgttgtgggatggagacatctggccttccataccctattcctggggagaaaaggagggaatcccttgtatggccccaccccaaattcagggaacttctagacttaatgatttcacagctttaagtttgcatttgaacacacacacacacacacacacacacacacacacacgaaattcagcacctcccaccccaaggtatcacagccctcatcttaaactcacttctgagtcattactgcccttctctggcatttccttccctgtctcccaaacaaaacctagcagtttgacattgcattctatcaggcgacacttggctagcaacagctagtaaatgagagaagtgaattctactagttataggagttaatggactgagggttgcaggacgatcaggccagattgtgaatatagaggcagttgctctaaaataccatgcagaggccgggctaggtggctcgtgcctgtaatcccagcactttaggaggctgaggcaggtggatcacttgaggtcagtagttcgagaccagcctggccaacatggtgaaacccctctctactaaaatacaaaaattagccgggcatggtggtgagcacctctaatcctggttactcgggaggctgaggcagaagaatggcttgaacctgggaggcggaggttgcagtgagcgagatcgtgttattgcactccagcctgggcgacagagtgagactccatctcaaaaataataataataataataataacaacaataaataaaataaaatgccatgcagatgagtggaactatgatcattaaagaaccaattcttccaggcaacaatttgttttttggggttttttggttttgtttttcagacaagttctcactctgttgctcaagctggagtgcagtgacacagtcatatcttactgcagtctcgaactcttgggctcaagtgatcctcctggttcagcctcccgagtagctaggactacaggtgcccaccaccgtgcccggcaaattgttttttttcatatttgtagagtccggggtctcacttagttgcccaggctgatctcaaactcctgggttcaaacagttctcctgtctgggcctcccaaagtgctgagactacaggcatgagctaccacgtgcatccagtttgtttatttttaaacttaaaaggctttatttttaattactatggatacataatagttgtacatatttttggagtacatgtgatatttcgagacaagtatgcaatgtataatgatcaaatcagggtaattagagtatccgtcatatcaagcatttatcatttccttgtgttaggaacatcccaattccactcctttaattattttgaaatacgcaataatttctgttaactctatcaccctattgtgctactgaacgttagatcttattccttctatctaactgtatttttgtacccattaaccatccctccaggcaacaatttggagagtagggctcttttagatccagaaagtggggataaaatggaaatacggggctgggcgcggtggctcacacctgtaatcccaacactttgggaggctgaggcgggcggatcacctgaggtggggagttcgagaccagcctgaccaacatggagaaaccccgtctctactaaaaatacaaaattagccaggcgtggttgcacatacctgtaatcccagctactcaggaggctgaggcaggagaatcacttgaacccaggaggcagaggttgcggtgagccgagatcgcaccattgcactccagcctgggcaacaagagcgaaacttggtctcaaaaaaaaaaaaaaaaaaaggaaatacagtcacgcattgcttaatgactgggatatgttcagagaaatgcagtgttaggccatttcctcactgtgtgaacatcatagagtaactttacataaacctaaacagtatagcctactgcacacctaggctatatgatacagcctattgctcctaggctacaaacctatacagcatgttaatgtactgaatactgtaggcaactataacacaatggtaagtatttgtgtatctaaatgtgtctaaacatagaaaaggtgcaatagccatggcacggtagctcacacctgtaatcccagcactttgggaggccgaggcaggtagatcacttgaggtcaggagttagagactagcctggccaacatggtgaaaccccatctctaaataaaaatacaaaaattaggtgggtgtggtggtgggtgcctgtaatcccagctacttgggaggctgaggcaggggaatcgtttgaacctgggagatggaggttgtgcctggacaagagagcaagactgtctcaaaaaaatagtatagtatagtaaatacataaccagtaacacagtcatgtcttatcaatatcaagtattatgtactgtatataattgtatgggctagacttttatacaactggtagtgcagcaggcttgtttacaccagcaatcaccataaacacttgagtaattcatttcactaacacattatgatggctacaatgttattaggtgataggaattttttatctccactacaatctttttttttttttttggagacaagttctcgctctgttgctcaggctagagtgcagtggcacaatcttggctcactgcaacccctctgcctcccaggttcaaacaattgttcttcttcagccttccgagtagctgggatttacaggtgtgagccaccatgcctggctaatatttttgtatttttaatagagacagggtttcaccatgttggccaggctggtctcgaactcctggcctcaagcaatccacctgccttggcctcccaaagtgctaagattacaggcataagccaccacaaccagcctccattacaatcttatgggaccacggttgtgtatgccgtctgttgactgaaacgttgctatgcggctcatgactaaaaatgtgttctggagcagttttgaggaggcaaagtcagtgacctacatatggaccagcagcaaaggcccatcagacctgctccaagtgcctggggataagggccaggaagcatttaagggattaggtgtctcatgggcattaaagggtagggcaccagggctggaacgagagcctgatggcttctcctccccaaatcgcaatccaacctgtacctctttcctggattcaacgcaaattagggctaccttgctctagaagaaccccaaacctttgtccaaaaatattctcaacccagggctttggtccctttcatccctaaaacccagcggccatggagagtatgcatagtacatcccttcctcccaaactgagtttgggttgtttttcctggtcactgggtccaaagcagggagggtatttttaggggtattcccaagacgaaaggaaatcatgctatccctgggttcctccctccctttttttttttttttttttttttttgagaaaggatcttagctctgcagcccaggctggagtggtggtatcatagctcactgcagcctcaaactcctaggctcaagcaatcctcccatctcaccatctcagcctcccaagtagctggtactacaggcacaagccaccatgcccagctaatttttaaattccttccggccttctttccttcttcttttcttcttttctctctctctctctctctcttctttctttgacagagtcttgctctgtcacccaggccggagtgcagtagcgtgatctcagctcactgtaacctccacctcctggattcaagcaattttcatgcctcaccgtcctaagtagctgggattacaggcatgtgccaccacacctggctaatttttgtatttttagcagagacggggtttcaccatgttggctaggctggtctcgaactcctgacctcaggtgatccacctgcctcagcctcccaaagtgctggaattacaggtgggagccactgtgctcagcctaaatgttttttctagagggtccccactgttgcttaggctggtctcgaattcctgggcataagtgatactcctgccttggccgcccaaagtgctggggttacaggcgtgagccacgcggcctttttatttttatttttcatattgtaaagatcaaagtcaaaagcatgaggagacagcaaagagtgggcaactgatagcaagaggggaggggatattattgctaagcaactaacctcagaagtaggagtggcttgggccatttttttttggaaatataacattgtgaatatttcatcattctgtcattagcattttttaaatttttcttttctttttttctttttttttgttttttgttttgttttgtttttctgagacagggtctcactctgttgcccaggctggaatgtagtggcagctgtcacggctcactgcagcccctgtcgtccgggctcaagcattcctcctgccttaggcccccaagtagctaggactacagacatgcaccaccacatccagctaatttttgtattttttggtggagatggggtttctccacattgcccagggtctccaattcctgagctcaagcaatccgtctgcctcagcctcccaaagtgctgggattacaggcgtgagccaccgcacccggcctcattatacacttcttgagctagaagacactttgcaaataatcaacttcaactccttcattttgtggtttaggaagctgaggcccagagaggggaagtcactagcctattgtcactcaagtgattgggagtcaaactggagccagaacccaggtgtacccttccgctacactcactgctccccaccccctccttccgttctgggctctgcagcagaaagctgagtgaagactagccctgacaaagctagggctagctgtggctccgacacttgctgagggagttagttgcacagtcctggccctggggctgctttgcgtgtgtatgggtgtttgtgtgtgtggcgttggctatgagtggccagcattggtggccattggctgcttagccttgtaagtgctgaagtagaggcaatgtgtggtcacctggcctggtgacagtgggaggggagaggtgtaggggaggggaggggtgtaggggagaaaagtgggggaaaggaggggtgttggggaggggaggggcgtaggggaggagtggagctcctggtcagccgtggtgtgccctggcagggactccacacatacctaggagagggacatgttgtttccctttctcttcagcctctctgccactcccacctcctccttgtaccctgcaggggaggttgggacctgctgctttgtttttttgagactacaggctttatcaaatactgctattgcctgagtccaatagtgccgccaagtgtccctctcgcctggcatctgattgctcaaagcaagaagcctatagtcttccacaactcagcccctcaggagagagagttgggggctgccgcagaggtccagaggtcccaggacataggctcacctgtcataaaaatagcttggttttgccaggcacagtgatgtgtgcctgtagtcccagctactcaataagttgaggcaggaagattgcttgagactaggagtttgaagccaggttggacagcatagagagactctatcttaaaaaaaaaaatagagcttggttttggatcctgatttctctgtgcattttagtgtatgatatcttaagtggtagcaagagggaggggtggcaagggagacagaaggtggggacgtggccaaagggaaattgccaggatgtccctcttccatgcctatccctctcctcatttggggtagcatcctgtgtccaccctaccccagggaagaatgaaaacagattaatctgggcacactggctcatgcctgtaatcccagcactttgggaggctgaggtgggtggaccacttgaggtcaggagttcaagaccagcctggccaacatggcgaaactccgtctctaccaaaaatacaaaaaaaattagccgggcatggtggcacacacctgtaatcccagctactagggaggctgaggcaggagaatcacttgaactcaggaggcagaggttgcagtgagccaagatcacgccactatactccacagcctgggtgacagagcaagactccgtctaaaaaaaaacaacaaccacaaaactgggattggcagagtctgactctgaagacagtgaagttgtgtgcctttgtatttactcatctgagcctccccagttataatccaacaactagccccttagaaaactccttatccacccctagtagcctccatagtcttcctatgattatggctggggaaggattggagatggaagcccaagaatgggactggggaatattgtagagggaggaagggaggggctcaattgaagctgtgtttattaattcttgggcttgagtttgccccaccccttttggccccagggacctctttttttttttttttttttttttttttttttttccctttttgagacagacttttgctctgtcgcccaggctaaagtgaagtggctggacctcagctcactgcaaacttcacctccctggttcaagtgattctcctgcctcagcctcccgagtagctgaaatttcaggcatgtgccaacaggccccactaacagtattttttgcagagacctgggcaatgttgctgaggctggtcttgaactcctgggctcaagggatccacccaccttggcctccaaagtgctgggattacagggattataggcgtgagccactgtgcccagctcccagggccctcttgatgaagcatttaaaatgagtttctaggccaggcatgatggtggctcatgcctgtaatcccagcactttgggagcccaaggagggcagatcacttgaggtcaggagttcaagaccagcctggccaacatggtgaaaccctatctctgctaaaatttcaaaatattagctgagcatggtggcacccacctgtaatcccagctacttgggaggctgaggcaggagaatcgcttgaacccaggaggcggaggttgcggtgaaccaagatcagagatcacgccactgcactacagtttagcagcctgggcgatagagagaggctccatctcaaaaaaataaataaaatagtccaggcgcagtggttcccgcctgtaatcccagcactttgggagaccgagacaagaggatcgcttgaggccaggagttcaagaccagcctggccaacacagtgaaacccggtctctactaaaagtataaaattagctgggcgtggtagcatgcacctgtaatcccagctattcgggaggctgaggcaggagaattacttgaacctgggaggtggaggttgcagtgagccaagatcgtgccactgcactccagcctgggcaacagagtgagactctgtctcaaaaaaataaaataaaatagtttctagcctgggcaacatagtgagaccttgtctctatcaaaaaaaaaaaaaaaattagccgggtttgatggtacacaactgtagtcctagatatttgggaggctgaggtaagaagattgcttgagcccaggagttggaggctgcagtgagctatgattgtgcactgcactacagcctgggcaacagagcaagacccccatttctaaaaaaaaaaaaaaaaaaaaagtggtttatttccatcctaatacattgtgctcccctccctactcaccagcccctactctgcatgccaccaaggcccttttttctcaactccttttgcacccaccccaagccccatttaaaccccttttcctcatcttagcccaagaagaagccgaatgcaccctctgacctggccacacccaagatcgtgccttttgacctgcctgagactgagccctttgacctgaagcctgagacagtgcctgagctcccaccttttgaccagacgcctagggcactgccatattacccagagcctgggccccggcccaccccagaggagctgacagccggtgggagtgtttagggtctcccggatcccgttctgtgggcgtggggtatctcttcagtccactgtcagcccacagcctagtggtgatcactgccaggagggccaccatccccagtcactgcccactgtggacgggtgccgggtactgtgagaacactggcgttatgcaaagttcaggagaatgggatgtatacagtggggatgggaggggaatggaaggggcctccccacttcttggtttctgtttttagtagtaaatgctcccccacacttaagctgcacccgaaggctgcctagcatgaaggcgcatggcttctggttctgccgttccccctgctgcctcctcctgagggacaacccagtttggctcaaagcctctttggagagggccatcatgaggctcaggcctggggtctgttctgtgccctctccacccactccctgcagctgaggaaaacatttacacgtattttctccagtttcttttagaaaatgctttgcaaagagatcgcttatgtttgccagatcctgccttgggtggggtgcccaaaagctgccaactcacagatgccaggagggggacattctagctggctctggctcaggacctctggccttcctatagcagagtgtttctcaacttaaaaaaaatgttaaaaaaattttttttagagatggggttggactgtgttgcccaggctggttctgaactcctggggctcaagtgatcctcccacctcagcttcccaaagtgctgggattacagatgtgagccacctcacccagccagtttttgttgttgatggttgttgttgttttcataatcactcccatatacagaccttttagtttttttttttttttttgagacatagtcctactctgtcacccaggttggagtgcagtggagtgatctcggctaactacaacctccacctcctcggttcaagtgattctcctgcctcagcctcccaagtagctgggattacaggcatgtgccaccacacccagctagtttttgtattttttggtagagacggggtttcgaactcctgacctcaggtgatctgccccgcctcccaaaattctgggattacaggcatgagccaccacccctggccctttcaattttttttttttttttttttttttttttttttttttttgagacagagtctcactccgtcgccaggctggattacagtggcacgatctcggctcactgcaatctccacctccctggttcagcctcccgagtagctgggattacaggctcgtgccaccacacccagctaatttttgtatttttagtagagacggagtttcaccatgttggccagaatggtctcgatctcctgacctcgtaatctgcccacctcggcctcccaaagtgctgggattacagccttgagccacagcacccggcccctttcagatatttttaaccaagctgttcatctccctcacgaaatgttaaaacaatagataaactgtatatctgtttatgtactgtggctcttgggagtggcacaaatcattgtgatatctaagtcccctcccaatctccaagaatcagttttcacccttttggatgcaatttcacccctgccaagaatgcacgctagagcagaagggagggacacttctgcccttggggtctctcatctccttgtcttagcaacctctagcagaggtcctgaattttaaagacgttctctccccttcacctctcccctcctctgcaccctcttctaacatacactctaagcagatcctgggtctctgaattacctctccagtctcctccagccattcccaggcagtggttggggacctagagctggtcagccttaagtcaagagagcctcctacccagacccatgtggcatctgtcccttctccctaacaaatgtgaagtgcctggccaagggccagatgtgtaataggtgctcaatgacattataatcttataaaaatcttcctaagccagatctcgagtttagacagcagggttggggggtggagtggaagaggctgggagcctccctgtggctcctctgcatccatcgaggattgaggagctatgtgtgcttggcaaagggcagggggaactgtaatgcttttgttcagagctggtcattaaaggtaactctggaaaccaagcctattgggagtggtggttttgtttaataaaaacgagaaacagaacaagcagagccgtccctccttgccatcccctgaggtccttcctttggcttaggagctctgccagagaagtaggggtgggacaagggtgcctggcctgtgtgcccagggtggagggagatgattttggattgtgtttgtggtggttgtaccagcaagcagatgggtgggaccaagacagcactccaaggctgtgagtcatgggggaaacctggcctggagtggagaggaaaagctaatggtgaaataccctctccagagccagcctattggacatcatcccaggattcagctcaagttgtttcgtctcctgtcccctgaccttccaccaagaaggcagctcctatggtgtaagccgtcactccaggggaagcctacgatgaatggggttagagcccccagatcccttactggtctggagagatttctaaatccaggaataaacctccccttggccaggcacagtggctcatgcgagaaatctcagcactttgggaggccgaggcaaggggatcacttgagcccaggagtttgagaccagcctgggcaacatagtgagactctgcctgtacaaaaactacaaaaattagctgggcatggtggcgtgcgcctgtagttccagctacttgggaggctgaggtgggaggatcactagagcccaggaggttgaggctgtagtgacctgtaatcatgccactgcacttcagcctggttgacagaacaagaccccatctcaaaacaaacaaacaaaaacacaaaaatggccagatgcggtggctcgtgcctgtaatcccagcactttgggaggccgaggtgggaggatcccttgaggtcaggagtttgagaacagcctggccaacatggtgaaaccctgtctctaccaaaaaatacaaaaactagccaggtgtggtggtgcatgcctgtaatcccagctacttgggaggctgaggcaggagaactgcttgaacccaggaggcagaggttgcagtgagccgagatcgcgccactgcactccaacctaggtgacagagtgagaccatgtctcaaaaaaaaaaaaaaaaaaggccgggcacggtggcttaggcttgtaatcccagcactttgggaggctgaggagggcgaatcacatgaggttgggagttcaagaccagcctgatcaacatggagaaaccccgtctctactaaaaatacaaaattagccaggtgtggtggcacatgcctgtaatctcagctatttgggaggcccaggcaggagaattgcttgaacctaggaggcggatgttgcggtgagccgagatcgtgtcattgcactccagcctggggagcaagagcaaaactccatctcaaaaaaaacaaaaaaactaaaaaacaaaaacctcctctcactaggattgcttccttatgttgtagtttgagacctttcatgaggcactaggaagatggcattggctataagaagtctgcagctctctccccagtcccattcacttccaagttcagctggcaaagacctttatctaggaagccacaggccaggaaaaccagccagctcagcctttaggggtggggtggagcaggcagcaggggtaggggagaagcgggggatgctgggggtagggcagagagacaaagtggatttgttttccagcagctggggctgtttcctgactattccctgcctgtcacccttaatccctccctagcctattcacattgtctgagtttagtgaaaggaggggctctctccaccctttggtggggcgtgggtgggtttttaagtctgggaggttatgaaggaaagatcagagctgaattttttttaatatatatatatactggggctgcaggtaaattctgggtggagatggatgaagagggtttagatcctacaatgatgtataagtcttaaatgatatgcactccagtagcaggcagggtaagggtgattgtaggagccccaaagtgaatgactggaaatcagagggaatgcaaagagaagttgagactctcctacactcagaacaaggaaaacacccttcaatgcctcagtgaatgataagaggcagagggataaacaaaatgccctaggattgcccatgatctgcactaagccgtctatggcaggtagagactacaagtcccagcatgccctgtgcccaggtcaccttgcctgctgggaggtgtagttgggtaacagagagctggaggtggcttggtgcccagcagagctgatgcccagatgagagaagaggtggagccagcattccctctttgtccctgctaatgctattgacaagaagggaaggaagttgggagagggtgttcctagttttcagtgtaaaccaggaaactctggggggatgttgatgggagggaagaggaaagaggttgggggctggggaaggaatttctgtagcaggtggtgcagagataccgagcagcctatgtataggggtgtgtgtgtgtggtgtcagtgagaggactcagtttatgttgccttaatttcttaggggacaggatttttcttataattctgggaaatggtcttcagaaaaccctttagaaaagcgactatctctcgttccttcatttgaagtcactaaagggagtctgggccaaagagatcctattgatgatcttggtctgccctttccctttctgtgctcagcctcccctgctctctgatgagggtggaggtgggggagggagtgatccagagattctgaaatgttggccagagtcaagatcttggaaaatcttcccggaatggaaagcagttctggtgctaagtcctccaaggagaagacagaaaagcaaagaactggttctgagctccagggtcatggggaatctgagaccacccatcctactcctagacccctgagctcaggctttgtgctgggggcagggggtagggaaagcaagaattgaaaagtaatctctcttctatcccaaagtagggaagtcacaaccagaagaaatgtccagactggggagaaaacatgtccgtggctcccagccagcttcttcctctttccaatatcaatttctaaatgtgggggagggggagaatgaccactggtggagccaaacagtatgaaggagtcaaagaaggtggaggtgggggagggtggaccacagttctccatctctaaggctggttctgaaatagcttgaggccaggagcagtggcttatgcctgtaatcccagcactttgggaggccaaggcgggtggatcatttgaggtcaggggttcgagaccaatctgaccaacatggtgaaaccccgagtctactaaaaatacaaaaaaattagccaggtgtggtggcgcatgcctgcagtcccagctactcaggaggctgaggcaggagaattgcttgaacctgggaggcagaggttgcagtcagcagagatcgtgtcactgcactgcagcctgggcgacaaagcgagactccatctcaaaaaaaaaaaaaaaaagaaatagcttgagtctaggaaatgcacttcctttactttacctgcttgggtgggtaggtgcagctgtgggctcaaacccctacccgctttcaacttagacactcccaccacttgcttttcggcctggctcgggcagtgtgcgatttgcggcaccagagggcgctccgcccatttccaaagtggagctgtgtctcccgagggccgtagtggtttaatatcatctacgggtggaaagggatcaggacagggttggacgaccacagagattttagcccaagtgcaattattacgcaaccccatctccgacccgaagtttggtcccctgctcccgcggtctgtttagtcaccattcgttcaacacacatgttttttagagaatggcggcgcgaggccgggcgcagtggctcacgcctgtaatcccagaactttgggaggccgaggcgggcggatcacctgaggtcgggagttcgagaccagcctgaccaatatggagaaacaccgtctctattaaagatacaaaatttagccggcgtggtggcgggcgcctgtaatcccagctattcgggaggctgaggcaggagaattgcttgaacccgggtggcggaggttgcggtgagccaagatcgcgcccactgcactccagcctcggcaacagatcgagactccgtttaaaaaaaaaaaaaaaaaaaaaaacaaggagagagagagagaaagaatggcgacgcggggttggggggccggagatgggtgatatgttgcccaggctggtctcgaactcctggactcaagccatcctcctgcctcggtctcccacagcgctaatattacaggcgtgagcctacacacatttattgagcgcccgccagtttcctggggaccacgagcgtcccatgctcccctctctccaggatcgtcaccaccctgactcaggctcgcccacatccgcctcctccgacgcctctcatgtcccttctcctcttcccttaggatgaagtgaagctgccggccaaactgagcatcagcaaatcgctgaaagagtcggaggcgctgccagagaaggagggcgaggagctgggcgagggcgagcggcccgaggaggacgcagcggcgctggagctttcgtcggacgaggcggtggaggttgaggaggttattgaggagtcccgcgcagagcgtatcaagcgcagcggcctgcggcgcgtggacgacttcaagaaggccttctccaaggagaagatggagaagaccaaggtgcgtacccgcgagaacctggagaagacgcgcctcaagaccaaggaaaacctggagaagacgcggcacaccctggagaagcgcatgaacaagctgggcacgcgcctggtgcccgccgagcggcgcgagaaactgaagacgtcgcgggacaagttgcgcaaatccttcacgcccgaccacgtggtgtacgcgcgctccaagaccgcggtctacaaggtgccacccttcaccttccacgtcaagaagatccgcgagggccaggtggaagtgctcaaggccaccgagatggtggaggtgggcgccgacgacgacgagggcggcgcggagcgcggggaggccggcgacctgcggcgcgggagcagccccgacgtgcacgcgctgctggagatcaccgaggagtcggacgccgtgctggtggacaagagcgacagcgactgagccgcccccgctgccacccaccccattcctcgctccttccgaacttcctctttcgcattctctctcggctcgagctggctgagatttttctaaattgaaaacacgcccccctccccacacctccaggaactccactcccagtcttagagctgttaggacccgatggggaggcagcccccgcagtggacaacccccgcttggacacagtccgagtggaatgggaagggaatggtcaatccctgtcctggttgtccaagtcgggatctcagaggaaattgcagtgattccacggttaggcccccctgggggggctgccttcccctcagcctctccccacaccacccacccagctgctgtcattccgctcactgagctcttcttcattctcaccctgatccctgggggactcaaagccaaaactgcccaaagaggaaagattgaatcctaaaggggatccctgcccccatgggaggccccctactagaaggacgtgaaagcagcttttgggggaaactgaggcagtggggaagacagagcagaatgagccctcaccctggctgggggtccagcacaggctgtatctgcagagggtcccagaggaacgctggagccaagagaagccctgggaaggaggggtggggaacgacatgcatgtgagggatggcacactgatgtgtttatgcacctgtacacaggagcgcatggccatggctttggaaaggagaatggaaaaatagaagaaggtcggccgggcttggtggcttatgcctgtaaccccagcactttgggaggccgaggtgggcggatcacctgaagtcaggagttcgggaccagcctggcaaacaccccatctctactaagcgaaaacccatctctactaaaattacaaaaattagctgggcatggttgcgcatgcctgtaatcccagctactttggaggctgaggtggggagaattgcttgaacctgggaggtggaggttgcagtgagccaaggtcgcgacactgcactccagcctgggtgacagagtgagactccatctcaacagaaggaaaaaaaaggaaaataggagaaggtggaaatgggtgaagagagaagtcccctcactagctgcatgagaaatctatcttactgtggttctccatgggcagcaggaccatttttcagaatcaagagggaggacagtgtgagaaggcgatgatccaaagaagacagagaggtcagccccacccgatccctcaaatgggctcttggaggcacccccaggggcagcccatttctcaaagtccagaaaattagggtcccagaagggggcagcagcaggctgggagttaggagggagagcagggtgccggccctgccaccaagttgagagctggaggggaggtggggagagaacatcacagagcagccagccctggttcactcctggcagtttcttctcaagctccttccctaggagcatggtggcacgtgcctgtggtctcagctacttgggaggctgaggcgggaggatcgccagagcccaggagtttgaggctgcagtgagctatgagggtgcctctgtgctccatcctaggcaacagagtgatacgctgtttaaaaaggaaaaaatccttccctagagctagtatcctaaagctgcagagctagcccagacctcattggtttccttgtccttggggtgcttttcctgaatctttgcgggtgaagggagtgttgctcccagtccagaggcctgattctgtttggactgggttctcaagacacgaccaggttctcaagacacgagtccccttgttcctccccattaaagggggtttgtcagaagcaagaacagcccctctccccagtcacagcctgaagggaggccccgagagcttcctccttccccccacctgctccttaccttctctgccctgctttttagaactgcagttcattgttttaagggattgggggagggagcctggggacacaaaccttttatacaatacaaagctttgctttttttttttttttcttccttttccctttctcggttctcttctctcctctgaatggctgaagacccctctgccgagggaggttggggattgtgggacaaggtcccttggtgctgatggcctgaaggggcctgagctgtgggcagatgcagttttctgtgggcttggggaacctctcacgttgctgtgtcctggtgagcagcccgaccaataaacctgcttttctaaaagga

SEQ ID NO: 9 - DNA, 36401 bp, Homo sapiens
gggagaaacgttctcactcgctctctgctcgctgcgggcgctccccgccctctgctgccagaaccttggggatgtgcctagacccggcgcagcacacgtccgggccaaccgcgagcagaacaaacctttggcgggcggccaggaggctccctcccagccaccgcccccctccagcgcctttttttccccccatacaatacaagatcttccttcctcagttcccttaaagcacagcccagggaaacctcctcacagttttcatccagccacgggccagcatgtctgggggcaaatacgtagactcggaggtaggcatccgtgggggggcgccggctcgggcgtgcggggagtgtccgcttctgctatctgcctctccaaatatcccgactgctgccctggccccagccctctctccacttcggagcactcctctggcgttggcaccgctgaggaatgggcctgggcggggaggtgaagagaagccaggaatgttttatgttttcctaatggagagggggcctagggagcccctgagctaggaggacacggaaaaggggattggggtcctgagattgggtctgttgggcccaggacgcgttttctggatgggtctaggatgctcccttgtcgcgggacccccgcggtccggccctgcctgctgggggttcgaagaggtggagtgcagggtggaggtgttatttacccgagtcctggggacagtccccgggactctccgccaggcgcccagaccggcaggtcccgcaggcggcgcgcggtgtgtttgcactttccaaagttcttgaaccatctcaagaactccttctgcatcttggcgtctggcaggggtgttccgagagaggtagacctcccctccccaaactgccaccatcacttccaacgccctccacgcgctggagctctgcccgggtgtggaaacctcgtcttccaacacgtagctgcccttcagccacccgcccgcagcctgggagtgccctgagggtgggtcgggggagctgcgcaggtgagactgagttctaggacatttagggggtctggtgcctggctccgccaaaaatggggactttcgggattgtgatcatcacggcggattgagcagggagagccgtggagggacaagagagggccgaggcagggtggggggcgcgggcaggtgcgagggggatgcggccaagaagcagcgataaagggaacattccacgggtcgggcggctgctgttggatcttagataaagctggaagggattaccggggcaggggtaatagggaccggggacgggaacgcgaaacaggtgaagcgctcagggcgagagcgactcggcttagggagtccgggagaagcctgcggctgccccctcgccgccgaggtcctgcgggtcctgcgggtcctgcgtgctgagccggggcgtgcgcgggcgggggccttcggaccgcgcggcggggcctgccctgacccctggcggcgggcgggggaggcaggcgcgccctgcagagtacagaggggtgtggtgtcctctgcgagatcctcttaaaaagctggctacgcgcaggcggtttctgtgcacggagccgtagctgtcggagcggttagttcgatttcgagctcgaggtttcccccgccgccaggctgacttctcatcgcttgtttttctttttgcatttttcctcccaccgccgttgccgccctccccgtcctggccgtccgccctccgccctctgcagggacatctctacaccgttcccatccgggaacagggcaacatctacaagcccaacaacaaggccatggcagacgagctgagcgagaagcaagtgtacgacgcgcacaccaaggagatcgacctggtcaaccgcgaccctaaacacctcaacgatgacgtggtcaaggtaagccaaggcgaccaacagggaagggctgggacagctctcctctggcagttagcccgtgcatccttctttagcattgccgtgtacgcacaccccaccccgccccctacacgcgcacacacacacacacacagagttttgtgggtttgatgtgtgggagctcccgcagtcggcagaaacgttacatctcccttcccccatctccccccaatagttagttcagctgaaattcagctaaagtgagttttgtagaagttcctataactacacttttatcctagcaaatgagcctattgacctcagcaacagacggcccatactccttgggacggtgagatggttcctatccattcccaggttgaaagtctagtgacaggtccccactgcacgtggcattaagacagtcagataattgtgtcaggtcttgtgctgaggatgagtcagaatacaagatgggcatgttcccccaactaaaacgatgggaagtgattttcttaaaaatactacagtggatggaaatgcctaggactaaagacaaagaaaatacgtacttattcatatacatatgaaagttactttaactagactaacaagtcacttgtgcacaactaagcaaatttacaaaaccaaaaacaatgtatgcctcttggtttcttctatctatggacacctgcacttagatgtggaaagctgcttctttagtagctacctgggtcagcctgccctgagctaatggcacattcaggttggagttccttttcatactttcaggatgtgcttggtgagattaaaaataattggactgggttattggccagacttagatctgactcagtggtcagttttaaattatcattgttattagattttgacccttttagccaatctagtgggaggaatttattgcctaaacacatctggattgggatatcatgggctagagccatccttggcaaagggttttctctgagaaatggagggctaaggaaaaatcctggctcagggactgcagtgtgaagatctactcctatacaacccccagcaatcaatgaggcggatgagcaatttccacccaccacgcctgctatctatggatgggaggagctatagttcacaaaccgtttacattcatgaataatatatttcaaaaggggaaacagtttaatctgtaactggaagggaaaaaaaaactgtcagaattgactcccttggcttcctggagtaggaaaaaggaaaattggagcatttgcagctttttttgactagctggattatggaatatttaaaagcaacagcaacaaaagtaccttataaactagaaaatagaattgctaaaaaactatttactaaaaacattaccttaaagggagaggatatttgtgttttcccccacccccacccttctcatgtggctttgaacaagaaggagagttgccaggaaaagaggcagatttcagagagggctggcttcactggatcctccctgttgttccactgcactgtgagtgagattccctggagcaagcgaatctcccgggatgagtcagagaggccaacagtgtggatgtgggtctccacacatagcatgactaagttgagaaagaaaggccccactgggaaaagagacttcaacacagatggaaaaaaaacataacaggcttggaggaaatagcagtttacaaaacagcatttcaaagagcaagtgtggggatcctcaaattaaagaaattaaaagaaaaagctagagcaagctcctgctagcctaaagaaaccaaaccctgactacttgctcatagaactgtgagcaaaacaagacagtcaaaccaaaaaatccacctagaaaagaatttggcagtctcactcagatgcctggcctagaggggacttcagagaatgccctacagagagacaccaagactacaaatgcaaattctgcccaaagagtgcctggccgatgaacagggtcctatctacatcttatggagactcctattttataaatatgtatcctcaagtccaagcacaaacaaaataacagaaacagggatgattctctcccagtttccatgacagtaaataataaatttccctaaattttactttcaacaacatagactttttttatttttatttttatttatttatttatttattttttgagacggagtctcactctgtcacccaggctggagtgcagtggcatgatctgggatcactgcaacctccacctcccaggttcaagcaattcttctgtctcagcctcctgagtagctgggactacaagtgcacgccaccatgccgggttaatatttgtatttttagtggagacggggtttcaccatgttggccaggctggtcttgaactcctgacctcaagtgatccactggtcttggcctcccaaagtgttgggattacagatgtgagccactacacctggccaacaacacagacttcttaaaaaaatcatgacaataattttgggtgcttcttaaaagcacccaaagctttactgctaatgcatggtagcttaaaacttcacataataagaaagaaccagtggccaatggaatctactgttaaaggtacccaatcaagtaaggaaaagttggtcctaaaagcaagcagccctgtaaaagctgctctgtccaatatggtaatcactagccatttgtgtttccatttaaatttcaagtaattaatatcaagtaaaatttaaaattcagttccttagtcacactagccacgttgtgagtgtgcaacaggtaaagctagtggcacagacatagaacatttccatcagcacagaaatctctattggacagtgccagattagggtgttctctgcattgtaaaagcatccccttgccaagttaaagaaaacaacaacaaaactctagagaagaaatgaaaccccagtttcatttctggagaggaaagaaaactcatgtgtggcatgagtttatattcaagaaggtgcagcattattacctattttactagtaataatgacacacattatagtatacaatccagttccaataaaattaatttctcatcttactaaaagcttgctgctccacattatgagacaatttacccaaatatagacatttacccaaaaatattaagtagcttgtgaatactttttaaaatttcctttaattaaagtggtcacaaactcaaacccttcattctccctctgagatttctgtgtcatcttttgttcacattgttattcacatgtttattatgtacttattttgattttctagataaataaaatggcttcaaatctataattctgataaaattagccatcaattaatttatttattaaacccatgcaatatgctagattagatgctttgctatgtaattcctacaataaatcctagcaatcacaaagattacagttagtgagacgacatgcacacaggtaaaaagtgtttttaaaaaatacatacatacaaccaaaacagtaagtcactgctacatggaaactgattggtcctttttcctttttttttttttgccttgactgccaggaagcagtttcaaatctatagctggattttaagtttcattaattcatgttcccacatatggttctgtattttcacttcccccttttaactgacatactgtcttatgtgatctctactgtaagccttctcatcattttggaaacagaccaaatataatatatatgataaggaatcaaaagtaaatacagtagtgttgaatattgcataacaaaaaggtttttaaatagggaatggtatcaatatgaagtgttagggagacccagccatgaaaaggatagcagggtcagagaaggaggatgtattgcagctggtttaatggagaatggtatgaaggaggtgcagtttgaattgggtcatggaggacagatggattgcaaatagctggggcaaaagcacaggaaggcattctaaacgagccaggcatggagacaagaatgtctcccacaagggagttgtagtagctcaatcagactgggatttgagatttcatgtggcagagtggtaggtgataaaggtgaaaagactgatcatagtaaaatgcggagtctgtaaatccagcactcatgataagtttggacatcatgtcaacagtggacagccataaatgactgcaagcatcggtgtggtataatgaaggtgacgtttttgtaaaatgactctggtgaaggtacagaaggtaatgaaaagtagccagtctagttgagcagaaaagagttcagatgtaattgcatcatggtccagatgtgaaatgaagacaatgcgaagtggcattgtggatcgaaacatacatgcacaaaatgacagaattttagaatttgaagggatcatcatggttaccaggctggcctccaattcctcttttgtaatattaatagaaattaagggctaacaagtttaaaatgttatccatctttttacatagttactgcccaaagtgaatattttgaaatgtatcattaaagaagaatagataagattatgtgattcaccatggactattgtcatgagaggaaaaatgtgtttagatgattctgttagcactgagacaaatcaggatatctgaaaggaggtctttgttgaaaaacagaaatatgcattcataacttgcttttctaaaattggaatgtaatgattcttaaatatgcacagacacaaatttttctttaacagtcaagaaaatgcacgcaggtgataatcagatcagttttggttatagtacaaaggtttaatgcctccgtgatccctttcaacttgaaagcattctagagcaattggtgattaatatcagtataacagtcatttataaaattattatttatttgatatacatctaatcaaagcataagatttatttttattattattattatactttaagttttagggtacatgtgcacaatgtgcaggttagttacatatgtatacatgtgccatgctggtgcgctgcacccactaactcgttgtctagcattaggtttaaaagatcagattgtctcggcaccatgttaatatctttttctgttggcattagtattagttttgcttgtgtatttgtttaggagatagcttcacaagttggtgattgatattctaccatgtatgaagtcatgcgtggaattcagaatccccagcttgtaaaattgcattatgatcatctttagtgggaaattgttctcagaatactgagcaaaggatgataccaaaatggcagctattattcattcttaagcatatgaaatgctttcaggttcaacccaaaattacatacattttaaatgcttactaaaagagtcttttccctcctccatctattaactgcaatcaaaaaacttcggttttaactgaacatgatttcatattatttattaaaatttaaggcaaggtgcaccaagtacccttgaattatgaaaagcttcatgatgtgggatattctttcagttaacggcagggttggctacacttttaaggggttcaaagtaggaacagctgcaatagtgagctgcatctggaaagtccagtaatttgaaaaaccacctgtttatgtatcctgcccactcaagtccataaaataacagacactttcatattccaaatgaaactgctttttagtttgccctacttttaaacataactctttgtgatggaatgaccagaaacagctggtctctaagaggacagggctatgtgcgctcacctgcggggttggaccttccataatccccctggctgtggggaaagttgagggctgctgtctttatacaaagatggtttattccaagatacacacactcttcttccacaccctggagaccttgcatatttagtatcttctttaccataatctgaggccctagagaaaaagatttgcaaactatacttgttttaaaacaactttctaaaaaagacactctcagcccctagaaattatgcctaacacatagatgctcagaggcaacctgttgtagtgcaagaggattgtgccaagattagaaaacaaatatttgcaacttttgtaactgtcttctctaaaacttgaatgtggtgattctaaagtaaagaccgacacaaaattctttttctttagcagtcaggaaaaggcatgcatgaagtaatcagatcaggtgtggtttcagcataatggcctaatgctttcatgatctctttcaactggaaagcgttctagtcccactggacaccaaggaggaagaagggacggaaaatattaggcccataggtttatcttcctcagtagtccacgagatttgagcttatatgtagggagcaaaattgtttgtctaaaagcagttaataaatgccccaaaaaggctgggcgcagtgactcactcctgtaatcccagcactttgggagctcaagattggtggatcatgaggttaggagagcaagatcatcctggccaacacggtgaaaccccatctctatgaaaaatacaaaaattagctgggtgtggtagcgcgtgtttaatcccagctactggggaagctgaggcaggagaatggcttgaacccaggaggccaagattgcagtgagccaagattgcgccactgcactccagcctggtgacacagcgagactccgtctcaaaaaataaaataaataaaataaaataaaataaaataaaataaaataaaataaaataaaataaaataaaataaaaataaaatgaacgccccaaaaatattttgggcaaactattttgtgtttcttttctttatttatttatttcttttgagacaaaatcttgctctgttgccccggctggagtgcaatggcacaatcttggctcactgtatcctcaacctcctgggctcaagcaactcctgagtaactgggaccacagggatgtgccacaattcccggctaattgttttagccaggatataaatgctgcctacatagagtttgtagctatctccttgactttctttatgcagattccttcacaaacttttgatggattcctttaccaaattctactgtctgttaaaatcttctatctttatatctttagtccaaacaacacgtcatttataaaccttaaaattgtttctgggcaaataaacaaggcaaaataggaatatatatttttaggcaatttacttctgttttggtctcataaaaaattgtaattaaattgtagaaaatatttcaattcctctttaatatcctctcctcacatactggctctcaacttctaatcctcctattgaaacattgattgggaggccaaggcaggcggatcaactgaggtcaggagtttgagaccagcctggccaacatggtgaaaccctgtctctactaaaaatacaaaagattagctgggcatggtggcatgcacctgtagtcccagctactttggtggctgaggcacgagaatcgctttaacccgggaggcagaagttacagtgtgccaagatcaagccactgaactccagcctgggcgacagagtgagactccatcacaaaaaaataaaaataaaaattgaaatttgcagcctttttaaaaccccatagcctctttataaacccaaaagcactatcaaatttggcgaggtgtcaaaagaatcagaggaatgtttacaaatacagatgcctgggcccacctcagatatatatatatatatatatatatatatatatattttttttttttttttttttttgagacgatgtcttgctctgtcacccaggctggagtgcagtggcatgatctcagctcactgcaagctccgtctcccgggttcacgccattctcctgcctcagcctcccaagtagctgggactacaggcgcccgccaccacggctggctaattttttctattttttagtagagacagggtgtcaccgtgttagccaggatggtctcaatctcctgaccttgtgatccgctcgcctcggcctctcaaagtgctgggattacaggcgtgagccactgcacccggcccagatatattaaattagaatatctagaggtggagcctgagtatctgtatttttcagagtttcaaatgatcgttcttcaaatgattacactgtgaagtcagatttagaaatgactgtacccaaggttggctaaaagatacacaccctggttgattctacctgaagagagcaaataagatacacagcaaagttgtagatgttttccctgccagtagaatacttgcgggttaggccatttaaaaccctgccagagagttttgaaacactgtggagggctcccaaatcaacttgctcaatggttctccatcccttcaggctacttgggcttaaagccaactgcaagcttagagcctcagagtgacctaggaatggggtgaccatatattctaggttgtctcatacagactagccagcactactcagccgcaagtaatagcatccaggcatgctcagaagtgtcccatttggaggaaaaaaacaatattgtcacaaatgaattggcaatggcctgtctctgattcttatacctggaatatactggaagtccctactcatgctattttctagcagaataggcaaaatttctacattccaggcatgtcaggcctttccctgattcctttctctaatgtcactcgtctgctgtcttttatcacagccattaaactgcaccctaacttaaagaggatcccttatgttccaatctactcatccctcagatctttctttctctgaaacacagggttaatgagactgacatccttccatcacatattttctcagctactcagtaaaagatgtaaatgtttaaaatagtttaaactatttttcagttagtccaggaaacataaaatggcatgcttgcacataaaccattgtttagggtgggggaagtgtttttaattttgccttaaaggaaatctgcatgatccacaggctatgcaactaccaagggaattagttggtagaacagaattacacctgcacagaatacaaatttcctgcctttcatgggaactatgttgatgtttcagatatgaaatacatcttgttttctttattgaacctcgagaagatgtctcttgttggtcattatttcatggcaggggaagtacatattcctaaagacacaaccgagtttccctttaaccatcattagttgggctggccattaagaaccagacgcttttattttcaaagagacttaagttttgatgttgtacatatgtgcctaatattctatctcatagcaatttaaaggtgacgttttaaaaagctgcattcagtgtataaacttctcctgatcccagcaaggatgttgtgatgattttatttaaaaaggtaagttgtgtctagatatggcagtgggtcatctcatgcatggtgcagatgtcaaacacaattacattttcttatttgcaatgactaaaaaaagaagctgagcccaagcagtgagaaagtaggagattgggaggacaagaagcaaaggaaaaaagtaacatgagcaccgttctccctgtcctgccacttgctccattatggactgggctgcgatatctcatatcccagctccacaactcccaacaaccatttatgtgcatggtgcttccatgtgtgatgacccaatcaggctcaggtgtggactgagtagttaaattataacccttgtctctgaagagtttagggcttagtggggaaacagacatgtaaacaaacctgagtgaggtcatgtaatcaaaggacaggccacagtcaaccacaaagaagagagttctcagcagtctccaaagccgaacatatgtttaccaggaacagggtcccagcagagggagcaacaggagcaaccagagccttgaggggtcgtggcctgttctgggcaccagcagtggatcaatgtggccagagccagggatactagcagaagccagagcagcagggccttccttgtccagcaaaggcatttgtctctttgtaggccacagcgacccacagagggctttttaggccagaaaaaagccattaaggccgggcgcggtggctcacgcctgtaatcccagcactttgggaggccgaggcgggtggatcacgaggtcaggagatcgagaccatcctggctaacaaggtgaaaccccgtctctactaaaaatacaaaaaattagccgggcgcggtggcgggcgcctgtagtcccagctactcgggaggctgaggcaggagaatggcgtgaacccaggaagcggagcttgcagtgagccgagattgcgccattgcagtccgcagtccggcctgggcaacagagcgagactccgtctcaaaaaaaaaaaaaaaaaaaaaaagccattaaaaagggagtcatgtctcttgttggtcattatttcatggcaggggaactacatattcttaaagacacaaccatttcctcttaatcctcattagctgtgctggcaattaaaaaaccaaaagtttttactttcaagaagatttaaataacttctgagggtgtacatatgtgcttaatattctgtctcacagtaatttaaaagtgaagttttgaaaagctgcatcctgcgcttgtcagaaccatgtctgatgagatatcccctttaaagggctctcggtgcaatggggcaaatcaagggggtttgtgcaagtgggagtgagacaggagatggggtgcttcttccagcactccctataggctgactgagtgacaaagatcattttactgacacctccaatggccctatgagatgggtactattattattatcaccatcatattccttttgcagataaggaaactcaggcttagcagattgccagaacaacacaggcaggaagtggtagagtcagggtttgaacccaggtagtgaaactccaaagcccggattcttaaccactgtcctccagtgcctctctgtaataagtcatgatcccagaagccattggtgtggccacaatatggaaagagatgacagtgtcctcacactgggtgagcagcttatggtgattccagacatgatctctgttgggagtgacaggtctgagcttctaggatcagaccctagatcttggcaagtggtttgaggaaagagaaggaccaatgtaaaaccccaggcttcaaggaatgtggatgctgggcagggaggattaagccccaaagaccagaaatggggtacacagggcaggtgtggccagagtagaactagagtagaacttccagtgactagaaatagaaccagacacgttgcagtggtggataaggtagaatcgcttaagtctttaaagtgcccctgatcacccaagttggccagagaccctggggtggggctgattctgtctggatatacggggaggggtaagcatgaggaaaggaagcaggtcctgacaggtactttgcactaaacagctccttataaggttctcaatttgcctgctcaatttctacagacatttgtgggaccacaccagtacattgtaaaagcaggaaacaattgagaaaaacctgagttttatgttggtaggagaaatgcctatggaatatggcaaatcgtttctctgagacttcctccctagtaattacatatttgttctcaaaaacaaatgccagaaggaagaagcagatttaatagtgcattttacaaggcaccattaatctctaagaagaacaattaaaatgtctcagcaatcatggttcactgtatatcttttctatcttcttagaagtaatatatggctggaaatgggcataccaaaatatgtcaaggaagtggaattgcgttcattagatttcaccactaattattttagttagcttcacagatctctcttccttgcttgttcttgagagcgaggctttttagtaggaagagaaattgtctaaaacgattaataaccacaaattcaccaaactattttgggtaagtccctctatttctctaggtctaaagctaggaataagagtcattctcatataatgtactgtcccagaaagggcattatattagtctgttttcacgctgctgataaagacatatccgggattgggtgatgtatttaaaaaaagaggtttaatggactcacagttccacatgcctggggaggcttcacaatcatggaggaaggtgaaaggcacatcttacatggtggcagacaagacagaattgagagccaatcaaaaggggaaaccccttataaaagcatcagatttcgtgggacttatcactaccacaagaacagtatgggggaaccgccaccatgattcaattatctcccacaaaatgggaaaattatgggaactacaattcaagatgagatttgggtggggacacagccaaaccatatcaggcattcaaccaatatttgggaagcaccagccctgcaccaggcacggagcacgtcatgagtcctgccgtaccacagcctgcctgacagacctcagtcatcctctggagcttgcctctgacatctggacctcctcagaatcagcatctcttctccttgcccccgccatcctttgtttttatctctgctgtggcattcatcaaagccttccaactatcctgcgtcactgtccttcagtgtcctctctcctctcccttccttctcaccccactttgtgcctgtatccttcaagcagagcaatggcaccctcacttctgtggctgcccagtgccccatgcagagtcagacatcagaaaatagatgctgaattcagttgacactctgaaattctttttaaagtaagttaatgtgtgctttgaatgaaaagacactgggattacattattgagtgtctttcttcctttgccacttttgtccctattggccatatttgaaaatcttgttggaaaaaaaaattcaagaacttaataaataaattcaaaaacatttagtctatttacttaggtgaagagaaaactcattctaatatgtgtgtatatttaaaatatttgttatttagactttttttttaagtctccaggttgaggaggacacaaatatatcctcctaaaccttccagtaagcaagctgtggcatccagatgatctcctgggtcatgggggataaggctaatctcctaggtgtctggcagacaggacaggcaaattcccagaatgccaaaatataccatctgctgctgtttggcattgcccttaagtccagagtgtggaggctgggggtgggtctctggctacaggagaagtcccctggcaagggaggggtgaaaggagtgcctgttgaaccccccatctatccccgcactatggcaagattgagaggaatgactagatcagggaatggcccgaaagaaaaatccaaaacctcccaaccctggacaaggccacagctttgagaaaccgaagcctctgcttccttctctttggctttactgcttctagatgcaaatacacagagctctgagattttgtgtgctgggaggtgataactgttaaccctctattccaatagcacagaaatttctctttgcctcagaagtggtttctcatagatctcagatctcttttcaggaaaaagaaaaacaacaacaataacaacacattaatgactctgaaagagtcagacaccattaattccattattggtgtctgtgccaagtgaaatgaacgtcagctcttttcccagatatgtttccttcttttgcctcctataataagagatgattttactgtaataatataagactcatcaatttgactccaaatagctttcctatcaacaggctaagtgtaaaataccaggatcattattcagttgagaatagatagaactaggaagtagccatcaaaaaagaatgatgaggtgcattgtggatttggggtgtaacttggtatctaacatacagccagaatcacagtcatagcacacttaatattttatcagaaacttgcgtgaacaagttaagaggactctcaacttaaaaatgacaccaattgcaatgatcttgttaacatttgtgatgaaaataatagcaaagtgacttagacaaattacaatagcccataaaaataagataaagtttaacacaaagtaagatgatgttaaaagacttgaaataaaacagatatgttaagtaggcaacacataggtaagcatataaaaacaagaagataccaggatagagctgtcatttttgtgggagcctgtgatgtggaaaaccaagatgcctggtgagtataatggatatggaaaccccccttgtaataattccacagttccaaggggccaaggtctccaggttgagtcactattgtaaacacacccatagatgaatccacatgccatacctccttgagtaagtggggactcaaactaggtctgtcaattgttccagaaaattaagcatctaaataatttaatgataatttaaaagaagcacaatgaaatatttcaaggaatgtcacatacaagattctgtacctcttctgctttggttagactcattcagaataggttcctgctttgatcttaagagggaggtagagattctggagaagccctagggaagagcaaaaggaaaggaataaggagccaagaggaaacccagggtaaggctgaggagggactgtttcgtgtaggtgatttattggaagggttggaaggaaacatggaatgacaattacctttggttattgtcaggttagtatgagacttacaagaaaagcactgctcagacgcaattaccattcaagataagaaataataggaaaggctagcacacttagctttttatttaaaaaagtgttaggtaggctgagcacggtggctcactcctgtaatcccagcactttgggaggccaaggtggatagatgacttgagcccagaagcttgagaccagcctggacaacatggtgaaacctcatgtctacaaaaaaatacaaaaattagccaggcatgatggcatgcacctgtagtctcagctacttggggggccaagaggtgggaagattgcttgagcccaggaagtcgaggctgcagtgagccatgattgtgccactgcatgacagcctgggcaaccgagtgagagcctgcctcaaaaaaaaaaaaaaaaaaagtgttaggtgacatgagagaagatcttccaagtaataagagtggctaatcccaggaatgtgtcaccagaggttattttgtaatagtcgtgtgttaaattccttatttgtctatataacttctcaaatccttctgcctctacagttatagtttaactggcgcataacagccttcacacacagcctcataattaaacatagacatacatatgaacactttcccctatgccagcaggatacttggtttgtttaggggcaaagaggaattgatgtggcgttgtttcaatcagtggttgaaaatgcaagtggtaaacattgaaaaatagaacactgcaaaaggcatgcattgtatataccaaaaggtcagcatgaagcattatctgtatggcaagcctgcccatccactccctcctacacgttgcatattcacacagttttgcagcttgtataaacccctattgtgatagaaactcatgaaagagtgtggtctctgcgaaagctggctgttctgtgaatttagaccagtggttcttcaccctggctgcaaatcatctggggaacatttaaaaacactgttttaaacaccccaaccctagaaattctgatttaattggtctgtggtggggcccagaactctgtattctttttttaaggctctcaggtgctgctaatgtatagctaaaattgggtctggtttagactctcagaatttcttaataattaaacactttatcatgacaagactttcaggaccttaaaggccacagtggggtagttatcatttcactaggtcctcatctggggaggtccttggcatttttactggaatatatttgtcactcaaatttctattacaaaaaattctttcttgcacactgctttagcaactacatgagatatactttgtacatagcacaaatctcatatcacttatgtaatccagctctgtggttccttcctttcctttgcctgtttatttttaattcttcccaagaggaagcttagccagttagaacaccagagtatcatccccctcccccttttcccacctgagttcatggcttagacatactaggaatgaagctgacaacatgcactagtttttttcgaaattatgcagcaaaattcccaaagtgcgagtggccacagagatcttcacagggcccagggacaggcagacatcattctttctccagttcctggcacagaaaagagaccttaggttactgagaagataccagtccctcctcagagcagacaaggaaactgagcctcagaatgaaagactgaatttcagtcctttcttgaacatggacctccagggttatattgggccttggaaaaggcacttacactctggactgtagtttcttcatctataaaatcaagaggcagaaacagacaatctctaagttgcctttatttataaaattccgagattctagttgaccagtattcatacaagagttgaagcctgtaagagtgcagaaagcccacacaaagagacagtggaagacctctcatcagtagtatttttattaccctcttcctaggttttaccagtcaacatcctcactgttaatatacagaccgtggtatttaattaaatcatctttgaaatactgagctatcaacagatggcatgctgaatgcaaaaggaccacaaataaatatttggtactgaagaagatcaagagttggagttcatttcccattctgatctgggctcagaactctgtggtcttccctctaatcatccttgccaccaaattggctgtatctgttctaagatggatcagaaaatcagttccaaagttggctacaaactttcaggtttgggttttgttttgtttttttgttttgttttgttttgtttttgcaaccagccaattcatcttagttcacatgacagagaagtgcataattacttgcaactttagttagagcagtggccttaagaaggtctagctaaataaaaagtgctcagactttctgagtgctgacagttgtcaaattcacctagttcacatggccccatttctatcgtttgttttgttttgtttttgttttttaacagcccatctgtgagcaataggatcagatgactaagagctacagggcagaaacactgttacttagagtcaaattttcccattacctagctgtaaagagtttgtttctctctgactcatataaagtttaccatttaggcccctgcatgattttaattccatcacttaacaccccagccatatgattctgaaggtaaacatgaaggcgtttgaattccagaccacctaaacattcttaaggaaatcatcatctccacgggcagagctatgccaaaatctgtaggttttaactcaaatttcatgataagcaaaaattgaattaatttgtcttccattttgttcacctttttgccaaaattatgcctggattagaataaataaattcaatcaatgaatgcaatcactaattcttacgccagataataacacattcagaattctcctttccctgggagattttatcaggttagtgttcttgtaaacaggagaaagagaaaaatataacttagtaaatagcagtattcactaattcattcatttattcaacaaatattaatttactacctactacattccagggagcttagagtctagtatcagaaataataaccacacacacacatacacacacactacattaaataaggatgtgataggctagatgaaataaataaataaataaaaggtccaggtgagaaaagaaggtgggggctagaaagaagtcattgaagaaaaaacatttaggttaaaacattatgaataacttagagtgagccaagtgcagagtgctgaaggagtgctccaggcaaaatcaacagcaaatggggagtccttgatgtagaaaagggtttgaggaattgtcctgggagaaatactcaagattccagtctgaattctagaggttagtgatttagagaggcaagtacgaaaatgacttcctctcttaccttaaaagtaagtgcaccatagaaggaaatcacccttccttggtaataattcctgagtgagcctgagaagccagaggccatctctattttataggcactgtccccttttcagttacccatggctagctcattgaccttgtcctggtcgtttcctcatttcacttactccatcctcaaaacgtagacgcttcataaatattgtataaatgaatgaactcacaaagtcacagtacagcaaggcaaaagtgcctgcaataaacaagcattctaggctagaaatatttctcaacttcaaattgtgtcttattacattgtattccgattttctagagtggtagttctcagtcaagggaaagtttttcttcccttccaggggatatttggcattgtctggagatagttttagttgtcacgatttgggggatgcttctggctcaacttgggtagagaagcggggatgcttataatcatcctacagtgcacaggacagtacccccacccacactccagtaatgaagaatcattagacctaaaatgttaatggtgtccaggtagaaaaaccctgttgtagaggttggggactgcgtcttgacagccacattatacagtgtatcaaacaattctgtataatgggctgtaattatccttgcctagattttgcaagaaccctagtgtgtatctttttcctcacttgccaagcaatgttcaaacctgcagagatttatttcattcattttctgtgtgtttagtaaacagactagaagcactggaggaaaaaatattccagcaatgaggtaagacgaaagctattagtaaccctagtttaacttagctgaatagtaggaaacaacctctaccgtgaggaagtgtattgtagaaactgaaaagacgctaatgatgtttaaaaagctgtagttcaaacaaatgtgcatgcagaccaatgggtagactgaaaatgatgaagacatttccgtttcttgtgtctttgatagaaaagaaagagcttttattttctttagtgtggcaatcattcagatttgtcccatgacatgcccagaaggttgaagaataacaaactcccaagtgtaaacacagaatttagcgaagaatccaggcctctggatgaatccctgtaattgcatgtttggataaaataagattttcatacattaaacaaggtaggatttttctatctgggacggaactttcaacacttggaggggttgtagttatttctcctcaaagatggcaaacatgagtgccccgagttatccctcctctctgttcaagttcgctaactaatcacccagtatccatgctatcgctggcccttctgtggcctatttttatactgttcactgttcagtgtcacttgtttggtaacactcaacatcaacatgtgctaccaaattgacaccagaggacaaaaaagaatcaagatatgtacagcctgctttgtactgagccagctgccactagatgttttttgtgataatgaacacgtgaggccatgtggacgcgagagatggctccgggttccctcagacggctcacagccagctggtctgcagtgcggttttagattccgatgtgggaaccccataaaaaagaatatgcaggccaggcgtggtggctcatgcctgtaatcccagcaatttgggagcctgaggcgggtggatcacctgaggtcaggagttcgagaccagcctcgccaacatggtgaaatcctgcctctactaaaaataaaaaaaaaaaaaattagtcaggtgtggtggcggatgcctgtaatcccagctacttgggaggctgaggcaggagaatcgcttgaacctgggaggcagaggttgcagtgagcaaagatcgcaccattgcacttcagactgggcaacaagaatgagactctgtcacaaaaaaaaaaaaaaaaagtctgcaggctgcataaagaggtatgaaaatgttccagaaatcccaaatcctatccctgaggttcattttggtgagggaatgtgtgtgcattttctagggcttccctaaaaaagtatcacaagctggatggcctaaagctacagaaatttcttggggacaaatttcatgattctggaagctagaggtccaaaatcaaggtgtcagcaaggctatgctttttctgaagcctatagggaaggccttccttgtctctcctagtttctggtggtttgctggcaatgtttggcattctgtggattgcagctacataactccactctgcctccatcattaatggccttctgcctgagtgttttcatatgaccatcttcatataaggacaccagtcatatttgatgagggttccaccctactccagtatgacctcatcttcactaactacatctgcaatgaccctatatccaaataaagtcacattctgagtgtctggggattagaacttcaacagagcttgttgaagggggcacaattcaatgcataacaggatggaaactagaaacgggtatgtttttatcagtgtagaaagatttagcttaatttttcaaagtgtaataaaaaccccaggaaaactcatactccctcctaagaagagcaaaagatggagaaacccgatggttaccttcaaacaaaaggaaaggaggaataagatgaaaaggaattaatccaaagcaaagagagtggcttatatggaatgttggtgcaactttctctgacacatctgtgcactcatcagctggggcatcatctccctggggtacatttggtcactgtgtgcctcatggtaataaactccagaagcctcattgacttgctagagatgagctcatccttcttgcttgcttaatggcaaaatacaaaataagcagtcactgacatggaacgatttcaggaatgccaaaaggttctccttttccaaaatatctcttccatcttcccaatactgttactgacatcactaacacctctccacttccggttgagacacctgggccagagctcctgatgtggcaggcagtgccctaaacgttttgcataaattaactgatgcccagagcaacaaccctaagatataggtactatcataccgcatcttacagataagaaacttaggcacaaagaggtttagtagtttagatgagataaccctgatgagcagagattcgaacccagcctccatgctattaaccaggacatcatattgcctttcatacatgctcttcaaaggcaacacagtaatcgattatcacactcactcacatctgattgtcacatttttcagatctgctctcctagcagagaatgaagcctaaggtatccttgtttctcaaagtgtcctccccagaccagctgcatcaaaatgaggggatgaggtgcaaatgcctggaccctgcccttggagcactgattcataatctcaagtcccaagaatctgcattttaacaagcatccccagaaatttcttaagtatactaatgtatgggaaccactgacactaaagaaatggaataaggggaacgtacaatgttacagtaaaccaggaaaagccagaaagacatgacaacacagtgaggactctggtagccaatggtcagtcaaatgcccaggggccctggccagaagagagttaggttgctgaggagtaagagtgatgctgaatgtggaggcttgagagcagaaggaagccagccagctatatcctcttgcttggatcacacaccctttccttggtggaaatggttatttgcagagttagagaaggcatgttttacagtttggatggcaggtatggatgtagacaataaagagcaaccagagtccatgggttcagaaatccccatgtgtttctgtttgaatgagacgcttgcataaacagcacaaggagtttggggtggggttaaagagaatggtgtggtatagggagagctgaatgaggaactgagagagcaaaatcctgtgtttggttcaatcactgattacaacctccctgaggctcggtctcctaatctgtaaaatggggggaaataatacctgccttgcaggtcctcacacacagggcatgatgtgaatccactgaggcatatagcactgtgtaacatgagttattgctattccaaggcccgtaaaaggctcttgccttggaatatatctgccacaccaatgcctgcagtccattaatgacacataaaggacactggagataacgatgtcccttgttctatgcatccctcccacccatgccagaaaagaaaacacagtcacctgaagtcattctaaagagtatgcctgcctcttttcctgcacagacacatatacacagacacgcacatacacagaccatgcacatacacacacatgggaaaacatgaggaaaagtggagacaagaggcaccaaaggacaaagtcacttttgtcgcctgtcccttccccagcagggctgggcctgggctgcttctcctgcctcctccctgaagccccctcctcatcatattccagtgcgtgtccaccactttggggccaggtctacacaactgcagtgattcaggtcacgggagaaaacccaaacaagcacaaaacatgcttcaacctatattttctaaattgtttttctttaaaggtgaagacttctgagcttgaattatccccttgtcagtgggctttccatgctgtccaagtgacctaagtgataatcaacctccatttcattttgagaatggttgtggtattttagagctatggtgaataagaaaatcatttaaaataaaatgatttttatttatttattgtttttatttattttatcttaaatgaattttaaatcatttaaaataaaataatgggataaaagaggatgctaaaaataataaatatatatgtatcaaagtgtgcttgtaataccaggcaaagaattaataagagataatattatggttggtgaaatgttatgtatggctacatcctttcaatgagcatttatagttcctttaaaatatgcctactgaagaaatatttacatgctaattaacatgtgcatagtaccactaggtattatagaggataccagatgtttgtagtagacacagaccttgccctaagtcctggtcttgatgtagtcactttttagtcactacaggtgactacatttagtcactacaagtgaccttccttcaatggggaaataaaggactttacaaaagacgtagaagacaattcttaatataaaagtgatttagatcttcacaagtttgtgaagagaagcagatgagtgaaatagaacactatcaatgtaaaatattattctgaggcctctgtaatgactgggaagcaacaagagggaggtcatttcagagagagaggctctaggttccaagctggatgctcaggtcagtgactgcaggtcccctccacacccatcaccccacaccctaaccctcttcagttgctcacaaaggtagataaatacccacatttttgccctcttccatcttgaaaccctggaaacccttgcttccgccaggggaggttacttagtatctgtcaccccaagggaaccaacgtcgaagcccaagaataagagtcaatactcctaccagaggtttacatttttcccaggggtctaggtggatattcctgggaacccccgtcaacacaggcatctacagtacaatccaggcctcctgttttcagcaggggctgcaagagcactgcagccttttccccagaggtgtcagtttggcccagtaaagattgcccctgagaaaacacatgggcaattagagcaaagttcctatgttctggtaacatttaattgtgctatttctcaacctcctctgcacccacacactcacacacaacatttattccactgacttcaaaggaagctcaacgtgttaaaaatatgtgtgggaacaaagaagggagtttgaaattggtctaaactctgtataactgggtttgacacgtacattaggattttacaagtatgtatttaatctttttttaaaaaaagcgtttacatagggttcagaataatgacaataaatcaacatttctattgtccatttgtgtgttttcatagtaaataatgctcatttatccttaaccagtaatacatacttatgggcttaaattagcaaaagcctctcaaaaagtagctccactcatttatccaccagtgtccagatgccatccagcacatgaggagctcccagaaaggagcagggaacaaactagggctgtcaggagtggaggagaaagaatggcatatgcaaaaaggagctgtaattaaatccaagggaacatggcacactctagtcttttgcacgagacaaagggcaatcctggtaaaaatacagatccccaggccccaccccaaagagtctgatctgattctgaaatggggccggagaatctgcattttaacaagcaccttcaccaggtgatccttttgctgagaacccctgagaaatgagaaccctgtgctagtgctgaatggagcattatattccagagttgaagtttggtgatcagttttccagatggagctggtccttggtgcatacctgggtataaatccaagccaattcaggtatatgagctgatatttcaaccgaaacactatctatagcctaaattttttctaatattctgtttggtatgaattctagaaagttgtaaatgctatatttccttctcatctatttctggactttgtcccaagaccaaatcccagggcatctgatagacattcattgcatacatttttctgtaaacatgaaaactgaattgtctaatagaaaagggcaaggaagtagaaaataagaaatcatcatcagaagtggtttgttttggaattatattgtccagctgcataacaaatcacccccaaaattgagtcgcttagaacaacaaacattgatcctccacagtttctgtgtgttaggaatcaaagtgatttaatttaatggttctgctcagggtctctcgggggctgcaatccaggtctcaggctgggatcctttcaaggctgagctggggaaagatccatgtctaagctcactcacatggccgatggcgggattcagttcctcttaggctgtcagactgagggcctccgtgtctcagtggttttagccagagccctctctcagttcctttccacatgggcctctccacagggcaactcacaacatggcagctggtttccagtagagcaagcgagtgagagaacaagaaaggcaagcaaggtgaatgtcccagtcttttgtaacctcatctcagaagtgttaacccatcacttttgccatgttttattatttagaagcaaatcactaagtccagcccacaattagagggatggcattacacaagggaatgaacaccagcagacagggtcattgaaagccatcttagatgctgtctatcgcatctaagtgtgatttttccagatgaaaagaatatattaatttgtttcagtcttagtcgatgtgccatcccatttgtgctttgctaaaacttgtatcaatgtaaagcaaacattttctgatacaatttaggtagtgtattgtggtaatagagaccagtagtgttgaaaagatatgttgaggtcagaaattaagctcatgtttctaaaagaggagatatgtacaactactatgcaagccaacaggaaagagtgttttaagaatgctttctgctacaggtaactaaaaacctaaacagctgtggctttaaaataaaggtatatctaagtcacataagcaaaagtctaggggtgggcagctgctggcattgcttcagtagcttgataatggcaaaagcagcatctcttctatttccttggccttctaatcatgcatgtcacctcacaatcacaacataggcaacacctcatattctaagcaagatgaaaagggcaaagagtcatgccatatgcctctgtctcttttcataaggaagacaaagcttccctggaagtcccctctagcagatttcacttagatctcattggccagaactgagtcacatgcctgccttaaaccaatcactcaccaagaagactaacattatcatggcaagtctaaaccaactgtgactcatctctgaaatcaaaggattattaccattacccgaatccatcaggatcctgttggcagagaagtgggactgtaaattttgagcaggcaacaaacaagtcttctgtaaacttcttatgtgttgttttttatgtgttctatatatccagtagaatcacaatttccaataacagtctaaaaagatattttccaatagaaacagaatgtgtaagatcattacttatgaaatcccaaatgtacttaaggtttccttcttgaaaattccttattcaaaataaaatgtccagattttgaaacccagaaaagattctatattttaaaaatcctgtgcacatgtaaactgtttttcaaatattgccttcagatacattgaacagaatgaaatcttctgagatttactacatcagccaagtattatcaaaacaaacaggacagattgcttttcttgacgtctgctgcttgatttgtgttaactcatgtttctgaaattgtagtatcataagccaatgctgcacaaaggtatttcatgtcatttataaaaatctagtaatgtaaactgttaactccttataaagcatctgttgacacacaaaaatatcactgaagtgcatttatgcctttcttctttaggtctgcataatacttccctccagaaggccaagttgttccataaattacagaacagaaagttggttgtgggaggaatagctcaacctcatctgaggcatcccactctaagaaactaatggcacctacacctcttgggcattgagtttttaagcccatttttaattcttgttctgctcatattctaagtgagcacataaagtgctgctccaagcaagaccagcccttgtagaagggcaagtgcagtcagtcccctaggaaacgggactggggagtgatcgtttcaatgagagataaatcaaactgatgctaaacatgaacaatgagcccattagagattgtgagaaagaggcatcatcatccactcaacaataggcctgtgggacctcttgatagcctgaggatgtttaatttcaggtgcaggtatccagaatgtagcagctagactgatcaaggatgtgtgatgacagcaagcagtagtggaagagcccaggagagttcctaagcctgaattgcaatcctgtgctgccataaaatgggaagatatacttggtccagtcatctgacagctttggtcatcaatttctctatctcatatgtgactctattgctttaagaatccctttagctttaaatatctatgaatctgctgaagcagctgtgctttgattgatgtggatctctgaactcccttaaatacaaagaccaattatttagccgagctttgttggattcagtgcattctgaatacatgtcaaaatatacttggatttgtaaaaaatattccttcctgtttttttcaccatagatagatgtacaaaaatgtccgtgttcacaccgtggaaaggacatttctcataaactcacacagagatacctttcaagtcaatgccttagaaagcaatgagagatttaaaggagacctagagatatgaatggagtaggcagagaaggtatgtgaggagaatgatgtaacttcctagggaaaaagtatgaagcacaaggctggacatagacctgggaatcaggaaattagagttctaattgcagcttttccattgattcacttgggatcttgagaatatctgtctcattttaatcattctgggccacagtttccatatctgtcaattagagtaagagtccctggctgggtgcccaggattgtgagaacataccattcagagccataaaaatgcaatcagtaccaataatgtactagtaccagtacctaggatgcaaaacatcctagatactaggtgtcctaacttaaagtggaaacattaacaagagtaattctttgaatcatcaaactgggaatattttaggaagcatatctatctgggtgaaaactaagcaaataagacaattgtaaaggcttgtgatctcaggaatacaaaggcaaaaatgcgcagacttgaaatatgacaagttctagttttgtcacttagcatctctgtgaccttggataatttcttaacccccggcagtattctcatctgtaaaatgggaataatgacatgcacttcagtggtttgtggtgaagattattacaaatagaaattagctcttttgagccactggtggggtttaaattcccagcccttatgtgctttgcagctgttagttcctcttattacaattgtctatttaaaaacctagtcacagcccggtgcagtagctcacgtctgtaatcccaacactttgggaggccaaggcaggagaactgcttgagctcaggcgttcaacatcagcctaggcaacatagtgagaccctctcatctctacaaaaagcaaaaaattagccagtgatgcatggctgtagtcccagctattctgagggctgaagttggaggattgcttgagcccaggaggtcaaggctgcagtgggcagtgatcatgccgctgcactctagcctggatgacagagcaagaacctgtctccaaaaaaagaaaggaaggaaggaaggaaggaaggaaggaggaaagaaaagaaagaaagaaagaaagaaagaaagaaagaaagagagagagagagagagagagagagaaagaaagaaagaaagaaagaaagaaagaaagaaagaaagaaagaaagaaagaaagaaagagaaagaaagaaagaagggagggagggagggagaggagagaaagaaaaaggaaggaaggaaggaagagagagagagagagagaaagacctagtcaccaaaagcaagagattttttaaatgctactattttttgggcatttactaatcatattgctatgctctgcacccaagctaagtaatttaaataaattatctcatgtactcctctaaaactaattactgctgtgtaaatggaggtagaaagaaactaagctttatttctgcctctattgtttctttaacctgccttgcttcctttttcagttgcacctaattggctgtacttttagttttctttaaaactgccttaaatttcaaagactaaagcagcaataactaactgaatatatttatataacatgttatttttgtcatgttgctttccacccctggagacctgctctaaattcacttggacgtttgaggataaatcatgctcactagcagtttctgaaaatgcagtttcactgaaaatgcaggcatccagaaatttagtaagcaacttaaaagaaagtgtaagaatctcctatgtattcattgaaaaataatttgaatttatgcttagaaaaatagaattattattaagaaatcttacacactcatgtttttaaatatcttcactaaggaccaattgtgtatatggtgtaacactgtcctcaaagaacatgccgggagaattgttgcagttaccagagggttaaatttggcaaactcttttttattaacgtgccttttaattatgaaatagcatactcaccttagataaaatttgaaaaccatttttgtaaagtggtacaatattgaagaaagttgataactttcagaccagatttaagcctcaaatctacctctcttttacctggacaactcattagcatttctgaacctcacattttttctataaagtgagaatactatattatagagttgttgtcagttaaatgagaacagtgtctgatcacaactagtcaacaaatgttcacaactcttcccctcctaggaaaagaatctcaaggcagacctgcttcgggtctgctctgtaaagaggtaggaatcctctgctcccggtaaattgcttcctaaccttctttggtaatagactattttttaataaaggtgatggatcatttcccattatacactcaaaatgtgtgtccatttcagggcagtcatggatgaccattgcccatcttttgaccccagattaagaacacctgctgtagtattttaattctgccttcaaatcctcttacaaaacaaagacatctttaaaaaataaaattctttaggtgtcttgcagttgaatgcaggaaaaccagagccccttatttttgatagttttgggaagaatgcagtgtcagaacacaaacccataatagacaaataatttgcacagaaacttcataaaagtattgacctgatttgccatgtatttgccaccttttaaaacacacaactaaatgtttaccctgtgtctagatccaaatgggtgaagaaaaatgagtgacaatacatctacttaagctcacttacataattgtggccatgccgtttttttcacattacattattagaacattggacaataagtcaagaaacagaatgttctacaaaataaactttaaaaattggtaagcatcatgtgctttttccagaagacattttattttgttgaatcaaaggtggctctttggcactgagtagctccgtggagtcatggcagtcctcattccctaatcctgagcctgcctgagtcgctgctgtcagtcatccacttgttgggatttcaaactgcattaaatcccctcctatagctgtcactgccaagcagttgcactggctctgtcctacctttctgttggtaattctgtttttaatcctgtgcttcagtgtagtttatataaatctttacagagggataaaacttcctgtaattaattgtttgggtgaacatgtacctgggagagctattgggaaaggggccaaatttgcattccagctcctttcatccccacccttgagctaaccaagtcctgtggattcttcccttagcatctctggaaccttcttttcttttcttttttttatgaccacctttccagtcctggcccttcaaacttgagtgacagcaacagtctccctgccttgagtctctttcctccttctcccagtgtgcatacggttgtcaaactcatcttgataaactactgcatcgattgtggctacactcccctgctcccacatcttccatagaccccactgtctgtaaaataatattcagtctggcctcaacctgtctttccagcctcggtgacacaggtctattctgcctgagacacttactatgacacccttgcttgttcctggggctttgacacatttccaacgtcccattgttcttcctctccaaatcagccaattgcccaagccctgctcaaatctcccacctcatgaagccttcttgatgcctcccagcacaccatgatctaatttcctgaagtaattatgctaattgggcatttgaagaattgttaaccgattatcaactaactgccccttaacattgcatgtgtagttgtcttcaaaggcagttaaattatgtcatgttccttacattgtactgagtgcctcgtatccttatccatgtttgggggttttactttaagtcaagaaatttaatcacatccatttggttttctctagagctgtagttctcaaccttttgtgtggtagagaaacacctagagaacatgtttaaaaatatcctgggttccacccttgagagataataaggtccaaggggaacccaaatatctgtgtttcaggtcagcttattggctcatcctattataccaactcctcagaaggccaaggtgggtggattccttgatctcaggcgttcaagaccagcctgggcaatatcgtgagactccatctcttaaaaaaaaaaaaaaaaaggattagccaagtgtggtggcatgaacctgtggtcccagctacttaagaggctgaggcagacagattgcttgagcctgggaagtcgaagctgcagtgagccatgatcatgccactgcactccagcctgggtgacagagcaagaccctgtctcaaaaaaataaaaatgaaaaaaatctgtgttcccaagttccaagtgatgctgatgctgctggttgcctttaagcatctcacaaagaacgaactcataaatgctaatacagtatatgtctatggatactgaatagtgggttttttttctcttttcttctattctgtgctcatgttgtgtcacttcttccttttagattgactttgaagatgtgattgcagaaccagaagggacacacagttttgacggcatttggaaggccagcttcaccaccttcactgtgacgaaatactggttttaccgcttgctgtctgccctctttggcatcccgatggcactcatctggggcatttacttcgccattctctctttcctgcacatctgggcagttgtaccatgcattaagagcttcctgattgagattcagtgcatcagccgtgtctattccatctacgtccacaccgtctgtgacccactctttgaagctgttgggaaaatattcagcaatgtccgcatcaacttgcagaaagaaatataaatgacatttcaaggatagaagtatacctgattttttttccttttaattttcctggtgccaatttcaagttccaagttgctaatacagcaacaatttatgaattgaattatcttggttgaaaataaaaagatcactttctcagttttcataagtattatgtctcttctgagctatttcatctatttttggcagtctgaatttttaaaacccatttaaatttttttccttacctttttatttgcatgtggatcaaccatcgctttattggctgagatatgaacatattgttgaaaggtaatttgagagaaatatgaagaactgaggaggaaaaaaaaaaaaaagaaaagaaccaacaacctcaactgcctactccaaaatgttggtcattttatgttaagggaagaattccagggtatggccatggagtgtacaagtatgtgggcagattttcagcaaactcttttcccactgtttaaggagttagtggattactgccattcacttcataatccagtaggatccagtgatccttacaagttagaaaacataatcttctgccttctcatgatccaactaatgccttactcttcttgaaattttaacctatgatattttctgtgcctgaatatttgttatgtagataacaagacctcagtgccttcctgtttttcacattttccttttcaaatagggtctaactcagcaactcgctttaggtcagcagcctccctgaagaccaaaattagaatatccatgacctagttttccatgcgtgtttctgactctgagctacagagtctggtgaagctcacttctgggcttcatctggcaacatctttatccgtagtgggtatggttgacactagcccaatgaaatgaattaaagtggaccaatagggctgagctctctgtgggctggcagtcctggaagccagctttccctgcctctcatcaactgaatgaggtcagcatgtctattcagcttcgtttattttcaagaataatcacgctttcctgaatccaaactaatccatcaccggggtggtttagtggctcaacattgtgttcccatttcagctgatcagtgggcctccaaggaggggctgtaaaatggaggccattgtgtgagcctatcagagttgctgcaaacctgacccctgctcagtaaagcacttgcaaccgtctgttatgctgtgacacatggcccctccccctgccaggagctttggacctaatccaagcatccctttgcccagaaagaagatgggggaggaggcagtaataaaaagattgaagtattttgctggaataagttcaaattcttctgaactcaaactgaggaatttcacctgtaaacctgagtcgtacagaaagctgcctggtatatccaaaagctttttattcctcctgctcatattgtgattctgcctttggggacttttcttaaaccttcagttatgatttttttttcatacacttattggaactctgcttgatttttgcctcttccagtcttcctgacactttaattaccaacctgttacctactttgactttttgcatttaaaacagacactggcatggatatagttttacttttaaactgtgtacataactgaaaatgtgctatactgcatactttttaaatgtaaagatatttttatctttatatgaagaaaatcacttaggaaatggctttgtgattcaatctgtaaactgtgtattccaagacatgtctgttctacatagatgcttagtccctcatgcaaatcaattactggtccaaaagattgctgaaattttatatgcttactgatatattttacaattttttatcatgcatgtcctgtaaaggttacaagcctgcacaataaaaatgtttaacggttaaacagtca